(19) United States
(12) Patent Application Publication
(10) Pub. No.: US 2014/0005164 A1
(43) Pub. Date: Jan. 2, 2014

(54) WNT PATHWAY ANTAGONISTS

(71) Applicants: Maurizio Varrone, Siena (IT); Massimiliano Travagli, Siena (IT); Giacomo Minetto, Siena (IT); Lucia Cesari, Siena (IT); Simone Galeazzi, Monteroni D'arbia (IT); Andrea Caricasole, Siena (IT); Antonio Chiumiento, Siena (IT); Massimiliano Salerno, Siena (IT)

(72) Inventors: Maurizio Varrone, Siena (IT); Massimiliano Travagli, Siena (IT); Giacomo Minetto, Siena (IT); Lucia Cesari, Siena (IT); Simone Galeazzi, Monteroni D'arbia (IT); Andrea Caricasole, Siena (IT); Antonio Chiumiento, Siena (IT); Massimiliano Salerno, Siena (IT)

(73) Assignee: Siena Biotech S.p.A., Siena (IT)

(21) Appl. No.: 14/004,292

(22) PCT Filed: Mar. 23, 2012
(86) PCT No.: PCT/EP2012/055199

§ 371 Date: Sep. 10, 2013

(30) Foreign Application Priority Data
Apr. 4, 2011 (EP) 11161025.9

(51) Int. Cl. C07D 401/12; C07D 471/04; C07D 473/32; C07D 491/10; C07D 235/26; C07D 417/12; C07D 401/08; C07D 491/08; C07D 403/12; C07D 413/14

(52) U.S. Cl. CPC C07D 401/12; C07D 403/12; C07D 471/04; C07D 473/32; C07D 413/14; C07D 235/26; C07D 417/12; C07D 401/08; C07D 491/08; C07D 491/10
USPC 514/210.18; 546/273.7; 514/338; 544/295; 514/252.19; 544/362; 514/253.04; 546/118; 514/303; 544/276; 514/263.22; 544/238; 514/252.04; 548/143; 514/364; 544/370; 514/254.06; 544/369; 514/254.02; 546/199; 514/322; 544/131; 514/234.5; 544/58.4; 514/228.2; 544/105; 514/230.5; 548/300.7

(57) ABSTRACT

The present invention relates to novel compounds of formula (I): as herein described and pharmaceutical compositions thereof. The compounds of formula (I) have inhibitory effect on the Wnt pathway and are therefore useful in the preparation of a medicament, in particular for the treatment of cancer.

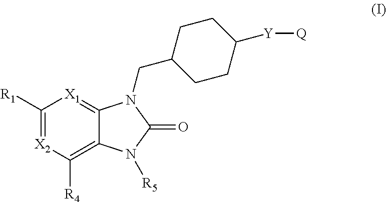

The present invention relates to novel compounds having inhibitory effect on the Wnt pathway, and to their pharmaceutical uses.

### BACKGROUND TO THE INVENTION

The Wnt gene family encodes a large class of secreted proteins related to the Int1/Wnt11proto-oncogene and Drosophila wingless (“Wg”), a Drosophila Wnt1 homologue (Cadigan et al. (1997) Genes & Development 11:3286-3305). Wnts are expressed in a variety of tissues and organs and are required for developmental processes, including segmentation in Drosophila; endoderm development in C. elegans; and establishment of limb polarity, neural crest differentiation, kidney morphogenesis, sex determination, and brain development in mammals (Parr, et al. (1994) Curr. Opinion Genetics & Devel. 4:523-528). The Wnt pathway is a master regulator in animal development, both during embryogenesis and in the mature organism (Eastman, et al. (1999) Curr Opin Cell Biol 11: 233-240; Peifer, et al. (2000) Science 287: 1606-1609). The variety of biological processes to which they take part during embryonic development and adult homeostasis is paralleled by the diversification within genomes into Wnt orthologues (19 identified Wnts in humans) and by the capacity to activate at least three intracellular signalling pathways (Moon et al., 2002; Nelson and Nusse, 2004; Seto and Bellen, 2004), the calcium-mediated and planar polarity pathways (Strutt, 2003; Veeman et al., 2003; Kuhl, 2004) and the canonical Wnt-β-catenin pathway. In the canonical Wnt pathway, Wnt ligands bind to their Frizzled receptor of a family of 10 reported Frizzled (“Fz”) seven transmembrane domain receptors (Bhanot et al. (1996) Nature 382:225-230). So doing, they activate the cytoplasmic protein Dishevelled (Dv1-1, 2 and 3 in humans and mice) (Boutros, et al. (1999) Mech Dev 83: 27-37) and phosphorylate LRP5/6. A signal is thereby generated which prevents the phosphorylation and degradation of Armadillol/β-catenin, in turn leading to an increase in cytoplasmic β-catenin (Perrimon (1994) Cell 76:781-784). This β-catenin translocates to the nucleus where it binds TCF (T cell factor) transcription factors (also known as lymphoid enhancer-binding factor-1 (LEF1)), serving as a coactivator of TCF/LEF-induced transcription (Bienz, et al. (2000) Cell 103: 311-320; Polakis, et al. (2000) and finally leading to the increased gene expression of Wnt target genes. In the absence of Wnt, cytoplasmic β-catenin protein is constantly degraded by the action of the Axin complex, which is composed of the scaffolding protein Axin, the tumor suppressor adenomatous polyposis coli gene product (APC), casein kinase 1 (CK1), and glycogen synthase kinase 3 (GSK3). CK1 and GSK3 sequentially phosphorylate the amino terminal region of β-catenin, resulting in β-catenin recognition by β-Trcp, an E3 ubiquitin ligase subunit, and subsequent β-catenin ubiquitination and proteasomal degradation (He et al., 2004). This continual elimination of β-catenin prevents β-catenin from reaching the nucleus, and Wnt target genes are thereby repressed by the DNA-bound T cell factor/lymphoid enhancer factor (TCF/LEF) family of proteins.

An increasing number of studies suggest how Wnt signalling related disorders can be initiated not only by mutations involving APC or Axin proteins (e.g., colorectal cancer), responsible for β-catenin degradation but also by alternative mechanisms. Hyperactivating mutations at the LRP5 co-receptor level are associated with high bone-density familial autosomal dominant syndrome (Boyden et al., N Engl J. Med. 2002; 346(20):1513-21). Autocrine Wnt signaling mediated by specific Wnt ligands was in fact linked to lung (Akiri et al. Oncogene 2009 28(21):2163-72), breast (Schlange et al., Breast Cancer Res. 2007; 9(5):R63 and Matsuda et al., Breast Cancer Res. 2009; 11(3):R32) and pancreatic (Nawroth et al., PLoS One. 2007 Apr. 25; 2(4):e392) tumors, but also malignant melanoma cells spreading (O'Connell et al., J Biol. Chem. 2009 Aug. 20., Epub ahead of print). Wnt signals form a class of paracrine growth factors act to influence multiple myeloma cell growth (Derksen et al., PNAS. 2004; 101(16):6122-7). The metastatic process, an ominous feature of most malignant tumors represents an additional area of intervention for Wnt inhibitors (Nguyen et al., Cell. 2009; 138(1):51-62) or tumor recurrence in glioblastoma patients (Sakarlassen et al., PNAS 2006, 103 (44) 16466) where different pathways seem to rule primary versus recurrent tumors. Moreover, there is strong evidence of the Wnt pathway involvement in cancers such as gastric cancer (Taniguchi et al, Oncogene. 2005 Nov. 24; 24(53):7946-52), medulloblastoma (Vibhakar et al., Neuro Oncol. 2007 April; 9(2):135-44), glioblastoma (Pu et al., Cancer Gene Ther. 2009 (4):351-61), hepatocellular carcinomas (Colnot et al., Proc Natl Acad Sci USA. 2004 Dec. 7; 101(49):17216-2), basal cell carcinoma (Yang et al., Nat. Genet. 2008 September; 40(9):1130-5), leukaemia (Staal, Blood, 109, 12, 5073-5074, 2007; Tickenbrock et al., Int. J. Oncol., 33,1215-1221, 2008; Zhao, Cancer Cell, 12, 528-541,2007), Wilm's tumours (Rivera et al., Science, 315,642-645, 2007 and Major et al., Science, 316,1043-1046, 2007) and Familial Adenomatous Polyposis (Kinzler et al., Science 253,661-665, 1991 and Nishisho et al., Science 253,665-669, 1991). There is also evidence that the inhibition of the Wnt pathway benefits pulmonary and renal fibrosis patients (Königshoff et al., PLoS One 3(5):e2142, 2008 and Henderson et al., PNAS, 107 (32), 14309-14314, 2010; Pulkkinen K. et al. Organogenesis 2008, 55-59, Brack et al., Science 2007, 317(5839), 807-10) and that Wnt inhibition can be used to treat diseases or conditions that involve myelin damage, such as ischemic neural injury and multiple sclerosis (Casaccia P. Nat. Neurosci. 2011, 14, 945-947; Fancy, S. P. J. et al Nat. Neurosci. 2011, 14, 1009-1016; Fancy, S. P. J. et al Genes Dev. 2009, 23, 1571-1585).

### DETAILED DESCRIPTION OF THE INVENTION

In one embodiment, there is provided compounds of formula I below

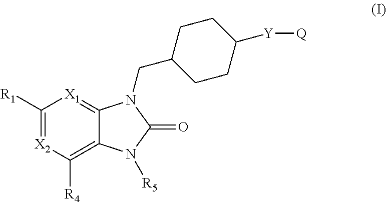

wherein, as valence and stability permit;

any carbon-bound hydrogen atom may be substituted with a fluorine atom;

X₁ is CR₂ or N;

X₂ is CR₃ or N;

-Y-Q is

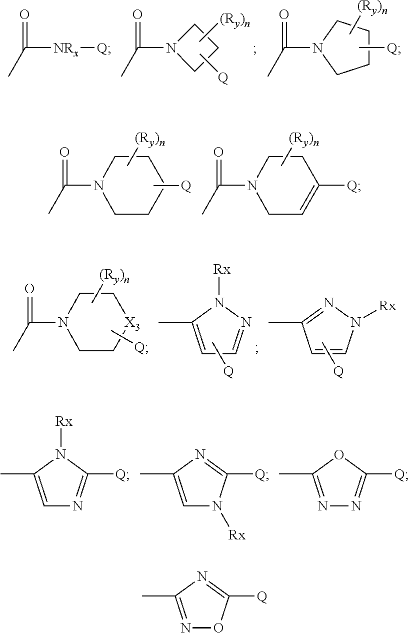

Q is C₁-C₆ linear branched or cyclic alkyl, alkylcarbonyl, oxalkyl, dioxalkyl, alkylaminocarbonyl, oxalkylamminocarbonyl group wherein any methylene group may be substituted with an oxo group; a C₅-C₁₀ aryl or heteroaryl group optionally substituted with 1, 2 or 3 groups selected from the list of C₁-C₆ linear branched or cyclic alkyl, oxalkyl, alkylamino, alkylamino carbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;

R₁ is H; F; Cl; Br; OH; CN; linear branched or cyclic C₁-C₆ alkyl, alkenyl, alkynyl, oxalkyl, oxalkenyl, oxalkynil, azalkenyl, azalkynyl, alkyloxy, alkenyloxy, oxalkyloxy, dioxalkyloxy, oxazalkyloxy, azalkyloxy, dialkylamino, oxalkylamino, azalkylamino, group optionally substituted with one or more F or CN; C₅-C₆ aryl- or heteroarylmethylammino or C₅-C₆ aryl- or heterorylmethyloxy group where the aryl or heteroaryl moiety may optionally be substituted with one or more C₁-C₃ alkyl, C₁-C₃ alkoxy, halogen or CN groups;

R₂ is H or Cl;

R₃ is H, Cl or F;

R₄ is H or Cl;

R₅ is a C₁-C₃ linear, branched or cyclic alkyl group;

Rx is H; a linear, branched or cyclic C₁-C₃ alkyl group;

n may be nil, 1, 2 or 3;

Ry is—independently from one another when n=2 or more—F; a linear, branched or cyclic C₁-C₃ alkyl group; or Ry, together with the carbon atom to which it is attached, forms an oxo group.

X₃ is either N, O or S;

tautomers, optical isomers and pharmaceutically acceptable salts thereof;

with the exception of

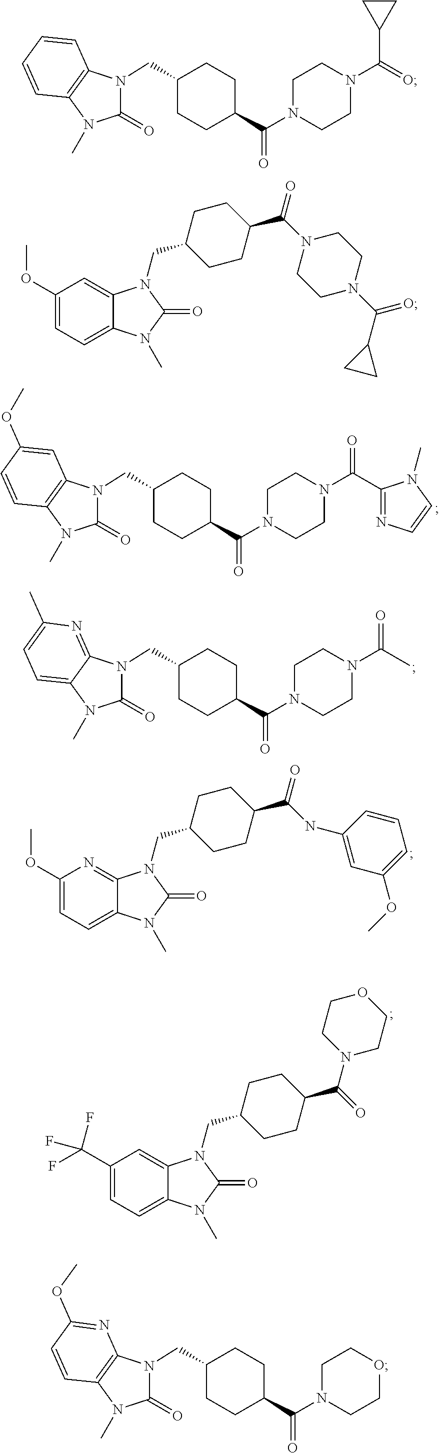

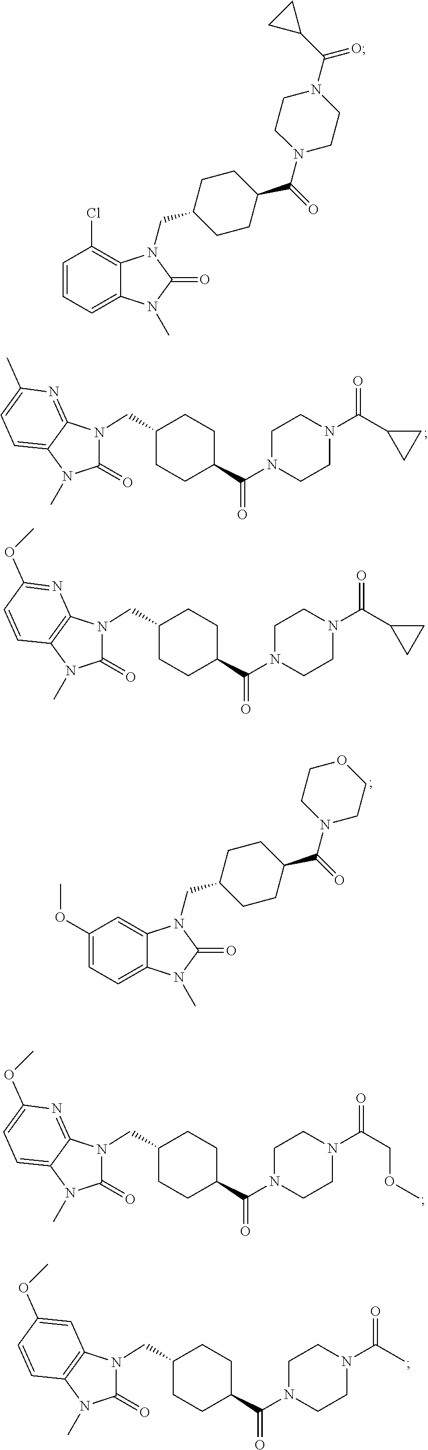

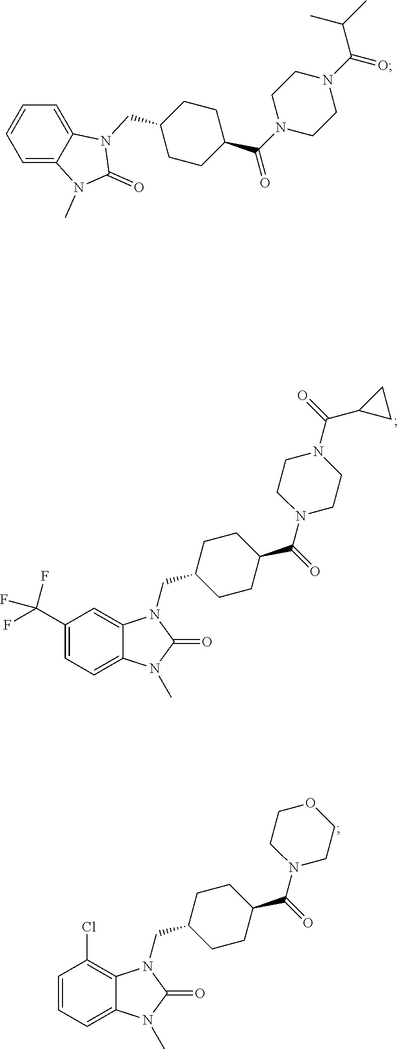 [SMILES: CC(C)C(=O)N1CCN(C(=O)[C@H]2CC[C@H](Cn3c(=O)n(C)c4ccccc43)CC2)CC1 ; Cn1c(=O)n(C[C@H]2CC[C@H](C(=O)N3CCN(C(=O)C4CC4)CC3)CC2)c2cc(C(F)(F)F)ccc21 ; Cn1c(=O)n(C[C@H]2CC[C@H](C(=O)N3CCOCC3)CC2)c2c(Cl)cccc21]

In one embodiment, there is provided compounds of formula (I-bis) below

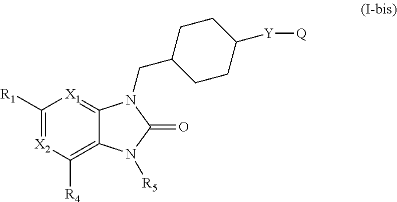

Wherein, as valence and stability permit;

any carbon-bound hydrogen atom may be substituted with a fluorine atom;

X₁ is CR₂;

X₂ is CR₃ or N;

-Y-Q is

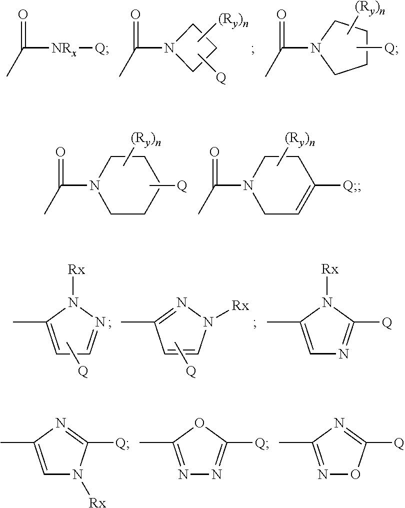

Q is C₁-C₆ linear branched or cyclic alkyl, alkylcarbonyl, oxalkyl, dioxalkyl, alkylaminocarbonyl, oxalkylamminocarbonyl group wherein any methylene group may be substituted with an oxo group; a C₅-C₁₀ aryl or heteroaryl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;

R₁ is H; F; Cl; Br; OH; CN; linear branched or cyclic C₁-C₆ alkyl, alkenyl, alkynyl, oxalkyl, oxalkenyl, oxalkynil, azalkenyl, azalkynyl, alkyloxy, alkenyloxy, oxalkyloxy, dioxalkyloxy, oxazalkyloxy, azalkyloxy, dialkylamino, oxalkylamino, azalkylamino, group optionally substituted with one or more F or CN; C₅-C₆ aryl- or heteroarylmethylammino or C₅-C₆ aryl- or heterorylmethyloxy group where the aryl or heteroaryl moiety may optionally be substituted with one or more C₁-C₃ alkyl, C₁-C₃ alkoxy, halogen or CN groups;

R₂ is H or Cl;

R₃ is H, Cl or F;

R₄ is H or Cl;

R₅ is a C₁-C₃ linear, branched or cyclic alkyl group;

Rx is H; a linear, branched or cyclic C₁-C₃ alkyl group;

n may be nil, 1, 2 or 3;

Ry is—independently from one another when n=2 or more—F; a linear, branched or cyclic C₁-C₃ alkyl group; or Ry, together with the carbon atom to which it is attached, forms an oxo group;

tautomers, optical isomers and pharmaceutically acceptable salts thereof.

In one embodiment,

Q is C₁-C₆ linear branched or cylic alkyl, alkylcarbonyl, oxalkyl, dioxalkyl, alkylaminocarbonyl, oxalkylamminocarbonyl group wherein any methylene group may be substituted with an oxo group; a C₅-C₆ aryl or heteroaryl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;

and X₁, X₂, X₃, Y, R₁, R₂, R₃, R₄, R₅, Rx, n, Ry are as defined under formula (I) or (I-bis) above

In another embodiment,

Q is C₁-C₆ linear branched or cylic alkyl, alkylcarbonyl, oxalkyl, dioxalkyl, alkylaminocarbonyl, oxalkylamminocarbonyl group wherein any methylene group may be substituted with an oxo group; a [1,2,4]oxadiazolyl, [1,3,4]thiadiazolyl, benzimidazolyl, benzothiazolyl, benzothiophenyl, benzoxazolyl, imidazolyl, 2H-indazolyl, isoxazolyl, oxazolyl, phenyl, pyrazinyl, pyrazolyl, pyridazinyl, pyridazinyl, imidazo[1,2-a]pyridine, pyridyl, pyrimidinyl, quinolyl or thiazolyl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a [1,3,4]oxadiazolyl, phenyl, furanyl or pyridyl group optionally substituted with halogen, C₁-C₃ alkyl, C₁-C₃ oxalkyl; and X₁, X₂, X₃, Y, R₁, R₂, R₃, R₄, R₅, Rx, n, Ry are as defined under formula (I) or (I-bis) above

In another embodiment,

Q is C₁-C₆ linear branched or cylic alkyl, alkylcarbonyl, oxalkyl, dioxalkyl, alkylaminocarbonyl, oxalkylamminocarbonyl group wherein any methylene group may be substituted with an oxo group; a [1,2,4]oxadiazolyl, benzothiazolyl, benzoxazolyl, isoxazolyl, phenyl, pyrazinyl, pyrazolyl, pyridazinyl, pyridazinyl, pyridyl, pyrimidinyl, quinolyl or thiazolyl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a [1,3,4]oxadiazolyl, group optionally substituted with halogen, C₁-C₃ alkyl, C₁-C₃ oxalkyl;

and X₁, X₂, X₃, Y, R₁, R₂, R₃, R₄, R₅, Rx, n, Ry are as defined under formula (I) or (I-bis)

In another embodiment

Q is C₁-C₆ linear branched or cylic alkyl, alkylcarbonyl, oxalkyl, dioxalkyl, alkylaminocarbonyl, oxalkylamminocarbonyl group wherein any methylene group may be substituted with an oxo group; a 2-benzothiazolyl, 2-oxazolyl, 2-pyrazinyl, 2-pyridyl, 2-pyrimidinyl, 2-thiazolyl, 3-isoxazolyl, 3-pyrazolyl, 3-pyridazinyl, 3-pyridyl, 4-pyrazolyl, 4-pyridazinyl, 4-pyridyl, 4-pyrimidinyl, 4-thiazolyl, 5-[1,2,4]oxadiazolyl, 5-[1,3,4]thiadiazolyl, 5-benzimidazolyl, 5-benzothiophenyl, 5-benzoxazolyl, 5-imidazolyl, 5-isoxazolyl, 5-pyrazolyl, 5-pyrimidinyl, 5-quinolyl, 6-benzothiazolyl, 8-quinolyl, 4-2H-indazolyl, phenyl or 3-imidazo[1,2-a]pyridine, group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a [1,3,4]oxadiazolyl, group optionally substituted with halogen, C₁-C₃ alkyl, C₁-C₃ oxalkyl;

and X₁, X₂, X₃, Y, R₁, R₂, R₃, R₄, R₅, Rx, n, Ry are as defined under formula (I) or (I-bis) above

In another embodiment,

Q is C₁-C₆ linear branched or cylic allyl, alkylcarbonyl, oxalkyl, dioxalkyl, alkylaminocarbonyl, oxalkylamminocarbonyl group wherein any methylene group may be substituted with an oxo group; a 2-benzothiazolyl, 2-pyrazinyl, 2-pyridyl, 2-pyrimidinyl, 2-thiazolyl, 3-isoxazolyl, 3-pyrazolyl, 3-pyridazinyl, 3-pyridyl, 4-pyrazolyl, 4-pyridazinyl, 4-pyridyl, 4-pyrimidinyl, 4-thiazolyl, 5-[1,2,4]oxadiazolyl, 5-benzoxazolyl, 5-isoxazolyl, 5-pyrazolyl, 5-pyrimidinyl, 5-quinolyl or phenyl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, or a [1,3,4]oxadiazolyl, group optionally substituted with halogen, C₁-C₃ alkyl, C₁-C₃ oxalkyl;

and X₁, X₂, X₃, Y, R₁, R₂, R₃, R₄, R₅, Rx, n, Ry are as defined under formula (I) or (I-bis) above

In another embodiment,

X₁ is CR₂; R₂ is H;

X₂ is CR₃;

-Y-Q is

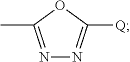

Q is a pyrazolyl group substituted with 1 to 3 C₁-C₃ alkyl wherein one or more carbon-bound hydrogen may be substituted by fluorine;

R₄ is H;

and R₁, R₃ and R₅ are as defined under formula (I) or (I-bis) above.

In another embodiment, there is provided a compound selected from the list of

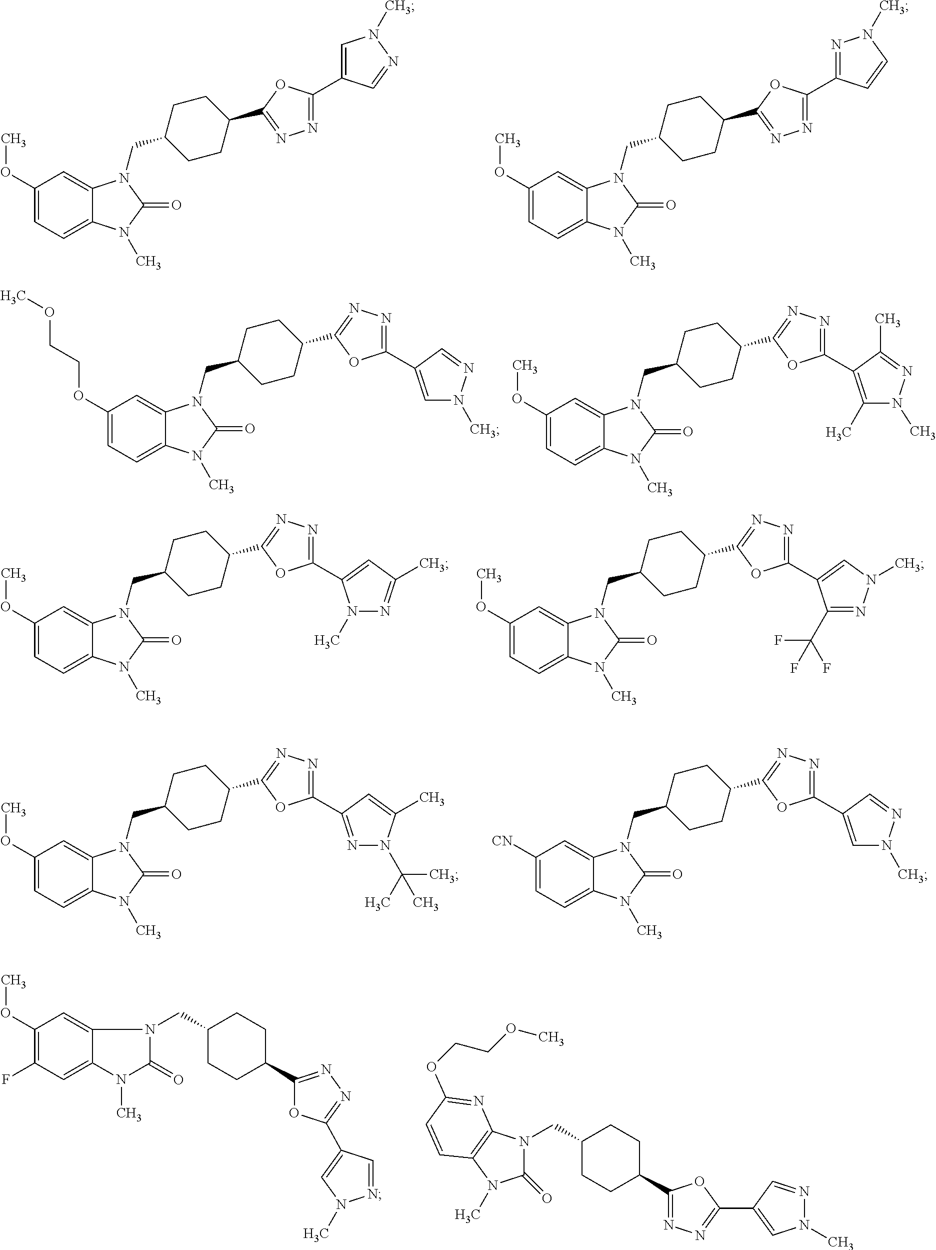

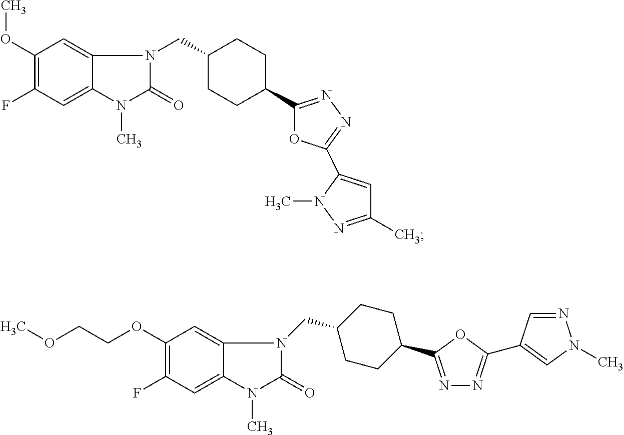

In another embodiment,

X₁ is CR₂; R₂ is H;

X₂ is CR₃;

-Q-Y is;

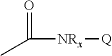

Q is pyridazinyl;

R₁ is a linear branched or cyclic C₁-C₆ oxalkyl, oxalkenyl, oxalkynyl, alkyloxy, oxalakyloxy, oxazalkyloxy, azalkyloxy group;

R₄ is H;

and R₃, R₅ and Rx as defined under formula (I) or (I-bis) above

In another embodiment, there is provided a compound selected from the list of

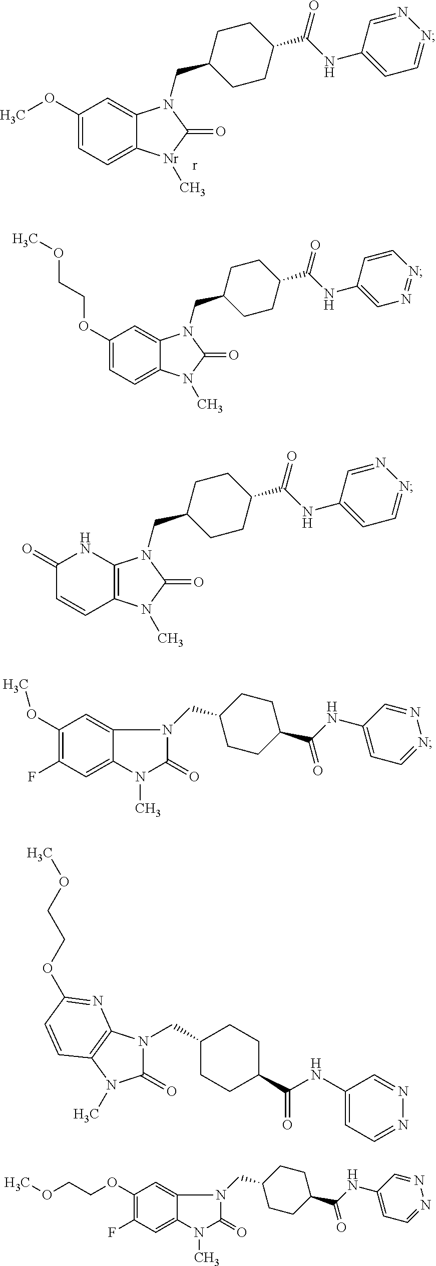

In another embodiment,

X₁ is CR₂; R₂ is H;

X₂ is CR₃;

-Q-Y is

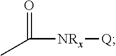

Q is 4-pyridyl;

R₁ is a linear, branched or cyclic C₁-C₆ alkyloxy, alkenyloxy, oxalkyloxy, dioxalkyloxy oxalkylammino, group optionally substituted with F or CN;

R₄ is H;

and R₅ is as defined under formula (I) or (I-bis) above

In another embodiment, there is provided a compound selected from the list of

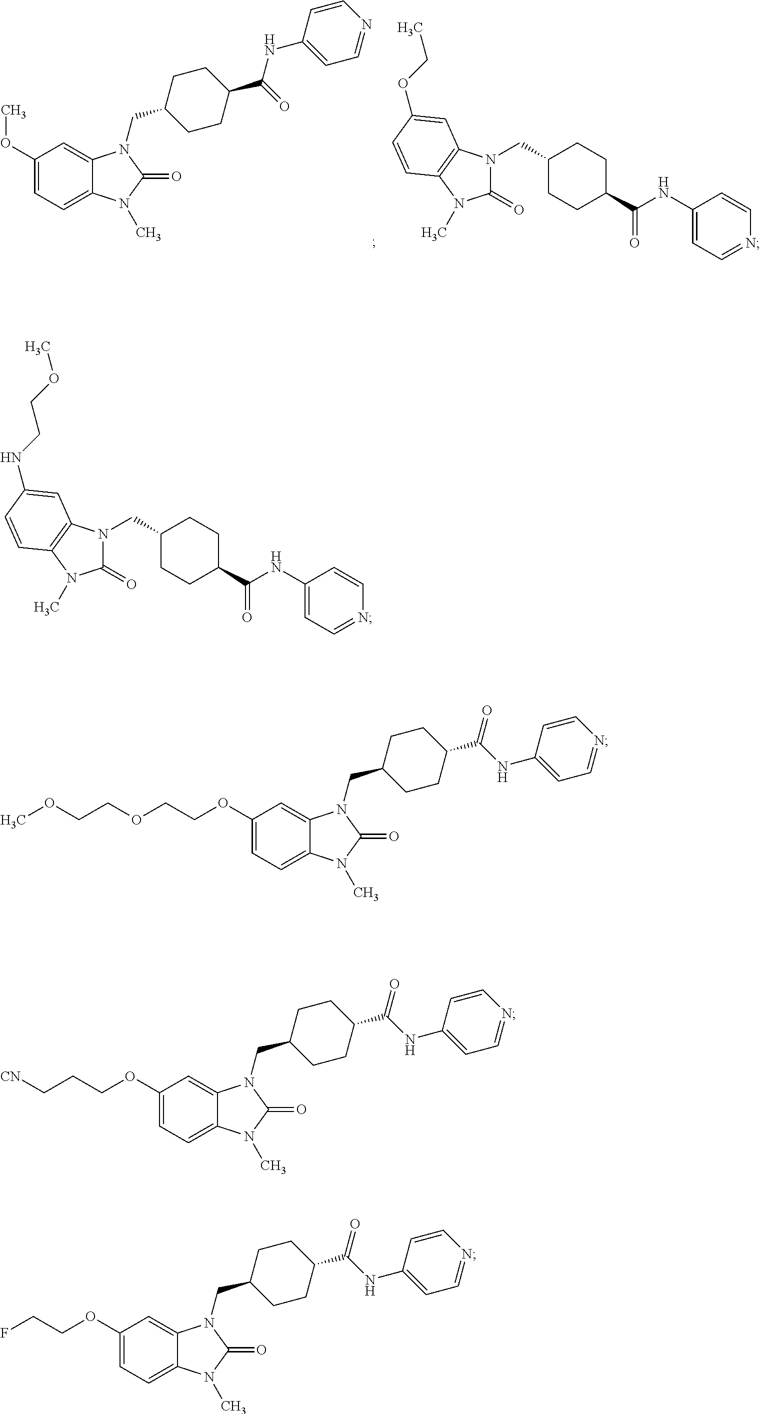

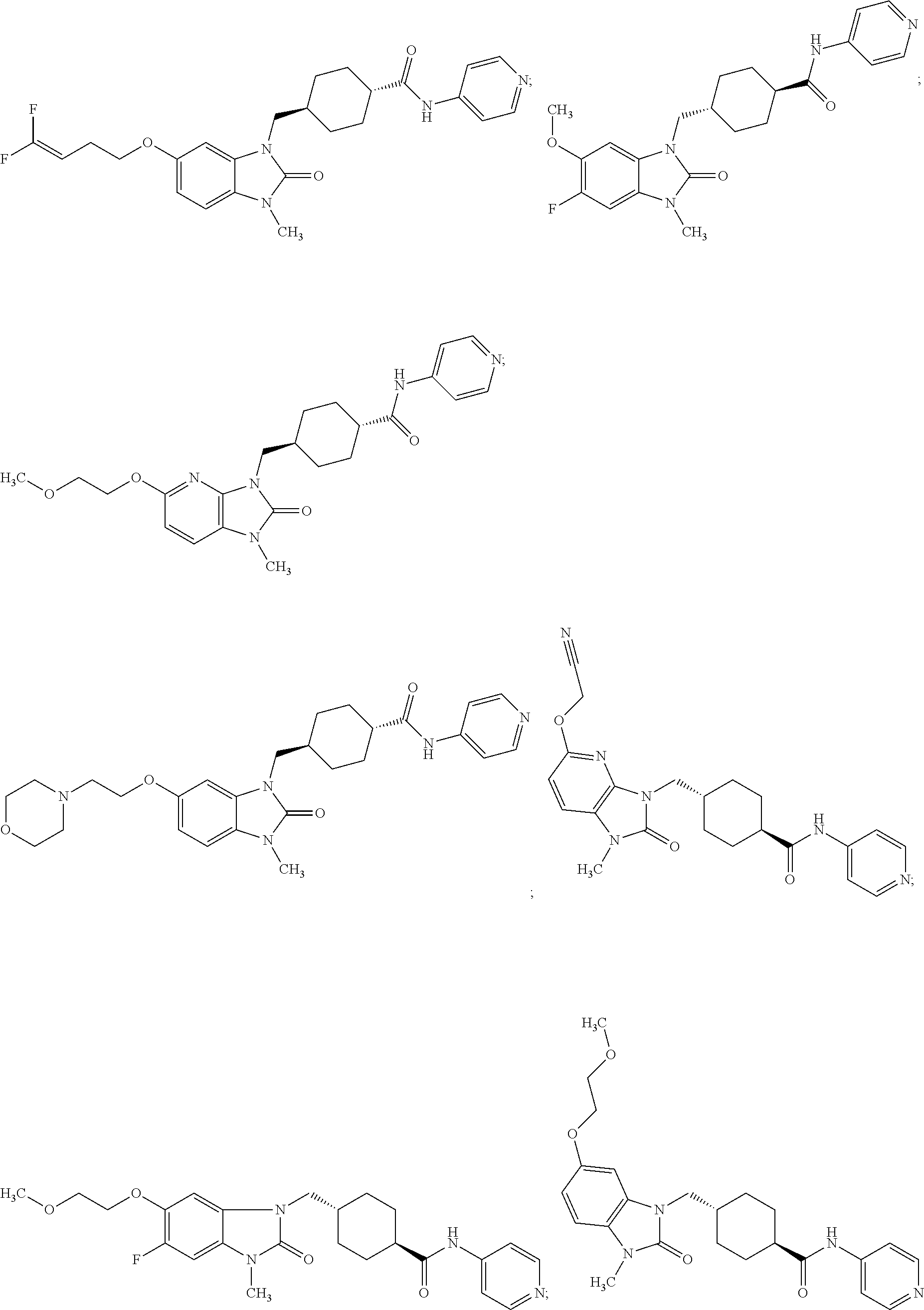

In another embodiment,

X₁ is CR₂; R₂ is H;

X₂ is CR₃;

R₁ is a linear, branched or cyclic C₁-C₆ alkoxy or oxalkyloxy;

R₃ is F;

R₄ is H; and X₃, Y-Q, R₅, Rx, n and Ry are as defined under formula (I) or (I-bis) above

In another embodiment, there is provided a compound selected from the list of

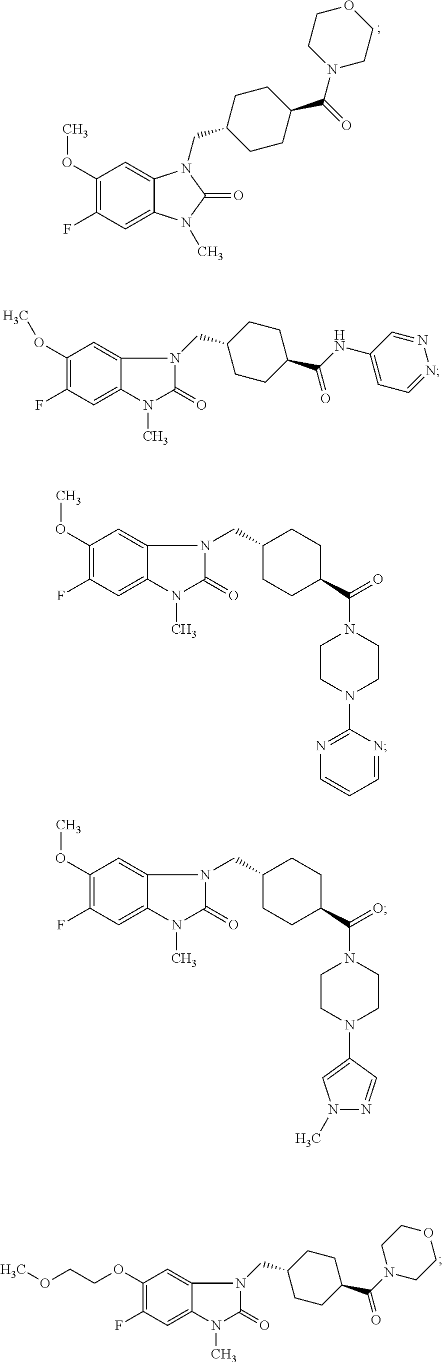 [SMILES: COc1cc2c(cc1F)n(C)c(=O)n2C[C@H]1CC[C@H](C(=O)N2CCOCC2)CC1 ; COc1cc2c(cc1F)n(C)c(=O)n2C[C@H]1CC[C@H](C(=O)Nc2ccnnc2)CC1 ; COc1cc2c(cc1F)n(C)c(=O)n2C[C@H]1CC[C@H](C(=O)N2CCN(c3ncccn3)CC2)CC1 ; COc1cc2c(cc1F)n(C)c(=O)n2C[C@H]1CC[C@H](C(=O)N2CCN(c3cnn(C)c3)CC2)CC1 ; COCCOc1cc2c(cc1F)n(C)c(=O)n2C[C@H]1CC[C@H](C(=O)N2CCOCC2)CC1]

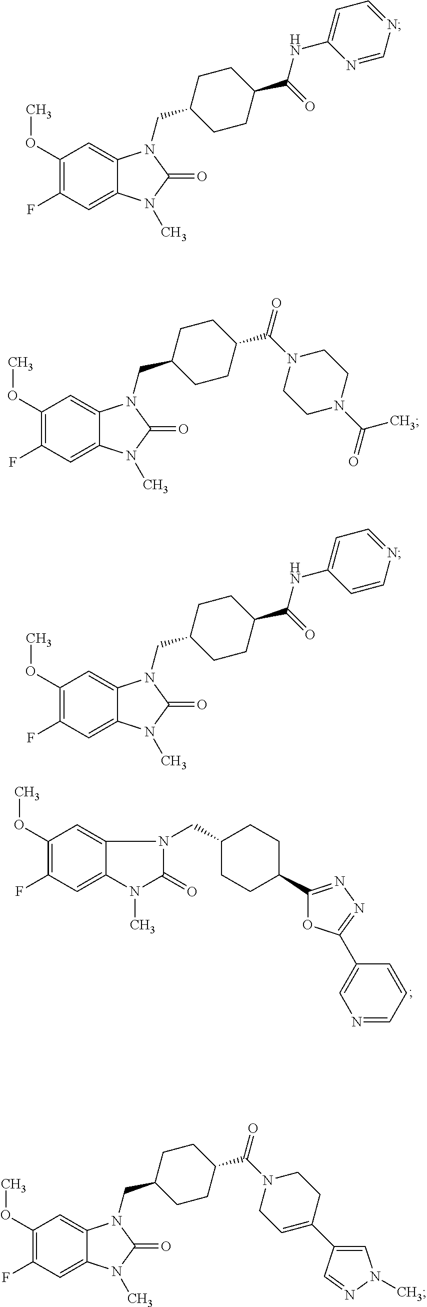 [SMILES: COc1cc2c(cc1F)n(C)c(=O)n2C[C@H]1CC[C@H](C(=O)Nc2ccncn2)CC1 ; COc1cc2c(cc1F)n(C)c(=O)n2C[C@H]1CC[C@H](C(=O)N2CCN(C(C)=O)CC2)CC1 ; COc1cc2c(cc1F)n(C)c(=O)n2C[C@H]1CC[C@H](C(=O)Nc2ccncc2)CC1 ; COc1cc2c(cc1F)n(C)c(=O)n2C[C@H]1CC[C@H](c2nnc(-c3cccnc3)o2)CC1 ; COc1cc2c(cc1F)n(C)c(=O)n2C[C@H]1CC[C@H](C(=O)N2CC=C(c3cnn(C)c3)CC2)CC1]

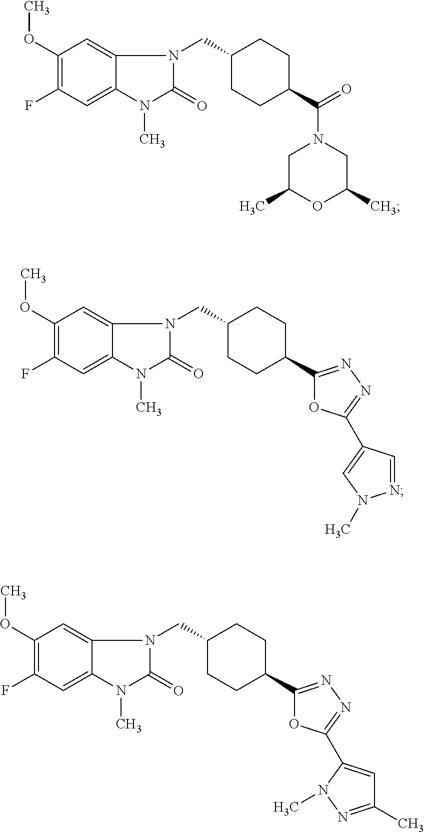 [SMILES: COc1cc2c(cc1F)n(C)c(=O)n2C[C@H]1CC[C@H](C(=O)N2C[C@@H](C)O[C@@H](C)C2)CC1 ; COc1cc2c(cc1F)n(C)c(=O)n2C[C@H]1CC[C@H](c2nnc(-c3cnn(C)c3)o2)CC1 ; COc1cc2c(cc1F)n(C)c(=O)n2C[C@H]1CC[C@H](c2nnc(-c3cc(C)nn3C)o2)CC1]

In another embodiment, there is provided compounds of formula (I-ter) below,

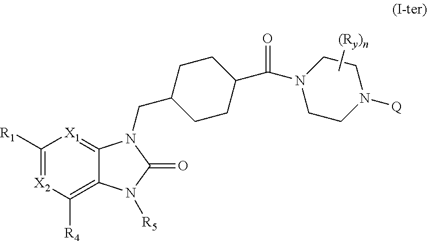

Wherein, as valence and stability permit;

any carbon-bound hydrogen atom may be substituted with a fluorine atom;

X₁ is CR₂; R₂ is H

X₂ is CR₃,

Q is a C₁-C₃ linear, branched or cyclic alkylcarbonyl;

R₁ is OH, linear branched or cyclic C₁-C₆ alkyl, alkenyl, alkynyl, oxalkyl, oxalkyloxy, oxalkylammino group;

R₄ is H;

R₃ is H, Cl or F;

R₅ is a C₁-C₃ linear, branched or cyclic alkyl group;

n may be nil, 1, 2 or 3;

Ry is—independently from one another when n=2 or more—F; a linear, branched or cyclic C₁-C₃ alkyl group; or Ry, together with the carbon atom to which it is attached, forms an oxo group;

tautomers, optical isomers and pharmaceutically acceptable salts thereof.

In an embodiment of compounds falling under formula (I-ter) above,

R₁ is a linear branched or cyclic C₁-C₆ alkyl group;

In another embodiment, there is provided a compound selected from the list of

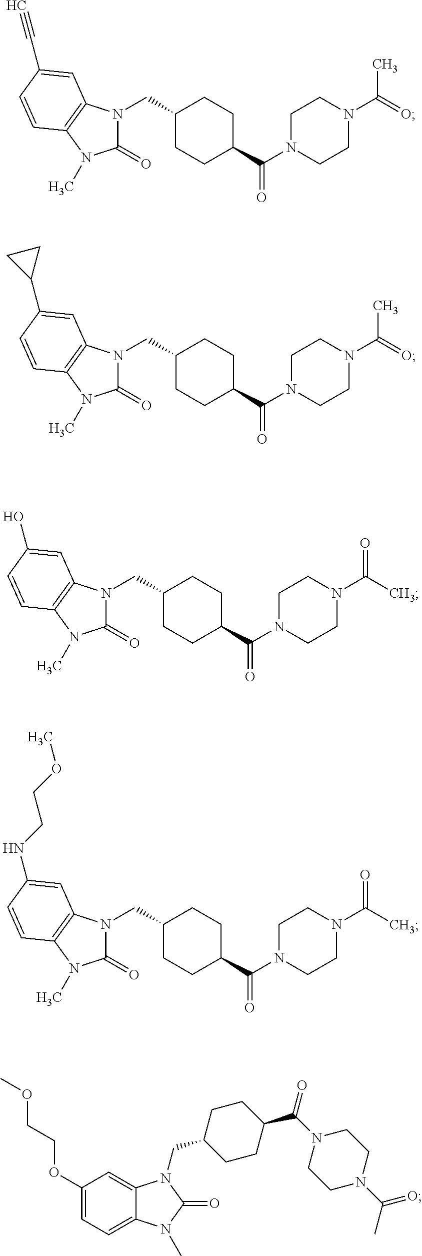 [SMILES: C#Cc1ccc2c(c1)n(C[C@H]1CC[C@H](C(=O)N3CCN(C(C)=O)CC3)CC1)c(=O)n2C ; CC(=O)N1CCN(C(=O)[C@H]2CC[C@H](Cn3c(=O)n(C)c4ccc(C5CC5)cc43)CC2)CC1 ; CC(=O)N1CCN(C(=O)[C@H]2CC[C@H](Cn3c(=O)n(C)c4ccc(O)cc43)CC2)CC1 ; COCCNc1ccc2c(c1)n(C[C@H]1CC[C@H](C(=O)N3CCN(C(C)=O)CC3)CC1)c(=O)n2C ; COCCOc1ccc2c(c1)n(C[C@H]1CC[C@H](C(=O)N3CCN(C(C)=O)CC3)CC1)c(=O)n2C]

In another embodiment,

X₁ is CR₂; R₂ is H

R₁ is a C₁-C₃ linear branched or cyclic alkoxy group

X₂ is CR₃,

R₃ is H;

R₄ is H;

-Q-Y is

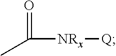

Q is a C₅-C₁₀ aryl or heteroaryl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;

and R₅, Rx and n are as defined under formula (I) or (I-bis) above

In another embodiment, there is provided a compound selected from the list of

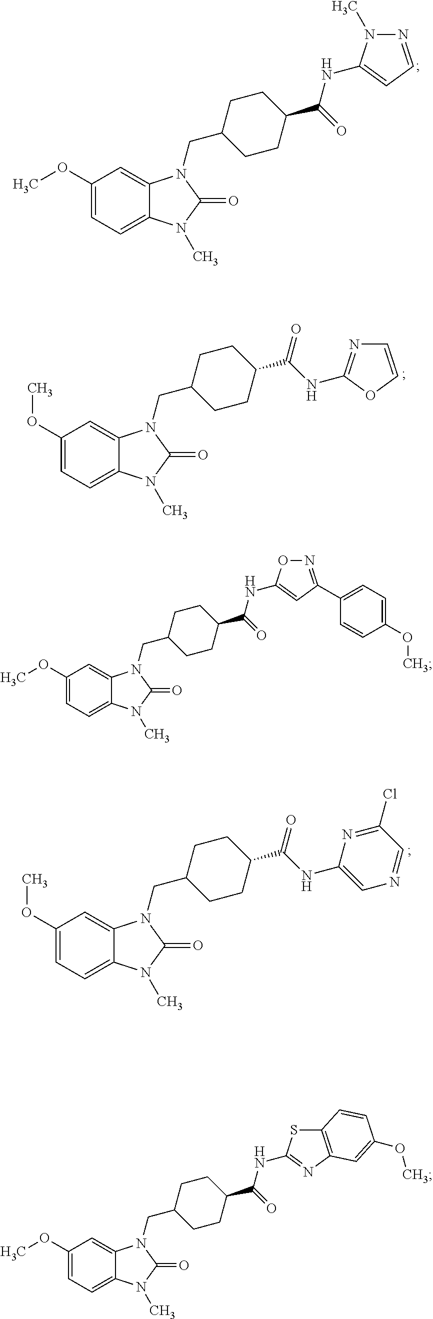

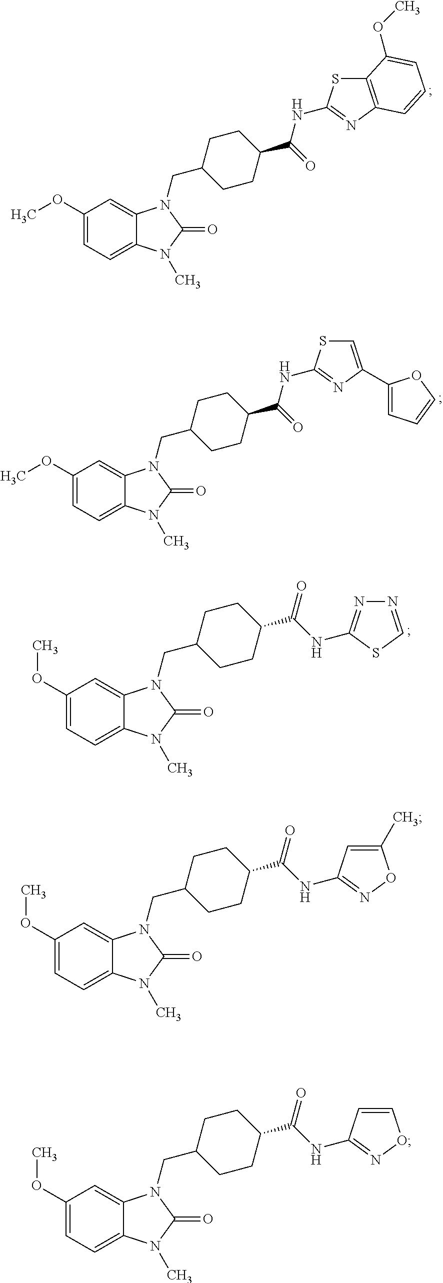

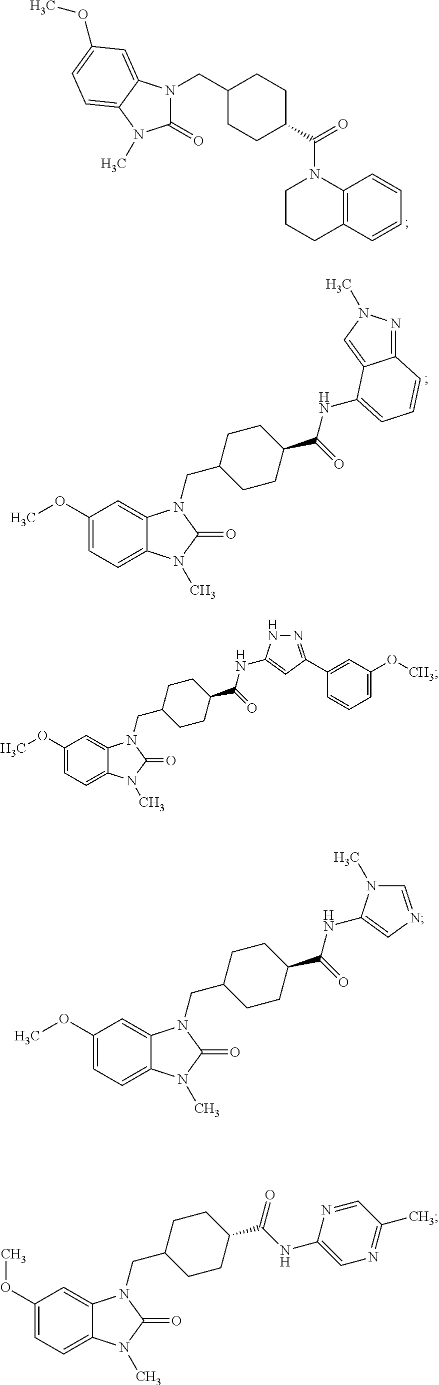

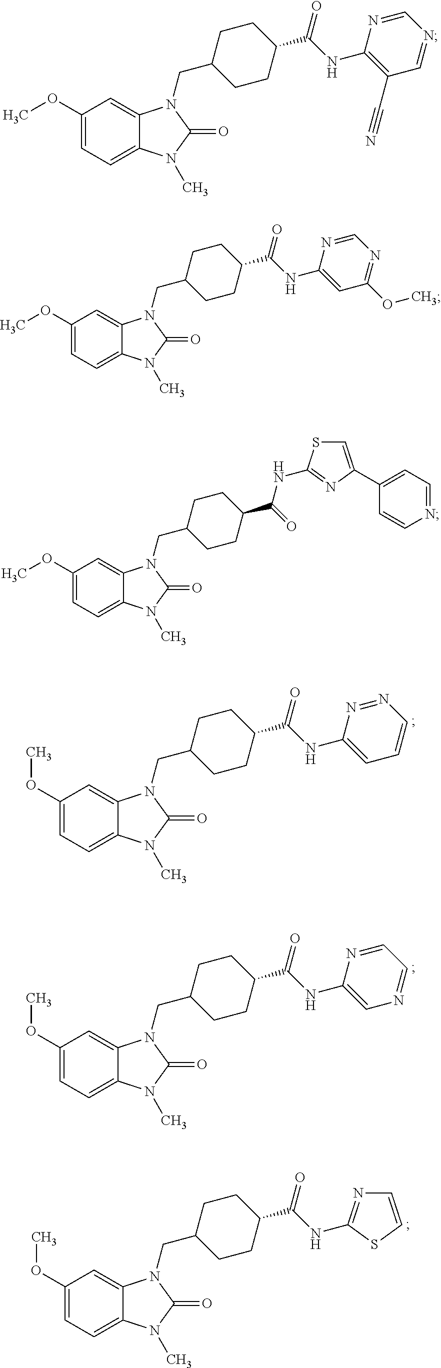

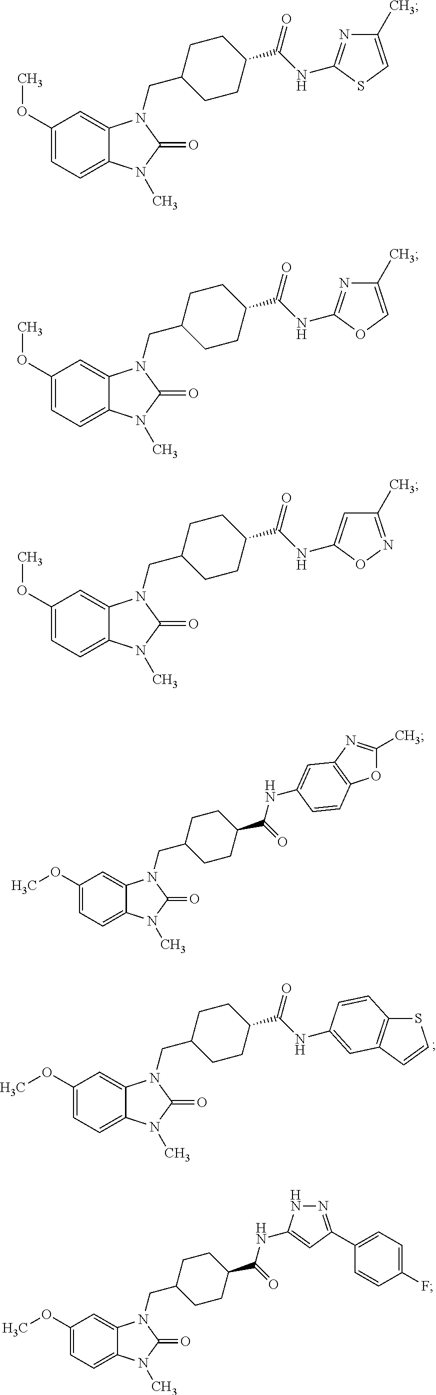

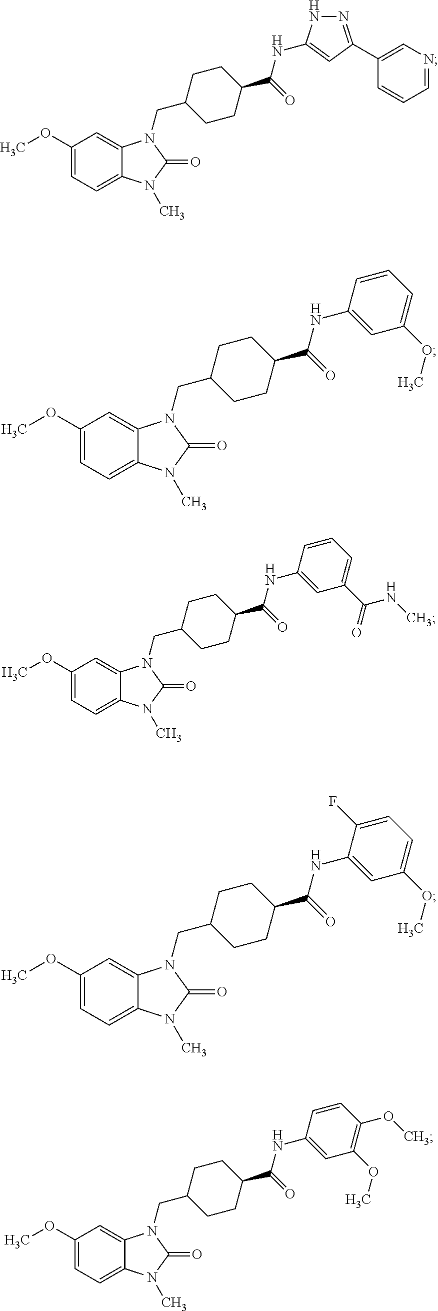

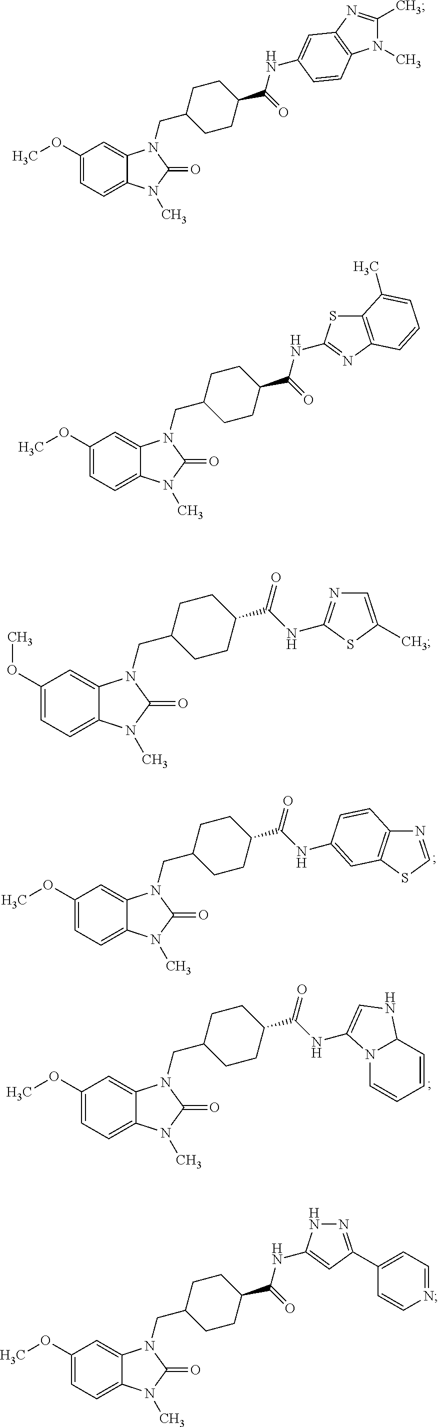

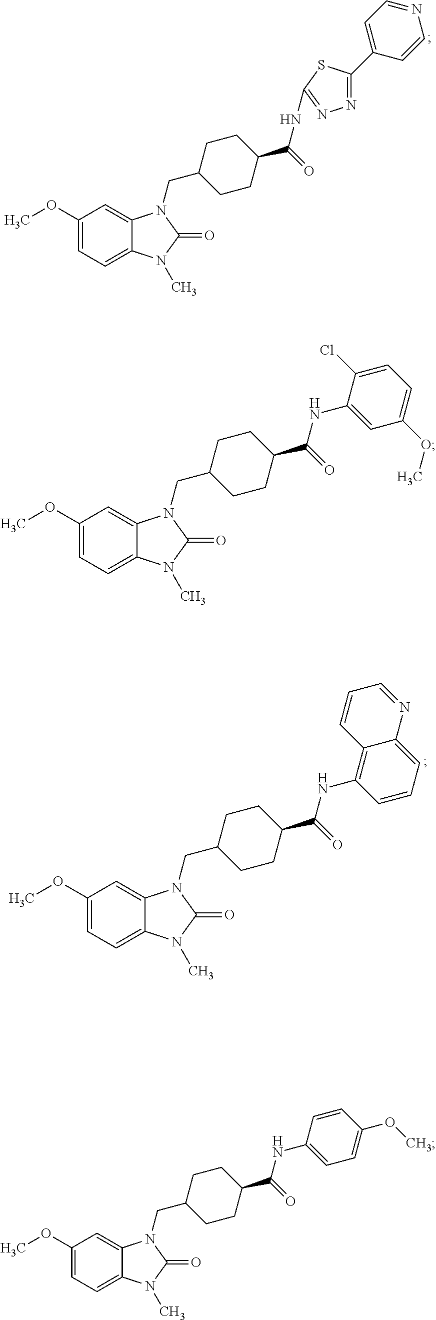

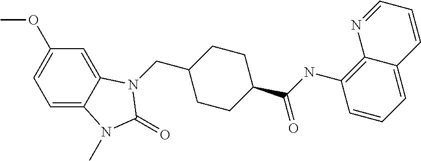

In another embodiment X₁ is CR₂; R₂ is H; X₂ is CR₃, R₄ is H, R₅ is methyl and X₃, Y-Q, R₁, R₃, R₄, Rx, n and Ry are as defined under formula (I) or (I-bis) above.

In another embodiment, X₁ is N; X₂ is CR₃; R₄ is H and X₃, Y-Q, R₁, R₂, R₃, R₄, R₅, Rx, n and Ry are as defined under formula (I) or (I-bis) above

In another embodiment X₁ is N; X₂ is CR₃; R₄ is H; -Y-Q is

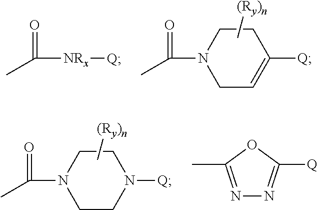

and X₃, R₁, R₂, R₃, R₄, R₅, Rx, n, Ry are as defined under formula (I) or (I-bis) above

In another embodiment X₁ is N; X₂ is CR₃; R₄ is H; -Y-Q is

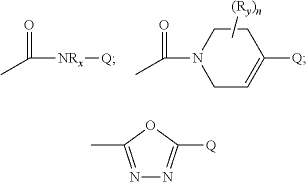

and X₃, R₁, R₂, R₃, R₄, R₅, Rx, n, Ry are as defined under formula (I) or (I-bis) above.

Within any embodiment, preferred compounds are those in which R₅ is methyl

All embodiments may be combined

Compounds Synthesis

Depending on the exact nature of the compound, compounds of the invention may be obtained under general schemes 1-13.

Compounds of formula Ia can be prepared according to Method A reported in Scheme 1.

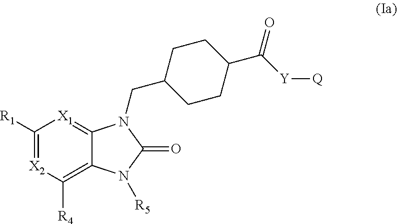

wherein -Y-Q is

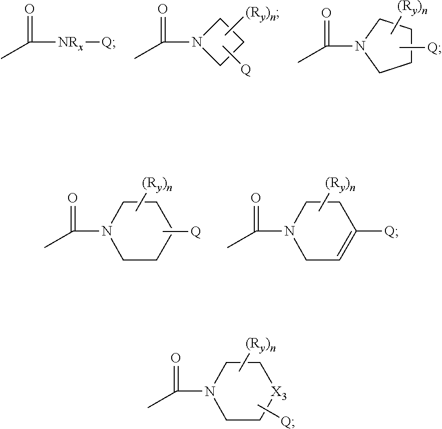

X₂ is CR₃ and R₁, R₄, R₅, Rₓ, R_{y}, X₁, X₃, Q and n are as defined under formula (I)

Reaction of 4-aminomethyl-cyclohexanecarboxylic acid methyl ester with the appropriate nitro-fluoro-benzenes or properly substituted halo-nitro-pyridine gives compounds of general formula 1 which can be reduced to the dianilines of general formula 2 using standard reaction procedures. Substituted nitro-fluoro-benzenes and nitro bromo pyridines are commercially available or have been described in literature or can be synthesized using standard procedures. 4-aminomethylcyclohexane carboxylic methyl ester can be synthesized from the corresponding acid in analogy to the reported methods (see for example WO07064273). Cyclization of 2 with CDI (1,1-carbonyldiimidazole), or triphosgene (Bis(trichloromethyl)carbonate) affords compounds of general formula 3. Such compounds can be hydrolysed to the corresponding carboxylic acid 4 and coupled with an amine in presence of an appropriate coupling agent to give compounds of general formula 5. Alkylation will give compounds of general formula 8. In addition compounds of general formula 3 can be alkylated to intermediates of general formula 6. Hydrolysis of 6 gives the corresponding carboxylic acids 7 which are coupled with an amine in presence of an appropriate coupling agent to afford compounds 8. Alternatively compounds of general formula 8 can be obtained starting from the amines 9 which are reacted with the appropriate nitro-fluoro-benzenes or substituted bromo-nitro-pyridine to give intermediates 10. Amines 9 can be synthesized according to standard reaction procedures starting from the 4-aminomethyl-cyclohexanecarboxylic acid. The nitro compounds 10 can be reduced to the corresponding dianilines 11 using standard reduction conditions and then reacted with triphosgene or CDI to afford compounds of general formula 12. Intermediates 12 can then be alkylated to compounds 8 using suitable alkylating agents in presence of a base.

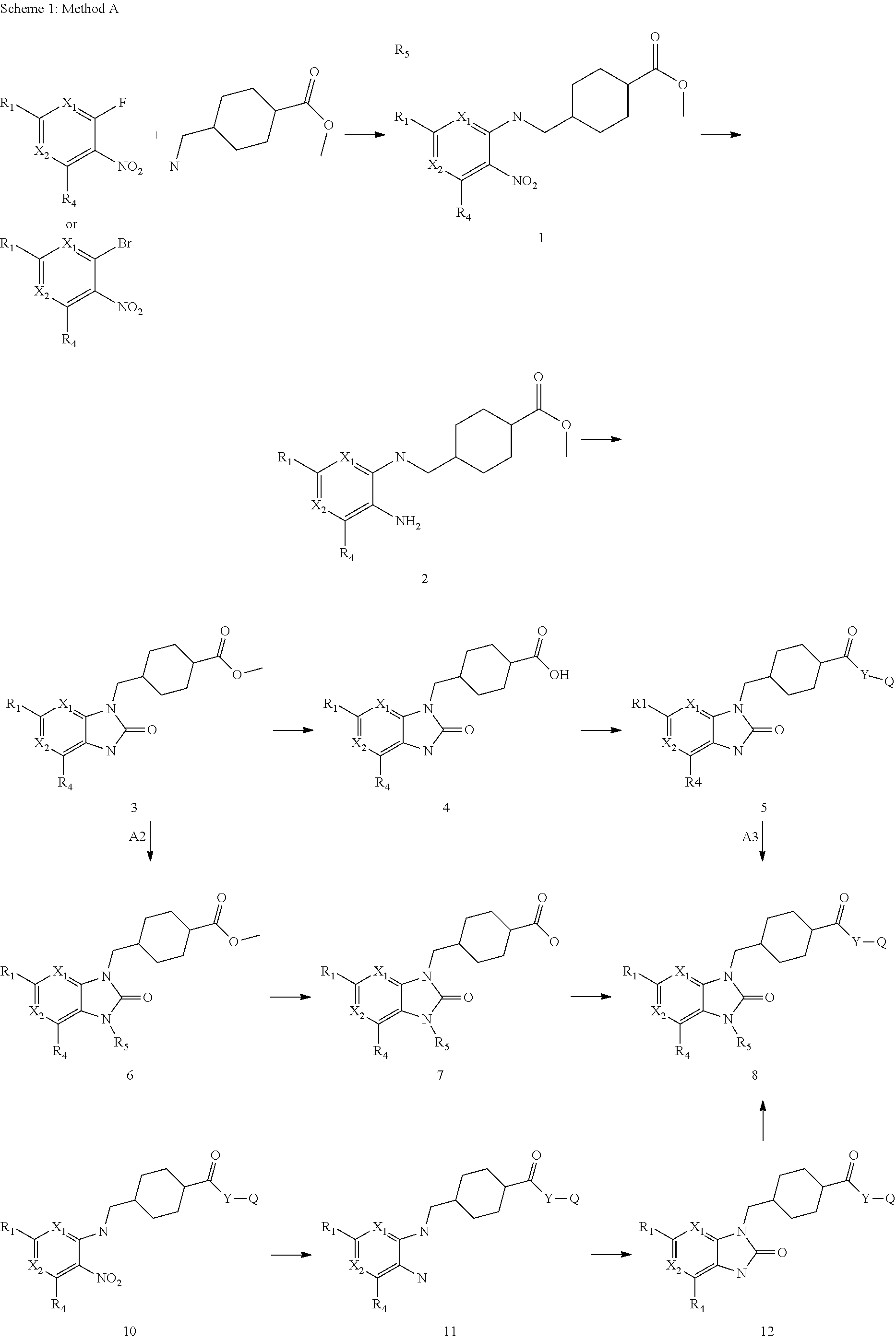

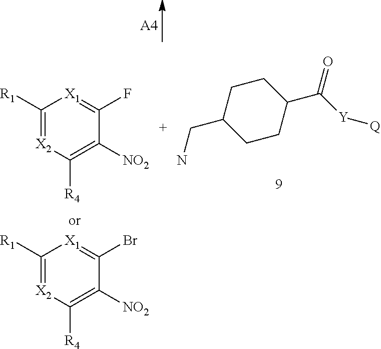

Compounds of formula Ib can be prepared according to Method B reported in Scheme 2.

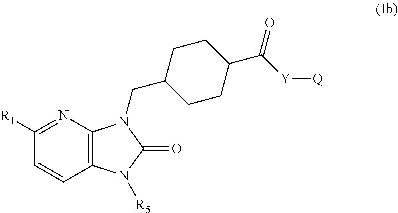

Wherein -Y-Q is

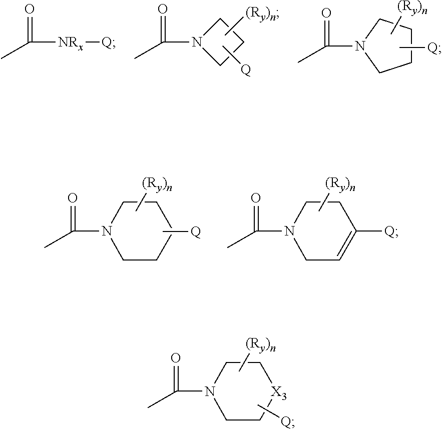

and R₁ is linear branched or cyclic C₁-C₆ alkyl, alkenyl, alkynyl, oxalkyl, oxalkenyl, oxalkynil, azalkenyl, azalkynyl, oxalkyloxy, oxazalkyloxy, azalkyloxy, alkylamino, dialkylamino, oxalkylammino, azalkylammino, group optionally substituted with one or more F or CN; C₅-C₆ aryl- or heteroarylmethylammino or C₅-C₆ aryl- or heterorylmethyloxy group where the aryl or heteroaryl moiety may optionally be substituted with one or more C₁-C₃ alkyl, C₁-C₃ alkoxy, halogen or CN groups and R₅, Qm Rₓ, R_{y}, X₃ and n are as defined under formula (I).

The bromo intermediates 13 can be converted to compounds of general structure 14 by methods known to those skilled in the art such as Suzuki, Buchwald and Sonogashira couplings. Compounds of general formula 13 can be synthesized according to general method A described in Scheme 1.

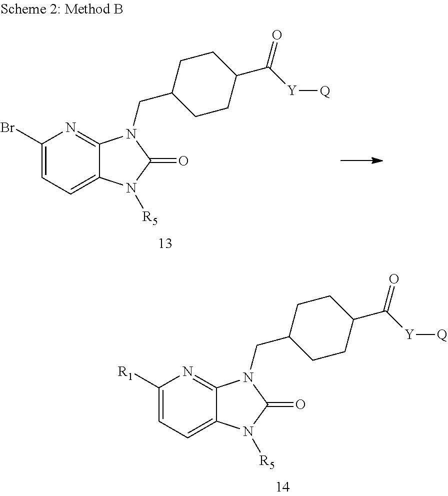

Compounds of formula Ic can be prepared according to Method C reported in Scheme 3.

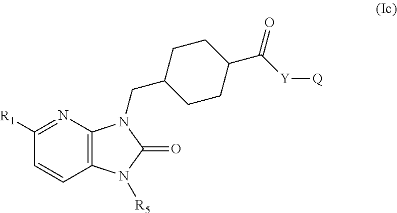

Wherein -Y-Q is

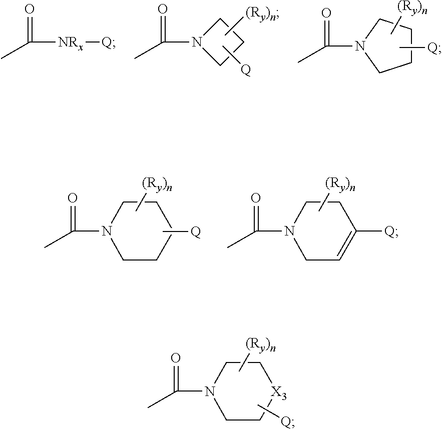

and R₁ is a dialkylamino, oxalkylamino or a azalkylamino and R₅, Q, R_{y}, Rₓ, X₃ and n are as defined under formula (I)

Reaction of 4-aminomethyl-cyclohexanecarboxylic acid methyl ester with the commercially available 2,6-dibromo-3-nitropyridine gives compound 15 which is reacted with an amine according to standard procedures to afford the intermediates of general formula 16. Reduction of 16 using standard methods affords the dianiline 17 which is cyclised with CDI or triphosgene to compounds 18. Intermediates 18 are alkylated, following standard procedures, to 19. Hydrolysis of 19 gives intermediate 20 which can be coupled with an amine in presence of an appropriate coupling agent to afford compounds of general formula 21.

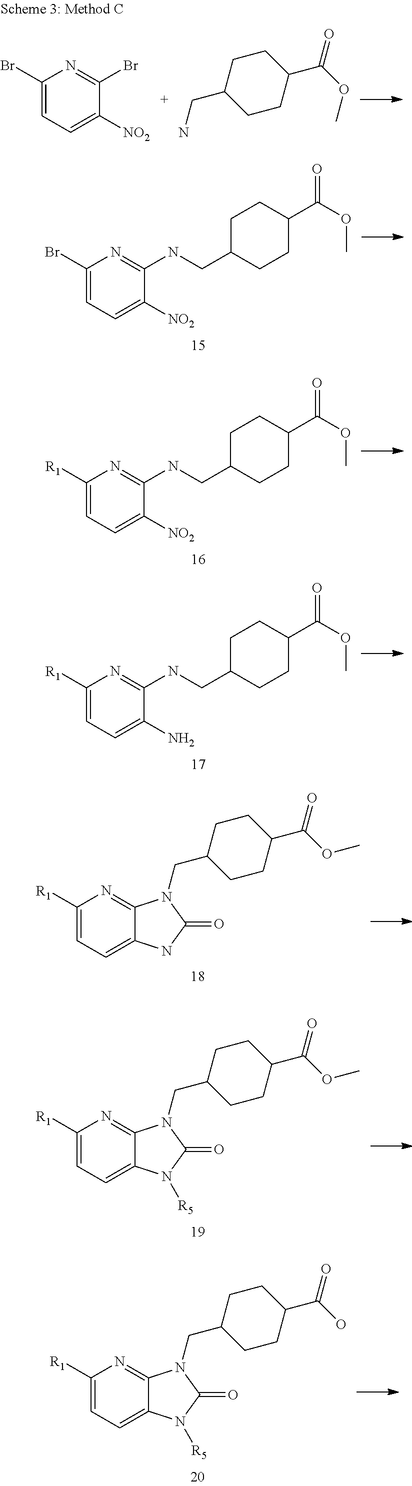

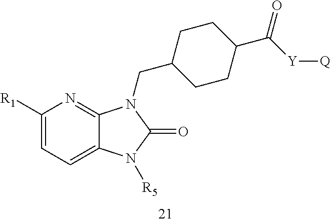

Compounds of formula Id can be prepared according to Method D reported in Scheme 4.

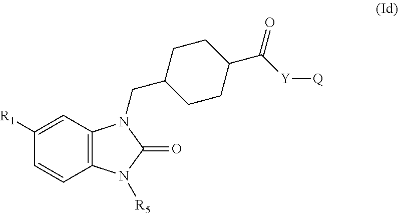

Wherein -Y-Q is

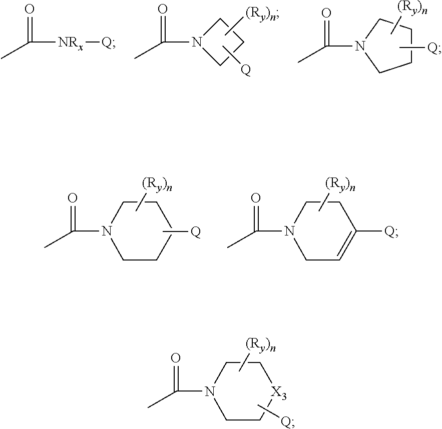

and R₁ is alkyloxy, oxalkyloxy, oxazalkyloxy, azalkyloxy group optionally substituted with one or more F or CN; or C₅-C₆ aryl- or heterorylmethyloxy group where the aryl or heteroaryl moiety may optionally be substituted with one or more C₁-C₃ alkyl, C₁-C₃ alkoxy, halogen or CN groups and R₅, Q, R_{y}, Rₓ, X₃, n are as defined under formula (I)

3-fluoro-4-nitro-phenol, O-protected with a suitable protecting group (Pg) such as THP, is reacted with 4-aminomethyl-cyclohexanecarboxylic acid methyl ester to afford compound 23 which can then be reduced to the dianiline 24 using standard reduction procedures. Cyclization of 24 with CDI or triphogene gives the intermediate 25 which can be alkylated to 26 using standard alkylation procedures. Hydrolysis of 26 to the corresponding carboxylic acid 27 and subsequent O-deprotection affords compound 28 which is converted into its methyl ester derivative 29 using standard conditions. Alkylation of the phenol group of 29 with appropriate alkylating agents in presence of a base such as NaOH or K₂CO₃ gives intermediates of general formula 30. When R₁═OCHF₂, the alkylation can be done using procedures described in the literature (see for example U.S. Pat. No. 5,731,477). Intermediate 30 is then hydrolyzed to the corresponding carboxylic acids 31 and coupled with an amine in presence of an appropriate coupling agent to afford compounds of general formula 32.

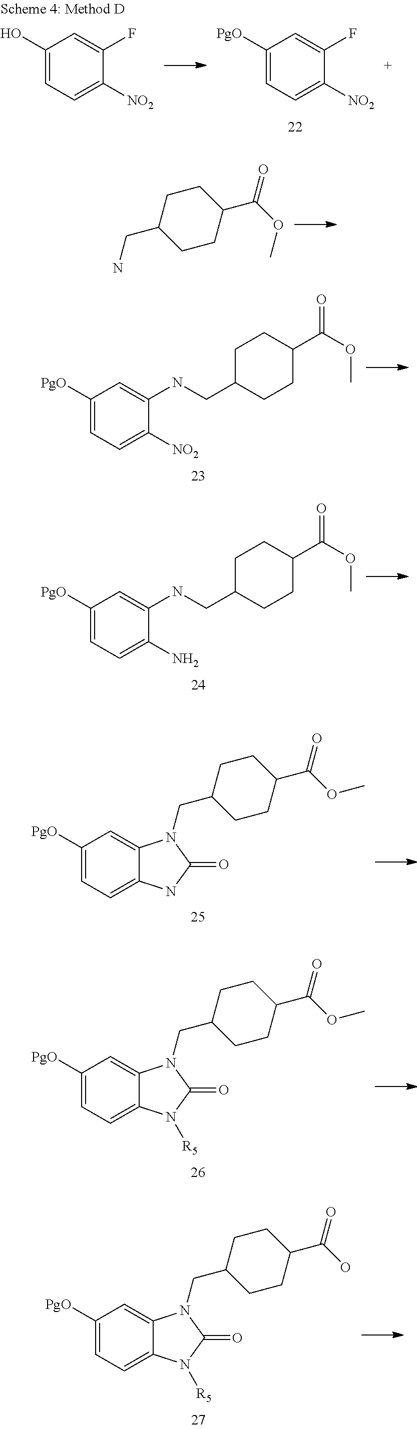

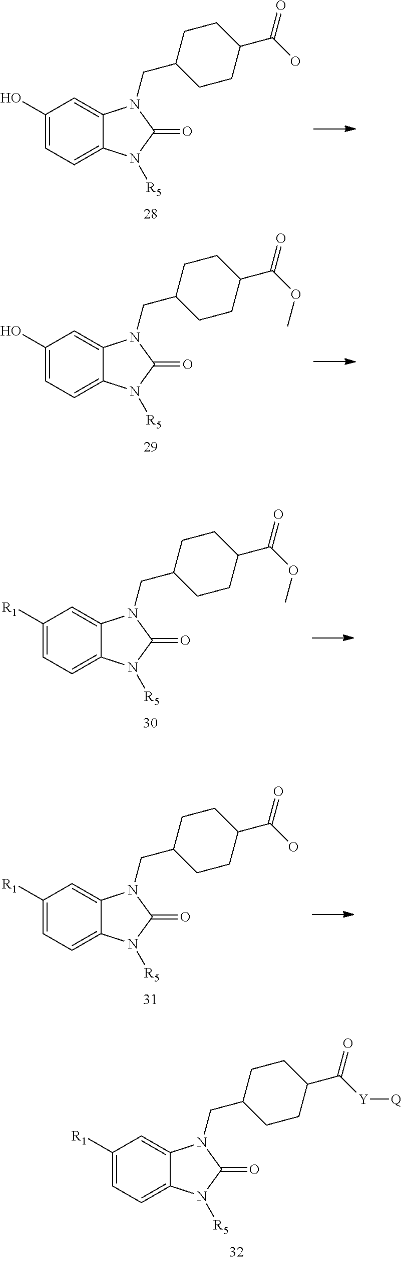

Compounds of formula Ie can be prepared according to Method E reported in scheme 5.

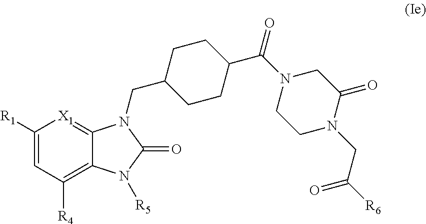

Wherein R₆ is a C₁-C₃ alkyl and R₁, X₁, R₄ and R₅ are as defined under formula (I)

Compounds of general formula 34 can be obtained by alkylation of intermediates 33, with the appropriate bromo alkyl ketone. Intermediates 33 can be obtained according method A reported in Scheme 1.

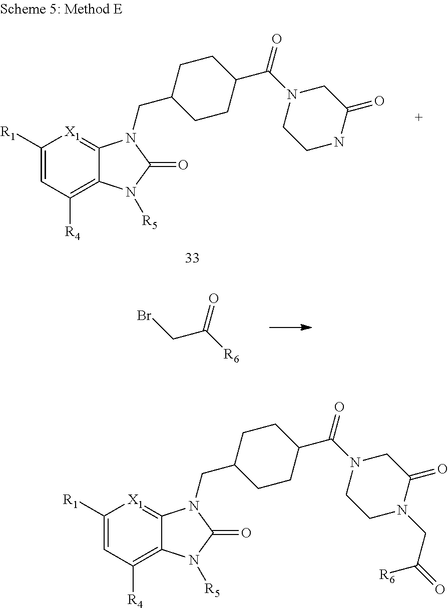

Compounds of formula If can be prepared according to Method F reported in scheme 6.

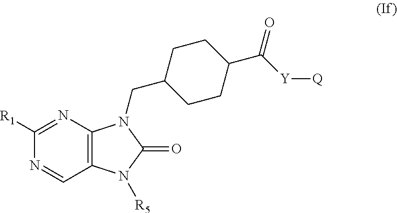

Wherein -Y-Q is

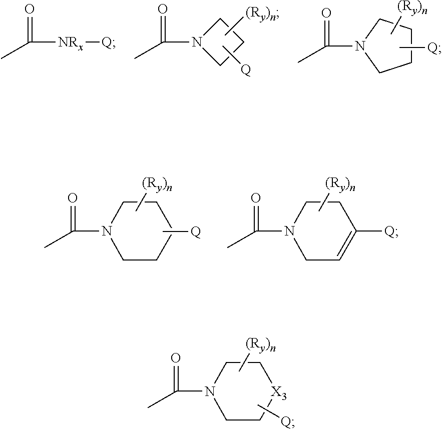

and R₁ is a dialkylamino, oxalkylammino, azalkylammino and R₅, Q, R_{y}, Rₓ, n are as defined under formula (I)

Intermediates 36 can be synthesized starting from 2,4-dichloro-5-nitro-pyrimidine 35 by two consecutive nucleophilic aromatic substitutions with a secondary amine (NR₇R₈) and 4-aminomethyl-cyclohexanecarboxylic acid methyl ester. Reduction of 36 to the dianiline 37 using standard reduction procedures followed by cyclization with triphosgene gives compounds of general formula 38 which are alkylated, following standard procedures, to 39. Hydrolysis of 39 gives intermediate 40 which can be coupled with an amine in presence of an appropriate coupling agent to afford compounds of general formula 41.

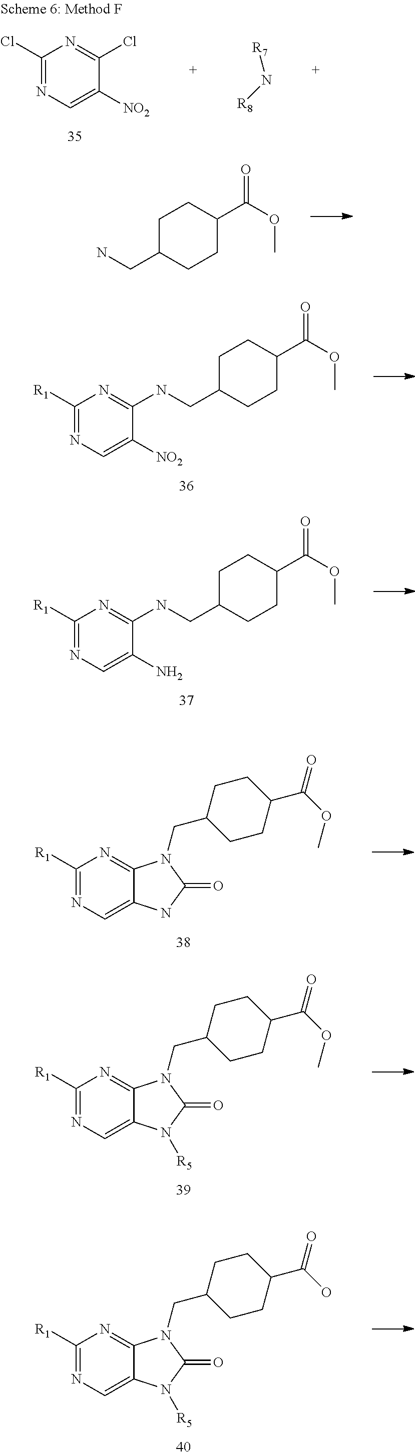

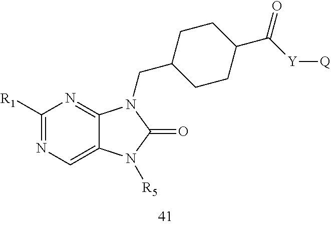

Compounds of formula Ig and Ih can be prepared according to Method G reported in scheme 7.

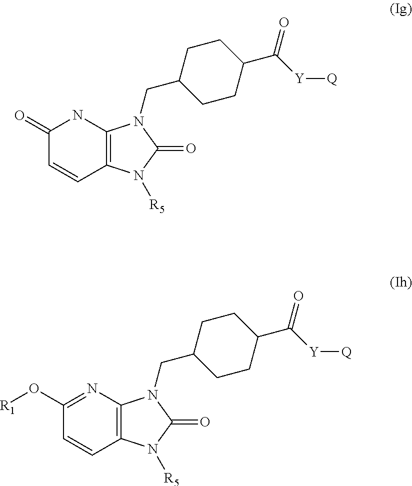

wherein Y-Q is

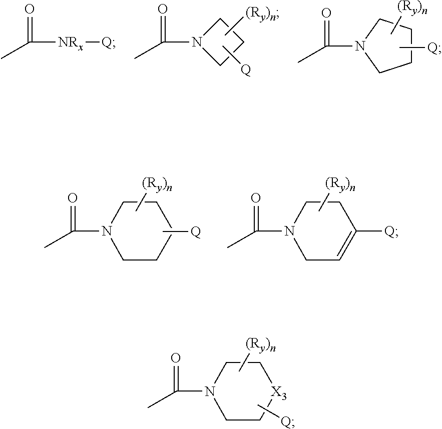

and R₁ is linear branched or cyclic C₁-C₆ alkyl, alkenyl, alkynyl, oxalkyl, oxalkenyl, oxalkynil, azalkenyl, azalkynyl, group optionally substituted with one or more F or CN; C₅-C₆ aryl- or heteroarylmethyl or C₅-C₆ aryl- where the aryl or heteroaryl moiety may optionally be substituted with one or more C₁-C₃ alkyl, C₁-C₃ alkoxy, halogen or CN groups and R₅, Q, R_{y}, Rₓ, n, X₃ are as defined under formula (I).

Compound 42 can be synthesized starting from the commercially available 2-chloro-6-methoxy-3-nitropyridine according to general method A described in scheme 1. Intermediate 43 can be obtained by reaction of 42 with chlorotrimetilsilane and then subjected to coupling with an amine in presence of an appropriate coupling agent to afford compounds of general formula 44. Alternatively intermediates 43 can be reacted with methanol in presence of a strong acid to give intermediates 45. O-Alkylation of the pyridone moiety, affords derivatives 46 which can then be hydrolysed to 47 and react with an amine to give compounds of general formula 48.

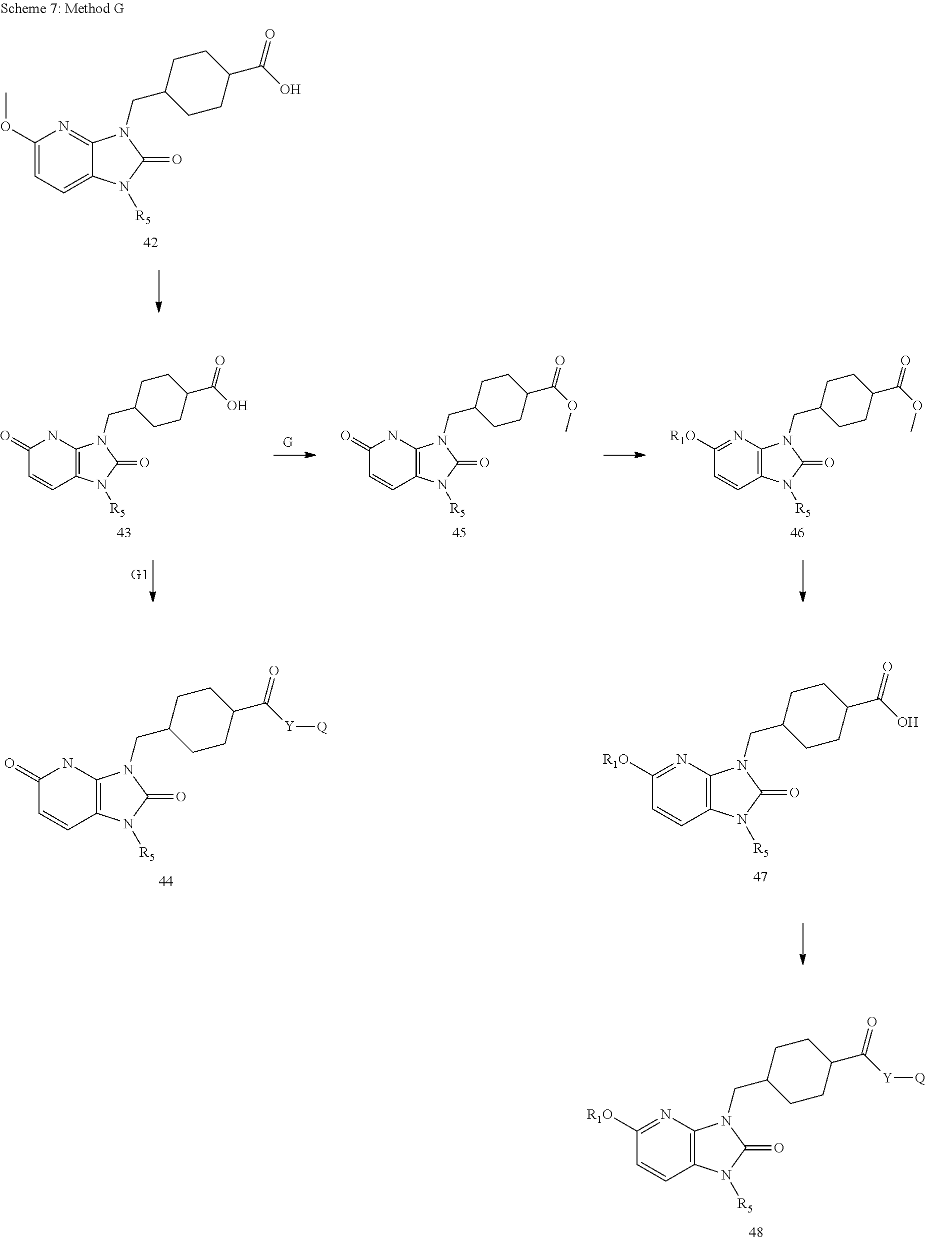

Compounds of formula Ii can be prepared according to Method H reported in scheme 8.

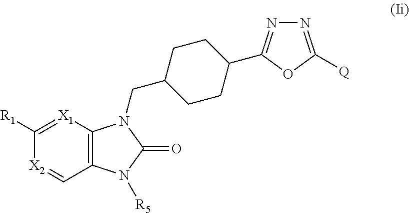

Wherein Q is C₁-C₆ linear branched or cylic alkyl, oxyalkyl, dioxalkyl; a C₅-C₁₀ aryl or heteroaryl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl and R₁, X₁, X₂, R₅ as defined under formula (I).

Coupling of compounds with general formula 49 with carboxylic acids hydrazides affords diacylhydrazides 52 which can be cyclization to give 53. Alternatively 49 is reacted with hydrazinecarboxylic acid tert-butyl ester to give intermediates 50 which, after deprotection to 51, is coupled with a carboxylic acid to give compounds of formula 52. Ring closure of 52 using standard literature procedures gave compounds of general formula 53. Compounds of general formula 49 can be synthesized according to the previously described method A reported in Scheme 1.

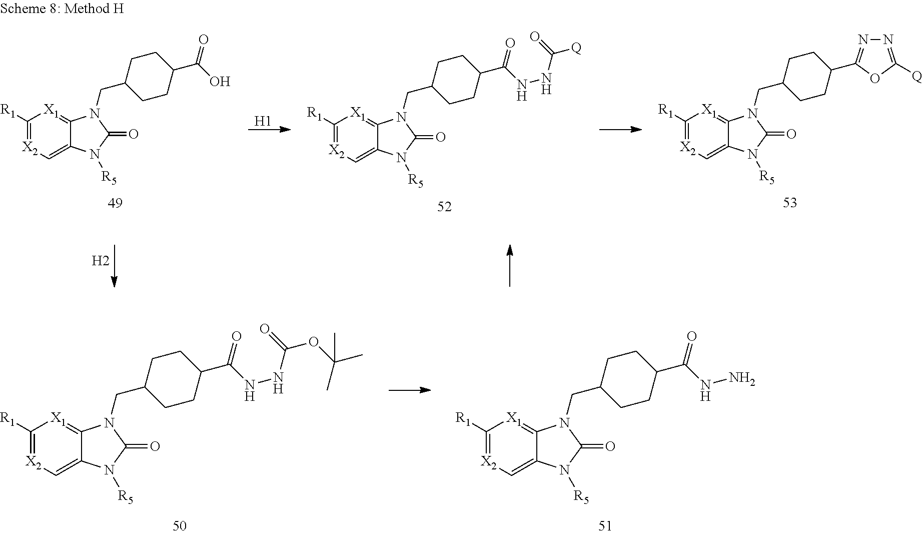

Compounds of formula Il can be prepared according to Method I reported in scheme 9.

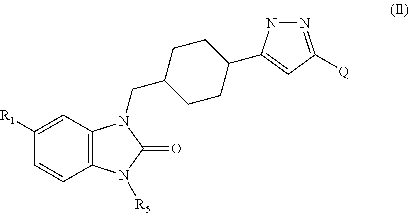

Wherein Q is a C₅-C₁₀ aryl or heteroaryl group optionally substituted with 1, 2 or 3 group selected from the list of C₁-C₆ linear branched or cyclic alkyl, oxalkyl, alkylamino, alkylamino carbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl and R₁, R₅ are as defined under formula (I).

Coupling of 49 with O-methyl-hydroxylamine gives the Weinreb amide intermediates 55 which are converted to ketones 56 following standard procedures known to those skilled in the art. Treatment of 56 with a strong base in presence an activated carboxylic acid affords the β-diketones 57 which can be cyclised to pyrazoles 58 by treatment with hydrazine. Compounds of general formula 49 can be synthesised according to previously described methods.

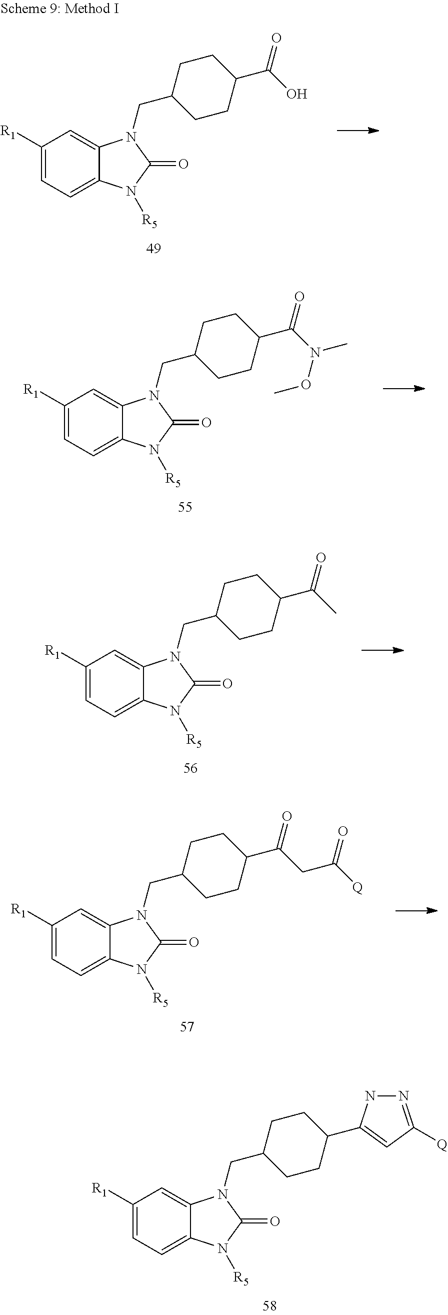

Compounds of formula Im can be prepared according to Method L reported in scheme 10.

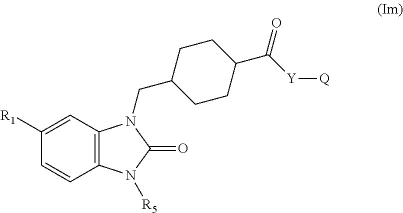

wherein Y-Q

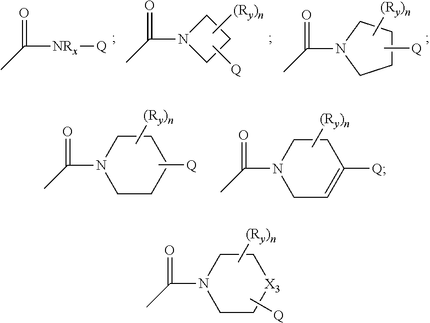

R₁ is OH; linear branched or cyclic C₁-C₆ alkyl, alkenyl, alkynyl, oxalkyl, oxalkenyl, oxalkynil, azalkenyl, azalkynyl, alkyloxy, oxalakyloxy, oxazalkyloxy, azalkyloxy, dialkylamino, oxalkylammino, azalkylammino, group optionally substituted with one or more F or CN; C₅-C₆ aryl- or heteroarylmethylammino or C₅-C₆ aryl- or heterorylmethyloxy group where the aryl or heteroaryl moiety may optionally be substituted with one or more C₁-C₃ alkyl, C₁-C₃ alkoxy, halogen or CN groups, and R₅, Q, Rₓ, R_{y}, X₃ and n are as defined under formula (I).

Compound 59 can be obtained according to procedures described in Scheme 1. Intermediate 60 can be obtained starting from the corresponding bromo intermediate 59 by methods known to those skilled in the art such as Sonogashira or Suzuki coupling. Hydrolysis of 60 gives compounds of general structure 61 which can be coupled with an amine in presence of a coupling agent to afford compounds 62. Alternatively 59 can be transformed in the corresponding boronate 63 which can be subjected to Chan-Lam coupling to obtain compounds of general formula 64. Hydrolysis of 64 gives the corresponding carboxylic acids 65 which can be coupled with an amine to give compounds 66. Intermediate 63 can be oxidized to give the corresponding phenol 67 which, after hydrolisys of the ester moiety, can be reacted with an amine to afford 69. Alternatively compound 67 can be O-alkylated to 70. Hydrolysis of 70 gives the carboxylic acid 71 which is reacted with an amine following standard procedures to afford 72.

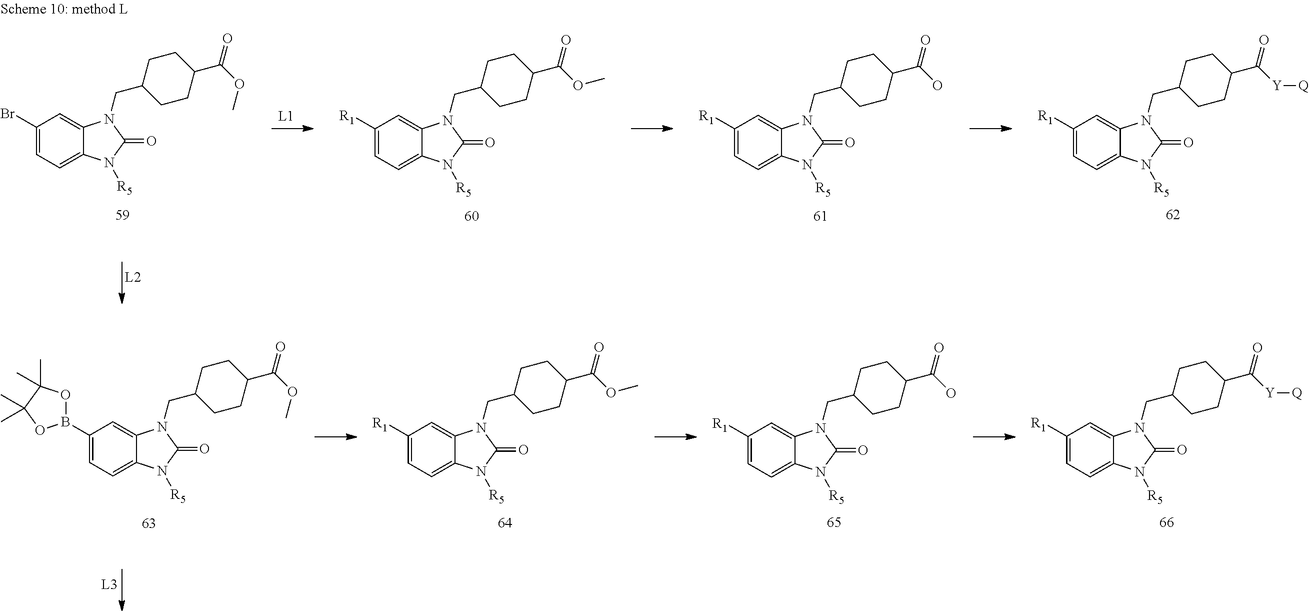

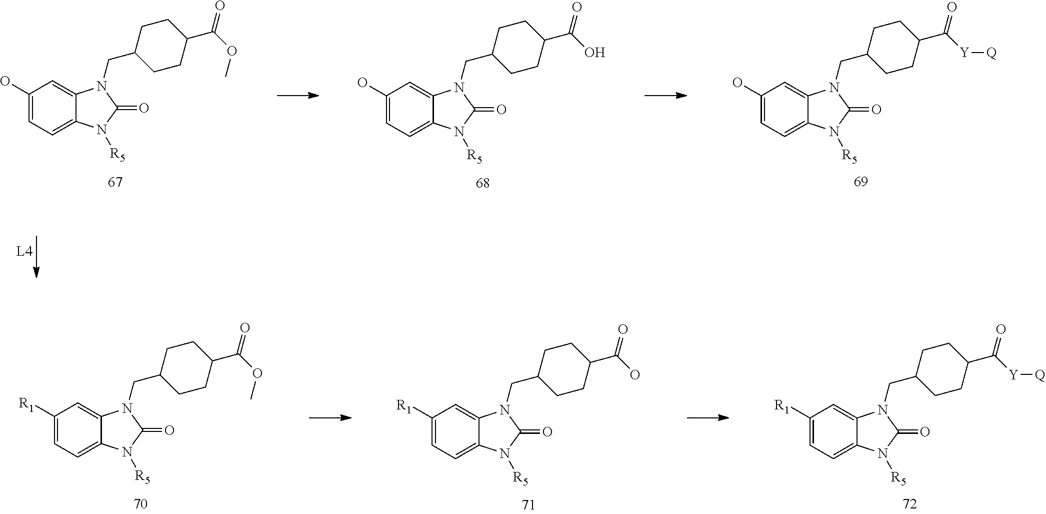

Compounds of formula In can be prepared according to Method M reported in scheme 11.

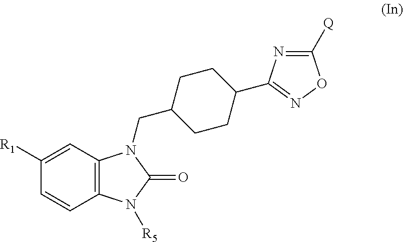

Wherein Q is C₁-C₆ a C₅-C₁₀ aryl or heteroaryl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, and R₁, R₅ are as defined under formula (I).

Synthesis of primary amides 74 followed by dehydration gives the intermediates 75 which can be converted into the amidoxime derivatives 76 by treatment with hydroxylamine. Coupling with a carboxylic acid followed by ring closure gives compounds of general formula 77. Intermediate 73 can be obtained using Method A reported in Scheme 1.

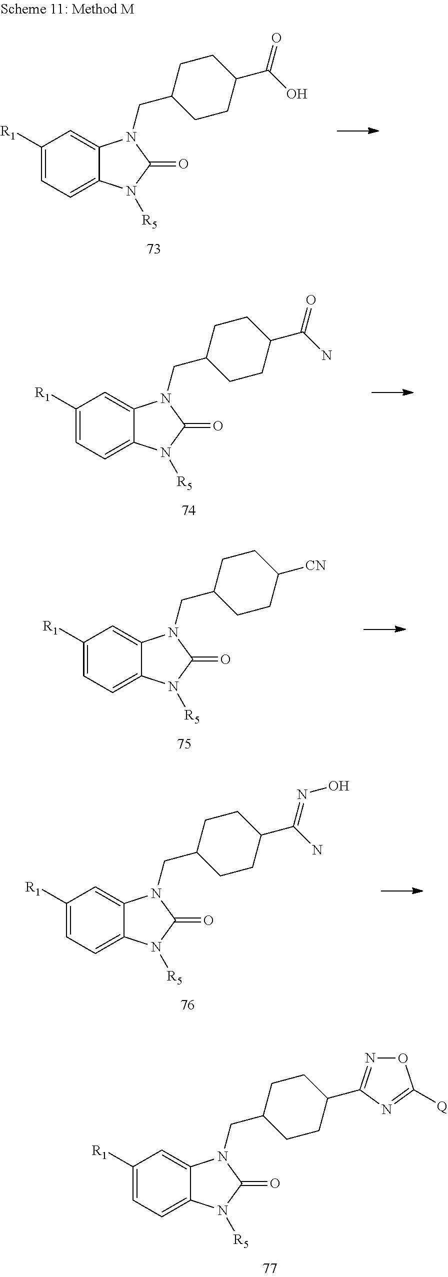

Compounds of formula Io can be prepared according to Method N reported in scheme 12.

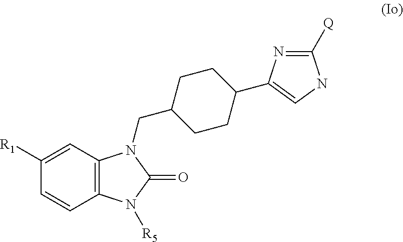

Wherein Q is an oxalkylamino, and R₁, R₅ are as defined under formula (I).

Reaction of the acyl chlorides 79 with trimethylsilyldiazomethane gives the intermediate 80 which can be converted into the α-bromo ketone 81 by treatment with hydrobromic acid. Reaction of 81 with an acylguanidine gives compounds of general formula 82. Intermediate 73 can be obtained using Method A reported in Scheme 1.

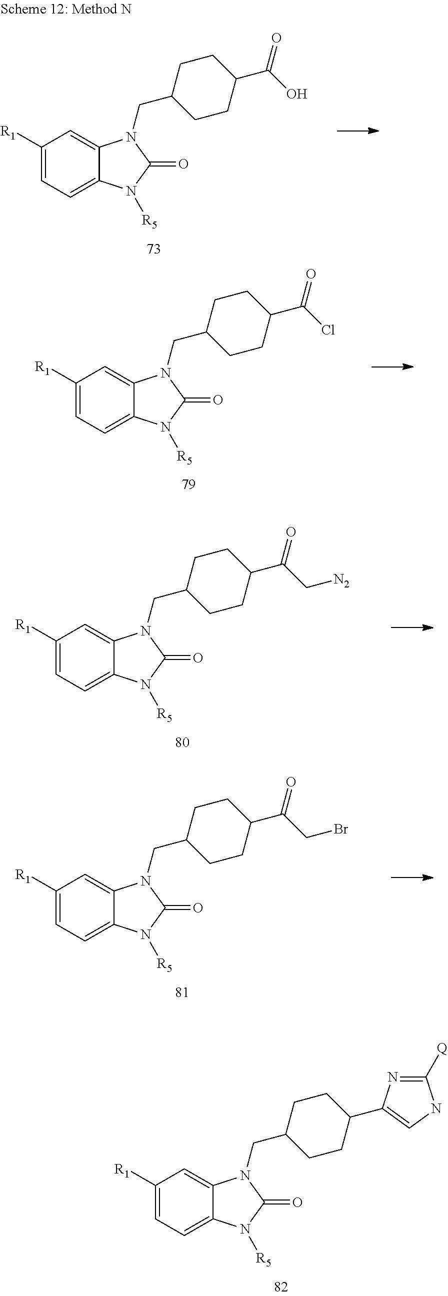

Compounds of formula Ip can be prepared according to Method P reported in scheme 13.

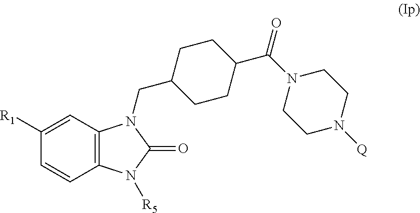

wherein Q is a C₅-C₁₀ aryl or heteroaryl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl and R₁ and R₅ are as defined under formula (I).

Coupling of 73 with 1-Boc-piperazine according to standard procedures gives compound of general formula 87. Deprotection of 87 affords the intermediate 88 that can then be functionalized by methods known to those skilled in the art such as, Buchwald couplings to give compounds of general formula 89. Intermediate 73 can be obtained using Method A2 reported in Scheme 1

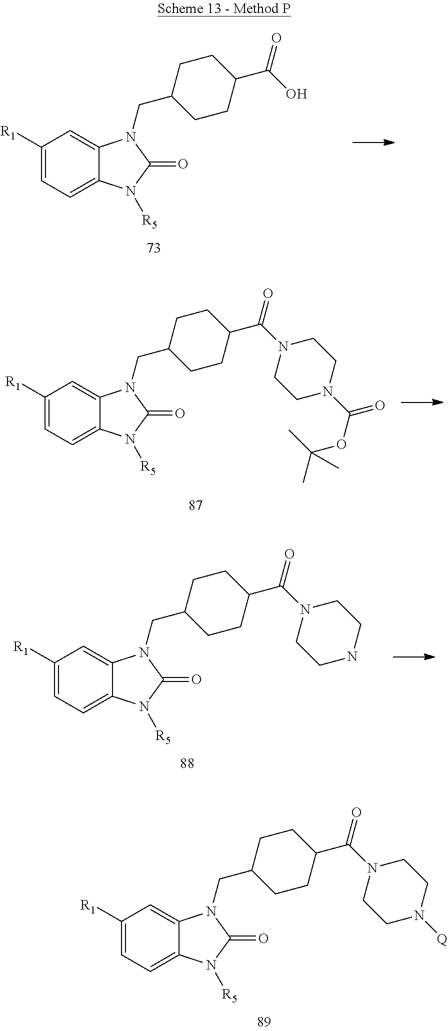

Assays Used to Identify Small Molecule Inhibitors of the Wnt Signaling Pathway.

The pharmacological activity of the exemplary compounds of the invention was first demonstrated in vitro in a Wnt reporter assay.

A Wnt-responsive Luciferase (TCF-Luciferase (Firefly) and a Wnt-independent (Renilla Luciferase (TA-Renilla) reporter plasmid (alone and in combination) were stably transfected in DBTRG-05MG glioblastoma cell line (ATCC) which according to the Wellcome Trust Sanger Institute Database showed no mutations involving APC, Axin and/or β-catenin genes and then considered to have an intact Wnt pathway cascade.

TCF-Luciferase:

Three copies of a 4×TCF responsive elements were cloned into the pcDNA3.1/Zeo(+) vector (Invitrogen) after deletion of the constitutive CMV promoter and the insertion of the Firefly Luciferase from Promega (phFL-TK) to measure the activity of the Wnt/β-catenin pathway. The resulting plasmid was sequenced for quality control.

TA-Renilla:

Both vectors (pcDNA3.1/Hygro(−) from Invitrogen) and phRL-TK were digested with restriction enzymes Mlu1 and BamH1 and ligated by T4-Ligase to form the final construct, containing the full length cDNA for hRL (human codons optimized Renilla Luciferase) with in 5′ the TA-minimal promoter and the backbone of the mammalian expression vector pcDNA3.1/Hygro(−) in which the constitutive CMV promoter was ablated. The construct was fully sequenced for quality control and used as internal control for cell number and toxicity.

Cells were grown in 20 μg/ml Zeocin and 20 μg/ml Zeocin plus 30 μg/ml Hygromicin respectively. The cells were plated at a density of 6500 cells/well in poly-D-lysine pre-treated 96 well-plates.

IC₅₀ Determination:

36 hours after plating cells were incubated with 8-points dilutions compound (0.6% DMSO (v/v)). Each compound was tested in triplicate in single plate. Luciferase detection was done with Dual-Luciferase Reporter Assay System (Promega). 24 hours after compound addition, media was removed and 30 μl of 1× lysis buffer was added to each well for 30 minutes. To each well 45 μl of Dual-Glo Luciferase reagent (Promega) were added and after 1 second delay Luciferase was detected for 1 second using Mithras LB940 instrument. After Firefly luciferase quantification 45 μl of Dual Stop & Glo reagent (Promega) were added to each well and Firefly Renilla was detected using the same parameters described before.

Data were expressed as % of control for Firefly and Renilla luciferase independently; values were calculated using XLFit version 4.2, with a four parameters sigmoid function (XLFit model 205).

A secondary screen using a luciferase biochemical assay enabled the identification of compounds acting directly on the enzyme (luciferase modulators and/or quenchers) rather than true inhibitors of the Wnt pathway.

Luciferase Assay:

Quantilum recombinant Luciferase (Promega) was diluted 10⁶-fold in 1× Cell Culture Lysis Reagent (Promega) containing 1 mg/ml acetylated BSA. Five microliters of compound dilution (10 μM final) was then mixed with 35 μl of diluted Quantilum recombinant Luciferase in a 96-well white plate. To each well 20 μl of LAR1 (Luciferase assay reagent from Promega) were added and luciferase was detected for 1 second with Mithras LB940 instrument. Each compound was tested in single data point on two different copy cell plates. Data were expressed as % of negative control (DMSO).

Other Assays

The pharmacological activity of the compounds of the invention may be tested in vitro for growth inhibition against tumour cell lines. Such cell lines may, for example be representative of glioblastoma (such as DBTRG-05MG), or colorectal (for example DLD-1, HCT116) cancer. The different genetic background of the cancer cell will facilitate to understand to which level of the pathway the compounds work. If the cells harbour a truncated APC allele, the destruction complex activity is affected; if cells carry a gain of function mutation in the β-catenin gene, which prevents β-catenin protein degradation, this leads to constitutive activation of downstream genes. There are many assays available for testing the growth inhibition. Such assays include the so called soft agar assay (Freedman et al., Cell 3 (1974), 355-359 and Macpherson et al., Virology 23 (1964), pp. 291-294) whereby the growth inhibition does not depend from adhesion of the cells to the plastic material of the well where the assay takes place.

Soft Agar Anchorage Independent Assay

DBTRG cells were seeded into a 24-well format in the presence of 25 carrier alone or compound (0.6% DMSO (v/v)). Each well is composed of two agar layers: the bottom layer consists of 0.6% Agar while the top has 0.35% Agar plus cells and compound. Cells (2500 per well) were incubated with 7 34 points dilution compound the day of the plating and the colonies were scored 3 weeks later after o/n staining with MTT solution. Imaging and counting of the colonies was done with the GelCount™ instrument (Oxford Optronix, UK). For IC50 determination the data were expressed as % of control, values were calculated using XLFit version 4.2, with a four parameters sigmoid 5 function (XLFit model 205).

The pharmacological activity of the compounds of the invention may further be tested in vivo in animal models mimicking the disease. These animal models may include those where the cancerous cells are implanted subcutaneously or orthotopically.

Formulation and Administration

Compounds under formula I are formulated preferably in admixture with a pharmaceutically acceptable carrier, excipient or the like. In general, it is preferable to administer the pharmaceutical composition in orally-administrable form, but certain formulations may be administered via a parenteral, intravenous, intramuscular, transdermal, buccal, subcutaneous, suppository, nasal or other route.

One of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications (salt formulation, esterification, etc.) which are well within the ordinary skill in the art. It is also well within the routineer's skill to modify the route of administration and dosage regimen of a particular compound in order to manage the pharmacokinetics of the present compounds for maximum beneficial effect in patients.

In certain pharmaceutical dosage forms, the pro-drug form of the compounds, especially including ester and ether derivatives, as well as various salt forms of the present compounds, are preferred. One of ordinary skill in the art will recognize how to readily modify the present compounds to pro-drug forms to facilitate delivery of active compounds to a targeted site within the host organism or patient. The routineer also will take advantage of favourable pharmacokinetic parameters of the pro-drug forms, where applicable, in delivering the present compounds to a targeted site within the host organism or patient to maximize the intended effect of the compound.

Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount effective to alleviate the symptoms of the subject being treated.

While human dosage levels have yet to be optimized for the compounds of the invention, generally, a daily dose is from about 0.05 mg/kg to about 100 mg/kg of body weight. The amount of active compound administered will, of course, be dependent on the subject and disease state being treated, the severity of the affliction, the manner and schedule of administration and the judgment of the prescribing physician.

For purposes of the present invention, a prophylactically or preventive effective amount of the compositions according to the present invention (i.e., an amount which substantially reduces the risk that a patient will either succumb to a disease state or condition or that the disease state or condition will worsen) falls within the same concentration range as set forth above for therapeutically effective amounts and is usually the same as a therapeutically effective amount.

In some embodiments of the present invention, one or more compounds of formula (I) are administered in combination with one or more other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition.

Rather, so long as both (or more) agents remain in the subject's body, they are considered to be administered “in combination”.

### EXAMPLES

All reagents and solvents were obtained commercially. Air and moisture sensitive liquid solutions were transferred via syringe. The course of reactions was followed by thin-layer chromatography (TLC) and/or liquid chromatography-mass spectrometry (HPLC-MS or UPLC-Ms). TLC analyses were performed on silica (Merck 60 F254) and spots revealed by UV visualisation at 254 nm and KMnO₄ or ninhydrin stain. Purifications by column chromatography were performed using silica cartridges isolute flash Si or silica (Merck 60) or with flash purification instruments from Biotage. Compounds purities were above 90%.

All nuclear magnetic resonance spectra were recorded using a Bruker Avance AV 400 System (400.13 MHz for ¹H) equipped with BBI a probe.

Analytical Methods

Method a

Analytical HPLC-MS were run using a Waters 2795 separation module equipped with a Waters Micromass ZQ (ES ionisation) and Waters PDA 2996, using a Gemini NH C18 3.0 μm 2.00×50 mm column Temperature: 40° C. UV Detection at 215 nm and 254. ESI+ detection in the 80-1000 m/z range. Gradient: 0.1% formic acid/water and 0.1% formic acid/acetonitrile with gradient 95/5 to 5/95 flow 1.0 ml/min over 10 minutes.

Method b

Analytical HPLC-MS were run using a Waters 2795 separation module equipped with a Waters Micromass ZQ (ES ionisation) and Waters PDA 2996, using a Gemini NH C18 3.0 μm 2.00×50 mm column Temperature: 40° C. UV Detection at 215 nm and 254. ESI+ detection in the 80-1000 m/z range. Gradient: 0.1% formic acid/water and 0.1% formic acid/acetonitrile with gradient 95/5 to 5/95 flow 1.0 ml/min over 5 minutes.

Method c

Analytical HPLC-MS were run using a Waters 2795 separation module equipped with a Waters Micromass ZQ (ES ionisation) and Waters PDA 2996, using a X-Bridge C18 3.5 μm 2.10×50 mm column Temperature: 40° C.UV Detection at 215 nm and 254. ESI+ detection in the 80-1000 m/z range Gradient: 0.1% ammonia/water and acetonitrile with gradient 85715 to 95/5 flow 0.8 ml/min over 10 minutes.

Method d

Analytical HPLC-MS were run using a Waters 2795 separation module equipped with a Waters Micromass ZQ (ES ionisation) and Waters PDA 2996, using a X-Bridge C18 3.5 μm 2.10×50 mm column Temperature: 40° C.UV Detection at 215 nm and 254. ESI+ detection in the 80-1000 m/z range Gradient: 0.1% ammonia/water and acetonitrile with gradient 85715 to 95/5 flow 0.8 ml/min over 5 minutes.

Method e

Analytical UPLC-MS were run using a Acquity Waters UPLC with equipped with a Waters SQD (ES ionization) and Waters Acquity PDA detector, using a column BEH C18 1.7 μm, 2,1×5.00. Temperature: 40° C. UV Detection at 215 nm and 254. ESI+ detection in the 80-1000 m/z range Gradient 0.1% ammonia/water and acetonitrile with a gradient 85/15 to 5/95 flow: 0.8 ml/min over 3 min.

Method f

Analytical UPLC-MS were run using a Acquity Waters UPLC with equipped with a Waters SQD (ES ionization) and Waters Acquity PDA detector, using a column BEH C18 1.7 μm, 2.1×5.00. Temperature: 40° C. UV Detection at 215 nm and 254. ESI+ detection in the 80-1000 m/z range. Gradient 0.1% formic acid/water and 0.1% formic acid/CH3CN with a gradient 95/5 to 5/95 flow: 0.6 ml/min over 3 minutes.

Preparative HPLC Method

Method a

Preparative HPLC was run using a Waters 2767 system with a binary gradient Module Waters 2525 pump and coupled to a Waters Micromass ZQ25 (ES) or Waters 2487 DAD, using a Gemini NX C18 5 μm, 100×21.2. Gradient 0.1% formic acid/water and 0.1% formic acid/methanol flow: 40 ml/min.

Method b

Preparative HPLC was run using a Waters 2767 system with a binary gradient Module Waters 2525 pump and coupled to a Waters Micromass ZQ 25 (ES) or Waters 2487 DAD, using a X-Bridge C18 5 μm 19×150. Gradient 0.1% ammonia/water and methanol flow: 17 ml/min.

Method c

Preparative HPLC was run using a Waters 2767 system with a binary gradient Module Waters 2525 pump and coupled to a Waters MS3100 SQ or Waters 2487 DAD, using a X-Bridge C18 5 μm 19×150. Gradient 0.1% formic acid/water and 0.1% formic acid/methanol flow: 17 ml/min.

### Example 1

### (Method A2): Trans-4-(5-Fluoro-6-Methoxy-3-methyl-2-oxo-2,3-dihydro-benzoimidazol-1-ylmethyl)-cyclohexanecarboxylic acid pyridine-4-ylamide

### 1,4-Difluoro-2-methoxy-5-nitro-benzene

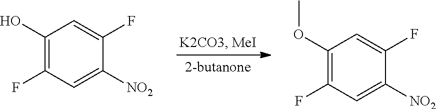

K₂CO₃ (4.77 g, 34.49 mmol) and a catalytic amount of 1,4,7,10,13,16-hexaoxacyclooctadecane were added to a stirred solution of 2,5-difluoro-4-nitro-phenol (3.02 g, 17.25 mmol) in 2-butanone (8 mL) at room temperature. After 30 minutes methyl iodide (2.25 mL, 36.22 mmol) was added and the reaction mixture was heated at 40° C. over weekend. The reaction mixture was concentrated under reduced pressure. AcOEt (50 mL) and H₂O (50 mL) were added. The organic phase was separated and the aqueous phase was back extracted with AcOEt (3×20 mL). The organic layers were collected, washed with brine (50 mL), dried over Na₂SO₄ and concentrated under reduced pressure to afford 3.04 g of the titled compound as yellow solid (yield 92%).

C7H5F2NO3, calculated [189.12] found: No Mass response RT=1.32, (method f)

¹HNMR (DMSO) δ: 3.97 (3H, s), 7.47-7.52 (1H, m), 8.13-8.18 (1H, m).

### Trans-4-[(4-Fluoro-5-Methoxy-2-nitro-phenylamino)-methyl]-cyclohexane carboxylic acid methyl ester

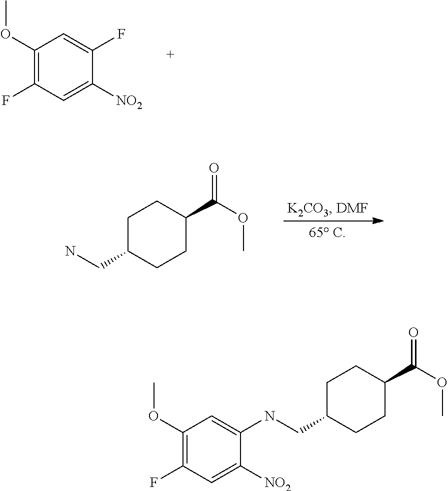 [SMILES: COc1cc(F)c([N+](=O)[O-])cc1F ; COC(=O)[C@H]1CC[C@H](CN)CC1 ; COc1cc(NC[C@H]2CC[C@H](C(=O)OC)CC2)c([N+](=O)[O-])cc1F]

K₂CO₃ (10.02 g, 72.51 mmol) was added to a stirred solution of 1,4-Difluoro-2-methoxy-5-nitro-benzene (2.77 g, 14.50 mmol) in DMF (15 mL). After 30 minutes trans-4-aminomethyl-cyclohexanecarboxylic acid methyl ester (3.00 g, 14.50 mmol) was added and the reaction mixture was heated at 65° C. 3 hours. The reaction mixture was concentrated under reduced pressure and crude was diluted with DCM (50 mL) and H₂O (50 mL). The organic phase was separated and the aqueous phase was back extracted with DCM (3×20 mL). The organic layers were collected, washed with brine (50 mL), dried over Na₂SO₄, filtered and concentrated under reduced pressure to afford 4.79 g of the titled compound (yield 98%).

¹HNMR (DMSO) δ: 1.02-1.11 (2H, m), 1.29-1.39 (2H, m), 1.61-1.70 (1H, m), 1.80-1.84 (2H, m), 1.89-1.94 (2H, m), 2.22-2.30 (1H, m), 3.25-3.28 (2H, m), 3.57 (3H, s), 3.96 (3H, s), 6.46-6.48 (1H, m), 7.83-7.86 (1H, m), 8.46-8.49 (1H, m).

C16H21FN2O5, Calculated [340.35]. found [M+H⁺] 341, RT=1.71 (method f).

### Trans-4-[(2-Amino-4-fluoro-5-Methoxy-phenylamino)-methyl]-cyclohexane carboxylic acid methyl ester

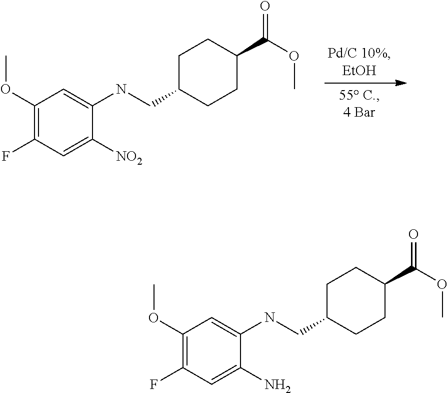 [SMILES: COc1cc(NC[C@H]2CC[C@H](C(=O)OC)CC2)c([N+](=O)[O-])cc1F ; COc1cc(NC[C@H]2CC[C@H](C(=O)OC)CC2)c(N)cc1F]

trans-4-[(4-Fluoro-5-Methoxy-2-nitro-phenylamino)-methyl]-cyclohexane carboxylic acid methyl ester (4.79 g, 14.09 mmol) was suspended in 50 mL of EtOH, mixed with Pd/C 10% (0.50 g) and transferred in a Eyela reactor. The mixture was left under 4 bar of hydrogen at 55° C. for 4 hours then it was filtered through cellulose. The cellulose was washed with EtOH (300 mL). The organic solution was concentrated under reduced pressure to give 4.28 g of the titled compound (yield 98%).

¹HNMR (DMSO) δ: 0.94-1.04 (2H, m), 1.25-1.36 (2H, m), 1.49-1.58 (1H, m), 1.88-1.91 (4H, m), 2.22-2.29 (1H, m), 2.80-2.83 (2H, m), 3.57 (3H, s), 3.67 (3H, s), 4.18-4.21 (1H, m), 4.39 (2H, bp), 6.13-6.15 (1H, m), 6.36-6.40 (1H, m).

### Trans-4-(5-Fluoro-6-Methoxy-2-oxo-2,3-dihydro-benzoimidazol-1-ylmethyl)-cyclohexanecarboxylic acid methyl ester

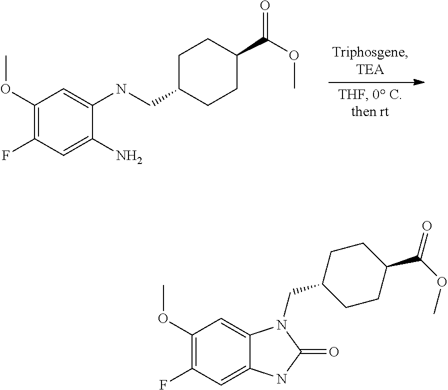 [SMILES: COc1cc(NC[C@H]2CC[C@H](C(=O)OC)CC2)c(N)cc1F ; COc1cc2c(cc1F)[nH]c(=O)n2C[C@H]1CC[C@H](C(=O)OC)CC1]

Triphosgene (4.10 g, 13.81 mmol) was added portionwise to a stirred solution of trans-4-[(2-Amino-4-fluoro-5-methoxy-phenylamino)-methyl]-cyclohexanecarboxylic acid methyl ester (4.28 g, 13.81 mmol) and TEA (1.92 mL, 13.81 mmol) in THF (40 mL) cooled to 0° C. The reaction mixture was left to warm to room temperature and it was left overnight. H₂O (50 mL) was slowly added to the reaction mixture then THF was removed under reduced pressure. The formed precipitate was filtered, washed with H₂O (3×20 mL) and dried to give 4.51 g of the titled compound (yield 97%).

¹HNMR (DMSO) δ: 1.01-1.10 (2H, m), 1.19-1.29 (2H, m), 1.60-1.63 (2H, m), 1.70-1.78 (1H, m), 1.85-1.88 (2H, m), 2.20-2.27 (1H, m), 3.55 (3H, s), 3.58-3.60 (2H, m), 3.81 (3H, s), 6.84-6.87 (1H, m), 7.00-7.02 (1H, m), 10.70 (1H, s)

C17H21FN2O4, Calculated [336.37]. found [M+H⁺] 337, RT=1.24 (method f).

### Trans-4-(5-Fluoro-6-Methoxy-3-methyl-2-oxo-2,3-dihydro-benzoimidazol-1-ylmethyl)-cyclohexanecarboxylic acid methyl ester

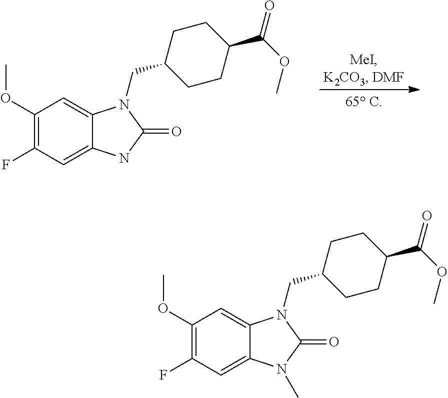 [SMILES: COc1cc2c(cc1F)[nH]c(=O)n2C[C@H]1CC[C@H](C(=O)OC)CC1 ; COc1cc2c(cc1F)n(C)c(=O)n2C[C@H]1CC[C@H](C(=O)OC)CC1]

MeI (1.11 mL, 17.86 mmol) was added to a stirred solution of trans-4-(5-Fluoro-6-methoxy-2-oxo-2,3-dihydro-benzoimidazol-1-ylmethyl)-cyclohexanecarboxylic acid methyl ester (1.50 g, 4.46 mmol) in DMF (16 mL) containing K₂CO₃ (0.80 g, 1.30 mmol). The reaction mixture was heated at 65° C. overnight then it was concentrated under reduced pressure. DCM (50 mL) and H₂O (50 mL) were added to the crude; the organic layer was separated and the aqueous phase was washed with DCM (3×20 mL). The organics layers were collected, washed with brine (50 mL), dried over Na₂SO₄, filtered and concentrated under reduced pressure. The crude material was purified by silica column (gradient of cyclohexane/AcOEt) to give 1.24 g of the titled compound (yield 81%).

¹HNMR (DMSO) δ: 1.01-1.11 (2H, m), 1.18-1.28 (2H, m), 1.60-1.63 (2H, m), 1.70-1.79 (1H, m), 1.84-1.88 (2H, m), 2.20-2.26 (1H, m), 3.25 (3H, s), 3.55 (3H, s), 3.63-3.65 (2H, m), 3.83 (3H, s), 7.07-7.09 (1H, m), 7.16-7.18 (1H, m).

C18H23FN2O4, Calculated [350.39]. found [M+H⁺] 351 RT=1.39 (method f).

### Trans-4-(5-Fluoro-6-Methoxy-3-methyl-2-oxo-2,3-dihydro-benzoimidazol-1-ylmethyl)-cyclohexanecarboxylic acid

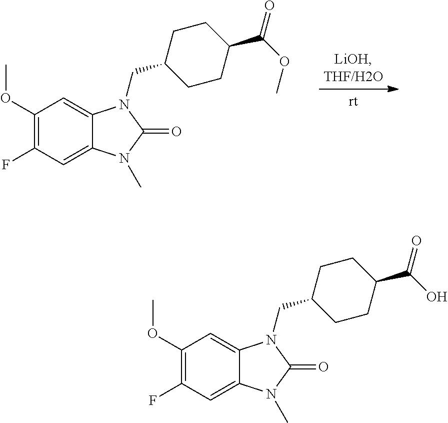 [SMILES: COc1cc2c(cc1F)n(C)c(=O)n2C[C@H]1CC[C@H](C(=O)OC)CC1 ; COc1cc2c(cc1F)n(C)c(=O)n2C[C@H]1CC[C@H](C(=O)O)CC1]

LiOH (0.13 g, 5.30 mmol) was added to a stirred solution of trans-4-(5-Fluoro-6-methoxy-3-methyl-2-oxo-2,3-dihydro-benzoimidazol-1-ylmethyl)-cyclohexanecarboxylic acid methyl ester (1.24 g, 3.53 mmol) in a mixture of THF (10 mL) and H₂O (3 mL). The reaction was stirred at room temperature overnight then it was concentrated under reduced pressure. The residue was diluted with H₂O (10 mL) and the pH adjusted to 3 using HCl 1.0 N. The solid obtained was filtered, washed with water (3×10 mL) and dried to give 1.19 g of the titled compound (yield quantitative).

¹HNMR (DMSO) δ: 1.00-1.09 (2H, m), 1.16-1.25 (2H, m), 1.59-1.62 (2H, m), 1.69-1.78 (1H, m), 1.84-1.87 (2H, m), 3.26 (3H, s), 3.63-3.65 (2H, m), 3.83 (3H, s), 7.07-7.09 (1H, m), 7.16-7.19 (1H, m), 12.00 (1H, bp).

C17H21FN2O4 Mass (calculated) [336.37]. found [M+H⁺]=337, RT=1.15 (method f).

### Trans-4-(5-Fluoro-6-Methoxy-3-methyl-2-oxo-2,3-dihydro-benzoimidazol-1-ylmethyl)-cyclohexanecarboxylic acid pyridine-4-ylamide

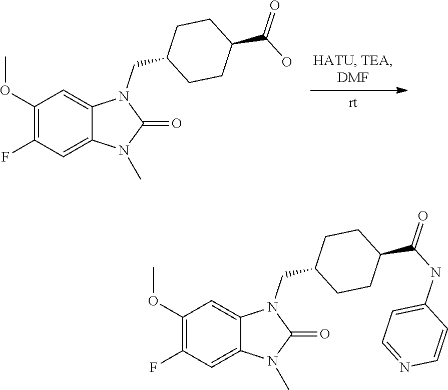 [SMILES: COc1cc2c(cc1F)n(C)c(=O)n2C[C@H]1CC[C@H](C(=O)O)CC1 ; COc1cc2c(cc1F)n(C)c(=O)n2C[C@H]1CC[C@H](C(=O)Nc2ccncc2)CC1]

A mixture of trans-4-(5-Fluoro-6-methoxy-3-methyl-2-oxo-2,3-dihydro-benzoimidazol-1-ylmethyl)-cyclohexanecarboxylic acid (110 mg, 0.33 mmol), TEA (55 μL, 0.39 mmol), HATU (149 mg, 0.39 mmol) and pyridin-4-ylamine (37 mg, 0.39 mmol) in DMF (2 mL) was stirred at room temperature overnight. The reaction mixture was concentrated under reduced pressure. The residue was dissolved in DCM (5 mL) washed with H₂O (5 mL) and then with NaOH 1.0 N (5 mL). The organic layer was concentrated under reduced pressure and crude material was tritured with CH₃CN to give 92 mg of the titled compound (yield 68%).

¹HNMR (DMSO) δ: 1.02-1.13 (2H, m), 1.29-1.39 (2H, m), 1.65-1.68 (2H, m), 1.76-1.85 (3H, m), 2.26-2.33 (1H, m), 3.26 (3H, s), 3.67-3.69 (2H, m), 3.84 (3H, s), 7.11-7.13 (1H, m), 7.17-7.19 (1H, m), 7.52-7.54 (2H, m), 8.36-8.37 (2H, m), 10.19 (1H, s).

C22H25FN4O3 Mass (calculated) [412.47]. found [M+H⁺]=413, RT=0.95 (method f).

### Example 2

### (Method A3): 5-Methoxy-1-methyl-3-[trans-4-(4-pyrimidin-2-yl-piperazine-1-carbonyl)-cyclohexylmethyl]-1,3-dihydro-benzoimidazol-2-one 2-Fluoro-4-methoxy-1-nitro-benzene

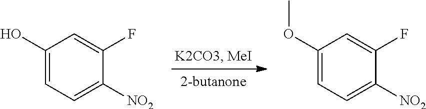

K₂CO₃ (35.20 g, 255 mmol) was added to a stirred solution of 3-Fluoro-4-nitro-phenol (20.00 g, 127.30 mmol) in 2-butanone (60 mL) at room temperature. After 30 minutes methyl iodide (8.72 mL, 140.00 mmol) was added and the reaction mixture was heated at 40° C. 22 hours. The mixture was concentrated under reduced pressure. AcOEt (400 mL) and H₂O (600 mL) were added. The organic phase was separated and the aqueous phase was back extracted with AcOEt (3×100 mL). The organic layers were collected, washed with brine (150 mL), dried over Na₂SO₄ and concentrated under reduced pressure. The obtained solid was dissolved in DCM (300 mL) and washed with NaOH 1N (200 mL). The DCM solution was concentrated under reduced pressure to afford 18.1 g of the titled compound (yield 83%).

¹HNMR (CDCl3) δ: 3.90 (s, 3H), 6.71-6.78 (2H, m), 8.07-8.12 (m, 1H).

### Trans-4-[(5-Methoxy-2-nitro-phenylamino)-methyl]-cyclohexanecarboxylic acid methyl ester

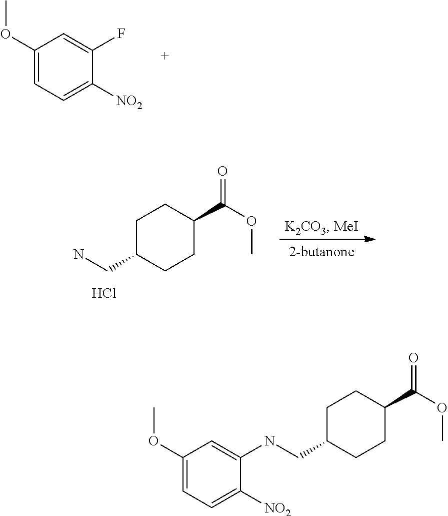 [SMILES: COc1ccc([N+](=O)[O-])c(F)c1 ; COC(=O)[C@H]1CC[C@H](CN)CC1 ; Cl ; COc1ccc([N+](=O)[O-])c(NC[C@H]2CC[C@H](C(=O)OC)CC2)c1]

K₂CO₃ (43.64 g, 315.80 mmol) was added to a stirred solution of 2-Fluoro-4-methoxy-1-nitro-benzene (18.00 g, 105.26 mmol) in DMF (60 mL). After 30 minutes trans-4-aminomethyl-cyclohexanecarboxylic acid methyl ester hydrochloride (21.79 g, 105.26 mmol) was added and the reaction mixture was heated at 50° C. 22 hours. The reaction mixture was filtered and the precipitate was washed with DCM (5×50 mL). The organic solution was concentrated to give 33.89 g of the titled compound (yield quantitative).

¹HNMR (DMSO) δ: 1.00-1.10 (2H, m), 1.27-1.38 (2H, m), 1.59-1.69 (1H, m), 1.79-1.83 (2H, m), 1.90-1.93 (2H, m), 2.22-2.29 (1H, m), 3.20-3.23 (2H, m), 3.56 (3H, s), 3.84 (3H, s), 6.26-6.31 (2H, m), 7.99-8.01 (1H, m), 8.38-8.41 (1H, m).

C16H22N2O5 Mass (calculated) [322.36]. found [M+H+]=323, RT=1.73 (method f).

### Trans-4-[(5-Methoxy-2-amino-phenylamino)-methyl]-cyclohexanecarboxylic acid methyl ester

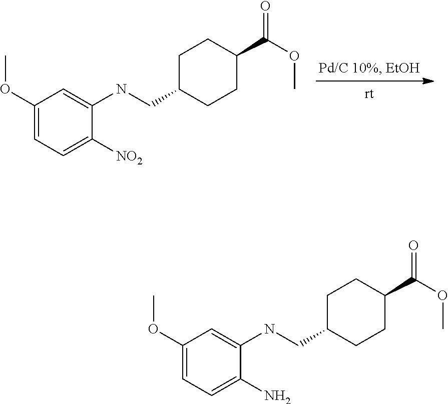 [SMILES: COc1ccc([N+](=O)[O-])c(NC[C@H]2CC[C@H](C(=O)OC)CC2)c1 ; COc1ccc(N)c(NC[C@H]2CC[C@H](C(=O)OC)CC2)c1]

Trans-4-[(5-Methoxy-2-nitro-phenylamino)-methyl]-cyclohexanecarboxylic acid methyl ester (33.90 g, 105.28 mmol) was dissolved in 350 mL of EtOH, mixed with Pd/C 10% (1.80 g) and transferred into an Ecoclave reactor. The mixture was left overnight with stirring under 5 bar of hydrogen then it was filtered through cellulose pads. The cellulose was washed with DCM (5×60 mL). The organic solution was concentrated under reduced pressure to give 27.46 g of the titled compound (yield 89%).

¹HNMR (DMSO) δ: 0.92-1.03 (2H, m), 1.23-1.34 (2H, m), 1.48-1.57 (1H, m), 1.85-1.92 (4H, m), 2.21-2.28 (1H, m), 2.1 (2H, d, J=6.0 Hz), 3.56 (3H, s), 3.57 (3H, s), 4.05 (2H, bp), 4.44 (1H, d, J=6.0 Hz), 5.93-5.95 (2H, m), 6.40-6.43 (1H, m).

### Trans-4-(6-Methoxy-2-oxo-2,3-dihydro-benzoimidazol-1-ylmethyl)-cyclo hexanecarboxylic acid methyl ester

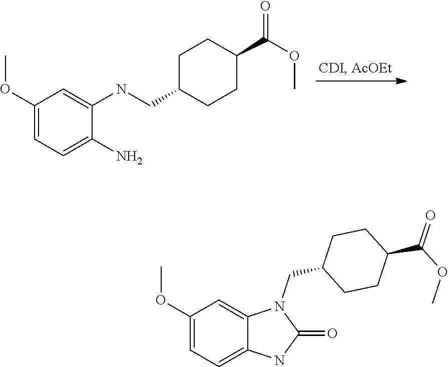 [SMILES: COc1ccc(N)c(NC[C@H]2CC[C@H](C(=O)OC)CC2)c1 ; COc1ccc2[nH]c(=O)n(C[C@H]3CC[C@H](C(=O)OC)CC3)c2c1]

CDI (38.13 g, 235.10 mmol) was added to a stirred solution of trans-4-[(5-Methoxy-2-amino-phenylamino)-methyl]-cyclohexanecarboxylic acid methyl ester (27.46 g, 94.04 mmol) in AcOEt (300 mL) under N₂. The reaction mixture was left overnight then H₂O (500 mL) was added. A precipitate formed and it was filtered, washed with AcOEt (3*30 mL) and discarded. The organic washes were collected to the mother liquors. The organic layer was separated and the aqueous phase was back extracted with AcOEt (3*100 mL). The organic layers were collected, washed with HCl 1.0N (300 mL) and brine (300 mL), dried over Na₂SO₄ and concentrated under reduced pressure. The dark brown solid was washed with Et₂O (3×100 mL) and dried under reduced pressure to give 23.19 g of the titled compound (yield 77%).

¹HNMR (DMSO) δ: 0.99-1.09 (2H, m), 1.17-1.28 (2H, m), 1.59-1.63 (2H, m), 1.67-1.77 (1H, m), 1.84-1.88 (2H, m), 2.18-2.26 (1H, m), 3.54 (3H, s), 3.6 (2H, d, J=7.2 Hz), 3.71 (3H, s), 6.52 (1H, dd, J=8.4 and 2.4 Hz), 6.73 (1H, d, J=2.4 Hz), 6.82 (1H, d, J=8.4 Hz), 10.56 (1H, s).

C17H22N2O4 Mass (calculated) [318.38]. found [M+H+]=319, RT=1.25 (method f).

### Trans-4-(6-Methoxy-2-oxo-2,3-dihydro-benzoimidazol-1-ylmethyl)-cyclo hexanecarboxylic acid

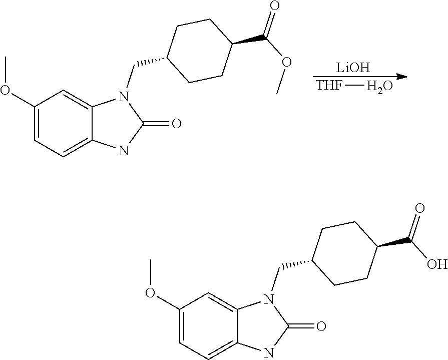

Trans-4-(6-Methoxy-2-oxo-2,3-dihydro-benzoimidazol-1-ylmethyl)cyclo hexanecarboxylic acid methyl ester (985 mg, 3.10 mmol) was dissolved in THF (6 mL), then a solution of LiOH (221 mg, 9.2 mmol) in H₂O (3 mL) was added and the resulting was stirred overnight at r.t. 5 mL of water were added, THF was removed under reduced pressure and HCl 1M was added to reach pH 4 with the formation of a white precipitate. The precipitate was filtered and washed with DCM (5 mL) and dried over reduced pressure to give 400 mg of the titled compound (yield 42%).

¹HNMR (DMSO) δ: 0.98-1.08 (2H, m), 1.14-1.24 (2H, m), 1.59-1.62 (2H, m), 1.68-1.76 (1H, m), 1.82-1.86 (2H, m), 3.26 (3H, s), 3.61 (2H, d, J=7.2 Hz), 3.73 (3H, s), 6.61 (1H, dd, J=2.0 and 8.4 Hz), 6.80 (1H, d, J=2.0 Hz), 6.99 (1H, d, J=8.4 Hz), 11.97 (1H, bs).

### 6-Methoxy-1-[trans-4-(4-pyrimidin-2-yl-piperazine-1-carbonyl)-cyclo hexylmethyl]-1,3-dihydro-benzoimidazol-2-one

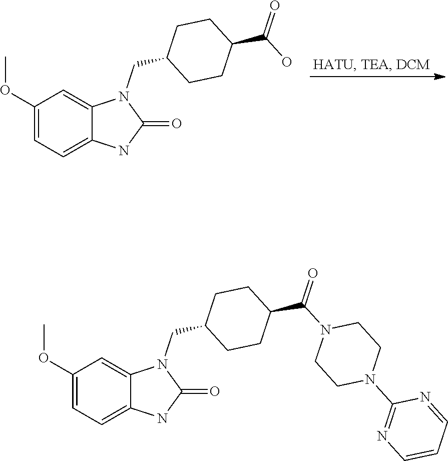 [SMILES: COc1ccc2[nH]c(=O)n(C[C@H]3CC[C@H](C(=O)O)CC3)c2c1 ; "HATU, TEA, DCM" ; COc1ccc2[nH]c(=O)n(C[C@H]3CC[C@H](C(=O)N4CCN(c5ncccn5)CC4)CC3)c2c1]

TEA (55 μL, 0.39 mmol), HATU (150 mg, 0.39 mmol) and 2-piperazin-1-yl-pyrimidine (65 mg, 0.39 mmol) were added to a solution of trans-4-(6-Methoxy-2-oxo-2,3-dihydro-benzoimidazol-1-ylmethyl)-cyclohexanecarboxylic acid (100 mg, 0.33 mmol) in DCM (2 mL). The mixture was heated at 35° C. for four hours. The solution was washed with 0.4 M Na₂CO₃ (2 mL), NH₄Cl (2 mL) and then with water (2 mL). The organic layer was concentrated under reduced pressure and crude was purified by silica column (ethyl acetate 95/MeOH 5) to give 85 mg of the title compound (yield 57%).

¹HNMR (CDCl3) δ: 1.13-1.24 (2H, m), 1.52-1.62 (2H, m), 1.79-1.88 (4H, m), 1.91-1.99 (1H, m), 2.45-2.53 (1H, m), 3.55-3.56 (2H, m), 3.67-3.72 (4H, m), 3.79-3.85 (7H, m), 6.53-6.57 (2H, m), 6.61-6.64 (1H, m), 6.96-6.98 (1H, m), 8.32-8.33 (2H, m), 9.01 (1H, s).

C24H30N6O3 Mass (calculated) [450.55]. found [M+H+]=451, RT=1.14 (method f).

### 5-Methoxy-1-methyl-3-[trans-4-(4-pyrimidin-2-yl-piperazine-1-carbonyl)-cyclohexylmethyl]-1,3-dihydro-benzoimidazol-2-one

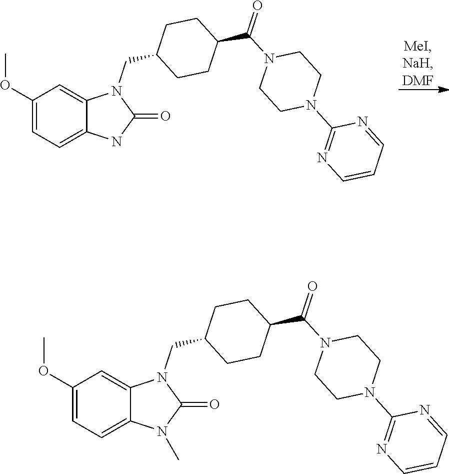

NaH, (60% dispersion in mineral oil, 12 mg, 0.3 mmol) and MeI (18.7 μL, 0.3 mmol) were added to a solution of 6-Methoxy-1-[trans-4-(4-pyrimidin-2-yl-piperazine-1-carbonyl)-cyclohexylmethyl]-1,3-dihydro-benzoimidazol-2-one (68 mg, 0.15 mmol) in DMF (1.5 mL). The mixture was stirred at room temperature 6 hours then was concentrated under vacuum. DCM (2 mL) and water (3 mL) were added to the crude material. The organic layer separated and then concentrated under reduced pressure. The crude was purified by silica column (ethyl acetate 9/MeOH 1) to afford 60 mg of the titled compound (yield 86%).

¹HNMR (CDCl3) δ: 1.13-1.22 (2H, m), 1.51-1.61 (2H, m), 1.78-1.86 (4H, m), 1.89-1.97 (1H, m), 2.44-2.51 (1H, m), 3.39 (3H, s), 3.53-3.55 (2H, m), 3.65-3.69 (2H, m), 3.70-3.72 (2H, m), 3.78-3.85 (7H, m), 6.52-6.55 (1H, m), 6.57-6.58 (1H, m), 6.64-6.67 (1H, m), 6.85-6.87 (1H, m), 8.32-8.33 (2H, m).

C25H32N6O3 Mass (calculated, for the acid) [464.57]. found [M+H+]=465, RT=1.27 (method f).

### Example 3

### (Method A4): 3-[trans-4-(4-acetyl-piperazine-1-carbonyl)-cyclo hexylmethyl]-5-bromo-1-methyl-1,3-dihydro-imidazo[4,5-b]pyridin-2-one 1-(4-{t cans 4-[(6-Bromo-3-nitro-pyridin-2-ylamino)-methyl]cyclohexane carbonyl}-piperazin-1-yl)-ethanone

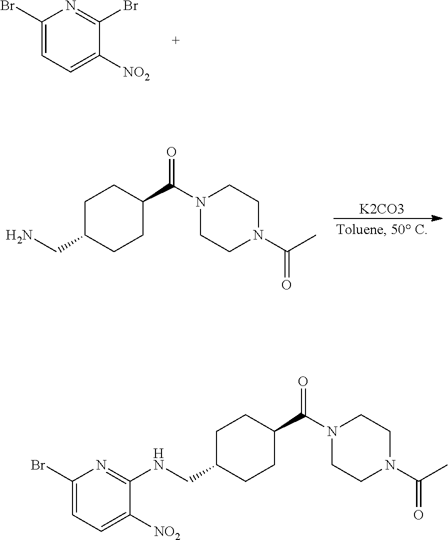

K₂CO₃ (0.677 g, 4.90 mmol) was added to a mixture of 2,6-Dibromo-3-nitro-pyridine (1.38 g, 4.90 mmol), and 1-[trans 4-(4-Aminomethyl-cyclohexanecarbonyl)-piperazin-1-yl]-ethanone (1.31 g, 4.90 mmol) in toluene (14 mL). The resulting mixture was stirred at 60° C. 5 h. The mixture was washed with water (10 mL) and the aqueous phase was extracted with DCM (5 mL). The combined organic layers were dried over Na₂SO₄ and concentrated under reduced pressure. The residue was purified by silica column with DCM:MeOH 95:5 as eluent to give 0.85 g of the titled compound (yield 37%).

¹HNMR (CDCl3) δ: 1.07-1.17 (2H, m), 1.59-1.84 (5H, m), 1.92-1.96 (2H, m), 2.12 (3H, s), 2.42-2.50 (1H, m), 3.43-3.55 (6H, m), 3.60-3.65 (4H, m), 6.76 (1H, d, J=8.4 Hz), 8.21 (1H, d, J=8.4 Hz), 8.39-8.41 (1H, m).

### 1-(Trans 4-{4-[(3-Amino-6-bromo-pyridin-2-ylamino)-methyl]cyclohexane carbonyl}-piperazin-1-yl)-ethanone

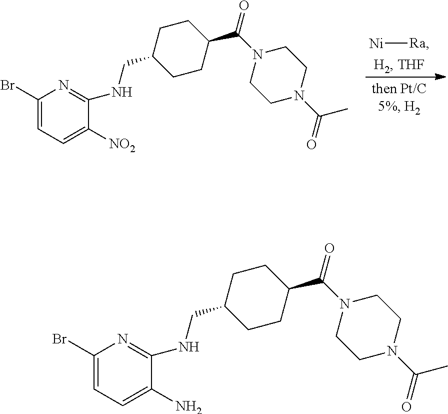

A solution of −1-(Trans 4-{4-[(6-bromo-3-nitro-pyridin-2-ylamino)-methyl]cyclohexanecarbonyl}-piperazin-1-yl)-ethanone (1.0 g, 2.14 mmol) in THF (20 mL) was added to Ni—Ra 50% suspension in water (350 μL). The reaction mixture was hydrogenated in an Eyela apparatus at 5 Bar at room temperature for 2 hours and at 45° C. for 4 hours. Since not complete conversion was observed the reaction mixture was filtered through a cellulose pad and 100 mg of Pt/C 5% were added. The mixture was then kept under 5 bar of hydrogene overnight at room temperature with stirring. The mixture was filtered through cellulose pad and concentrated under reduced pressure. The residue was purified by silica column (AcOEt 9/MeOH 1) to give 740 mg of titled compound (yield 79%).

C19H28BrN5O2 Mass (calculated) [438.37]. found [M+H⁺]=438/440, RT=1.14 (method f)

### 3-[trans 4-(4-Acetyl-piperazine-1-carbonyl)-cyclohexylmethyl]-5-bromo-1,3-dihydro-imidazo[4,5-b]pyridin-2-one

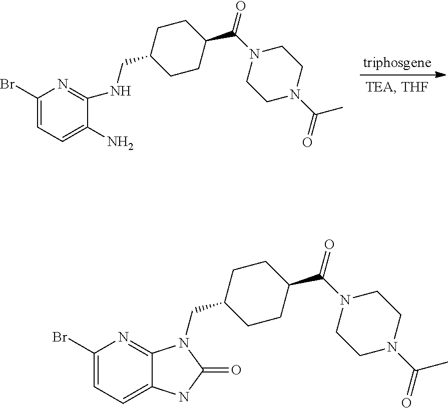 [SMILES: CC(=O)N1CCN(C(=O)[C@H]2CC[C@H](CNc3nc(Br)ccc3N)CC2)CC1 ; CC(=O)N1CCN(C(=O)[C@H]2CC[C@H](Cn3c(=O)[nH]c4ccc(Br)nc43)CC2)CC1]

1-(trans 4-{4-[(3-Amino-6-bromo-pyridin-2-ylamino)-methyl]cyclohexane carbonyl}-piperazin-1-yl)-ethanone (0.740 g, 1.69 mmol) was suspended in THF (15 mL) with TEA (0.170 mL, 1.69 mmol) at 0° C. Triphosgene (165 mg, 0.56 mmol) was added portionwise in 30 minutes. The mixture was allowed to reach r.t. and then an additional equivalent of triphosgene (165 mg, 0.56 mmol) was added. The mixture was heated at 60° C. until complete conversion of the starting material occurred. The reaction mixture was then allowed to reach r.t. and water (5 mL) was added. The solvent was removed under reduced pressure and the residue was redissolved in DCM (20 mL). The solution was dried over Na₂SO₄, filtered, and concentrated to give 0.710 g of the titled compound as a pale brown residue that was used without further purification (yield 91%).

C20H26BrN5O3 Mass (calculated) [464.37]. found [M+H⁺]=464/466, RT=1.04 (method f)

### 3-[trans 4-(4-Acetyl-piperazine-1-carbonyl)cyclohexylmethyl]-5-bromo-1-methyl-1,3-dihydro-imidazo[4,5-b]pyridin-2-one

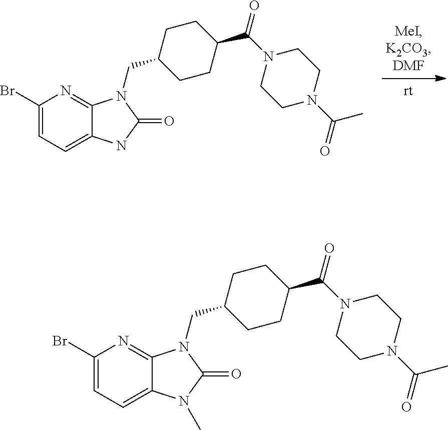 [SMILES: CC(=O)N1CCN(C(=O)[C@H]2CC[C@H](Cn3c(=O)[nH]c4ccc(Br)nc43)CC2)CC1 ; CC(=O)N1CCN(C(=O)[C@H]2CC[C@H](Cn3c(=O)n(C)c4ccc(Br)nc43)CC2)CC1]

K₂CO₃ (0.46 g, 1.99 mmol) was added to a solution of trans-3-[4-(4-Acetyl-piperazine-1-carbonyl)-cyclohexylmethyl]-5-bromo-1,3-dihydro-imidazo[4,5-b]pyridin-2-one (0.71 g, 1.53 mmol) in DMF (10 mL). After 10 minutes MeI (0.12 mL, 1.99 mmol) was added and the mixture was stirred at r.t. for 4 hours then it was concentrated under reduced pressure. DCM (10 mL) and H₂O (5 mL) were added to the crude; the organic layer was separated and concentrated under reduced pressure. The crude was purified by silica column (gradient of AcOEt:MeOH, 95:5) to give 0.61 g of the titled compound (yield 83%).

¹HNMR (CDCl3) δ: 1.10-1.20 (2H, m), 1.50-1.59 (2H, m), 1.74-1.82 (4H, m), 1.96-2.06 (1H, m), 2.12 (3H, s), 2.39-2.47 (1H, m), 3.42-3.52 (7H, m), 3.59-3.62 (4H, m), 3.81 (2H, d, J=7.2 Hz), 7.03 (1H, d, J=8.0 Hz), 7.17 (1H, d, J=8.0 Hz).

C21H28BrN5O3 Mass (calculated) [478.39]. found [M+H⁺]=478/480, RT=1.14 (method f)

### Example 4

### (Method B): 3-[Trans-4-(4-Acetyl-piperazine-1-carbonyl)-cyclohexylmethyl]-5-((E)-3-methoxy-propenyl)-1-methyl-1,3-dihydro-imidazo[4,5-b]pyridin-2-one

### 3-[Trans 4-(4-Acetyl-piperazine-1-carbonyl)-cyclohexylmethyl]-5-((E)-3-methoxy-propenyl)-1-methyl-1,3-dihydro-imidazo[4,5-b]pyridin-2-one

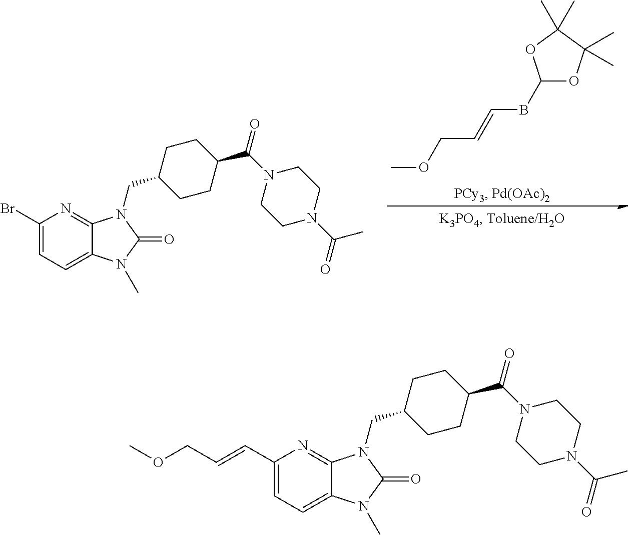

3-[trans 4-(4-Acetyl-piperazine-1-carbonyl)cyclohexylmethyl]-5-bromo-1-methyl-1,3-dihydro-imidazo[4,5-b]pyridin-2-one (70 mg, 0.15 mmol), ((E)-3-Methoxy-propenyl)-(4,4,5,5-tetramethyl-[1,3]dioxolan-2-yl)-borane (87 mg, 0.44 mmol), K₃PO₄ (109 mg, 0.51 mmol), were dissolved in a mixture of toluene and water (20:1, 2.1 ml)), then tricyclohexyl-phosphine (4.0 mg, 0.01 mmol) and Pd(OAc)₂ (3 mg, 0.01 mmol) were added. The resulting mixture was irradiated at 90° C. in microwave apparatus for 10 minutes. Water (2 ml) was added, layers were separated and the water phase was additionally washed with DCM (2 mL). The organic phases were collected, dried over Na₂SO₄, filtered and the solvent evaporated. The residue was purified first by silica column (AcOEt/MeOH 9:1) and then by preparative HPLC (method b) to give 18 mg of the titled compound (yield 21%).

¹HNMR (CDCl₃) δ: 1.11-1.20 (2H, m), 1.50-1.60 (2H, m), 1.74-1.77 (2H, m), 1.81-1.85 (2H, m), 1.99-2.07 (1H, m), 2.12 (3H, s), 2.41-2.48 (1H, m), 3.41-3.51 (10H, m), 3.58-3.62 (4H, m), 3.85 (2H, d, J=7.2 Hz), 4.14-4.16 (2H, m), 6.62-6.79 (2H, m), 6.94 (1H, d, J=8.0 Hz), 7.07 (1H, d, J=8.0 Hz).

C25H35N5O4 Mass (calculated) [469.59]. found [M+H⁺]=470, RT=1.11 (method f)

### Example 5

### (Method C): Trans-4-{5-[(2-Dimethylamino-ethyl)-methyl-amino]-1-methyl-2-oxo-1,2-dihydro-imidazo[4,5-b]pyridin-3-ylmethyl}-cyclohexanecarboxylic acid pyridin-4-ylamide

### Trans-4-[(6-Bromo-3-nitro-pyridin-2-ylamino)-methyl]-cyclohexanecarboxylic acid methyl ester

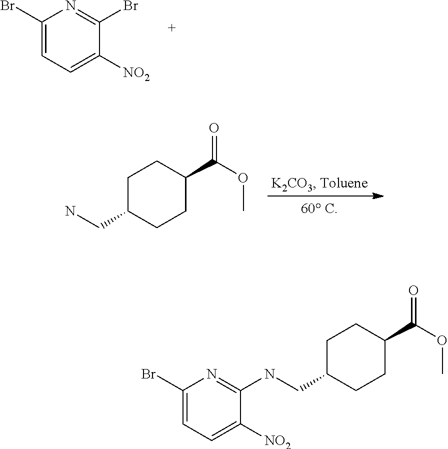 [SMILES: O=[N+]([O-])c1ccc(Br)nc1Br ; COC(=O)[C@H]1CC[C@H](CN)CC1 ; COC(=O)[C@H]1CC[C@H](CNc2nc(Br)ccc2[N+](=O)[O-])CC1]

K₂CO₃ (2.27 g, 16.4 mmol) and trans-4-aminomethyl-cyclohexanecarboxylic acid methyl ester (1.70 g, 8.2 mmol) were added to a stirred solution of 2,2-Dibromo-3-nitropyridine (2.1 g, 7.45 mmol) in toluene (20 ml). The reaction mixture was heated at 60° C. overnight. H₂O (15 mL) was added, the organic phase was separated, dried over Na₂SO₄, filtered and concentrated under reduced pressure. The residue was purified by silica column (Cyclohexane/DCM 3:2) to afford 1.45 g of the titled compound as a yellow crystalline solid (yield 52%).

¹HNMR (CDCl₃) δ: 1.03-1.13 (2H, m), 1.41-1.51 (2H, m), 1.62-1.72 (1H, m), 1.89-1.94 (2H, m), 2.02-2.06 (2H, m), 2.24-2.31 (1H, m), 3.51 (2H, t, J=6.0 Hz), 3.66 (3H, s), 6.76 (1H, d, J=8.4 Hz), 8.2 (1H, d, J=8.4 Hz), 8.38 (1H, brs)

C14H18BrN3O4, Calculated [372.22], No mass response, RT=1.88 (method f).

### Trans-4-({6-[(2-Dimethylamino-ethyl)-methyl-amino]-3-nitro-pyridin-2-ylamino}-methyl)-cyclohexanecarboxylic acid methyl ester

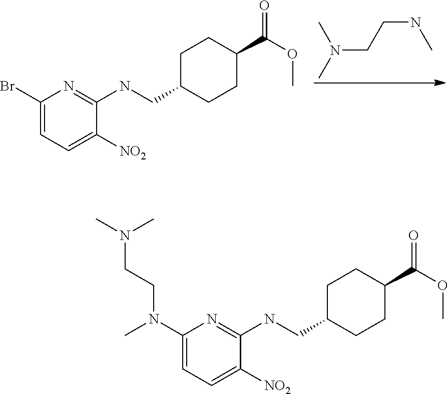

N,N,N′-trimethylethylethlenediammine (2 mL) was added to trans-4-[(6-Bromo-3-nitro-pyridin-2-ylamino)-methyl]-cyclohexanecarboxylic acid methyl ester (350 mg, 1.42 mmol) and the mixture was stirred at 60° C. 2 hours. The resulting solution was concentrated under reduced pressure and crude was purified by silica column (AcOEt/NH₃ 2.0 N solution in MeOH 9:1) to afford 340 mg of the titled compound (yield 92%).

¹HNMR (CDCl₃) δ: 0.99-1.09 (2H, m), 1.38-1.48 (2H, m), 1.67 (1H, bs), 1.88-1.92 (2H, m), 2.00-2.04 (2H, m), 2.22-2.31 (7H, m), 2.52-2.56 (2H, t, J=6.4 Hz), 3.15 (3H, s), 3.42 (2H, t, J=6.4 Hz), 3.66 (3H, s), 3.76 (2H, bs), 5.92 (1H, d, J=9.6 Hz), 8.16 (1H, d, J=9.6 Hz), 8.88 (NH, brs).

C19H31N5O4, Calculated [393.49]. found [M+H⁺] 394, RT=1.09 (method f).

### Trans-4-{5-[(2-Dimethylamino-ethyl)-methyl-amino]-2-oxo-1,2-dihydro-imidazo[4,5-b]pyridin-3-ylmethyl}-cyclohexanecarboxylic acid methyl ester

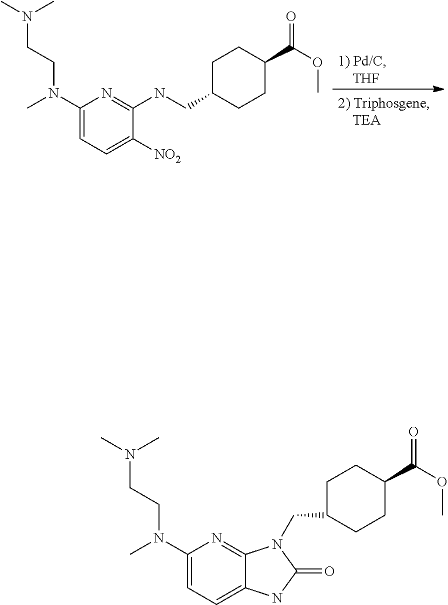

Trans-4-({6-[(2-Dimethylamino-ethyl)-methyl-amino]-3-nitro-pyridin-2-ylamino}methyl)-cyclohexanecarboxylic acid methyl ester (340 mg, 0.86 mmol) was reduced in presence of Pd/C (30 mg 10% w/w) in THF (15 mL) using an Eyela apparatus at 60° C. at 4 bar of hydrogen. After overnight 70% of conversion was observed, the reaction mixture was filtered through a cellulose pad and triphosgene (127 mg, 0.5 eq) and TEA (1 eq) were added. The mixture was left stirring for 16 h at r.t. Water (1 ml) was added, THF was evaporated and DCM (10 mL) was added. The DCM solution was washed with Na₂CO₃ (0.4 M, 2×15 ml), the organic layer was collected, dried over Na₂SO₄, filtered and the solvent remove under reduced pressure. The crude was purified by column chromatography using a silica-NH₂ cartridge and AcOEt as eluent. 145 mg of the titled compound were isolated (yield 43% over 2 steps).

C20H31N5O3, Calculated [389.50]. found [M+H⁺] 390, RT=0.85 (method f).

### Trans-4-{5-[(2-Dimethylamino-ethyl)-methyl-amino]-1-methyl-2-oxo-1,2-dihydro-imidazo[4,5-b]pyridin-3-ylmethyl}-cyclohexanecarboxylic acid methyl ester

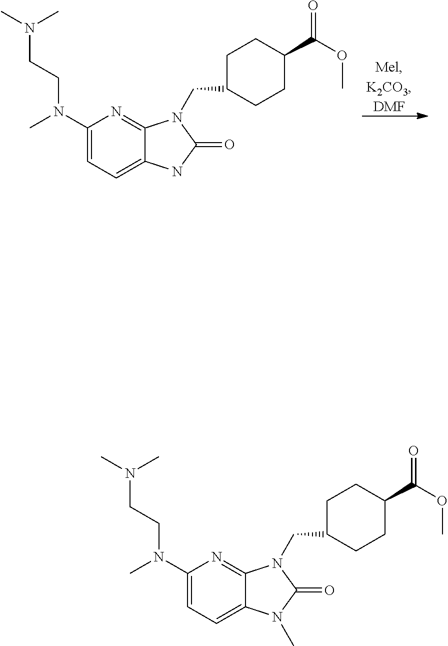

To a solution of trans-4-{5-[(2-Dimethylamino-ethyl)-methyl-amino]-2-oxo-1,2-dihydro-imidazo[4,5-b]pyridin-3-ylmethyl}-cyclohexanecarboxylic acid methyl ester (145 mg, 0.37 mmol) in DMF (2 mL), K₂CO₃ (67 mg, 48 mmol) and MeI (25 μL, 0.41 mmol) were added. The mixture was stirred at r.t. overnight. The solvent was removed under reduced pressure and the residue was dissolved in DCM (6 ml). Water (4 ml) was added, the organic layer was separated, dried over Na₂SO₄, filtered and the solvent removed under reduced pressure. The residue was purified by chromatography using a silica-NH₂ cartridge and DCM/MeOH 9:1 as eluent phase to afford 170 mg of the titled compound containing 60% of its N-Methyl quaternary salt.

C21H33N5O3, Calculated [403.53]. found [M+H⁺] 404, RT=0.84 (method f)

### Trans-4-{5-[(2-Dimethylamino-ethyl)-methyl-amino]-1-methyl-2-oxo-1,2-dihydro-imidazo[4,5-b]pyridin-3-ylmethyl}-cyclohexane lithium carboxylate

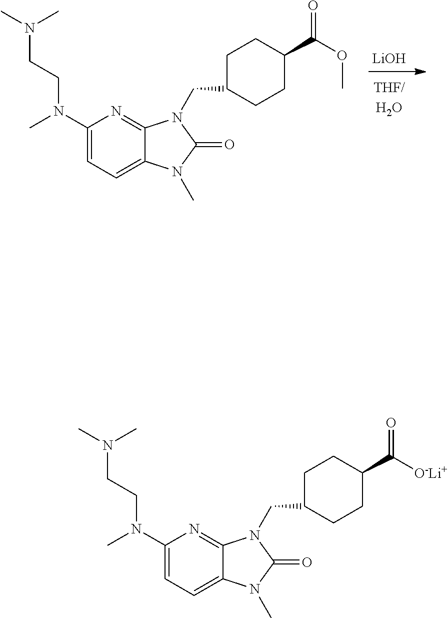

LiOH (11.1 mg, 0.46 ml) in water (1 mL) was added to a solution of trans-4-{5-[(2-Dimethylamino-ethyl)-methyl-amino]-1-methyl-2-oxo-1,2-dihydro-imidazo[4,5-b]pyridin-3-ylmethyl}-cyclohexanecarboxylic acid methyl ester (170 mg, 0.42 mmol) in THF (4 mL). The solution was stirred at r.t. overnight. The solution was concentrated under reduced pressure and the residue used in the next steps without further purification. Obtained 126 mg of white solid (yield 99%).

C20H30N5O3Li Mass (calculated, for the acid) [389.50]. found [M+H⁺]=390.

RT=0.70 (method f)

### Trans-4-{5-[(2-Dimethylamino-ethyl)-methyl-amino]-1-methyl-2-oxo-1,2-dihydro-imidazo[4,5-b]pyridin-3-ylmethyl}-cyclohexanecarboxylic acid pyridin-4-ylamide

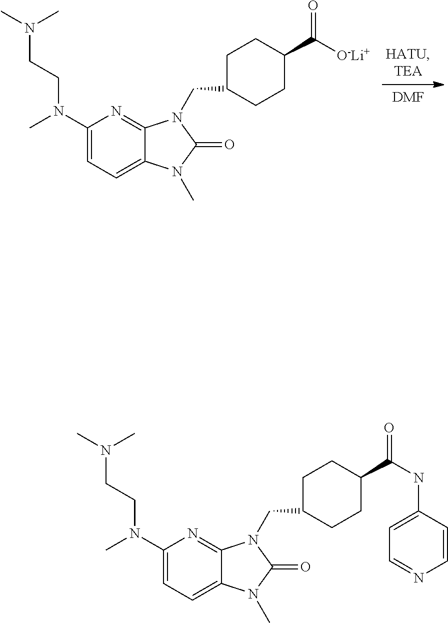

A mixture of trans-4-{5-[(2-Dimethylamino-ethyl)-methyl-amino]-1-methyl-2-oxo-1,2-dihydro-imidazo[4,5-b]pyridin-3-ylmethyl}-cyclohexane lithium carboxylate (58 mg, 0.15 mmol), TEA (18 mg, 0.18 mmol), HATU (68.4 mg, 0.18 mmol) and 4-aminopyridine (17 mg, 0.18 mmol) in DMF (2 mL) was left stirring at r.t. for 4 h. The reaction mixture was concentrated under reduced pressure and the residue was purified by SCX cartridge, and then by two silica columns using AcOEt/NH₃ in MeOH and DCM/MeOH 9:1 as eluant systems. Obtained 33 mg of the titled compound (yield 49%).

¹HNMR (CDCl₃) δ: 1.11-1.21 (2H, m), 1.49-1.59 (2H, m), 1.85-1.89 (2H, m), 1.97-2.08 (3H, m), 2.21-2.29 (1H, m), 2.35 (6H, s), 2.54 (2H, t, J=7.2 Hz), 3.01 (3H, s), 3.36 (3H, s), 3.70 (2H, t, J=7.2 Hz), 3.77 (2H, d, J=7.2 Hz), 6.14 (1H, d, J=8.8 Hz), 7.04 (1H, d, J=8.8 Hz), 7.48 (2H, d, J=5.2 Hz), 7.63 (1H, bs), 8.46 (2H, d, J=5.2 Hz).

C25H35N7O2 Mass (calculated) [465.60]. found [M+H⁺]=466. RT=0.69 (method f)

### Example 6

### (Method D): Trans-4-[6-(4-Methoxy-benzyloxy)-3-methyl-2-oxo-2,3-dihydro-benzoimidazol-1-ylmethyl]-cyclohexanecarboxylic acid pyridin-4-ylamide

### 2-(3-fluoro-4-nitrophenoxy)tetrahydro-2H-pyran

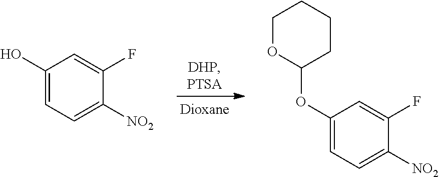 [SMILES: O=[N+]([O-])c1ccc(O)cc1F ; O=[N+]([O-])c1ccc(OC2CCCCO2)cc1F]

In a 500 ml four necked round bottom flask 3,4-dihydro-2H-pyran (19.6 g, 216.4 mmol) and PTSA (1.0 g, 5.4 mmol) were dissolved in dry dioxane (170 ml) then cooled to 10° C. A solution of 3-fluoro-4-nitrophenol (17.0 g, 108.2 mmol) in dry dioxane (80 ml) was added dropwise keeping temperature below 10° C., then the reaction mixture was stirred for 2 h at rt. The reaction was quenched by adding a saturated solution of Na₂CO₃ (300 ml) and the organic phase was extracted with DCM (2×500 ml). The organic layer was washed with a saturated solution of Na₂CO₃ (2×500 ml) and then with brine (2×500 ml). The DCM solution was dried over Na₂SO₄, filtered and evaporated under reduced pressure to give 26.6 g of a brownish solid. This was triturated with MTBE (70 ml) and filtered to give 14.5 g of the titled compound as a pale yellow crystalline solid. The mother liquors were evaporated under reduced pressure to give a dark oil (9.6 g) that was purified by silica column using a PE/EtOAc 9/1 mixture as eluent, to give 3.0 g of the titled compounds. This batch was added to the previous one to give 17.5 g (72.6 mmol, yield 67%) of titled compound as a pale yellow crystalline solid.

TLC: (EDP/EtOAc 9/1) R_{f}=0.54 (UV).

### Trans-4-{[2-nitro-5-(tetrahydro-2H-pyran-2-yloxy)phenylamino]methyl}cyclo hexanecarboxylic acid methyl ester

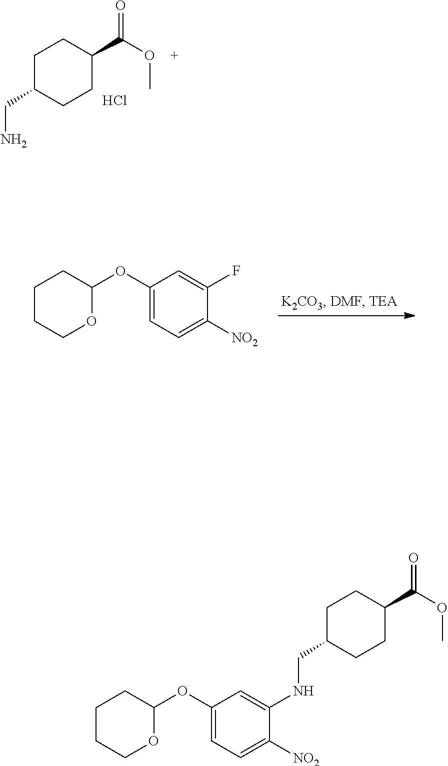 [SMILES: COC(=O)[C@H]1CC[C@H](CN)CC1 ; Cl ; O=[N+]([O-])c1ccc(OC2CCCCO2)cc1F ; "K2CO3, DMF, TEA" ; COC(=O)[C@H]1CC[C@H](CNc2cc(OC3CCCCO3)ccc2[N+](=O)[O-])CC1]

In a 500 ml four necked round bottom flask 2-(3-fluoro-4-nitrophenoxy)tetrahydro-2H-pyran (16.3 g, 67.6 mmol) was dissolved in dry DMF (150 ml) then K₂CO₃ (18.72 g, 135.2 mmol) was added. In the meantime in a 250 ml two necked round bottom flask, methyl trans-4-(aminomethyl)cyclohexane carboxylate hydrochloride (14.0 g, 67.6 mmol) was dissolved in dry DMF (100 ml) then TEA (9.4 ml, 67.6 mmol) was added. After few minutes the suspension was filtered under Argon and the filtrate was added to the first flask. The suspension was stirred at 50° C. overnight. The reaction mixture was quenched with water (300 ml) then extracted with DCM (2×500 ml). The collected organic solutions were washed with water (2×500 ml) and brine (2×500 ml), dried over Na₂SO₄, filtered and evaporated under reduced pressure to give 25.6 g (65.2 mmol, yield 97%) of the titled compound as a yellow solid. This was used in the next step with no further purification.

### Trans-4-{[2-amino-5-(tetrahydro-2H-pyran-2-yloxy)phenylamino]methyl}cyclo hexanecarboxylic acid methyl ester

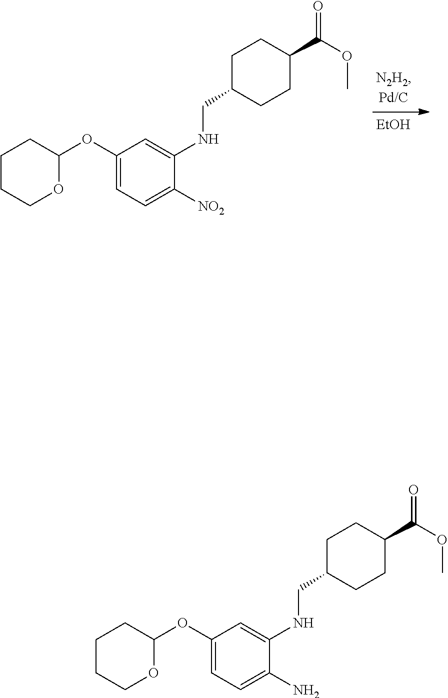

In a 1 L four necked round bottomed flask trans-4-{[2-nitro-5-(tetrahydro-2H-pyran-2-yloxy)phenylamino]methyl}cyclohexanecarboxylic acid methyl ester (25.1 g, 64.0 mmol) was suspended in EtOH (600 ml) then it was completely dissolved by heating before adding Pd/C (1.4 g, 12.8 mmol) and hydrazine monohydrate (6.9 ml, 140.8 mmol). The system was refluxed for 5 hours. The reaction mixture was allowed to reach room temperature, filtered on a celite pad and the mother liquors evaporated under reduced pressure. The residue was taken up with DCM (500 ml), washed with water (2×500 ml), 5% citric acid (2×500 ml) and then brine (2×500 ml). The organic solution was dried over Na₂SO₄, filtered and evaporated under reduced pressure to give 20.0 g of the titled compound as a brown solid (yield 86%).

TLC: (Cy/EtOAc 2/8) R_{f}=0.68 (UV).

### Trans-4-[2-oxo-6-(tetrahydro-2H-pyran-2-yloxy)-2,3-dihydrobenzoimidazol-1-ylmethyl]-cyclohexanecarboxylic acid methyl ester

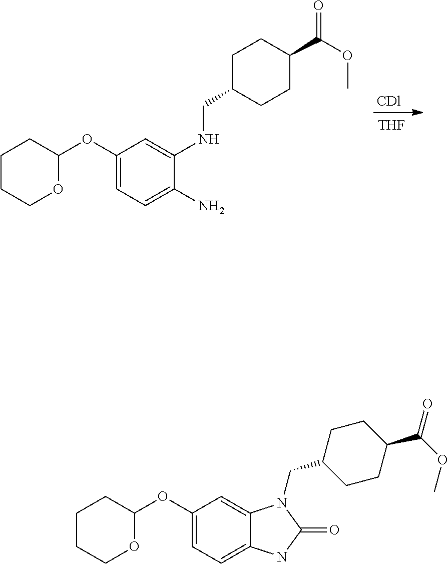

CDI (11.7 g, 72.1 mmol) was added to a dry THF (500 ml) solution of trans-4-{[2-amino-5-(tetrahydro-2H-pyran-2-yloxy)phenylamino]methyl}cyclohexane carboxylic acid methyl ester (13.1 g, 36.1 mmol) in a 1 L four necked round bottom flask. The reaction mixture was stirred at room temperature. The solvent was evaporated under reduced pressure and the residue was taken up with DCM (500 ml) then washed with water (2×500 ml) and brine (2×500 ml). The organic layer was dried over Na₂SO₄, filtered and evaporated under reduced pressure to give 14.5 g (yield quantitative) of crude intermediate as a brown solid. This was used in the next step with no further purification.

### Trans-4-[3-methyl-2-oxo-6-(tetrahydro-2H-pyran-2-yloxy)-2,3-dihydrobenzo imidazol-1-ylmethyl]-cyclohexanecarboxylic acid methyl ester

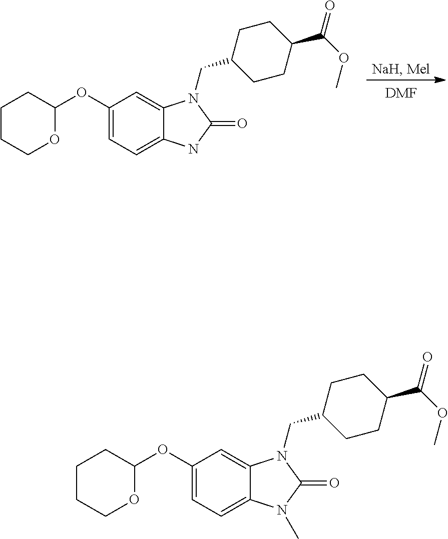

NaH (6.5 g, 162.0 mmol) was added to a dry DMF solution (300 ml) of trans-4-[2-oxo-6-(tetrahydro-2H-pyran-2-yloxy)-2,3-dihydrobenzoimidazol-1-ylmethyl]-cyclohexane carboxylic acid methyl ester (21.0 g, 54.0 mmol) in a 1 L four necked round bottom flask. The mixture was stirred for 1 h at rt then iodomethane (10.1 ml, 162.0 mmol) was added. The mixture was stirred for 18 h at rt and then quenched with water (500 ml) and extracted with DCM (2×500 ml). Collected organic layers were washed with water (2×500 ml) and brine (2×500 ml), dried over Na₂SO₄, filtered and evaporated under reduced pressure to give 18.3 g of a brown oil. This was purified by flash-chromatography with a Cy/EtOAc 2/8 mixture as eluent to give 13.1 g of the titled compound as a pale yellow foam (yield 60%).

TLC: (Cy/EtOAc 3/7) R_{f}=0.41 (UV).

### Trans-4-(6-hydroxy-3-methyl-2-oxo-2,3-dihydro-benzimidazol-1-ylmethyl)cyclohexanecarboxylic acid

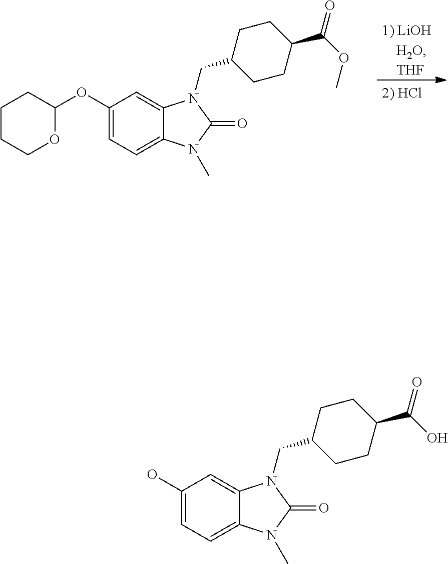

a solution of LiOH*H₂O (4.1 g, 97.5 mmol) in water (75 ml) was added to a THF (150 ml) solution of trans-4-[3-methyl-2-oxo-6-(tetrahydro-2H-pyran-2-yloxy)-2,3-dihydrobenzoimidazol-1-ylmethyl]-cyclohexanecarboxylic acid methyl ester (13.1 g, 32.5 mmol) in a 500 ml one necked round bottom flask The mixture was refluxed for 2 h. The THF was evaporated under reduced pressure and HCl (6N, 150 ml) was added. The solid was isolated by filtration redissolved in THF (300 ml) and 6M HCl (21.0 ml, 130.0 mmol) was added. The mixture was refluxed overnight. The THF was evaporated under reduced pressure and the residue was triturated with MTBE (100 ml) and then filtered. 7.6 g (yield 77%) of the titled compound were isolated as a light grey solid.

¹H NMR (DMSO) δ: 1.1 (m, 2H); 1.2 (m, 2H); 1.7 (m, 3H); 1.9 (m, 2H); 2.1 (m, 1H); 3.5 (s, 3H), 3.6 (dd, 2H), 6.5 (dd, 1H), 6.6 (d, 1H), 6.9 (d, 1H), 9.1 (bs, 1H), 12.0 (bs, 1H).

### Trans-4-(6-Hydroxy-3-methyl-2-oxo-2,3-dihydro-benzoimidazol-1-ylmethyl)-cyclohexanecarboxylic acid methyl ester

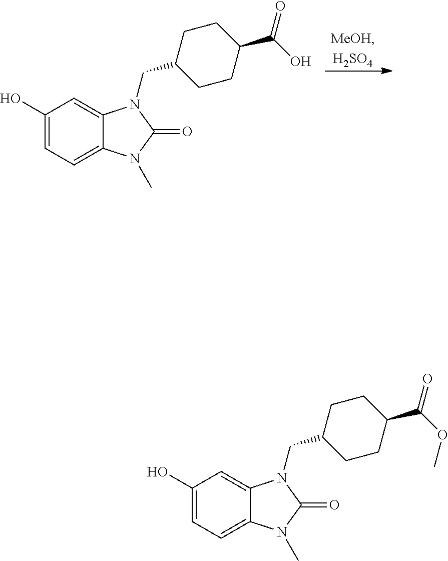

trans-4-(6-hydroxy-3-methyl-2-oxo-2,3-dihydro-benzimidazol-1-ylmethyl)cyclo hexanecarboxylic acid (500 mg, 1.64 mmol) was dissolved in MeOH (5 ml) with H₂SO₄ (0.05 ml). The solution was left refluxing for 2 h. The solvent was evaporated and the residue was washed with Et₂O and filtered. 470 mg of the titled compound were obtained (yield 89%).

C17H22N2O4 Mass (calculated) [318.38]. found [M+H⁺]=319 RT=1.09 (method f)

### Trans-4-[6-(4-Methoxy-benzyloxy)-3-methyl-2-oxo-2,3-dihydro-benzo imidazol-1-ylmethyl]-cyclohexanecarboxylic acid methyl ester

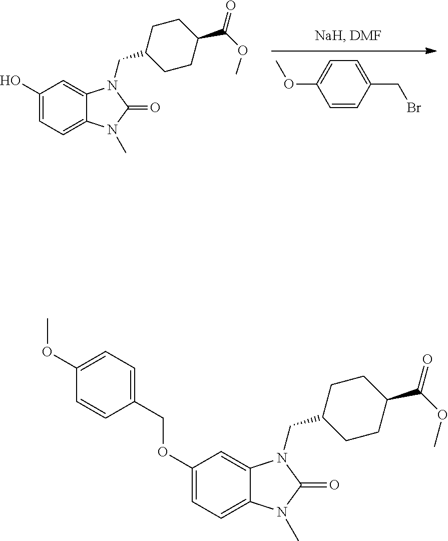

At 0° C., NaH (13 mg, 0.35 mmol) was added portionwise to a solution of trans-4-(6-Hydroxy-3-methyl-2-oxo-2,3-dihydro-benzoimidazol-1-ylmethyl)-cyclohexane carboxylic acid methyl ester (100 mg, 0.31 mmol) in DMF (4 ml). The mixture was left stirring for 1 h at room temperature then, 1-bromomethyl-4-methoxy-benzene (0.054 ml, 0.38 mmol) was added and the mixture was stirred overnight at room temperature. Water (5 ml) was added and the solution was extracted with DCM (5 ml). The organic layer was washed with NaOH 1 N (5 ml), dried over Na₂SO₄, filtered and then the solvent was evaporated under reduced pressure. The crude was purified by silica column using Cyclohexane/AcOEt 1:1 as eluent. Obtained 85 mg of the titled compound (yield 62%).

C25H30N2O5 Mass (calculated) [438.53]. found [M+H⁺]=439 RT=1.66 (method f)

### Trans-4-[6-(4-Methoxy-benzyloxy)-3-methyl-2-oxo-2,3-dihydro-benzo imidazol-1-ylmethyl]-cyclohexanecarboxylic acid

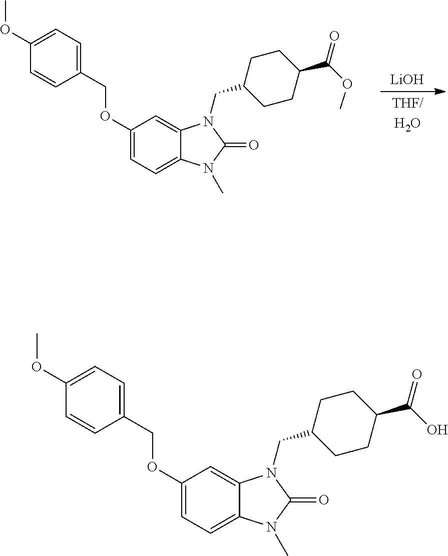

LiOH (11 mg, 0.49 mmol) was added to the solution of trans-4-[6-(4-Methoxy-benzyloxy)-3-methyl-2-oxo-2,3-dihydro-benzoimidazol-1-ylmethyl]-cyclohexane carboxylic acid methyl ester (85 mg, 0.19 mmol) dissolved in THF:H₂O (5 ml each) and the resulting suspension was left stirring for 16 h at 50° C. The THF was evaporated under reduced pressure, the aqueous solution was extracted with DCM (5 ml) then it was acidified with HCl 1N; the precipitated material was filtered and collected. 45 mg of the titled compound were isolated (yield 56%).

C24H28N2O5 Mass (calculated) [424.50]. found [M+H⁺]=425 RT=1.44 (method f)

### Trans-4-[6-(4-Methoxy-benzyloxy)-3-methyl-2-oxo-2,3-dihydro-benzo imidazol-1-ylmethyl]-cyclohexanecarboxylic acid pyridin-4-ylamide

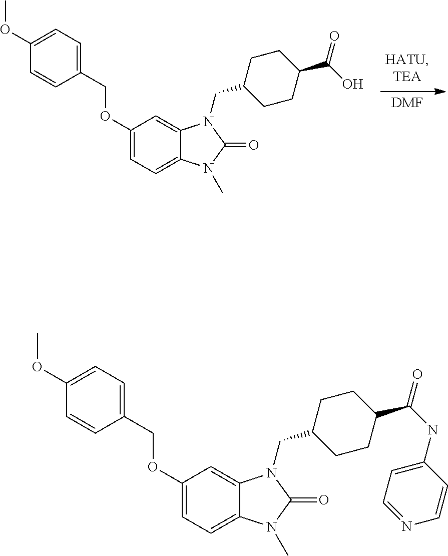

Trans-4-[6-(4-Methoxy-benzyloxy)-3-methyl-2-oxo-2,3-dihydro-benzoimidazol-1-ylmethyl]-cyclohexanecarboxylic acid (45 mg, 0.11 mmol), TEA (33 μL, 0.21 mmol), HATU (49 mg, 0.13 mmol) and pyridin-4-ylamine (12 mg, 0.13 mmol) in DMF (5 mL) were stirred at r.t. for 16 h. Water (5 mL) was added and the mixture extracted with DCM (2*5 mL). The organic layers were collected, dried over Na₂SO₄, filtered and the solvent removed under reduced pressure. The residue was purified by preparative HPLC (method c) to give 28 mg of the titled compound (yield 51%).

¹HNMR (MeOD) δ: 0.96-1.41 (2H, m), 1.25-1.39 (2H, m), 1.59-1.87 (5H, m), 2.26-2.36 (1H, m), 3.26 (3H, s), 3.60-3.64 (2H, m), 3.72 (3H, s), 6.71-6.76 (1H, m), 6.92 (1H, d, J=8.4 Hz), 6.93 (1H, brs), 6.99 (1H, d, J=8.2 Hz), 7.37 (2H, d, J=8.4 Hz), 7.53 (2H, d, J=1.2 Hz), 8.21 (1H, brs) 8.37 (2H, brs), 10.2 (1H, brs).

C29H32N4O4 Mass (calculated) [500.60]. found [M+H⁺]=501, RT=1.28 (method f)

### Example 7

### (Method E): 5-Methoxy-1-methyl-3-{trans-4-[3-oxo-4-(2-oxo-butyl)-piperazine-1-carbonyl]-cyclohexylmethyl}-1,3-dihydro-benzoimidazol-2-one

### 5-Methoxy-1-methyl-3-[trans-4-(3-oxo-piperazine-1-carbonyl)-cyclo hexylmethyl]-1,3-dihydro-benzoimidazol-2-one

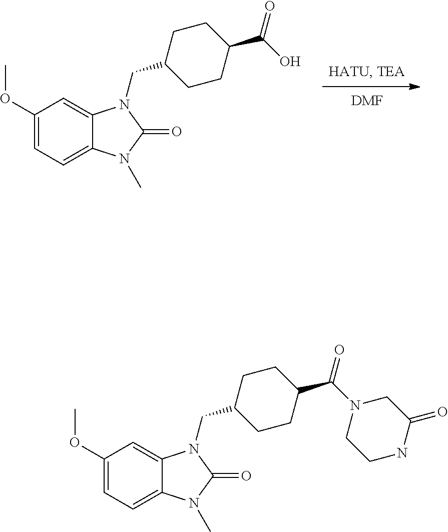

TEA (350 μL, 2.52 mmol), HATU (574 mg, 1.51 mmol) and piperazin-2-one (151 mg, 1.51 mmol) were added to a solution of trans-4-(6-Methoxy-3-methyl-2-oxo-2,3-dihydro-benzoimidazol-1-ylmethyl)-cyclohexane carboxylic acid (400 mg, 1.26 mmol) in DMF (5 mL). The reaction was stirred at r.t. for 2 h. Water (5 ml) was added and the mixture was extracted with DCM (3×5 ml). The organic layers were collected, dried over Na₂SO₄ and concentrated. The residue was purified by silica column (Cyclohexane/AcOEt 1:1, then AcOEt/MeOH 4:1) to give 380 mg of the titled compound (Yield 59%).

¹H NMR (DMSO) δ: 1.02-1.31 (4H, m), 1.59-1.78 (5H, m), 2.51-2.56 (1H, m), 3.07-3.20 (2H, m), 3.26 (3H, s), 3.52-3.56 (1H, m), 3.59-3.63 (3H, m), 3.73 (3H, s), 3.85 (1H, bs), 4.04 (1H, bs), 6.61 (1H, dd, J=2.4 and 8.4 Hz), 6.79 (1H, d, J=2.4 Hz), 7.00 (1H, d, J=8.4 Hz), 8.01-8.05 (1H, m).

C21H28N4O4 Mass (calculated) [400.48]. found [M+H⁺]=401, RT=1.00 (method f)

### 5-Methoxy-1-methyl-3-{trans-4-[3-oxo-4-(2-oxo-butyl)-piperazine-1-carbonyl]cyclohexylmethyl}-1,3-dihydro-benzoimidazol-2-one

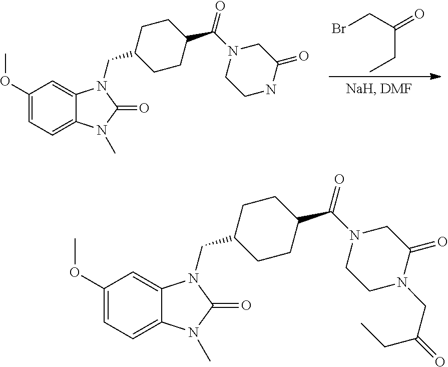

To a solution of 5-Methoxy-1-methyl-3-[trans-4-(3-oxo-piperazine-1-carbonyl)-cyclohexylmethyl]-1,3-dihydro-benzoimidazol-2-one (320 mg, 0.80 mmol) in dry DMF (4 ml), under N₂, NaH (37 mg, 0.96 mmol) was added at 0° C. The resulting mixture was stirred for 1 h at room temperature then 1-bromo-butan-2-one (0.163 mL, 1.60 mmol) was added, and the mixture was stirred for 2 h. Water (4 ml) was added and the reaction mixture was extracted with DCM (3×5 ml). The organic layers were collected, dried over Na₂SO₄, concentrated and residue was purified by silica column (100% DCM) to obtain 261 mg of titled compound (yield 70%).

C25H34N4O5 Mass (calculated) [470.57]. found [M+H⁺]=471, RT=1.13 (method d)

### Example 8

### (Method F): Trans-4-{2-[(2-Methoxy-ethyl)-methyl-amino]-7-methyl-8-oxo-7,8-dihydro-purin-9-ylmethyl}-cyclohexanecarboxylic acid pyridine-4-ylamide

### trans-4-({2-[(2-Methoxy-ethyl)-methyl-amino]-5-nitro-pyrimidin-4-ylamino}-methyl)-cyclohexanecarboxylic acid methyl ester

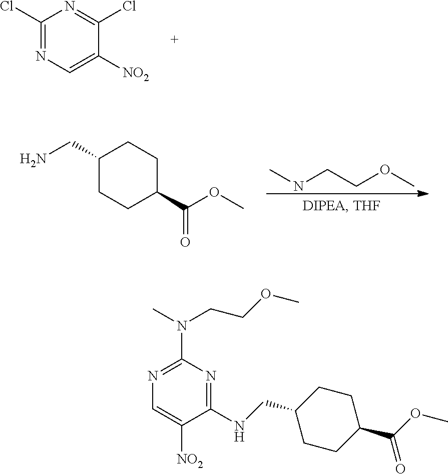

2,4-Dichloro-5-nitro-pyrimidine (547 mg, 2.82 mmol) was dissolved in THF (16 mL) and the resulting solution was cooled to −78° C. A solution of 4-aminomethyl-cyclohexanecarboxylic acid methyl ester (483 mg, 2.82 mmol) and DIPEA (0.75 mL, 4.24 mmol) in THF was added dropwise. The solution was stirred 1 h at −78° C. The reaction mixture was allowed to reach room temperature and then DIPEA (0.75 mL, 4.24 mmol) and (2-Methoxy-ethyl)-methyl-amine (0.362 mL, 3.38 mmol) were added. The mixture was stirred for 16 h. The solution was concentrated under reduced pressure and the crude material was triturated with MeOH. The solid was filtered and dried to give 502 mg of titled compound as pale yellow solid (yield 47%).

¹HNMR (CDCl3) δ: 1.05 (2H, dd J=12.8 and 9.0 Hz), 1.43 (2H, dd, J=12.8 and 9.0 Hz), 1.56 (3H, s), 1.61-1.74 (1H, m), 1.84-1.93 (2H, m) 1.99-2.07 (2H, m) 2.20-2.28 (2H m), 3.24 (2H, s), 3.34-3.47 (3H, m), 3.58-3.68 (2H, m), 3.66 (2H, m), 3.81 (2H, t), 3.81 (2H, t, J=10.8 and 4 Hz), 8.97 (1H, s)

C17H27N5O5 Mass (calculated) [381.44]. found [M+H⁺]=382 RT=1.68 (method f)

Trans-4-{2-[(2-Methoxy-ethyl)-methyl-amino]-8-oxo-7,8-dihydro-purin-9 ylmethyl}-cyclohexanecarboxylic acid methyl ester

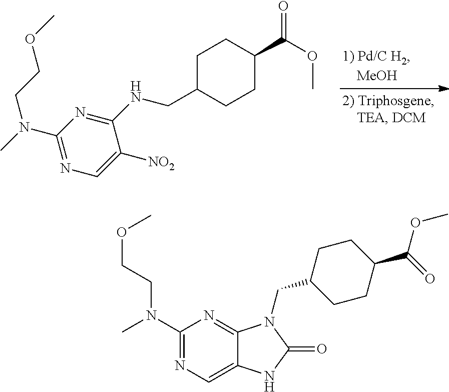

Trans-4-({2-[(2-Methoxy-ethyl)-methyl-amino]-5-nitro-pyrimidin-4-ylamino}-methyl)-cyclohexanecarboxylic acid methyl ester (502 mg, 1.31 mmol) was dissolved in MeOH (5 mL) and the solution hydrogenated using an H-CUBE apparatus (flow 1 mL, full H₂) with a Pd/C cartridge. The collected solution was concentrated and the residue was dissolved in dry DCM (10 mL) under N₂. TEA (0.182 mL, 1.57 mmol) was added and the solution cooled to 0° C. Triphosgene (117 mg, 0.38 mmol) was added and the reaction was stirred overnight at room temperature. H₂O was added, the organic solution was separated and concentrated under reduced pressure. The crude was purified by silica column (gradient of DCM/MeOH 0-6%) to give 150 mg of the titled compound as white solid (yield 30%).

¹HNMR (CDCl3) δ: 1.12 (2H, dd J=12.0, 9.1 Hz), 1.41 (2H, dd, J=12.0, 9.1),1.82 (2H, d, J=12 Hz), 1.91-1.99 (1H, m), 2.01 (2H, d, J=12 Hz), 2.27 (1H, ddt, 1H, J=12 and 6.8 Hz) 3.20 (3H, s), 3.39 (3H, s), 3.62 (2H, t, J=6 Hz), 3.66 (3H, s), 3.72 (2H, d J=7.2 Hz), 3.81 (2H, t), 7.92 (1H, s)

C18H27N5O4 Mass (calculated, for the acid) [377.45]. found [M+H⁺]=378

RT=0.96 (method f)

### Trans-4-{2-[(2-Methoxy-ethyl)-methyl-amino]-7-methyl-8-oxo-7,8-dihydro-purin-9-ylmethyl}-cyclohexanecarboxylic acid methyl ester

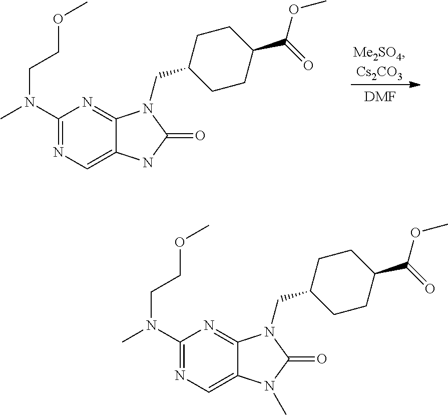

Trans-4-{2-[(2-Methoxy-ethyl)-methyl-amino]-8-oxo-7,8-dihydro-purin-9 ylmethyl}-cyclohexanecarboxylic acid methyl ester (146 mg, 0.38 mmol) was dissolved in dry DMF (15 mL) under N₂ atmosphere. Cs₂CO₃ (189 mg, 0.58 mmol) was added and mixture was stirred for 30 min at r.t. Dimethylsulphate (0.036 mL, 0.38 mmol) was added and the reaction was stirred 4 h. H₂O and DCM were added, the organic layer was separated and concentrated under reduced pressure to give 136 mg of the titled compound as white solid (yield 91%).

¹HNMR (CDCl3) δ: 1.08 (2H, dd J=12.0 and 9.2 Hz), 1.36 (2H, dd, J=12.0 and 9.2 Hz), 1.62 (1H, s), 1.78 (2H, d, J=12.0 Hz), 1.85-1.96 (1H, m), 1.92 (2H, d, J=12.0 Hz), 2.20-2.26 (1H, m,) 3.81 (3H, s), 3.35 (3H, s), 3.37 (3H, s) 3.60 (2H, m), 3.62 (3H, s), 3.69 (2H, d J=7.2 Hz), 3.75-3.80 (2H, m), 7.79 (1H, s)

C19H29N5O4 Mass (calculated) [391.47]. found [M+H+]=392. RT=1.04 (method f)

### Trans-4-{2-[(2-Methoxy-ethyl)-methyl-amino]-7-methyl-8-oxo-7,8-dihydro-purin-9-ylmethyl}-cyclohexane lithium carboxylate

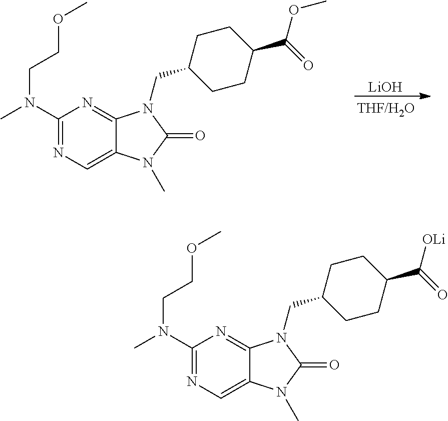

Trans-4-{2-[(2-Methoxy-ethyl)-methyl-amino]-7-methyl-8-oxo-7,8-dihydro-purin-9-ylmethyl}-cyclohexanecarboxylic acid methyl ester (136 mg, 0.34 mmol) was dissolved in THF/H₂O (6 mL, 1:1, v/v). LiOH (9 mg, 0.41 mmol) was added and the solution was stirred at room temperature overnight. The solvent was removed under reduced pressure and the lithium salt was used without further purification. Obtained 126 mg of the titled compound as white solid (yield 99%).

C18H27N5O4 Mass (calculated, for the acid) [377.45]. found [M+H⁺]=378.

RT=0.82 (method f)

### Trans-4-{2-[(2-Methoxy-ethyl)-methyl-amino]-7-methyl-8-oxo-7,8-dihydro-purin-9-ylmethyl}-cyclohexanecarboxylic acid pyridine-4-ylamide

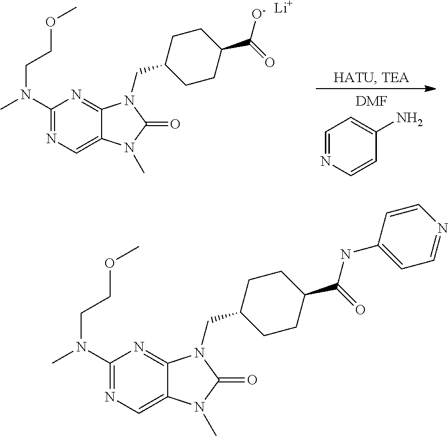

Trans-4-{2-[(2-Methoxy-ethyl)-methyl-amino]-7-methyl-8-oxo-7,8-dihydro-purin-9-ylmethyl}-cyclohexane lithium carboxylate (126 mg, 0.33 mmol) was dissolved in DMF (1 mL) then TEA (0.054 mL, 0.39 mmol) and HATU (150 mg, 0.39 mmol) were added. The solution was stirred for 30 min, then 4-aminopyridine (36 mg, 0.39 mmol) was added and the resulting solution was stirred over the weekend. The reaction mixture was concentrated under reduced pressure. The crude was dissolved in MeOH and passed through silica —NH₂ cartridge eluting with MeOH. The solution was concentrated and the residue was purified by preparative HPLC (method c) obtaining 26 mg of titled compound as white solid (yield 17%).

¹HNMR (DMSO) δ: 1.03 (2H, dd J=10.8 and 6.0 Hz), 1.32 (2H, dd, J=10.8 and 6.0 Hz), 1.63-1.71 (2H, m), 1.76-1.87 (3H, m), 2.23-2.35 (1H, m), 3.23-3.25 (5H, s), 3.46-3.60 (10H, m), 3.66-3.77 (2H, m), 7.52 (2H, d J=8.0 Hz), 7.69 (1H, s), 8.35 (2H, dd).

C23H31N7O3 Mass (calculated) [453.55]. found [M+H⁺]=454.6

RT=0.72 (method f)

### Example 9

### (Method G1): Trans-4-(1-Methyl-2,5-dioxo-1,2,4,5-tetrahydro-imidazo[4,5-b]pyridin-3-ylmethyl)-cyclohexanecarboxylic acid pyridazin-4-ylamide

### Trans-4-(1-Methyl-2,5-dioxo-1,2,4,5-tetrahydro-imidazo[4,5-b]pyridin-3-ylmethyl)-cyclohexanecarboxylic acid

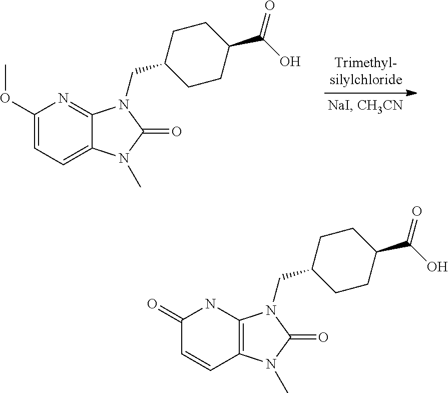 [SMILES: COc1ccc2c(n1)n(C[C@H]1CC[C@H](C(=O)O)CC1)c(=O)n2C ; Cn1c(=O)n(C[C@H]2CC[C@H](C(=O)O)CC2)c2[nH]c(=O)ccc21]

A suspension of trans-4-(5-Methoxy-1-methyl-2-oxo-1,2-dihydro-imidazo[4,5-b]pyridin-3-ylmethyl)-cyclohexanecarboxylic acid (0.500 g, 1.57 mmol) and NaI (704 mg, 4.70 mmol) in CH₃CN (10 mL) was heated at 80° C. into a pressure tube then chlorotrimethylsilane (1.02 g, 9.40 mmol) was added and the mixture was stirred at 100° C. 2 h. The solvent was removed under reduced pressure, and the residue was washed with 1N HCl (10 mL) and DCM (10 mL). Obtained 454 mg of the titled compound as red-brown solid (yield 95%).

C15H19N3O4 Mass (calculated) [305.34]. found [M+H⁺]=306, RT=0.80 (method f)

### Trans-4-(1-Methyl-2,5-dioxo-1,2,4,5-tetrahydro-imidazo[4,5-b]pyridin-3-ylmethyl)-cyclohexanecarboxylic acid pyridazin-4-ylamide

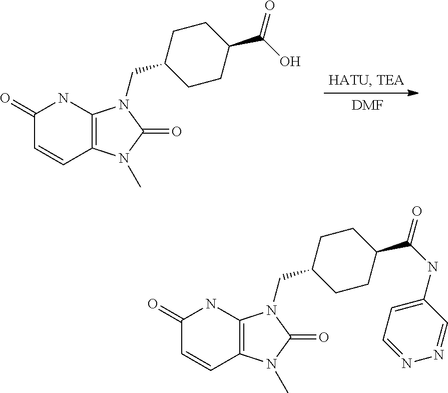

Trans-4-(1-Methyl-2,5-dioxo-1,2,4,5-tetrahydro-imidazo[4,5-b]pyridin-3-ylmethyl)-cyclohexanecarboxylic acid (100 mg, 0.33 mmol) was dissolved in DMF (1 mL) then TEA (91μ, mL, 0.39 mmol), HATU (149 mg, 0.39 mmol) and 4 aminopyridazine (37.4 mg, 0.39 mmol) were added and the mixture was stirred at r.t. overnight. The reaction mixture was concentrated under reduced pressure and crude was purified by silica column (AcOEt/MeOH 9:1) then by preparative HPLC (method b) to give 21 mg of the titled compound (yield 17%).

¹HNMR (CD3OD) δ: 1.13-1-23 (2H, m), 1.44-1.54 (2H, m), 1.78-1.81 (2H, m), 1.94-2.05 (3H, m), 2.35-2.44 (1H, m), 3.39 (3H, s), 3.76 (2H, d, J=7.2 Hz), 6.39 (1H, d, J=8.0 Hz), 7.36 (1H, d, J=8.0 Hz), 8.05-8.08 (1H, m), 8.93-8.95 (1H, m), 9.23-9.24 (1H, m).

C19H22N6O3 Mass (calculated) [382.43]. found [M+H+]=383. RT=0.76 (method f)

### Example 10

### (Method G): Trans-4-[5-(3-Methoxy-benzyloxy)-1-methyl-2-oxo-1,2-dihydro-imidazo[4,5-b]pyridin-3-ylmethyl]-cyclohexane carboxylic acid pyridin-4-ylamide

### Trans-4-(1-Methyl-2,5-dioxo-1,2,4,5-tetrahydro-imidazo[4,5-b]pyridin-3-ylmethyl)-cyclohexanecarboxylic acid methyl ester

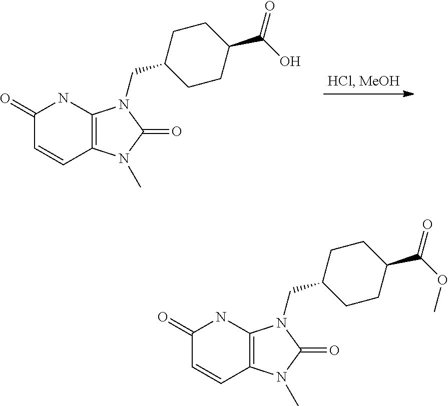 [SMILES: Cn1c(=O)n(C[C@H]2CC[C@H](C(=O)O)CC2)c2[nH]c(=O)ccc21 ; COC(=O)[C@H]1CC[C@H](Cn2c(=O)n(C)c3ccc(=O)[nH]c32)CC1]

Trans-4-(1-Methyl-2,5-dioxo-1,2,4,5-tetrahydro-imidazo[4,5-b]pyridin-3-ylmethyl)-cyclohexanecarboxylic acid (750 mg, 2.46 mmol) was suspended in HCl 1.25 M solution in MeOH (15 mL) and stirred at 50° C. for 3 h. The solvent was removed under reduced pressure to give 780 mg of titled compound (yield 99%).

¹HNMR (CDCl3) δ: 1.10-1.20 (2H, m), 1.30-1.40 (2H, m), 1.72-1.77 (2H, m), 1.87-1.98 (3H, m), 2.19-2.26 (1H, m), 3.40 (3H, s), 3.58 (3H, s), 3.80-3.82 (2H, m), 6.53 (1H, m), 7.36-7.38 (1H, m).

C16H21N3O4 Mass (calculated) [319.36]. found [M+H+]=320. RT=1.03 (method f)

### Trans-4-[5-(3-Methoxy-benzyloxy)-1-methyl-2-oxo-1,2-dihydro-imidazo[4,5-b]pyridin-3-ylmethyl]-cyclohexanecarboxylic acid methyl ester

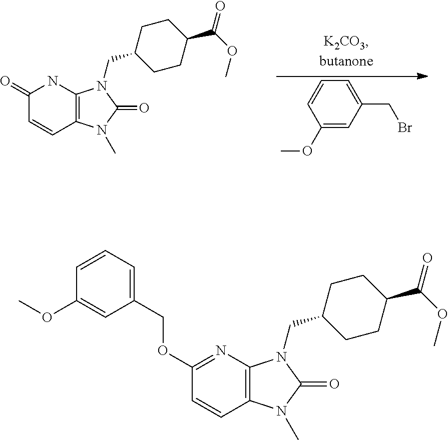

To a suspension of trans-4-(1-Methyl-2,5-dioxo-1,2,4,5-tetrahydro-imidazo[4,5-b]pyridin-3-ylmethyl)-cyclohexanecarboxylic acid methyl ester (100 mg, 0.31 mmol) in 2-butanone (2 mL), K₂CO₃ (87 mg, 0.63 mmol) and 3-methoxy benzylbromide (189 mg, 0.94 mmol) were added and the mixture was stirred at 60° C. overnight. The solvent was concentrated under reduced pressure and the crude was dissolved in DCM (3 ml) and washed with water (3 ml). The organic phase was dried over Na₂SO₄ and concentrated. The crude was purified by silica column eluting with Cyclohexane/AcOEt 2:1. Obtained 120 mg of the titled compound (yield 88%).

¹HNMR (CDCl3) δ: 0.92-1.03 (2H, m), 1.23-1.33 (2H, m), 1.65-1.69 (2H, m), 1.77-1.89 (3H, m), 2.11-2.20 (1H, m), 3.32 (3H, s), 3.58 (3H, s), 3.68-3.69 (2H, m), 3.74 (3H, s), 5.26 (2H, s), 6.43-6.45 (1H, m), 6.75-6.78 (1H, m), 6.92-6.96 (2H, m), 7.05-7.07 (1H, m), 7.19-7.23 (1H, m).

C24H29N3O5 Mass (calculated) [439.52]. found [M+H+]=440. RT=1.72 (method f)

### Trans-4-[5-(3-Methoxy-benzyloxy)-1-methyl-2-oxo-1,2-dihydro-imidazo[4,5-b]pyridin-3-ylmethyl]-cyclohexyl lythium carboxylate

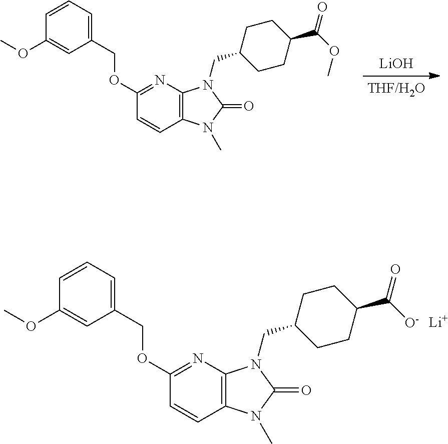

Trans-4-[5-(3-Methoxy-benzyloxy)-1-methyl-2-oxo-1,2-dihydro-imidazo[4,5-b]pyridin-3-ylmethyl]-cyclohexanecarboxylic acid methyl ester (120 mg, 0.27 mmol) was dissolved in THF (2 mL). A solution of LiOH (7.2 mg, 0.30 mmol) in H₂O (1 mL) was added and the solution was stirred overnight. The solution was concentrated under reduced pressure to afford the titled compound (126 mg, yield 99%).

C23H26N3O5L1. Mass (calculated) [425.49]. found [M+H⁺]=426. RT=1.50 (method f)

### Trans-4-[5-(3-Methoxy-benzyloxy)-1-methyl-2-oxo-1,2-dihydro-imidazo[4,5-b]pyridin-3-ylmethyl]-cyclohexanecarboxylic acid pyridin-4-ylamide

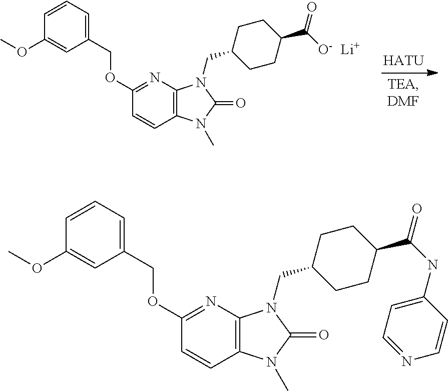

Trans-4-[5-(3-Methoxy-benzyloxy)-1-methyl-2-oxo-1,2-dihydro-imidazo[4,5-b]pyridin-3-ylmethyl]-cyclohexyl lithium carboxylate (115 mg, 0.27 mmol) was dissolved in DMF (1 mL). TEA (0.032 mL, 0.39 mmol), HATU (123 mg, 0.32 mmol) and 4 aminopyridine (30.5 mg, 0.32 mmol) were added, the resulting solution was stirred at r.t. overnight. The mixture was concentrated under reduced pressure, crude was dissolved in DCM (3 mL) and the solution was washed with Na₂CO₃ (2 mL, 0.4 M). The organic phase was separated, dried over Na₂SO₄, filtered and concentrated. The residue was purified on silica column using DCM/MeOH (95:05) as eluent to afford 80 mg of the titled compound (yield 59%).

¹HNMR (DMSO) δ: 0.95-1.09 (2H, m), 1.25-1.38 (2H, m),1.59-1.71 (2H, m), 1.71-1.86 (3H, m), 2.21-2.33 (1H, m), 3.29 (3H, s), 3.64 (2H, d, J=7.4 Hz), 3.72 (3H, s), 6.53 (1H, d, J=8.4 Hz), 6.84 (1H, dd, J=8.4 and 1.0 Hz), 6.98 (2H, s), 7.27 (1H, t, 7.6 Hz), 7.54 (2H, d, J=8.4 Hz), 7.54 (2H, d, J=6.4 Hz), 8.36 (2H, d, J=6.4 Hz), 8.35 (2H, d, J=6.4 Hz), 10.71 (1H, brs).

C28H31N5O4 Mass (calculated) [501.59]. found [M+H⁺]=502, RT=1.21 (method f)

### Example 11

### (Method 112): 5-Methoxy-1-methyl-3-{trans 4-[5-(1-methyl-3-trifluoromethyl-1H-pyrazol-4-yl)-[1,3,4]oxadiazol-2-yl]-cyclohexylmethyl}-1,3-dihydro-benzoimidazol-2-one

### Trans-N′-4-(6-Methoxy-3-methyl-2-oxo-2,3-dihydro-benzoimidazol-1-ylmethyl)-cyclohexanecarboxylic acid hydrazide

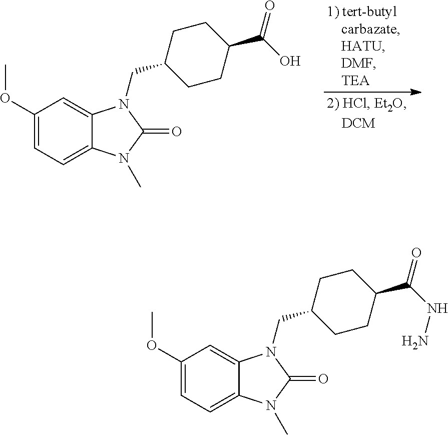

Trans-4-(6-Methoxy-3-methyl-2-oxo-2,3-dihydro-benzoimidazol-1-ylmethyl)cyclohexanecarboxylic acid (600 mg, 1.9 mmol), HATU (860 mg, 2.3 mmol), TEA (0.31 mL, 2.3 mmol) and hydrazinecarboxylic acid tert-butylester (110 mg, 0.83 mmol) were dissolved in DMF (2 mL) and the mixture was left stirring for 3 h at r.t. The solvent was removed in vacuo and the crude was washed with MeOH to give 610 mg of the boc protected intermediate. The solid was dissolved in DCM (10 mL) and HCl 2M in Et₂O was added slowly. The solution was left stirring at r.t. overweekend. The precipitate was filtered and washed with Et₂O (3×5 mL) to give the titled compound as a pale pink solid. (460 mg, yield 72%).

¹HNMR (DMSO) δ: 1.00-1.10 (2H, m), 1.26-1.36 (2H, m), 1.62-1.66 (2H, m), 1.72-1.75 (3H, m), 2.18-2.25 (1H, m), 3.27 (3H, s), 3.64 (2H, d, J=8.0 Hz)), 3.74 (3H, s), 6.63 (1H, dd, J=8.0 and 1.6 Hz), 6.83 (1H, d, J=1.6 Hz), 7.01 (d, 1H, J=8.0 Hz), 10.29, (2H, bs), 10.88 (1H, s)

C17H24N4O3 Mass (calculated) [332.41]. found [M+H⁺]=333, RT=0.88 (method f)

### 1-Methyl-3-trifluoromethyl-1H-pyrazole-4-carboxylic acid N′-[trans-4-(6-methoxy-3-methyl-2-oxo-2,3-dihydro-benzoimidazol-1-ylmethyl)-cyclohexanecarbonyl]-hydrazide

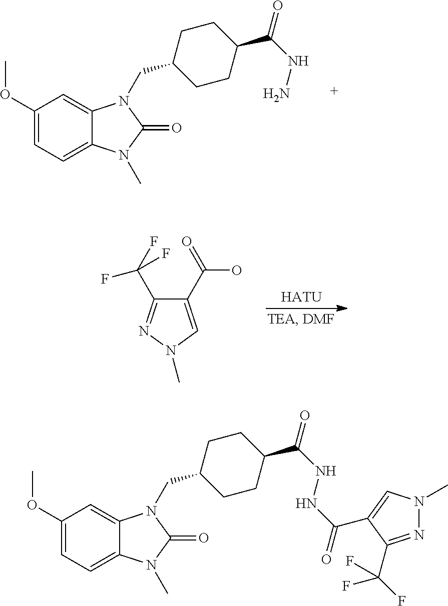

Trans-N′-4-(6-Methoxy-3-methyl-2-oxo-2,3-dihydro-benzoimidazol-1-ylmethyl)-cyclohexanecarboxylic acid hydrazide (180 mg, 0.49 mmol), HATU (223 mg, 0.59 mmol), TEA (0.15 mL, 1.08 mmol) and 3-(trifluoromethyl)-1H-pyrazol-4-carboxylic acid (114 mg, 0.59 mmol) were dissolved in DMF (2 mL) and the mixture was left stirring overnight at r.t. The solvent was removed in vacuo and the crude was washed with MeOH to give the titled compound (75 mg, yield 33%).

¹HNMR (DMSO) δ: 1.00-1.10 (2H, m), 1.27-1.37 (2H, m), 1.63-1.66 (2H, m), 1.73-1.76 (3H, m), 2.13-2.20 (1H, m), 3.27 (3H, s), 3.64 (2H, d, J=8.0 Hz), 3.74 (3H, s), 3.95 (3H, s), 6.62 (1H, dd, J=8.0 and 1.6 Hz), 6.85 (1H, d, J=1.6 Hz), 7.01 (1H, d, J=8.0 Hz) 8.32 (1H, s), 9.77 (1H, bs), 10.06 (1H, bs).

C22H25F₃N6O4 Mass (calculated) [508.50]. found [M+H⁺]=509, RT=1.17 (method f)

### 5-Methoxy-1-methyl-3-{trans-4-[5-(1-methyl-3-trifluoromethyl-1H-pyrazol-4-yl)-[1,3,4]oxadiazol-2-yl]-cyclohexylmethyl}-1,3-dihydro-benzoimidazol-2-one

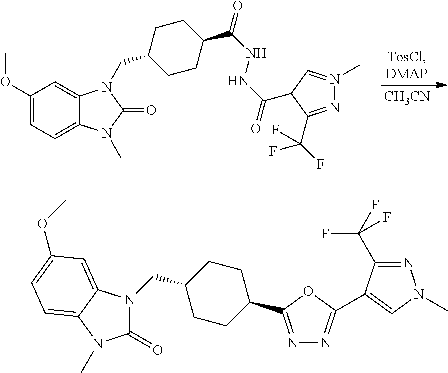

1-Methyl-3-trifluoromethyl-1H-pyrazole-4-carboxylic acid N′-[trans-4-(6-methoxy-3-methyl-2-oxo-2,3-dihydro-benzoimidazol-1-ylmethyl)cyclohexane carbonyl]-hydrazide (154 mg, 0.30 mmol), DMAP (185 mg, 0.97 mmol), tosylchloride (92.5 mg, 0.49 mmol) were mixed in a microwave tube and irradiated at 140° C. for 15 minutes. The crude was dissolved in DCM (10 mL), washed with NaOH 1N (10 mL) and then with HCl 1N (10 mL). The organic layers were collected, concentrated under reduced pressure and the crude purified by reverse phase chromatography using H₂O:CH₃CN as eluents with a gradient 05:95 to 95:05 and with 0.1% formic acid as phase modifier. The titled compound was isolated as a powder (43 mg yield 29%).

¹HNMR (DMSO) δ: 1.21 (2H, dd, J=12.4 and 3.6 Hz), 11.48 (2H, dd, 12.4 and 3.6 Hz), 1.72 (2H, d, J=12.6 Hz), 1.78-1.87 (1H, m), 2.01 (2H, d, J=12.6 Hz), 2.88-2.98 (1H, m), 3.27 (3H, s), 3.68 (2H, d, J=7.4 Hz)), 3.75 (3H, s), 3.998 (3H, s), 6.63 (1H, dd, J=8.2 and 2.4 Hz), 6.85 (1H, d, J=2.4 Hz), 7.02 (1H, d, J=8.2 Hz) 8.69 (1H, s).

C23H25F₃N6O3 Mass (calculated) [490.49]. found [M+H⁺]=491, RT=1.46 (method f)

### Example 12

### (Method H1): 3-{Trans 4-[5-(1-tert-Butyl-5-methyl-2H-pyrazol-3-yl)[1,3,4]oxadiazol-2-yl]-cyclohexylmethyl}-5-methoxy-1-methyl-1,3-dihydro-benzoimidazol-2-one

### 1-tert-Butyl-5-methyl-1H-pyrazole-3-carboxylic acid N′-[trans-4-(6-methoxy-3-methyl-2-oxo-2,3-dihydro-benzoimidazol-1-ylmethyl)-cyclohexane carbonyl]-hydrazide

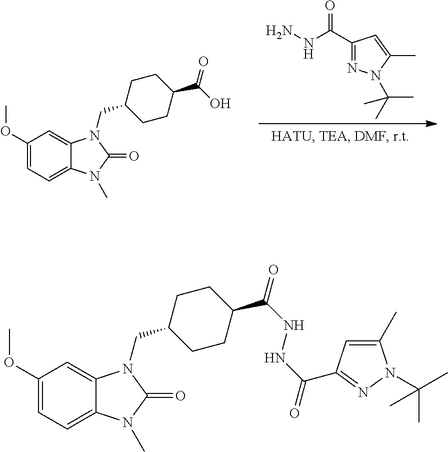 [SMILES: COc1ccc2c(c1)n(C[C@H]1CC[C@H](C(=O)O)CC1)c(=O)n2C ; Cc1cc(C(=O)NN)nn1C(C)(C)C ; COc1ccc2c(c1)n(C[C@H]1CC[C@H](C(=O)NNC(=O)c3cc(C)n(C(C)(C)C)n3)CC1)c(=O)n2C]

Trans-4-(6-Methoxy-3-methyl-2-oxo-2,3-dihydro-benzoimidazol-1-ylmethyl)cyclohexanecarboxylic acid (190 mg, 1.5 mmol), HATU (580 mg, 1.5 mmol), TEA (0.73 mL, 1.5 mmol) and 1-tert-Butyl-1H-pyrazole-3-carboxylic acid hydrazide (250 mg, 1.3 mmol) were dissolved in DMF (7 mL) and the mixture was left stirring overnight at r.t. The solvent was removed in vacuo and the crude dissolved in MeOH (1 mL). The titled compound was purified by reverse phase chromatography using H₂O:CH₃CN as eluents with a gradient 05:95 to 95:05 and with 0.1% formic acid as phase modifier. The titled compound was isolated as a pale yellow solid (242 mg, yield 38%).

¹HNMR (DMSO) δ: 1.00-1.10 (2H, m), 1.27-1.37 (2H, m), 1.57-1.67 (11H, m), 1.73-1.76 (3H, m), 2.13-2.20 (1H, m), 2.42 (3H, s), 3.27 (3H, s), 3.64 (2H, d, J=8.0 Hz)), 3.74 (3H, s), 6.43 (1H, s), 6.62 (1H, d, J=8.0 Hz), 6.85 (1H, d, J=0.6 Hz), 7.01 (1H, d, J=8.0 Hz) 9.57 (1H, s), 9.65 (1H, s).

C26H36N6O4 Mass (calculated) [496.61]. found [M+H⁺]=497, RT=1.32 (method f)

### 3-{trans 4-[5-(1-tert-Butyl-5-methyl-2H-pyrazol-3-yl)-[1,3,4]oxadiazol-2-yl]-cyclohexylmethyl}-5-methoxy-1-methyl-1,3-dihydro-benzoimidazol-2-one

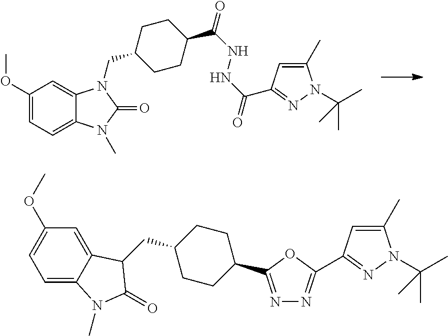

1-tert-Butyl-5-methyl-1H-pyrazole-3-carboxylic acid N′-[trans-4-(6-methoxy-3-methyl-2-oxo-2,3-dihydro-benzoimidazol-1-ylmethyl)-cyclohexanecarbonyl]-hydrazide, (240 mg, 0.48 mmol) DMAP (296 mg, 2.42 mmol) tosylchloride (230 mg, 1.21 mmol) were mixed in a microwave tube and irradiated at 140 C for 15 minutes twice. The crude was dissolved in DCM (10 mL), washed with NaOH 1N (10 mL), and then with HCl 1N (10 mL). The organic layers were collected, concentrated and purified by reverse phase chromatography using H₂O:CH₃CN as eluents with a gradient 05:95 to 95:05 and with 0.1% formic acid as phase modifier. The titled compound was isolated as a powder (47 mg, yield 20%).

¹HNMR (DMSO) δ: 1.15-1.26 (2H, m), 1.40-1.51 (2H, m), 1.59 (9H, s), 1.70-1.73 (2H, m), 1.82-1.88 (1H, m), 2.06-2.09 (2H, m), 2.88-2.96 (1H, m), 3.28 (3H, s), 3.68 (2H, d, J=8.0 Hz), 3.75 (3H, s), 6.62-6.64 (2H, m), 6.85-6.86 (1H, m), 7.00-7.03 (1H, m).

C26H34N6O3 Mass (calculated) [478.60]. found [M+H⁺]=479, RT=3.68 (method c)

### Example 13

### (Method I): 5-Methoxy-1-methyl-3-[trans-4-(5-pyridin-4-yl-2H-pyrazol-3-yl)-cyclohexylmethyl]-1,3-dihydro-benzoimidazol-2-one

### Trans-4-(6-Methoxy-3-methyl-2-oxo-2,3-dihydro-benzoimidazol-1-ylmethyl)-cyclohexanecarboxylic acid methoxy-methyl-amide

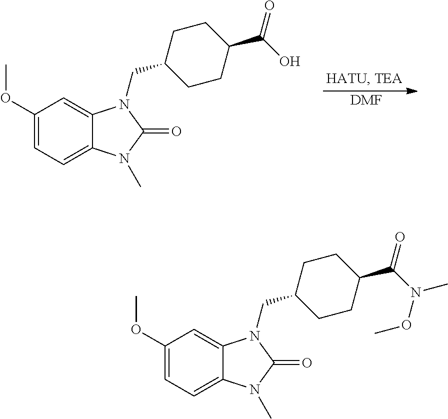

Trans-4-(6-Methoxy-3-methyl-2-oxo-2,3-dihydro-benzoimidazol-1-ylmethyl)-cyclohexanecarboxylic acid (1.5 g, 4.72 mmol) was dissolved in DMF (10 ml). HATU (2.15 g, 5.66 mmol), TEA (1.57 ml, 11.32 mmol) and N,O dimethyl hydroxylamine hydrochloride (552 mg, 5.67 mmol) were added and the mixture was stirred at r.t. overnight. The solvent was removed under reduced pressure and the crude was dissolved in 20 ml of DCM and the solution was washed with H₂O (50 ml), Na₂CO₃ (0.4 M, 50 ml) and then with 1N HCl (50 ml). The organic phase was dried over Na₂SO₄, filtered and the solvent removed. The titled compound was purified by reverse phase chromatography using H₂O:CH₃CN as eluents with a gradient 05:95 to 95:05 and with 0.1% formic acid as phase modifier. The titled compound was isolated as a powder (1.40 g, yield 82%).

¹HNMR (DMSO) δ: 1.02-1.12 (2H, m), 1.19-1.29 (2H, m), 1.60-1.79 (5H, m), 2.55-2-62 (1H, m), 3.03 (3H, s), 3.26 (3H, s), 3.62-3.63 (5H, m), 3.73 (3H, s), 6.61 (1H, dd, J=8.0 and 3.0 Hz), 6.82 (1H, d, J=3.0 Hz), 6.99 (1H, d, J=8.0 Hz)

C19H27N3O4 Mass (calculated) [361.44]. found [M+H⁺]=362, RT=1.24 (method f)

### 3-(trans-4-Acetyl-cyclohexylmethyl)-5-methoxy-1-methyl-1,3-dihydro-benzoimidazol-2-one

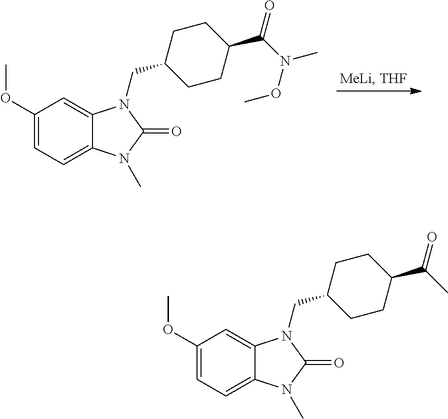 [SMILES: COc1ccc2c(c1)n(C[C@H]1CC[C@H](C(=O)N(C)OC)CC1)c(=O)n2C ; COc1ccc2c(c1)n(C[C@H]1CC[C@H](C(C)=O)CC1)c(=O)n2C]

MeLi (2.8 mL, 1.6 M in Et₂O) was added to a solution of trans-4-(6-Methoxy-3-methyl-2-oxo-2,3-dihydro-benzoimidazol-1-ylmethyl)-cyclohexanecarboxylic acid methoxy-methyl-amide (1.15 g, 3.2 mmol) in anhydrous THF (6 mL) under N₂ at −78° C. After 90 minutes the solution was allowed to reach r.t. and the solvent was removed under reduced pressure. The crude was dissolved in DCM (15 ml) and washed with brine (10 ml). The organic layer was collected, dried over Na₂SO₄, filtered and the solvent removed under reduced pressure. The crude was purified by silica column using 88:12 to 0:100 Cyclohehane/AcOEt. The titled compound was isolated as a white solid (860 mg, yield 85%).

¹HNMR (DMSO) δ: 0.99-1.15 (4H, m), 1.62-1.73 (3H, m), 1.80-1.83 (2H, m), 2.05 (3H, s), 2.23-2-30 (1H, m), 3.26 (3H, s), 3.63 (2H, d, J=8.0 Hz), 3.73 (3H, s), 6.61 (1H, dd, J=8.0 and 3.0 Hz), 6.82 (1H, d, J=3.0 Hz), 7.00 (1H, d, J=8.0 Hz).

C18H24N2O3 Mass (calculated) [316.40]. found [M+H⁺]=317, RT=1.30 (method f)

### 1-[trans-4-(6-Methoxy-3-methyl-2-oxo-2,3-dihydro-benzoimidazol-1-ylmethyl)-cyclohexyl]-3-pyridin-4-yl-propane-1,3-dione

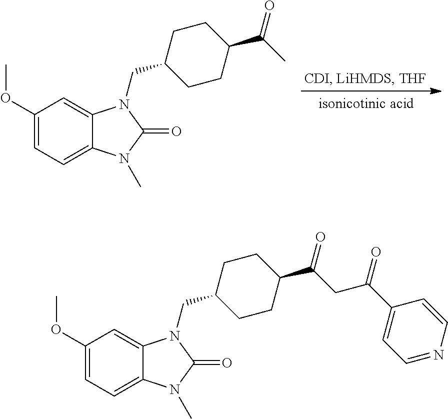 [SMILES: COc1ccc2c(c1)n(C[C@H]1CC[C@H](C(C)=O)CC1)c(=O)n2C ; COc1ccc2c(c1)n(C[C@H]1CC[C@H](C(=O)CC(=O)c3ccncc3)CC1)c(=O)n2C]

CDI (154 mg, 0.95 mmol), was added to a stirred suspension of isonicotinic acid (117 mg, 0.95 mmol) in anhydrous THF (2 ml). The mixture was stirred for 1 h until complete dissolution. In a separated round bottom flask LiHMDS (1.04 ml, 1.04 mmol) was added to a solution of 3-(trans 4-Acetyl-cyclohexylmethyl)-5-methoxy-1-methyl-1,3-dihydro-benzoimidazol-2-one (300 mg, 0.95 mmol) in anhydrous THF (2 mL) at −78° C. under nitrogen. The mixture was left to react for 30 minutes. Finally the CDI activated isonicotinic acid solution was added and the resulting mixture was left stirring for 16 h at r.t. Water was added, the THF was evaporated under reduced pressure and DCM was added. The organic phases were separated, dried on Na₂SO₄, filtered, and the solvent evaporated. The residue was purified by silica column using as eluent first Cyclohexane/AcOEt (gradient 88:12 to 0:100) then 1:1 AcOEt/MeOH. The titled compound was isolated as an oil (120 mg, yield 59%).

C24H27N3O4 Mass (calculated) [421.50]. found [M+H⁺]=422, RT=1.47 (method f)

### 5-Methoxy-1-methyl-3-[trans-4-(5-pyridin-4-yl-2H-pyrazol-3-yl)-cyclohexylmethyl]-1,3-dihydro-benzoimidazol-2-one

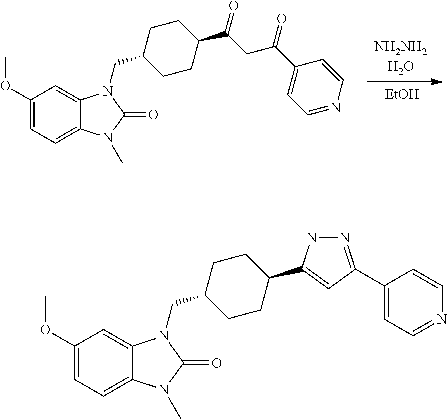 [SMILES: "EtOH" ; COc1ccc2c(c1)n(C[C@H]1CC[C@H](C(=O)CC(=O)c3ccncc3)CC1)c(=O)n2C ; COc1ccc2c(c1)n(C[C@H]1CC[C@H](c3cc(-c4ccncc4)n[nH]3)CC1)c(=O)n2C]

Hydrazine monohydrate (0.017 ml, 0.35 mmol) was added to a stirred solution of -1-[trans-4-(6-Methoxy-3-methyl-2-oxo-2,3-dihydro-benzoimidazol-1-ylmethyl)-cyclohexyl]-3-pyridin-4-yl-propane-1,3-dione (120 mg, 0.29 mmol) in EtOH (2 mL). The resulting mixture was left stirring for 16 h at 70° C. The solvent was evaporated, and the residue was purified by SCX (DCM-MeOH 1:1, then 2.0 N NH₃ in MeOH). The solvent was removed under reduced pressure and the residue was dissolved in DCM (10 ml) and then washed Na₂CO₃ 0.4 M. The organic layer was separated, dried over Na₂SO₄, filtered, and the solvent removed. The titled compound was isolated as a powder (58 mg yield 48%).

¹HNMR (DMSO) δ: 0.98-1.13 (2H, m), 1.23-1.30 (2H, m), 1.56-1.66 (2H, m), 1.67-1.81 (1H, m), 1.82-1.90 (2H, m), 2.16-2-27 (1H, m), 3.28 (3H, s), 3.64-3.72 (2H, m), 3.74 (3H, s), 6.62 (1H, dd, J=8.4 and 2.0 Hz), 6.84 (1H, d, J=2.0 Hz), 6.98 (1H, d, J=8.4 Hz)

C24H27N5O2 Mass (calculated) [417.52]. found [M+H⁺]=418, RT=1.00 (method f)

### Example 14

### (Method L1): 3-[trans-4-(4-Acetyl-piperazine-1-carbonyl)-cyclohexylmethyl]-5-ethynyl-1-methyl-1,3-dihydro-benzoimidazol-2-one

### Trans-4-(6-Bromo-3-methyl-2-oxo-2,3-dihydro-benzoimidazol-1-ylmethyl)-cyclohexanecarboxylic acid methyl ester

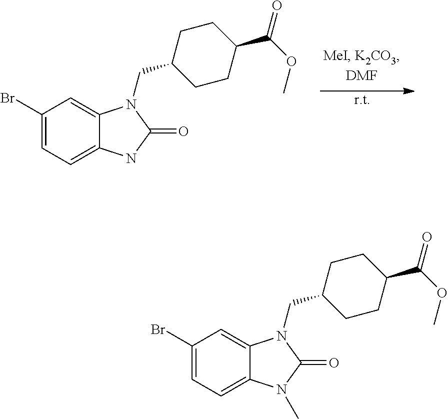 [SMILES: COC(=O)[C@H]1CC[C@H](Cn2c(=O)[nH]c3ccc(Br)cc32)CC1 ; "r.t." ; COC(=O)[C@H]1CC[C@H](Cn2c(=O)n(C)c3ccc(Br)cc32)CC1]

MeI (1.1 mL, 17.32 mmol) was added dropwise to a suspension of trans-4-(6 bromo-2-oxo-2,3-dihydro-benzoimidazol-1-ylmethyl)-cyclohexanecarboxylic acid methyl ester (4.7 g, 13.32 mmol) in DMF (10 mL) containing K₂CO₃ (3.99 g, 17.32 mmol). The reaction mixture was stirred at r.t. 2 h. The crude was concentrated under reduced pressure. The residue was dissolved in DCM (20 mL) and washed with water (2×20 mL). The organic layer was separated, dried over Na₂SO₄ and concentrated to give 5.0 g of the titled compound as a white solid (yield 85%).

¹HNMR (CDCl₃) δ: 1.05-1.16 (2H, m), 1.33-1.44 (2H, m), 1.77-1.89 (3H, m), 1.97-2.02 (2H, m), 2.21-2.29 (1H, m), 3.39 (3H, s), 3.64 (3H, s), 3.67 (2H, d, J=8 Hz), 6.83 (1H, d, J=8.0 Hz), 7.08 (1H, d, J=2.0 Hz), 7.20 (1H, dd, J=8.0 and 2.0 Hz).

### Trans-4-(3-Methyl-2-oxo-6-trimethylsilanylethynyl-2,3-dihydro-benzoimidazol-1-ylmethyl)-cyclohexanecarboxylic acid methyl ester

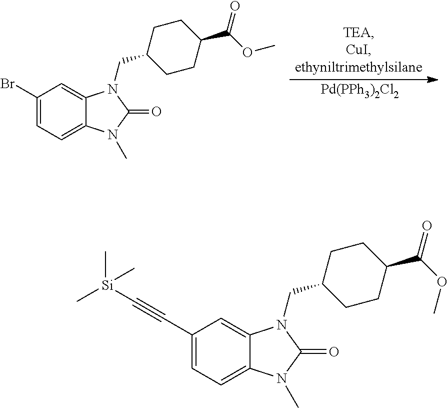

TEA (20 mL), ethinyltrimethylsilane (0.335 g, 3.41 mmol), CuI (50 mg, 0.26 mmol) and Pd(PPh₃)₂Cl₂ (184 mg, 0.26 mmol) were added to trans-4-(6-Bromo-3-methyl-2-oxo-2,3-dihydro-benzoimidazol-1-ylmethyl)-cyclohexanecarboxylic acid methyl ester (1.0 g, 2.62 mmol) and the mixture was stirred at 100° C. overnight. The mixture was concentrated under reduced pressure, AcOEt (20 mL) was added and the organic solution was washed with water (10 mL). The organic phase was dried over Na₂SO₄ filtered and concentrated. The crude was purified by silica column using Cyclohexane/AcOEt 1:1 as eluent system. Obtained 0.835 g of the titled compound solid (yield 80%).

¹HNMR (CDCl₃) δ: 1.04-1.13 (2H, m), 1.31-1.41 (2H, m), 1.74-1.87 (3H, m), 1.94-1.98 (2H, m), 2.20-2.26 (1H, m), 3.38 (3H, s), 3.62 (3H, s), 3.65 (2H, d, J=7.6 Hz), 6.84-6.86 (1H, m), 7.01 (1H, bs), 7.20-7.23 (2H, m).

### Trans-4-(6-Ethynyl-3-methyl-2-oxo-2,3-dihydro-benzoimidazol-1-ylmethyl)-cyclohexanecarboxylic acid

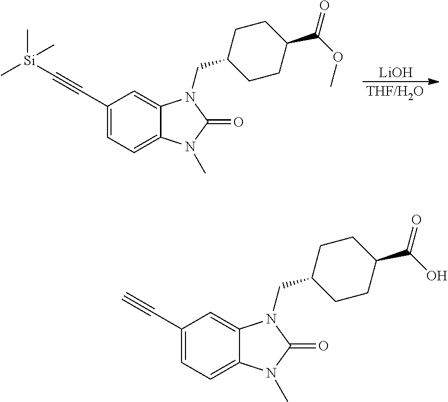

Li(OH) (150 mg, 6.28 mmol) was added to a stirred solution of Trans-4-(3-Methyl-2-oxo-6-trimethylsilanylethynyl-2,3-dihydro-benzoimidazol-1-ylmethyl)-cyclohexanecarboxylic acid methyl ester (835 mg, 2.09 mmol) in a mixture of THF/Water 2:1 (15 mL). The reaction was heated at 60° C. 16 hours. The reaction mixture was concentrated under reduced pressure and the acqueous solution was acidified to pH 3 with HCl 6 N to afford a dark brown solid that was filtered and dried. Obtained 630 mg of the titled compound (yield 97%).

C18H20N2O3 Mass (calculated) [312.37]. found [M+H+]=313 RT=1.24 (method f)

### 3-[trans-4-(4-Acetyl-piperazine-1-carbonyl)-cyclohexylmethyl]-5-ethynyl-1-methyl-1,3-dihydro-benzoimidazol-2-one

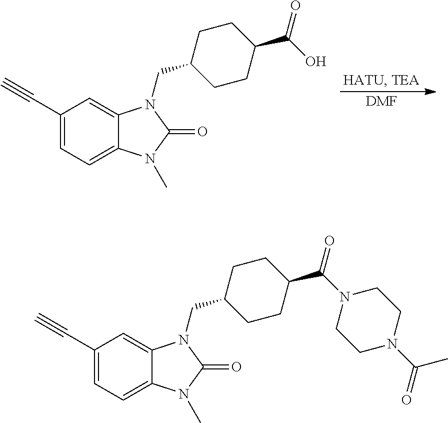

Trans-4-(6-Ethynyl-3-methyl-2-oxo-2,3-dihydro-benzoimidazol-1-ylmethyl)-cyclohexanecarboxylic acid (60 mg, 0.19 mmol) was dissolved in DMF (2 mL) then TEA (0.032 mL, 0.23 mmol), HATU (73 mg, 0.19 mmol) and 1-acetylpiperazine (0.26 mL, 0.23 mmol) were added. The solution was stirred for 16 h then it was concentrated under reduced pressure. The crude was purified by silica column eluting with AcOEt/MeOH 9:1. to afford the titled compound that was further purified by HPLC (method c). Obtained 20 mg of the titled compound as white solid (yield 25%).

¹HNMR (CDCl₃) δ: 1.10-1.20 (2H, m), 1.49-1.59 (2H, m), 1.75-1.85 (4H, m), 1.87-1.97 (1H, m), 2.12 (3H, s), 2.41-2.47 (1H, m), 3.06 (1H, s), 3.40-3.50 (7H, m) 3.58-3.62 (4H, m), 3.72 (2H, d), J=8.0 Hz), 6.91 (1H, d, J=8.0 Hz), 7.09 (1H, d, J=1.6 Hz), 7.26-7.29 (1H, m).

C24H30N4O3 Mass (calculated) [422.53]. found [M+H⁺]=423 RT=1.15 (method f)

### Example 15

### (Method L2): 3-[trans-4-(4-Acetyl-piperazine-1-carbonyl)-cyclohexylmethyl]-5-(2-methoxy-ethylamino)-1-methyl-1,3-dihydro-benzoimidazol-2-one

### Trans-4-[3-Methyl-2-oxo-6-(4,4,5,5-tetramethyl-[1,3,2]dioxaborolan-2-yl)-2,3-dihydro-benzoimidazol-1-ylmethyl]-cyclohexanecarboxylic acid methyl ester

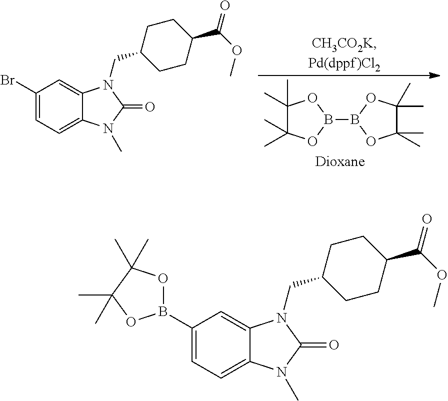

Trans-4-(6-Bromo-3-methyl-2-oxo-2,3-dihydro-benzoimidazol-1-ylmethyl)-cyclohexanecarboxylic acid methyl ester (1.0 g, 2.62 mmol), bis(pinacolato)diboron (0.733 g, 2.89 mmol), Pd(dppf)Cl₂ (0.214 g, 0.26 mmol), CH₃COOK (0.90 g, 9.2 mmol) were mixed together, then dioxane (10 mL) was added. The mixture was left stirring at 90° C. 4 h. AcOEt (15 mL) and water (15 mL) were added. The organic phase was separated, dried over Na₂SO₄ and concentrated under reduced pressure. The residue was purified by silica column using Cyclohexane/AcOEt 4:1 as eluent system. Obtained 1.08 g of the titled compound (yield quantitative).

C23H33BN2O5 Mass (calculated)=428.34. found [M+H⁺]=429, RT 1.74 (method f)

### Trans-4-[6-(2-Methoxy-ethylamino)-3-methyl-2-oxo-2,3-dihydro-benzoimidazol-1-ylmethyl]-cyclohexanecarboxylic acid methyl ester

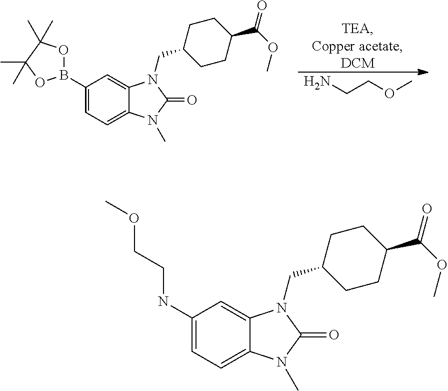

Trans-4-[3-Methyl-2-oxo-6-(4,4,5,5-tetramethyl-[1,3,2] dioxaborolan-2-yl)-2,3-dihydro-benzoimidazol-1-ylmethyl]-cyclohexanecarboxylic acid methyl ester (0.8 g, 1.87 mmol), Cu(Ac)₂ (0.51 g, 2.8 mmol), TEA (0.52 mL, 3.74 mmol) and 2-methoxyethylamine (0.65 mL, 7.47 mmol) in DCM (10 ml) were stirred at r.t. over weekend. Water (10 ml) was added. The organic layer was dried over Na₂SO₄, filtered and concentrated under reduced pressure. The residue was purified on silica column using Cyclohexane/AcOEt 3:7 as eluent system. Obtained 150 mg of the titled compound (yield 21%).

¹HNMR (CDCl3) δ: 1.02-1.12 (2H, m), 1.28-1.39 (2H, m), 1.75-1.85 (3H, m), 1.93-1.97 (2H, m), 2.17-2.25 (1H, m), 3.25 (2H, t, J=10.4 Hz), 3.32 (3H, s), 3.37 (3H, s), 3.59-3.63 (7H, m), 6.28 (1H, d, J=2.0 Hz), 6.37 (1H, dd, J=8.4 and 2.0 Hz), 6.75 (1H, d, J=8.4 Hz).

### Trans-4-[6-(2-Methoxy-ethylamino)-3-methyl-2-oxo-2,3-dihydro-benzoimidazol-1-ylmethyl]-cyclohexanecarboxylic acid

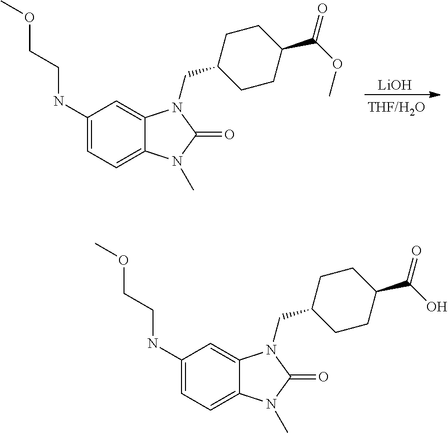

To a solution of trans-4-[6-(2-Methoxy-ethylamino)-3-methyl-2-oxo-2,3-dihydro-benzoimidazol-1-ylmethyl]-cyclohexanecarboxylic acid methyl ester (100 mg, 0.27 mmol) in THF (2 mL), LiOH (19 mg, 0.80 mmol) in water (1 mL) was added. The mixture was left stirring for 3 h then it was concentrated under reduced pressure. HCl 1N (2 mL) was added and the solution was extracted with DCM (2×10 mL). Organic layers were collected and concentrated to give 65 mg of the titled compound as white solid (yield 67%).

C19H27N3O4 Mass (calculated) [361.44]. found [M+H⁺]=361 RT=0.88 (method f)

### 3-[trans-4-(4-Acetyl-piperazine-1-carbonyl)-cyclohexylmethyl]-5-(2-methoxy-ethylamino)-1-methyl-1,3-dihydro-benzoimidazol-2-one

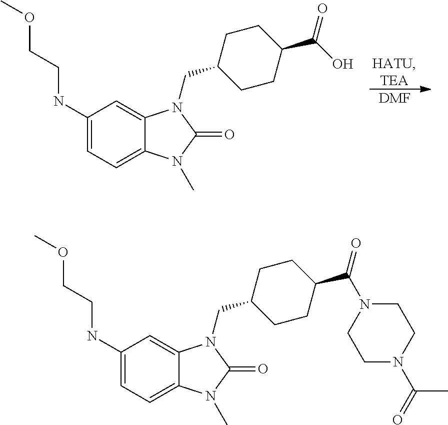

Trans-4-[6-(2-Methoxy-ethylamino)-3-methyl-2-oxo-2,3-dihydro-benzoimidazol-1-ylmethyl]-cyclohexanecarboxylic acid (65 mg, 0.18 mol) was dissolved in DMF (2 mL); TEA (0.034 mL, 0.22 mmol), HATU (125 mg, 0.33 mmol) and 1-Acetylpiperazine (28 mg, 0.22 mmol) were added. The solution was stirred overnight then it was concentrated under reduced pressure. The crude was purified first by silica column (AcOEt/MeOH 9:1) then by preparative HPLC (method c) obtaining 10 mg of the titled compound (yield 12%).

¹HNMR (CDCl₃) δ: 1.04-1.13 (2H, m), 1.42-1.50 (2H, m), 1.68-1.88 (5H, m), 2.05 (3H, s), 2.32-2.40 (1H, m), 3.22 (2H, t, J=7.2 Hz), 3.29 (3H, s), 3.34 (3H, s), 3.36-3.43 (4H, m), 3.52-3.62 (8H, m), 3.90 (1H, bs), 6.25 (1H, d, J=2.0 Hz), 6.35 (1H, dd, J=2.0 and 8.4 Hz), 6.72 (1H, d, J=8.4 Hz).

C25H37N5O4 Mass (calculated) [471.60]. found [M+H⁺]=472, RT=0.75 (method f)

### Example 16

### (Method L3): 3-[trans-4-(4-Acetyl-piperazine-1-carbonyl)-cyclohexylmethyl]-5-hydroxy-1-methyl-1,3-dihydro-benzoimidazol-2-one

### Trans-4-(6-Hydroxy-3-methyl-2-oxo-2,3-dihydro-benzoimidazol-1-ylmethyl)-cyclohexanecarboxylic acid methyl ester

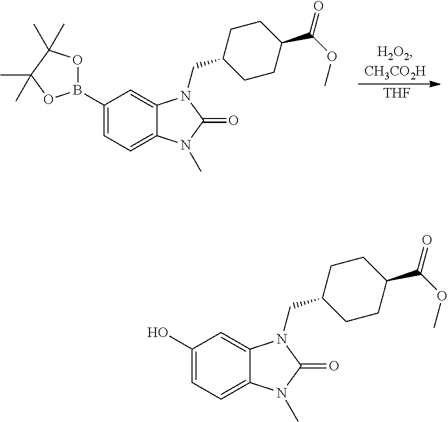

Trans-4-[3-Methyl-2-oxo-6-(4,4,5,5-tetramethyl-[1,3,2] dioxaborolan-2-yl)-2,3-dihydro-benzoimidazol-1-ylmethyl]-cyclohexanecarboxylic acid methyl ester (0.8 g, 1.87 mmol), was dissolved in THF (10 ml). H₂O2 (0.5 ml) and CH₃CO₂H (0.10 ml) were added and the mixture was stirred at r.t. over weekend. The mixture was concentrated under reduced pressure then water (10 ml) and DCM (10 mL) were added. The organic layer was separated, dried over Na₂SO₄ and concentrated. The residue was purified by silica column using Cyclohexane/AcOEt 3:7 as eluent system. Obtained 300 mg of the titled compound (yield 51%).

¹HNMR (CDCl3) δ: 1.05-1.15 (2H, m), 1.32-1.42 (2H, m), 1.77-1.88 (3H, m), 1.97-2.01 (2H, m), 2.21-2.29 (1H, m), 3.38 (3H, s), 3.65 (3H, s), 3.66 (2H, d, J=7.2 Hz), 6.54-6.55 (1H, m), 6.56-6.59 (1H, m), 6.78-6.80 (1H, m).

C17H22N2O4 Mass (calculated) [318.38]. found [M+H⁺]=319 RT=1.09 (method f)

### Trans-4-(6-Hydroxy-3-methyl-2-oxo-2,3-dihydro-benzoimidazol-1-ylmethyl)-cyclohexanecarboxylic acid

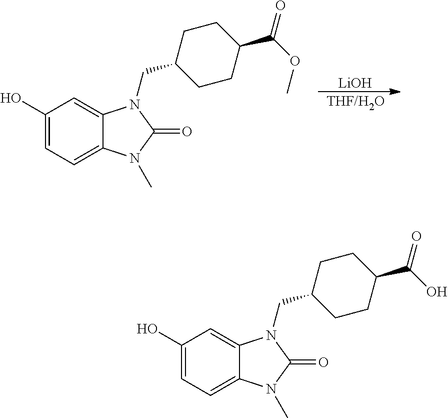

LiOH (19 mg, 0.80 mmol) in water (1 mL) was added to a solution of trans-4-(6-Hydroxy-3-methyl-2-oxo-2,3-dihydro-benzoimidazol-1-ylmethyl)-cyclohexane carboxylic acid methyl ester (85 mg, 0.27 mmol) in THF (2 mL). The mixture was left stirring for 3 h then it was concentrated under reduced pressure. HCl 1N (2 mL) was added and the precipitate was filtered to give 55 mg of the titled compound as white solid (yield 67%).

C16H20N2O4 Mass (calculated) [304.35]. found [M+H⁺]=305 RT=0.88 (method f)

### 3-[trans-4-(4-Acetyl-piperazine-1-carbonyl)-cyclohexylmethyl]-5-hydroxy-1-methyl-1,3-dihydro-benzoimidazol-2-one

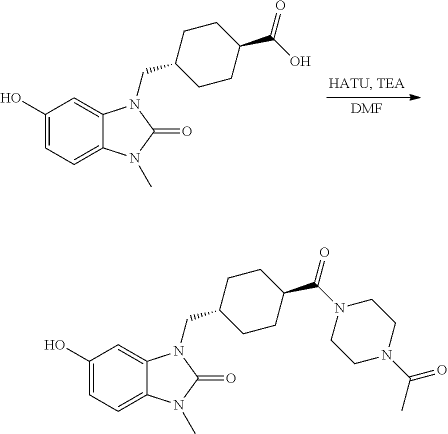

Trans-4-(6-Hydroxy-3-methyl-2-oxo-2,3-dihydro-benzoimidazol-1-ylmethyl)-cyclohexanecarboxylic acid (55 mg, 0.18 mol) was dissolved in DMF (2 mL) then TEA (30 μL, 0.22 mmol), HATU (82 mg, 0.22 mmol) and 1-Acetyl-piperazine (28 mg, 0.22 mmol) were added. The solution was stirred at r.t. overnight. The solution was stirred overnight then it was concentrated under reduced pressure. The crude was purified first by silica column (AcOEt/MeOH 9:1) then by preparative HPLC (method c) obtaining 55 mg of the titled compound (yield 74%).

¹HNMR (CDCl₃) δ: 1.11-1.20 (2H, m), 1.48-1.58 (2H, m), 1.75-1.92 (5H, m), 2.13 (3H, s), 2.41-2.47 (1H, m), 3.37 (3H, s), 3.44-3.54 (4H, m), 3.61-3.69 (6H, m), 6.55-6.56 (1H, m), 6.58-6.61 (1H, m), 6.77-6.79 (1H, m).

C22H30N4O4 Mass (calculated) [414.51]. found [M+H⁺]=415, RT=0.84 (method f)

### Example 17

### (Method L4): Trans-4-(6-ethoxy-2-oxo-2,3-dihydro-benzoimidazol-1-ylmethylcyclohexanecarboxylic acid pyridin-4-ylamide

### Trans-4-(6-Ethoxy-3-methyl-2-oxo-2,3-dihydro-benzoimidazol-1-ylmethyl)-cyclohexanecarboxylic acid methyl ester

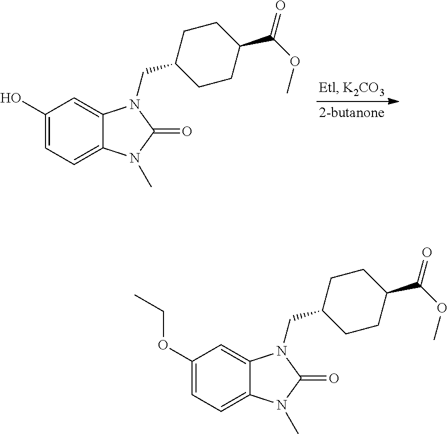

Ethyliodide (36.5 μL, 0.45 mmol) was added to a suspension of trans-4-(6-Hydroxy-3-methyl-2-oxo-2,3-dihydro-benzoimidazol-1-ylmethyl)-cyclohexanecarboxylic acid methyl ester (120 mg, 0.38 mmol) and K₂CO₃ (104 mg, 0.75 mmol) in 2-butanone (2 ml) and the mixture was left stirring at 50° C. overnight. Ethyl iodide was added again (62 μL, 0.76 mmol) and the mixture was heated at 60° C. 24 hours. The reaction mixture was concentrated under reduced pressure and the crude dissolved in DCM (5 ml) and washed with water (7 ml). The organic solution was dried over Na₂SO₄, filtered and filtrate concentrated to give 120 mg of the titled compound (yield 92%).

¹HNMR (CDCl₃) δ: 1.06-1.16 (2H, m), 1.32-1.44 (5H, m), 1.78-1.98 (3H, m), 1.96-2.01 (2H, m), 2.21-2.29 (1H, m), 3.38 (3H, s), 3.64 (3H, s), 3.67 (2H, d, J=7.2 Hz), 4.01 (2H, q, J=7.2 Hz), 6.57 (1H, d, J=2.4 Hz), 6.64 (1H, dd, J=8.4 and 2.4 Hz), 6.84 (1H, d, J=8.4 Hz).

### Trans-4-(6-ethoxy-3-methyl-2-oxo-2,3-dihydro-benzoimidazol-1-ylmethyl)-cyclohexanecarboxylic acid

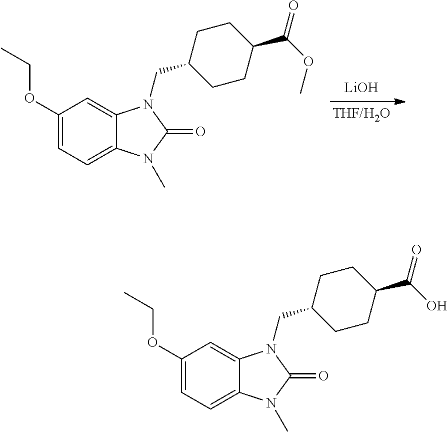

To a solution of trans-4-(6-Ethoxy-3-methyl-2-oxo-2,3-dihydro-benzoimidazol-1-ylmethyl)-cyclohexanecarboxylic acid methyl ester (121 mg, 0.35 mmol) in THF (2 ml), LiOH (25 mg, 1.05 mmol) in water (1 mL) was added. The mixture was left stirring for 4 h. HCl 1N (3 mL) was added and the solution extracted with DCM (5 mL). The organic solution was dried over Na₂SO₄, filtered, and concentrated to give 111 mg of the titled compound as white solid (yield 96%).

C18H24N2O4 Mass (calculated) [332.40]. found [M+H⁺]=333 RT=1.26 (method f)

### Trans-4-(6-ethoxy-2-oxo-2,3-dihydro-benzoimidazol-1-ylmethylcyclohexanecarboxylic acid pyridin-4-ylamide

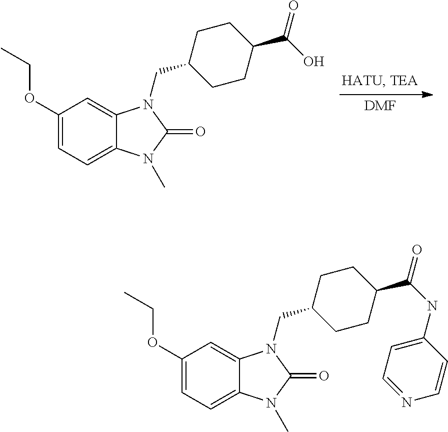

A mixture of trans-4-(6-ethoxy-3-methyl-2-oxo-2,3-dihydro-benzoimidazol-1-ylmethyl)-cyclohexanecarboxylic acid (55 mg, 0.18 mmol), TEA (30 μL, 0.22 mmol), HATU (82 mg, 0.22 mmol) and pyridin-4-ylamine (20 mg, 0.22 mmol) in DMF (2 mL) was stirred at r.t. 4 h. The solution was concentrated under reduced pressure and crude was purified by silica column (AcOEt:MeOH 9:1). The titled compound was dissolved in DCM (3 mL) and washed with Na₂CO₃ saturated solution (3 mL) to afford 50 mg of the titled compound (59 mg, Yield 68%).

¹HNMR (MeOD) δ: 1.11-1.21 (2H, m), 1.44 (3H, t, J=6.8 Hz), 1.50-1.61 (2H, m), 1.85-2.03 (5H, m), 2.23-2.29 (1H, m), 3.39 (3H, s), 3.71 (2H, d, J=7.2 Hz), 4.05 (2H, q, J=6.8 Hz), 6.59 (1H, d, J=2.4 Hz), 6.66 (1H, dd, J=2.4 and 8.4 Hz), 6.86 (1H, d, J=8.4 Hz), 7.46-7.48 (2H, m), 7.71 (1H, bs), 8.46-8.47 (2H, m).

C21H24N4O3 Mass (calculated) [408.50]. found [M+H⁺]=409, RT=1.11 (method f)

### Example 18

### (Method M): 5-Methoxy-3-{trans-4-[5-(4-methoxy-phenyl)[1,2,4]oxadiazol-3-yl]-cyclohexylmethyl}-1-methyl-1,3-dihydro-benzoimidazol-2-one

### Trans-4-(6-Methoxy-3-methyl-2-oxo-2,3-dihydro-benzoimidazol-1-ylmethyl)-cyclohexanecarboxylic acid amide

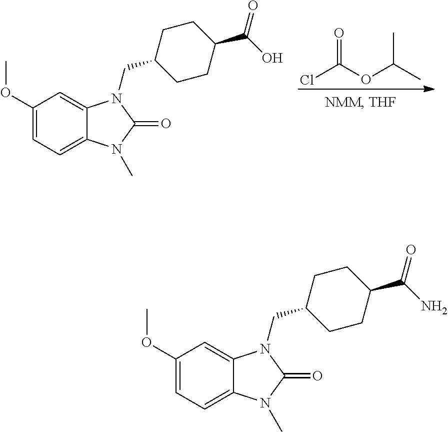

A suspension of trans-4-(6-Methoxy-3-methyl-2-oxo-2,3-dihydro-benzoimidazol-1-ylmethyl)-cyclohexanecarboxylic acid (5.0 g, 15.7 mmol) and NMM (2 mL, 15.7 mmol) in THF (7 ml) was cooled to 0° C. in ice bath. Isoprenylchloroformiate (1M in toluene, 15.7 mL) was added and the mixture was stirred 30 min at 0° C., then NH₄OH solution (25% in water) was added and the mixture was allowed to reach r.t. and stirred for additional 2 h. AcOEt was added and the precipitate was filtered, washed with AcOEt and dried to afford 4.9 g of the titled compound as grey solid (yield 98%).

¹HNMR (CDCl3) δ: 1.01-1.12 (2H, m), 1.30-1.41 (2H, m), 1.75-1.85 (3H, m), 1.88-1.91 (2H, m), 2.01-2.09 (1H, m), 3.32 (3H, s), 3.62 (2H, d, J=8.0 Hz), 3.76 (3H, s), 5.22-5.35 (2H, m), 6.49 (1H, d, J=4.0 Hz), 6.58 (1H, dd, J=8.0 Hz, J=4.0 Hz), 6.79 (1H, d, J=8.0 Hz).

C17H23N3O3 Mass (calculated) [317.39]. found [M+H⁺]=318, RT=1.02 (method f)

### Trans-4-(6-Methoxy-3-methyl-2-oxo-2,3-dihydro-benzoimidazol-1-ylmethyl)-cyclohexanecarbonitrile

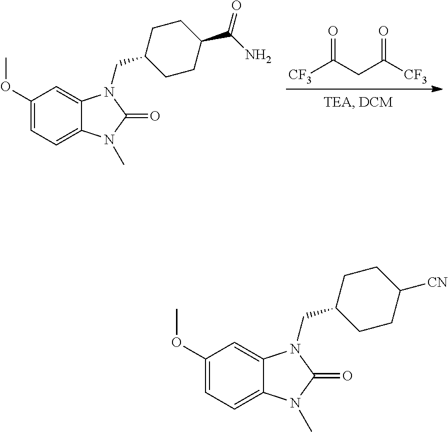

A suspension of trans-4-(6-Methoxy-3-methyl-2-oxo-2,3-dihydro-benzoimidazol-1-ylmethyl)-cyclohexanecarboxylic acid amide (4 g, 12.6 mmol) and TEA (11.4 mL, 82 mmol) in DCM (80 mL) was cooled to 0° C., TFAA (2.2 mL, 15.7 mmol) was added slowly and the resulting mixture was stirred for further 2 h to reach r.t.

The organic phase was washed with water (2×80 mL), and Na₂CO₃ ss (2×80 ml). The organic phase was dried over Na₂SO₄ and solvent evaporated under reduced pressure. The crude was dissolved in CH₃CN (30 mL) and water (35 mL) was added dropwise under vigorous stirring. The mixture was left in an ice bath to give 2.4 g of the titled compound as a grey solid (yield 64%).

¹HNMR (CDCl3) δ: 1.07-1.17 (2H, m), 1.48-1.59 (2H, m), 1.81-1.95 (3H, m), 2.11-2.14 (2H, m), 2.36-2.43 (1H, m), 3.39 (3H, s), 3.68 (2H, d, J=8.0 Hz), 3.83 (3H, s), 6.53-6.54 (1H, m), 6.64-6.67 (1H, m), 6.85-6.87 (1H, m).

### N-Hydroxy-trans-4-(6-methoxy-3-methyl-2-oxo-2,3-dihydro-benzoimidazol-1-ylmethyl)-cyclohexanecarboxamidine

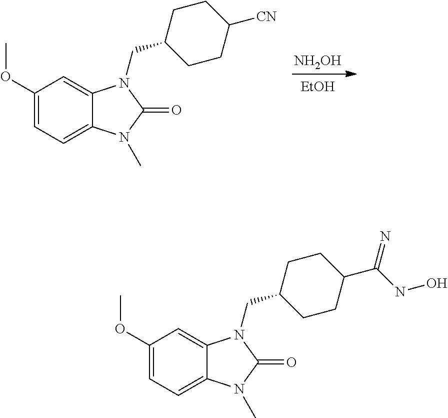

Hydroxylammine (50% wt solution in water, 0.21 Ml) was added to a solution of trans-4-(6-Methoxy-3-methyl-2-oxo-2,3-dihydro-benzoimidazol-1-ylmethyl)-cyclohexanecarbonitrile (500 mg, 1.67 mmol) in EtOH (10 ml). The solution was refluxed overnight then other 1.5 equivalents of hydroxylamine were added and the reaction mixture was refluxed overnight to get complete conversion.

The solvent was evaporated under reduced pressure to afford 554 mg of the titled compound (yield quantitative).

C17H24N4O3 Mass (calculated) [332.41]. found [M+H⁺]=333, RT=0.83 (method f)

### 5-Methoxy-3-{trans-4-[5-(4-methoxy-phenyl)[1,2,4]oxadiazol-3-yl]-cyclohexylmethyl}-1-methyl-1,3-dihydro-benzoimidazol-2-one

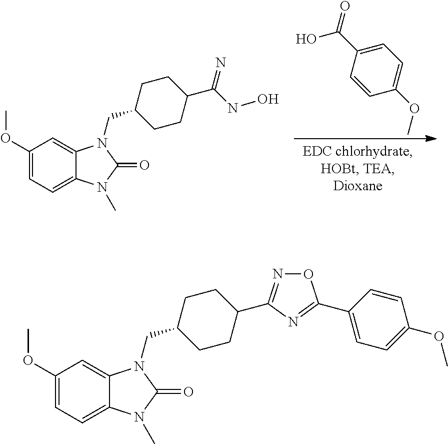

TEA (0.116 ml, 0.83 mmol), HOBT (63 mg, 0.47 mmol) and EDC chloridrate (90 mg, 0.47 mmol) were added to a stirred solution of N-hydroxy-trans-4-(6-methoxy-3-methyl-2-oxo-2,3-dihydro-benzoimidazol-1-ylmethyl)-cyclohexane carboxamidine (120 mg, 0.36 mmol) in dioxane (15 Ml) and the resulting mixture was left overnight at room temperature and then refluxed overnight. The solvent was evaporated under reduced pressure and DCM (10 Ml) was added to the crude. The organic solution was washed with water (10 Ml). The organic phase was separated, dried over Na₂SO₄ filtered, and concentrated. The crude was purified by preparative HPLC (method b), to give 50 mg of the titled compound (yield 31%).

¹HNMR (DMSO) δ: 1.15-1.25 (2H, m), 1.39-1.49 (2H, m), 1.69-1.73 (2H, m), 1.80-1.89 1H, m), 1.99-2.03 (2H, m), 2.72-2.80 (1H, m), 3.27 (3H, s), 3.68 (2H, d, J=8.0 Hz), 3.74 (3H, s), 3.83 (3H, s), 6.62 (1H, dd, J=8.0 Hz, J=2.4 Hz), 6.85 (1H, d, J=8.4 and 2.4 Hz), 7.0 (1H, d, J=8.0 Hz), 7.10-7.13 (2H, m), 7.97-8.00 (2H, m).

C25H28N4O4 Mass (calculated) [448.53]. found [M+H⁺]=449, RT=1.73 (method f)

### Example 19

### (Method N): N-{4-[trans-4-(6-Methoxy-3-methyl-2-oxo-2,3-dihydro-benzoimidazol-1-ylmethyl)-cyclohexyl]-1H-imidazol-2-yl}-acetamide

### 3-[trans-4-(2-Bromo-acetyl)-cyclohexylmethyl]-5-methoxy-1-methyl-1,3-dihydro-benzoimidazol-2-one

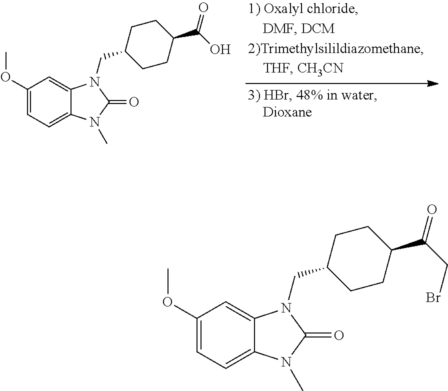 [SMILES: COc1ccc2c(c1)n(C[C@H]1CC[C@H](C(=O)O)CC1)c(=O)n2C ; COc1ccc2c(c1)n(C[C@H]1CC[C@H](C(=O)CBr)CC1)c(=O)n2C]

Oxalyl chloride (0.38 Ml, 4.53 mmol) and DMF (0.03 Ml) were added to a stirred solution of trans-4-(6-Methoxy-3-methyl-2-oxo-2,3-dihydro-benzoimidazol-1-ylmethyl)-cyclohexanecarboxylic acid (1.2 g, 3.77 mmol) in dry DCM (30 Ml). The resulting solution was stirred 16 h at r.t. The solvent was removed under reduced pressure. The crude containing was dissolved in THF/CH₃CN (8 Ml, 1:1 v/v) and the solution cooled to 0° C. Trimethylsilildiazomethane (2.0 M solution in Et₂O, 5.7 Ml, 11.34 mmol) was added dropwise and the resulting mixture was allowed to warm to r.t. and stirred for 3 h. The solvent was removed under reduced pressure. Dioxane (7 Ml) was added to the crude and then HBr (48% solution in water) was slowly added. The mixture was stirred 1 h at r.t. Iced water was added and the mixture was extracted with DCM (5*10 Ml). The organic layers were collected, dried over Na₂SO₄ filtered and concentrated. The residue was purified by silica column (Cyclohexane/AcOEt 95:05 to 05:95) to afford 920 mg of the titled compound (yield 62%).

¹HNMR (DMSO) δ: 1.00-1.21 (4H, m), 1.62-1.65 (2H, m), 1.68-1.78 (1H, m), 1.83-1.86 (2H, m), 2.49-2.58 (1H, m), 3.26 (3H, s), 3.63 (2H, d, J=8.0 Hz), 3.73 (3H, s), 4.44 (2H, s), 6.62 (1H, dd, J=8.0 and 2.4 Hz), 6.81 (1H, d, J=2.4 Hz), 7.00 (2H, d, J=8.0 Hz).

C18H23BrN2O3 Mass (calculated) [395.30]. found [M+H⁺]=395/397, RT=1.46 (method f)

### N-{4-[trans-4-(6-Methoxy-3-methyl-2-oxo-2,3-dihydro-benzoimidazol-1-ylmethyl)-cyclohexyl]-1H-imidazol-2-yl}-acetamide

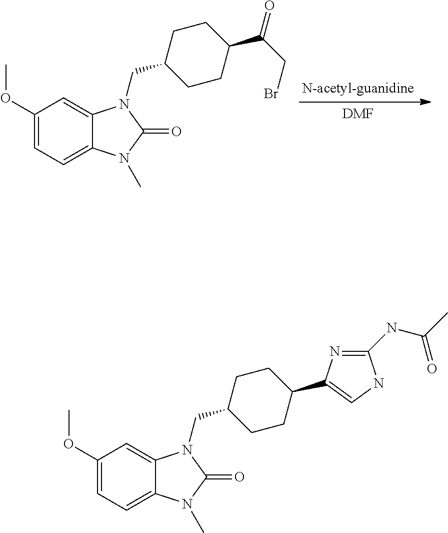

3-[trans-4-(2-Bromo-acetyl)-cyclohexylmethyl]-5-methoxy-1-methyl-1,3-dihydro-benzoimidazol-2-one (250 mg, 0.63 mmol) was added to a stirred solution of N-acetylguanidine (192 mg, 1.9 mmol) in DMF (10 Ml). The resulting mixture was left stirring for 2 days at r.t. The reaction mixture was concentrated under reduced pressure, the crude was dissolved in DCM (10 Ml) and washed with water (10 Ml). The organic layer was separated, dried over Na₂SO₄ filtered and concentrated under reduced pressure. The residue was triturated with CH₃CN, the solid was filtered and dried to give 79 mg of the titled compound (yield 31%).

¹HNMR (DMSO) δ: 1.06-1.23 (4H, m), 1.62-1.658 (2H, m), 1.72-1.80 (1H, m), 1.89-1.92 (2H, m), 1.98 (3H, s), 2.25-2.34 (1H, m), 3.27 (3H, s), 3.65 (2H, d, J=8.0 Hz), 3.74 (3H, s), 6.35 (1H, brs), 6.62 (1H, dd, J=8.0 and 2.4 Hz), 6.83 (1H, d, J=2.4 Hz), 7.00 (2H, d, J=8.0 Hz), 10.83-11.15 (2H, m).

C₂₁H27N5O3 Mass (calculated) [397.48]. found [M+H⁺]=398, RT=0.94 (method f)

### Example 20

### (Method P): 5-Methoxy-1-methyl-3-[trans-4-(4-pyrimidin-5-yl-piperazine-1-carbonyl)-cyclohexylmethyl]-1,3-dihydro-benzoimidazol-2-one

4-[trans-4-(6-Methoxy-3-methyl-2-oxo-2,3-dihydro-benzoimidazol-1-ylmethyl)-cyclohexanecarbonyl]-piperazine-1-carboxylic acid tert-butyl ester

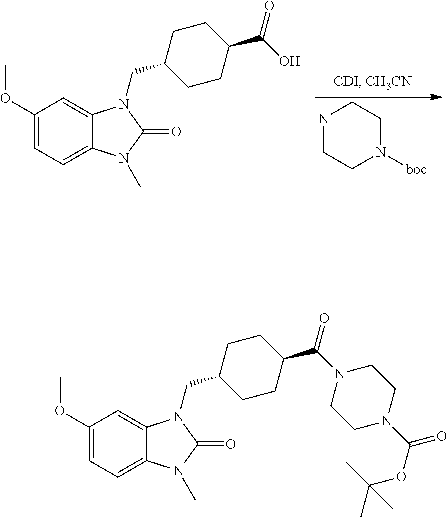

CDI (993 mg, 6.12 mmol) was added to a solution of trans-4-(6-Methoxy-3-methyl-2-oxo-2,3-dihydro-benzoimidazol-1-ylmethyl)-cyclohexanecarboxylic acid (1.5 g, 4.71 mmol) in CH₃CN (12 ml). The mixture was stirred at 50° C. one hour then tert-butyl-1-piperazinecarboxylate (965 mg, 5.18 mmol) was added. The mixture was refluxed 2 hours. The solvent was removed under reduced pressure and the crude was dissolved in DCM (10 ml) and washed with Na₂CO₃ (0.4 M solution, 5 Ml). The organic solution was washed with NH₄Cl (saturated solution, 2×5 Ml), dried over Na₂SO₄, filtered and concentrated to afford 2.28 g of the titled compound (yield 99%).

C26H38N4O5; Mass calculated [486.62]. found [M+H]⁺=487.4 m/z; RT=1.48 min (method f)

### 5-Methoxy-1-methyl-3-[trans-4-(piperazine-1-carbonyl)cyclohexylmethyl]-1,3-dihydro-benzoimidazol-2-one

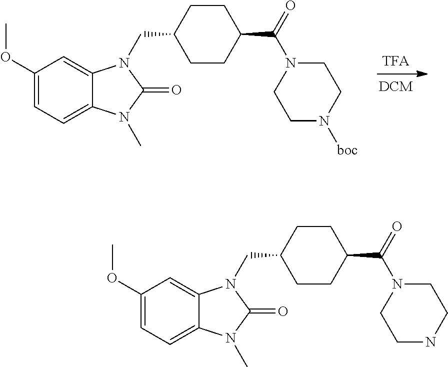

Trifluoro acetic acid (8 ml) was added to a solution of 4-[trans-4-(6-Methoxy-3-methyl-2-oxo-2,3-dihydro-benzoimidazol-1-ylmethyl)-cyclohexanecarbonyl]-piperazine-1-carboxylic acid tert-butyl ester (2.29 g, 4.70 mmol) in DCM (20 ml). The solution was stirred at room temperature overnight and then the reaction mixture was concentrated under reduced pressure. DCM (10 Ml) was added to the crude and the organic solution was washed with NaOH 1N (7 Ml). The organic layer was concentrated to afford 1.82 g of the titled compound as a white foam (yield quantitative).

C21H30N4O3; Mass calculated [386.50]. found [M+H]⁺=387.2 m/z;

RT=0.89 min (method f)

### 5-Methoxy-1-methyl-3-[trans-4-(4-pyrimidin-5-yl-piperazine-1-carbonyl)-cyclohexylmethyl]-1,3-dihydro-benzoimidazol-2-one

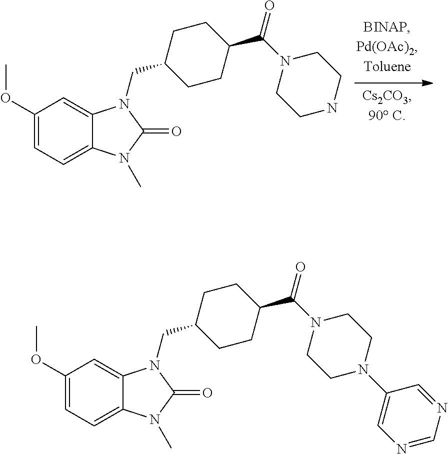 [SMILES: COc1ccc2c(c1)n(C[C@H]1CC[C@H](C(=O)N3CCNCC3)CC1)c(=O)n2C ; COc1ccc2c(c1)n(C[C@H]1CC[C@H](C(=O)N3CCN(c4cncnc4)CC3)CC1)c(=O)n2C]

Toluene (2 Ml) was added to a mixture of Pd(Oac)₂ (6.0 mg, 0.03 mol) and BINAP (16 mg, 0.03 mmol). The resulting mixture was transfer in a vial containing Cs₂CO₃ (252 mg, 0.78 mmol), trans-4-(6-Methoxy-3-methyl-2-oxo-2,3-dihydro-benzoimidazol-1-ylmethyl)-cyclohexanecarboxylic acid piperazin-1-yl ester (100 mg, 0.26 mmol) and 5-bromopyrimidine (53 mg, 0.34 mmol). The resulting mixture was heated under stirring at 90° C. 6 hours. Water (3 Ml) was added, the organic phase was separated, the acqueous phase was extracted twice with DCM (2*3 mL). The organic layers were collected, dried over Na₂SO₄, filtered and concentrated under reduced pressure. The crude was purified by silica column to afford 55 mg of the titled compound (yield 46%).

¹H NMR (CDCl₃) δ: 1.11-1.22 (2H, m), 1.50-1.60 (2H, m), 1.77-1.97 (5H, m), 2.43-2.50 (1H, s), 3.23 (4H, bs), 3.39 (3H, s), 3.65-3.72 (4H, m), 3.79 (2H, bs), 3.83 (3H, s), 6.57 (1H, d, J=2.4 Hz), 6.66 (1H, dd, J=8.4 and 2.4 HZ), 6.86 (1H, d, J=8.4 Hz), 8.38 (2H, s), 8.75 (1H, s).

C25H32N6O3; Mass calculated [464.57]. found [M+H]⁺=465 m/z;

RT=1.12 min (method f)

Examples 21-188 listed in table were made according to the method of column 3 and characterised by NMR (data not shown), and HPLC-MS (columns 4, 5, 6, 7 and 8)

TABLE

| | | Gen- | | | Found | | ana- |
|---|---|---|---|---|---|---|---|
| | | eral | Ex- | Reten- | MW | | lytical |
| Exam- | | meth- | pected | tion | (M + | Pu- | meth- |
| ple | Structure | ods | MW | time | 1) | rity | od |
| 21 | 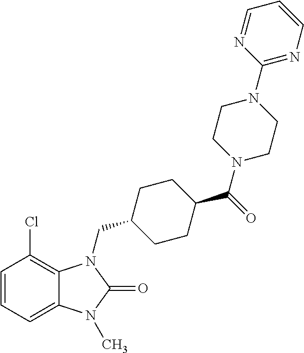 [SMILES: Cn1c(=O)n(C[C@H]2CC[C@H](C(=O)N3CCN(c4ncccn4)CC3)CC2)c2c(Cl)cccc21] | A2 | 468 | 1.46 | 469 | 100 | f |
| 22 | 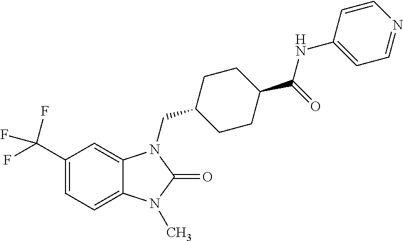 [SMILES: Cn1c(=O)n(C[C@H]2CC[C@H](C(=O)Nc3ccncc3)CC2)c2cc(C(F)(F)F)ccc21] | A2 | 432 | 1.36 | 433 | 98 | f |
| 23 | 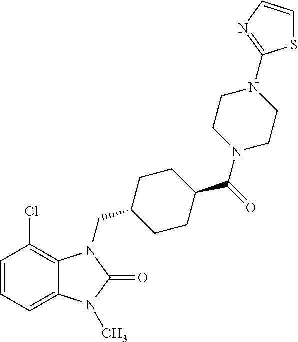 [SMILES: Cn1c(=O)n(C[C@H]2CC[C@H](C(=O)N3CCN(c4nccs4)CC3)CC2)c2c(Cl)cccc21] | A2 | 474 | 1.38 | 475 | 100 | f |
| 24 | 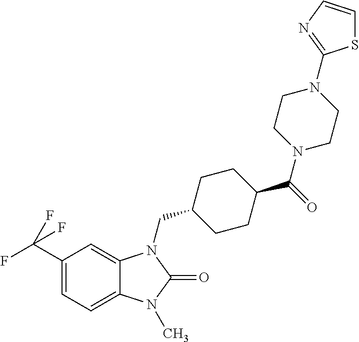 [SMILES: Cn1c(=O)n(C[C@H]2CC[C@H](C(=O)N3CCN(c4nccs4)CC3)CC2)c2cc(C(F)(F)F)ccc21] | A2 | 507 | 1.45 | 508 | 95 | f |
| 25 | 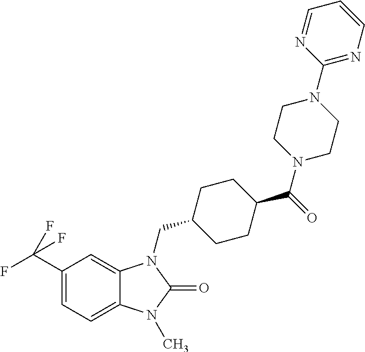 [SMILES: Cn1c(=O)n(C[C@H]2CC[C@H](C(=O)N3CCN(c4ncccn4)CC3)CC2)c2cc(C(F)(F)F)ccc21] | A2 | 502 | 1.5 | 503 | 100 | f |
| 26 | 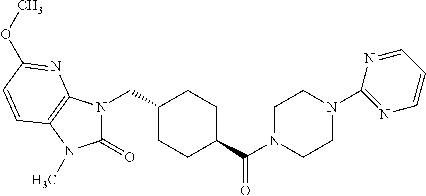 [SMILES: COc1ccc2c(n1)n(C[C@H]1CC[C@H](C(=O)N3CCN(c4ncccn4)CC3)CC1)c(=O)n2C] | A2 | 465 | 1.33 | 466 | 92 | e |
| 27 | 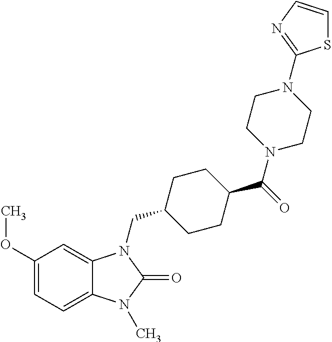 [SMILES: COc1ccc2c(c1)n(C[C@H]1CC[C@H](C(=O)N3CCN(c4nccs4)CC3)CC1)c(=O)n2C] | A3 | 469 | 1.17 | 470 | 100 | f |
| 28 | 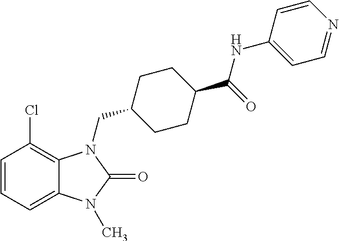 [SMILES: Cn1c(=O)n(C[C@H]2CC[C@H](C(=O)Nc3ccncc3)CC2)c2c(Cl)cccc21] | A2 | 398 | 1.31 | 399 | 100 | f |
| 29 | 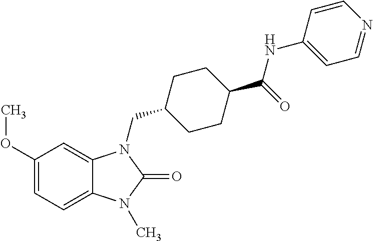 [SMILES: COc1ccc2c(c1)n(C[C@H]1CC[C@H](C(=O)Nc3ccncc3)CC1)c(=O)n2C] | A2 | 394 | 0.96 | 395 | 100 | f |
| 30 | 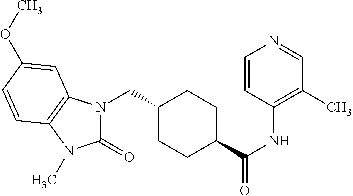 [SMILES: COc1ccc2c(c1)n(C[C@H]1CC[C@H](C(=O)Nc3ccncc3C)CC1)c(=O)n2C] | A2 | 408 | 1.1 | 409 | 97 | f |
| 31 | 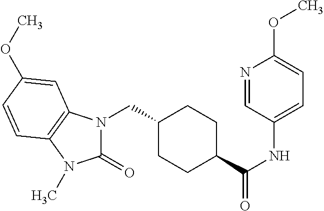 [SMILES: COc1ccc2c(c1)n(C[C@H]1CC[C@H](C(=O)Nc3ccc(OC)nc3)CC1)c(=O)n2C] | A2 | 424 | 1.28 | 425 | 100 | f |
| 32 | 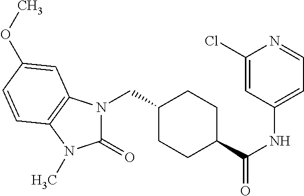 [SMILES: COc1ccc2c(c1)n(C[C@H]1CC[C@H](C(=O)Nc3ccnc(Cl)c3)CC1)c(=O)n2C] | A2 | 428 | 1.39 | 429 | 96 | f |
| 33 | 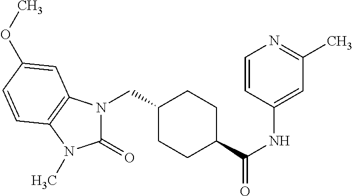 [SMILES: COc1ccc2c(c1)n(C[C@H]1CC[C@H](C(=O)Nc3ccnc(C)c3)CC1)c(=O)n2C] | A2 | 408 | 1.1 | 409 | 99 | f |
| 34 | 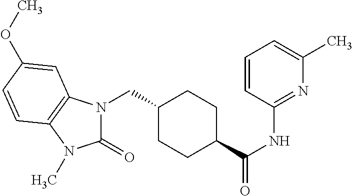 [SMILES: COc1ccc2c(c1)n(C[C@H]1CC[C@H](C(=O)Nc3cccc(C)n3)CC1)c(=O)n2C] | A2 | 408 | 1.24 | 409 | 97 | f |
| 35 | 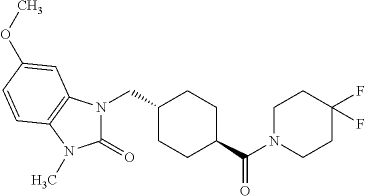 [SMILES: COc1ccc2c(c1)n(C[C@H]1CC[C@H](C(=O)N3CCC(F)(F)CC3)CC1)c(=O)n2C] | A2 | 421 | 1.37 | 422 | 96 | f |
| 36 | 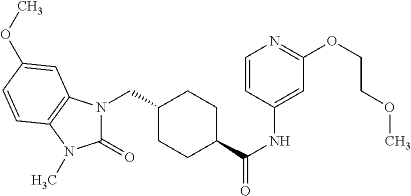 [SMILES: COCCOc1cc(NC(=O)[C@H]2CC[C@H](Cn3c(=O)n(C)c4ccc(OC)cc43)CC2)ccn1] | A2 | 468 | 1.27 | 469 | 96 | f |
| 37 | 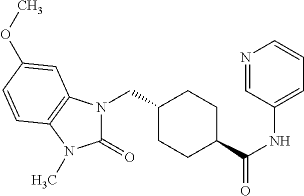 [SMILES: COc1ccc2c(c1)n(C[C@H]1CC[C@H](C(=O)Nc3cccnc3)CC1)c(=O)n2C] | A2 | 394 | 1.04 | 395 | 97 | f |
| 38 | 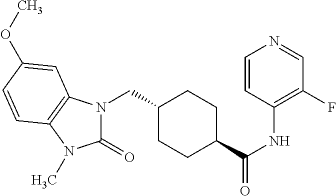 [SMILES: COc1ccc2c(c1)n(C[C@H]1CC[C@H](C(=O)Nc3ccncc3F)CC1)c(=O)n2C] | A2 | 412 | 1.18 | 413 | 95 | f |
| 39 | 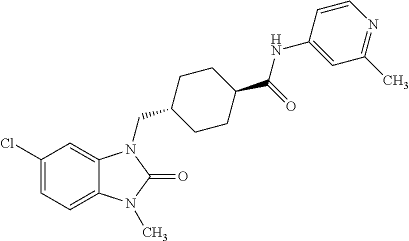 [SMILES: Cc1cc(NC(=O)[C@H]2CC[C@H](Cn3c(=O)n(C)c4ccc(Cl)cc43)CC2)ccn1] | A2 | 412 | 1.21 | 413 | 100 | f |
| 40 | 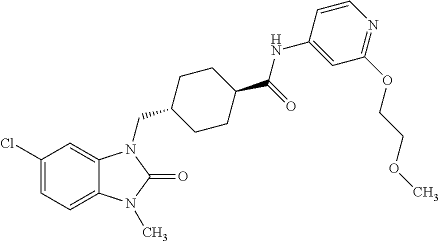 [SMILES: COCCOc1cc(NC(=O)[C@H]2CC[C@H](Cn3c(=O)n(C)c4ccc(Cl)cc43)CC2)ccn1] | A2 | 472 | 1.42 | 473 | 97 | f |
| 41 | 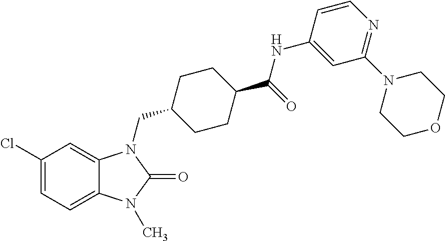 [SMILES: Cn1c(=O)n(C[C@H]2CC[C@H](C(=O)Nc3ccnc(N4CCOCC4)c3)CC2)c2cc(Cl)ccc21] | A2 | 484 | 1.27 | 484 | 95 | f |
| 42 | 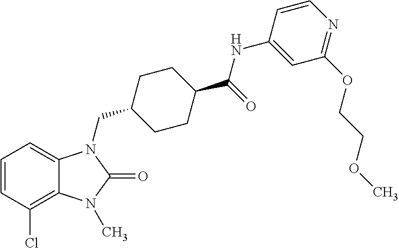 [SMILES: COCCOc1cc(NC(=O)[C@H]2CC[C@H](Cn3c(=O)n(C)c4c(Cl)cccc43)CC2)ccn1] | A2 | 472 | 1.48 | 473 | 100 | f |
| 43 | 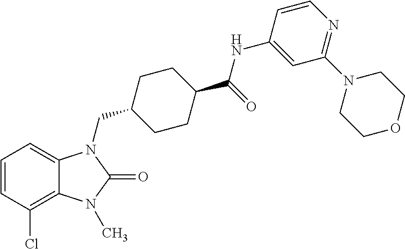 [SMILES: Cn1c(=O)n(C[C@H]2CC[C@H](C(=O)Nc3ccnc(N4CCOCC4)c3)CC2)c2cccc(Cl)c21] | A2 | 484 | 1.33 | 484 | 100 | f |
| 44 | 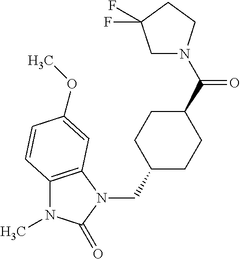 [SMILES: COc1ccc2c(c1)n(C[C@H]1CC[C@H](C(=O)N3CCC(F)(F)C3)CC1)c(=O)n2C] | A2 | 407 | 1.31 | 408 | 95 | f |
| 45 | 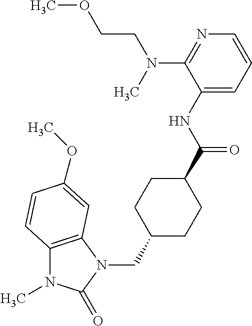 [SMILES: COCCN(C)c1ncccc1NC(=O)[C@H]1CC[C@H](Cn2c(=O)n(C)c3ccc(OC)cc32)CC1] | A2 | 481 | 1.22 | 482 | 95 | f |
| 46 | 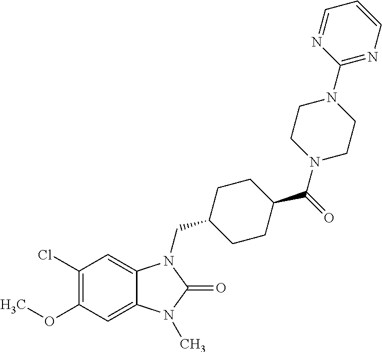 [SMILES: COc1cc2c(cc1Cl)n(C[C@H]1CC[C@H](C(=O)N3CCN(c4ncccn4)CC3)CC1)c(=O)n2C] | A2 | 499 | 1.37 | 500 | 100 | f |
| 47 | 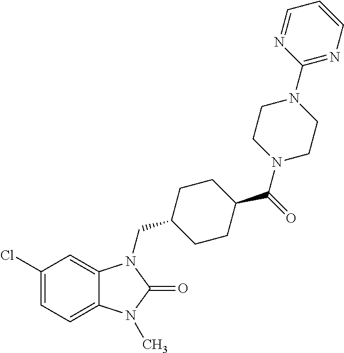 [SMILES: Cn1c(=O)n(C[C@H]2CC[C@H](C(=O)N3CCN(c4ncccn4)CC3)CC2)c2cc(Cl)ccc21] | A2 | 468 | 1.44 | 469 | 95 | f |
| 48 | 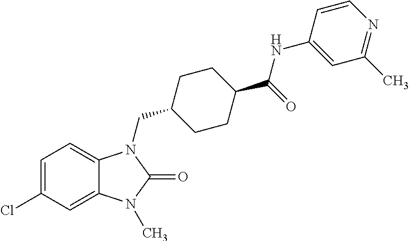 [SMILES: Cc1cc(NC(=O)[C@H]2CC[C@H](Cn3c(=O)n(C)c4cc(Cl)ccc43)CC2)ccn1] | A2 | 412 | 1.25 | 413 | 98 | f |
| 49 | 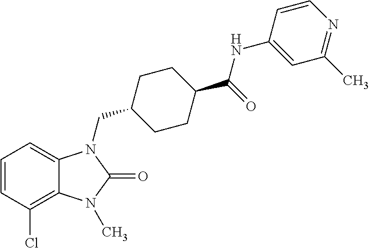 [SMILES: Cc1cc(NC(=O)[C@H]2CC[C@H](Cn3c(=O)n(C)c4c(Cl)cccc43)CC2)ccn1] | A2 | 412 | 1.28 | 413 | 100 | f |
| 50 | 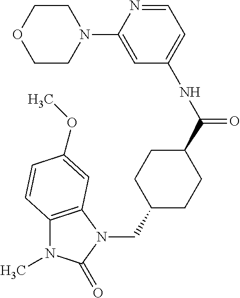 [SMILES: COc1ccc2c(c1)n(C[C@H]1CC[C@H](C(=O)Nc3ccnc(N4CCOCC4)c3)CC1)c(=O)n2C] | A2 | 479 | 1.12 | 480 | 99 | f |
| 51 | 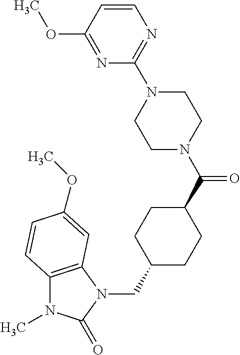 [SMILES: COc1ccc2c(c1)n(C[C@H]1CC[C@H](C(=O)N3CCN(c4nccc(OC)n4)CC3)CC1)c(=O)n2C] | A2 | 494 | 1.2 | 495 | 98 | f |
| 52 | 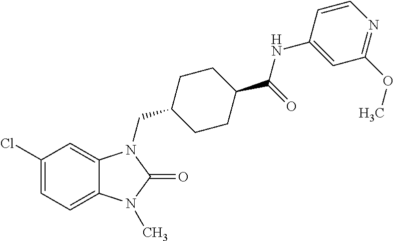 [SMILES: COc1cc(NC(=O)[C@H]2CC[C@H](Cn3c(=O)n(C)c4ccc(Cl)cc43)CC2)ccn1] | A2 | 428 | 1.42 | 429 | 99 | f |
| 53 | 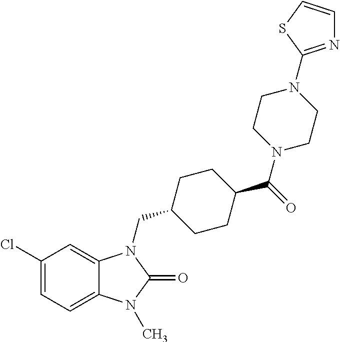 [SMILES: Cn1c(=O)n(C[C@H]2CC[C@H](C(=O)N3CCN(c4nccs4)CC3)CC2)c2cc(Cl)ccc21] | A2 | 474 | 1.31 | 475 | 97 | f |
| 54 | 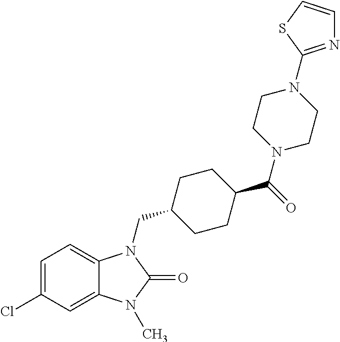 [SMILES: Cn1c(=O)n(C[C@H]2CC[C@H](C(=O)N3CCN(c4nccs4)CC3)CC2)c2ccc(Cl)cc21] | A2 | 474 | 1.37 | 475 | 100 | f |
| 55 | 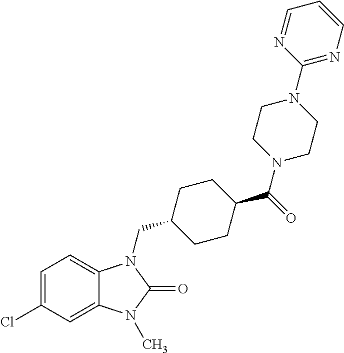 [SMILES: Cn1c(=O)n(C[C@H]2CC[C@H](C(=O)N3CCN(c4ncccn4)CC3)CC2)c2ccc(Cl)cc21] | A2 | 468 | 1.47 | 469 | 97 | f |
| 56 | 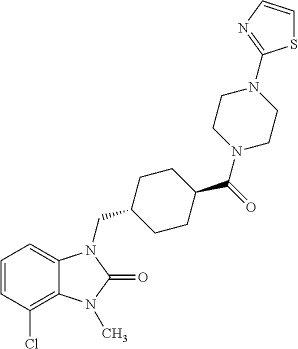 [SMILES: Cn1c(=O)n(C[C@H]2CC[C@H](C(=O)N3CCN(c4nccs4)CC3)CC2)c2cccc(Cl)c21] | A2 | 474 | 1.36 | 474 | 96 | f |
| 57 | 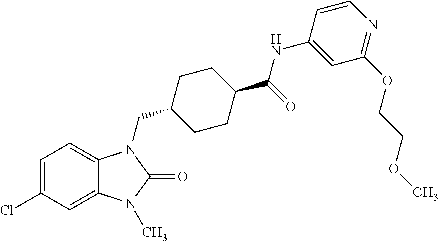 [SMILES: COCCOc1cc(NC(=O)[C@H]2CC[C@H](Cn3c(=O)n(C)c4cc(Cl)ccc43)CC2)ccn1] | A2 | 472 | 1.45 | 473 | 98 | f |
| 58 | 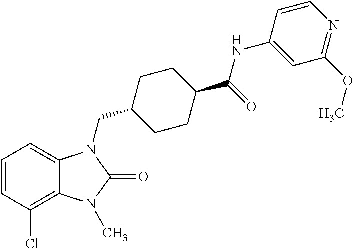 [SMILES: COc1cc(NC(=O)[C@H]2CC[C@H](Cn3c(=O)n(C)c4c(Cl)cccc43)CC2)ccn1] | A2 | 428 | 1.49 | 429 | 95 | f |
| 59 | 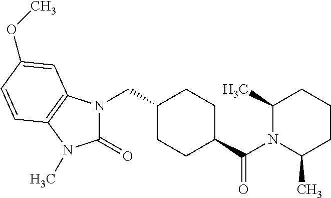 [SMILES: COc1ccc2c(c1)n(C[C@H]1CC[C@H](C(=O)N3[C@H](C)CCC[C@@H]3C)CC1)c(=O)n2C] | A2 | 413 | 4.56 | 414 | 97 | f |
| 60 | 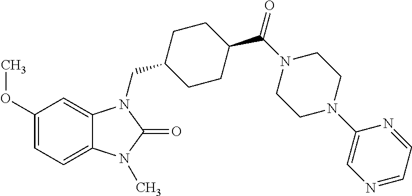 [SMILES: COc1ccc2c(c1)n(C[C@H]1CC[C@H](C(=O)N3CCN(c4cnccn4)CC3)CC1)c(=O)n2C] | A2 | 464 | 1.22 | 465 | 100 | f |
| 61 | 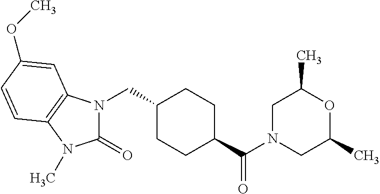 [SMILES: COc1ccc2c(c1)n(C[C@H]1CC[C@H](C(=O)N3C[C@@H](C)O[C@@H](C)C3)CC1)c(=O)n2C] | A2 | 415 | 1.31 | 416 | 97 | f |
| 62 | 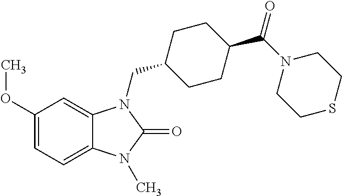 [SMILES: COc1ccc2c(c1)n(C[C@H]1CC[C@H](C(=O)N3CCSCC3)CC1)c(=O)n2C] | A2 | 403 | 1.31 | 404 | 100 | f |
| 63 | 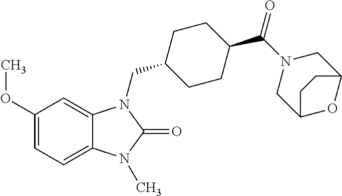 [SMILES: COc1ccc2c(c1)n(C[C@H]1CC[C@H](C(=O)N3CC4CCC(C3)O4)CC1)c(=O)n2C] | A2 | 413 | 1.19 | 414 | 100 | f |
| 64 | 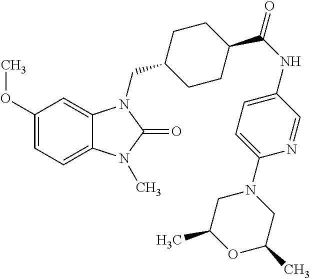 [SMILES: COc1ccc2c(c1)n(C[C@H]1CC[C@H](C(=O)Nc3ccc(N4C[C@@H](C)O[C@@H](C)C4)nc3)CC1)c(=O)n2C] | A2 | 507 | 1.19 | 508 | 100 | f |
| 65 | 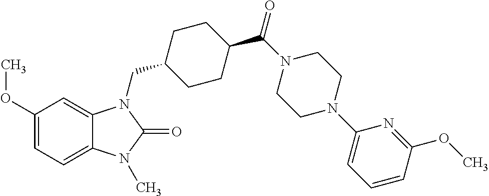 [SMILES: COc1ccc2c(c1)n(C[C@H]1CC[C@H](C(=O)N3CCN(c4cccc(OC)n4)CC3)CC1)c(=O)n2C] | A2 | 493 | 1.55 | 494 | 100 | f |
| 66 | 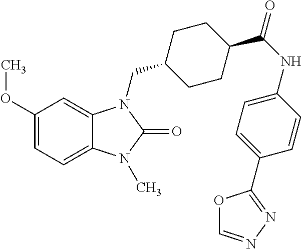 [SMILES: COc1ccc2c(c1)n(C[C@H]1CC[C@H](C(=O)Nc3ccc(-c4nnco4)cc3)CC1)c(=O)n2C] | A2 | 4361 | 1.3 | 462 | 97 | f |
| 67 | 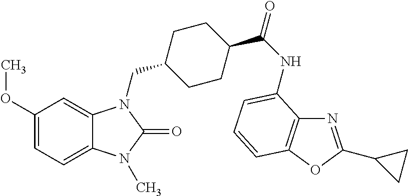 [SMILES: COc1ccc2c(c1)n(C[C@H]1CC[C@H](C(=O)Nc3cccc4oc(C5CC5)nc34)CC1)c(=O)n2C] | A2 | 474 | 1.63 | 475 | 100 | f |
| 68 | 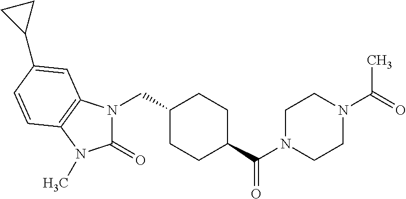 [SMILES: CC(=O)N1CCN(C(=O)[C@H]2CC[C@H](Cn3c(=O)n(C)c4ccc(C5CC5)cc43)CC2)CC1] | L1 | 438 | 1.26 | 439 | 100 | f |
| 69 | 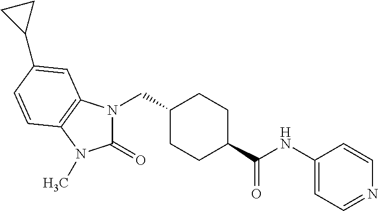 [SMILES: Cn1c(=O)n(C[C@H]2CC[C@H](C(=O)Nc3ccncc3)CC2)c2cc(C3CC3)ccc21] | L1 | 404 | 1.21 | 405 | 97 | f |
| 70 | 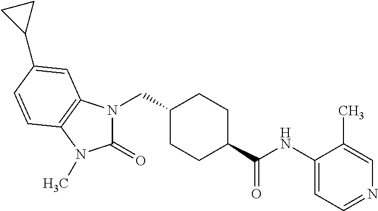 [SMILES: Cc1cnccc1NC(=O)[C@H]1CC[C@H](Cn2c(=O)n(C)c3ccc(C4CC4)cc32)CC1] | L1 | 418 | 1.24 | 419 | 100 | f |
| 71 | 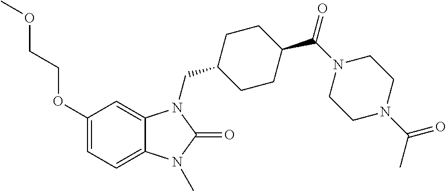 [SMILES: COCCOc1ccc2c(c1)n(C[C@H]1CC[C@H](C(=O)N3CCN(C(C)=O)CC3)CC1)c(=O)n2C] | D | 472 | 1.04 | 473 | 91 | f |
| 72 | 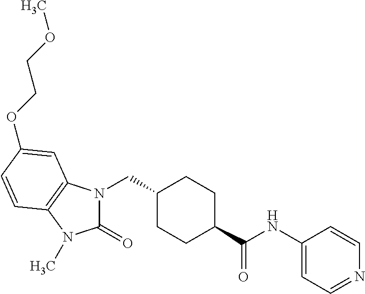 [SMILES: COCCOc1ccc2c(c1)n(C[C@H]1CC[C@H](C(=O)Nc3ccncc3)CC1)c(=O)n2C] | D | 438 | 1 | 439 | 100 | f |
| 73 | 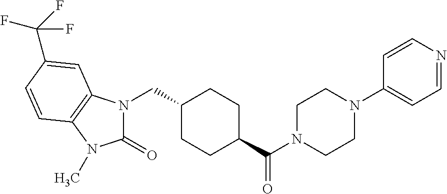 [SMILES: Cn1c(=O)n(C[C@H]2CC[C@H](C(=O)N3CCN(c4ccncc4)CC3)CC2)c2cc(C(F)(F)F)ccc21] | A2 | 501 | 1.17 | 502 | 100 | f |
| 74 | 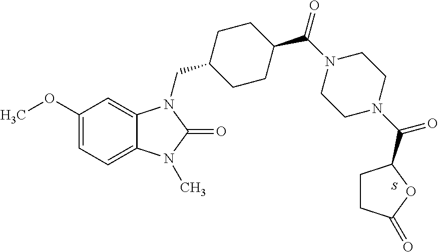 | A2 | 498 | 1.10 | 499 | 100 | f |
| 75 | 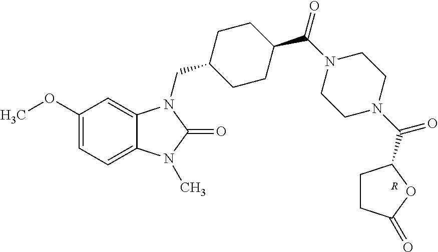 | A2 | 498 | 1.10 | 499 | 95 | f |
| 76 | 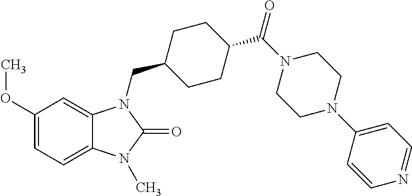 [SMILES: COc1ccc2c(c1)n(C[C@H]1CC[C@H](C(=O)N3CCN(c4ccncc4)CC3)CC1)c(=O)n2C] | A2 | 463 | 0.92 | 464 | 100 | f |
| 77 | 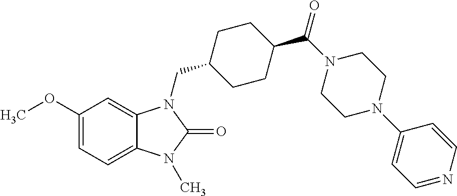 [SMILES: COc1ccc2c(c1)n(C[C@H]1CC[C@H](C(=O)N3CCN(c4ccncc4)CC3)CC1)c(=O)n2C] | A2 | 463 | 0.97 | 464 | 100 | f |
| 78 | 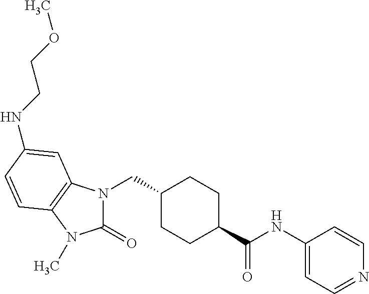 [SMILES: COCCNc1ccc2c(c1)n(C[C@H]1CC[C@H](C(=O)Nc3ccncc3)CC1)c(=O)n2C] | L2 | 437 | 0.72 | 438 | 95 | f |
| 79 | 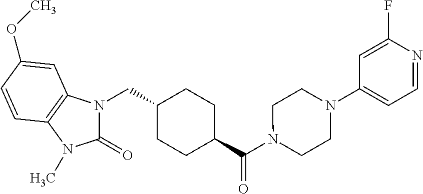 [SMILES: COc1ccc2c(c1)n(C[C@H]1CC[C@H](C(=O)N3CCN(c4ccnc(F)c4)CC3)CC1)c(=O)n2C] | P | 481 | 1.27 | 482 | 100 | f |
| 80 | 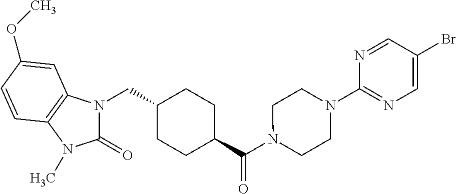 [SMILES: COc1ccc2c(c1)n(C[C@H]1CC[C@H](C(=O)N3CCN(c4ncc(Br)cn4)CC3)CC1)c(=O)n2C] | A2 | 543 | 1.59 | 544 | 100 | f |
| 81 | 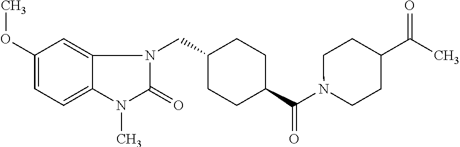 [SMILES: COc1ccc2c(c1)n(C[C@H]1CC[C@H](C(=O)N3CCC(C(C)=O)CC3)CC1)c(=O)n2C] | A2 | 427 | 1.22 | 428 | 100 | f |
| 82 | 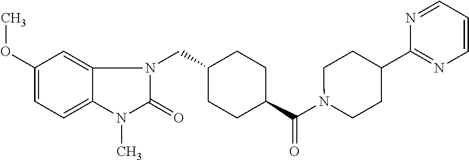 [SMILES: COc1ccc2c(c1)n(C[C@H]1CC[C@H](C(=O)N3CCC(c4ncccn4)CC3)CC1)c(=O)n2C] | A2 | 463 | 1.19 | 464 | 93 | f |
| 83 | 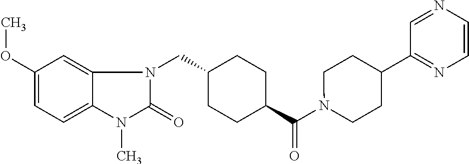 [SMILES: COc1ccc2c(c1)n(C[C@H]1CC[C@H](C(=O)N3CCC(c4cnccn4)CC3)CC1)c(=O)n2C] | A2 | 463 | 1.21 | 464 | 93 | f |
| 84 | 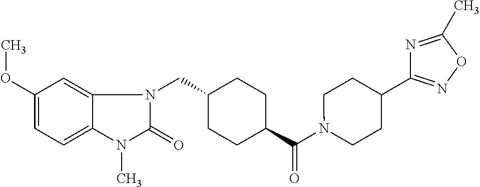 [SMILES: COc1ccc2c(c1)n(C[C@H]1CC[C@H](C(=O)N3CCC(c4noc(C)n4)CC3)CC1)c(=O)n2C] | A2 | 467 | 1.27 | 468 | 100 | f |
| 85 | 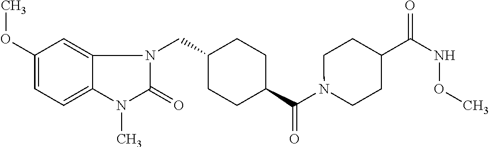 [SMILES: CONC(=O)C1CCN(C(=O)[C@H]2CC[C@H](Cn3c(=O)n(C)c4ccc(OC)cc43)CC2)CC1] | A2 | 458 | 1.05 | 459 | 100 | f |
| 86 | 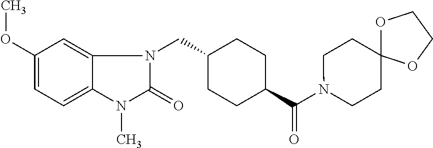 [SMILES: COc1ccc2c(c1)n(C[C@H]1CC[C@H](C(=O)N3CCC4(CC3)OCCO4)CC1)c(=O)n2C] | A2 | 443 | 1.25 | 444 | 98 | f |
| 87 | 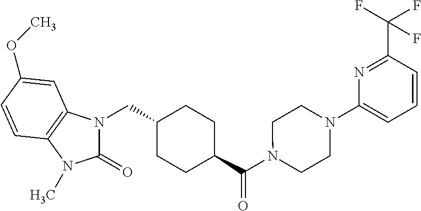 [SMILES: COc1ccc2c(c1)n(C[C@H]1CC[C@H](C(=O)N3CCN(c4cccc(C(F)(F)F)n4)CC3)CC1)c(=O)n2C] | P | 531 | 1.66 | 532 | 95 | f |
| 88 | 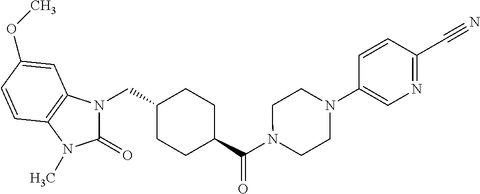 [SMILES: COc1ccc2c(c1)n(C[C@H]1CC[C@H](C(=O)N3CCN(c4ccc(C#N)nc4)CC3)CC1)c(=O)n2C] | P | 488 | 1.3 | 489 | 100 | f |
| 89 | 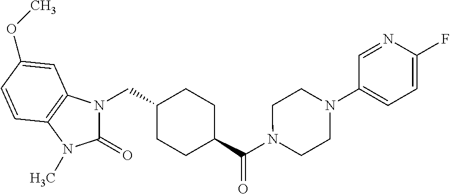 [SMILES: COc1ccc2c(c1)n(C[C@H]1CC[C@H](C(=O)N3CCN(c4ccc(F)nc4)CC3)CC1)c(=O)n2C] | P | 481 | 1.34 | 482 | 92 | f |
| 90 | 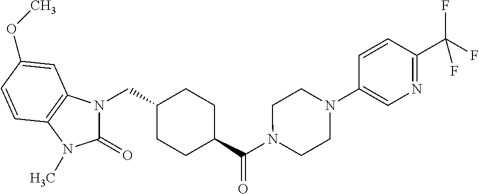 [SMILES: COc1ccc2c(c1)n(C[C@H]1CC[C@H](C(=O)N3CCN(c4ccc(C(F)(F)F)nc4)CC3)CC1)c(=O)n2C] | P | 531 | 1.49 | 532 | 100 | f |
| 91 | 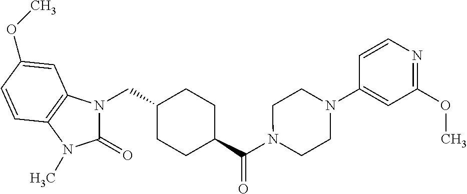 [SMILES: COc1ccc2c(c1)n(C[C@H]1CC[C@H](C(=O)N3CCN(c4ccnc(OC)c4)CC3)CC1)c(=O)n2C] | P | 493 | 1.02 | 494 | 100 | f |
| 92 | 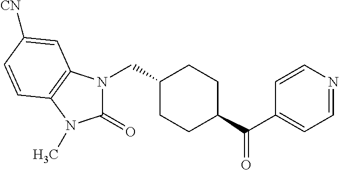 | A2 | 389 | 0.96 | 390 | 99 | f |
| 93 | 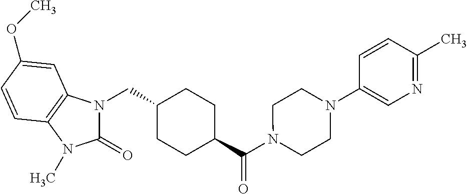 [SMILES: COc1ccc2c(c1)n(C[C@H]1CC[C@H](C(=O)N3CCN(c4ccc(C)nc4)CC3)CC1)c(=O)n2C] | P | 477 | 0.99 | 478 | 100 | f |
| 94 | 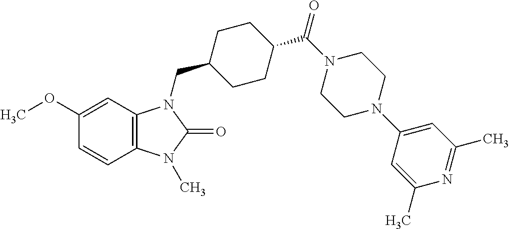 | P | 491 | 1.04 | 492 | 100 | f |
| 95 | 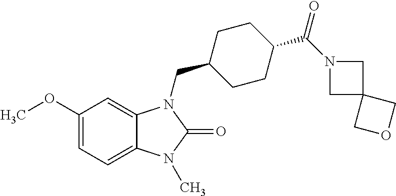 [SMILES: COc1ccc2c(c1)n(C[C@H]1CC[C@H](C(=O)N3CC4(COC4)C3)CC1)c(=O)n2C] | A2 | 399 | 1.08 | 400 | 100 | f |
| 96 | 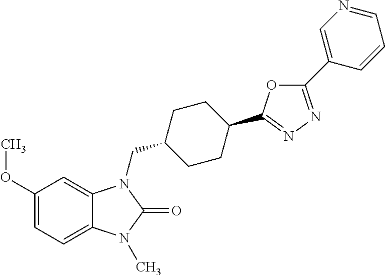 [SMILES: COc1ccc2c(c1)n(C[C@H]1CC[C@H](c3nnc(-c4cccnc4)o3)CC1)c(=O)n2C] | H1 | 419 | 1.26 | 420 | 100 | f |
| 97 | 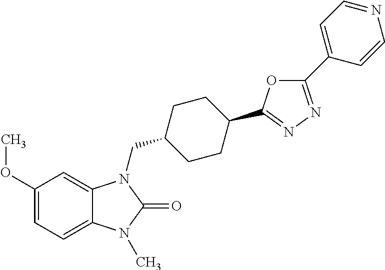 [SMILES: COc1ccc2c(c1)n(C[C@H]1CC[C@H](c3nnc(-c4ccncc4)o3)CC1)c(=O)n2C] | H1 | 419 | 1.24 | 420 | 100 | f |
| 98 | 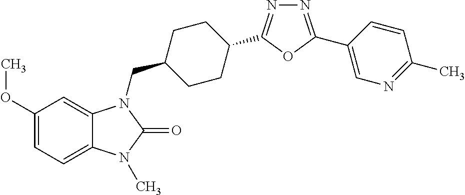 [SMILES: COc1ccc2c(c1)n(C[C@H]1CC[C@H](c3nnc(-c4ccc(C)nc4)o3)CC1)c(=O)n2C] | H1 | 433 | 1.29 | 434 | 96 | f |
| 99 | 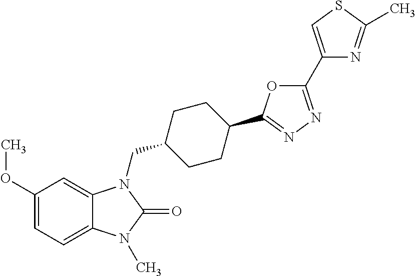 [SMILES: COc1ccc2c(c1)n(C[C@H]1CC[C@H](c3nnc(-c4csc(C)n4)o3)CC1)c(=O)n2C] | H1 | 439 | 1.33 | 440 | 95 | f |
| 100 | 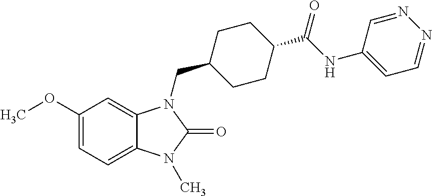 [SMILES: COc1ccc2c(c1)n(C[C@H]1CC[C@H](C(=O)Nc3ccnnc3)CC1)c(=O)n2C] | A2 | 395 | 1.07 | 396 | 100 | f |
| 101 | 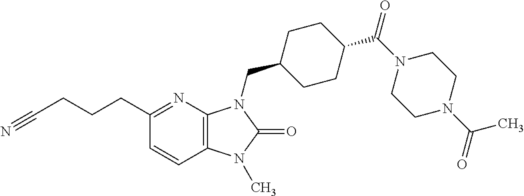 [SMILES: CC(=O)N1CCN(C(=O)[C@H]2CC[C@H](Cn3c(=O)n(C)c4ccc(CCCC#N)nc43)CC2)CC1] | B | 466 | 1.02 | 467 | 100 | f |
| 102 | 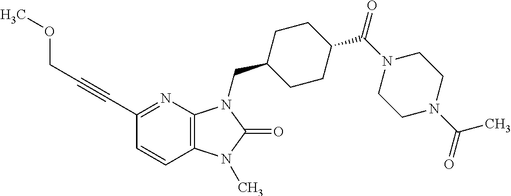 [SMILES: COCC#Cc1ccc2c(n1)n(C[C@H]1CC[C@H](C(=O)N3CCN(C(C)=O)CC3)CC1)c(=O)n2C] | B | 467 | 1.07 | 468 | 95 | f |
| 103 | 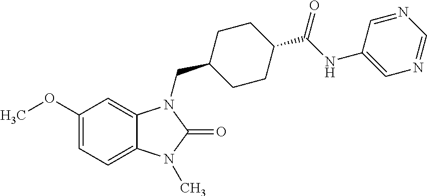 [SMILES: COc1ccc2c(c1)n(C[C@H]1CC[C@H](C(=O)Nc3cncnc3)CC1)c(=O)n2C] | A2 | 395 | 1.13 | 396 | 100 | f |
| 104 | 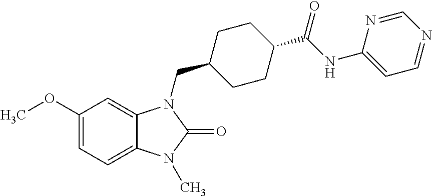 [SMILES: COc1ccc2c(c1)n(C[C@H]1CC[C@H](C(=O)Nc3ccncn3)CC1)c(=O)n2C] | A2 | 395 | 1.18 | 396 | 100 | f |
| 105 | 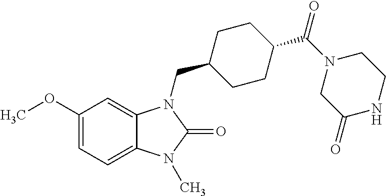 [SMILES: COc1ccc2c(c1)n(C[C@H]1CC[C@H](C(=O)N3CCNC(=O)C3)CC1)c(=O)n2C] | A2 | 400 | 1 | 401 | 100 | f |
| 106 | 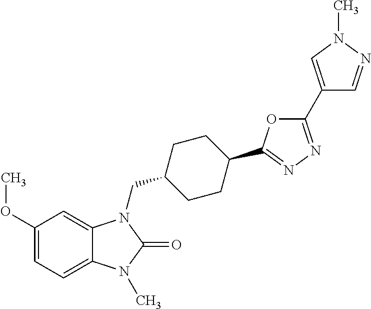 [SMILES: COc1ccc2c(c1)n(C[C@H]1CC[C@H](c3nnc(-c4cnn(C)c4)o3)CC1)c(=O)n2C] | H2 | 422 | 1.23 | 423 | 100 | f |
| 107 | 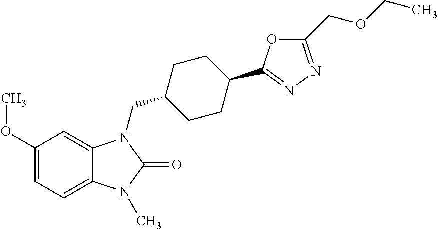 [SMILES: CCOCc1nnc([C@H]2CC[C@H](Cn3c(=O)n(C)c4ccc(OC)cc43)CC2)o1] | H2 | 400 | 1.33 | 401 | 97 | f |
| 108 | 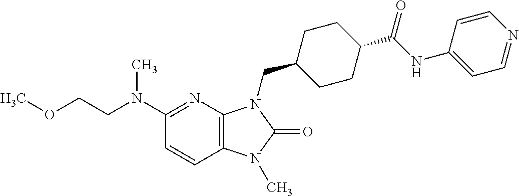 [SMILES: COCCN(C)c1ccc2c(n1)n(C[C@H]1CC[C@H](C(=O)Nc3ccncc3)CC1)c(=O)n2C] | C | 452 | 1 | 453 | 99 | f |
| 109 | 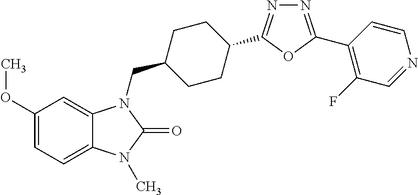 [SMILES: COc1ccc2c(c1)n(C[C@H]1CC[C@H](c3nnc(-c4ccncc4F)o3)CC1)c(=O)n2C] | H2 | 437 | 1.33 | 438 | 100 | f |
| 110 | 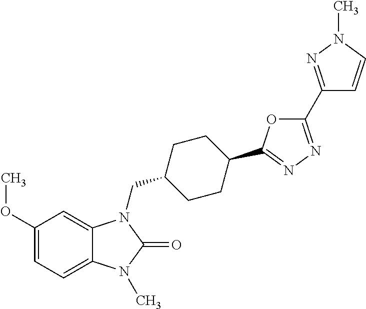 [SMILES: COc1ccc2c(c1)n(C[C@H]1CC[C@H](c3nnc(-c4ccn(C)n4)o3)CC1)c(=O)n2C] | H1 | 422 | 1.24 | 423 | 100 | f |
| 111 | 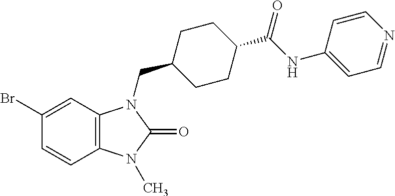 [SMILES: Cn1c(=O)n(C[C@H]2CC[C@H](C(=O)Nc3ccncc3)CC2)c2cc(Br)ccc21] | A2 | 443 | 1.13 | 444 | 94 | f |
| 112 | 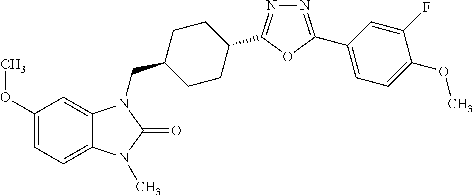 [SMILES: COc1ccc2c(c1)n(C[C@H]1CC[C@H](c3nnc(-c4ccc(OC)c(F)c4)o3)CC1)c(=O)n2C] | H2 | 466 | 1.56 | 467 | 100 | f |
| 113 | 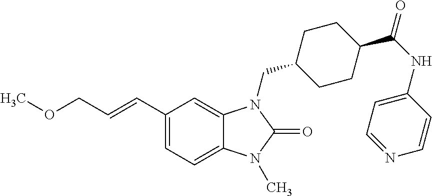 [SMILES: COC/C=C/c1ccc2c(c1)n(C[C@H]1CC[C@H](C(=O)Nc3ccncc3)CC1)c(=O)n2C] | B | 435 | 1.02 | 436 | 95 | f |
| 114 | 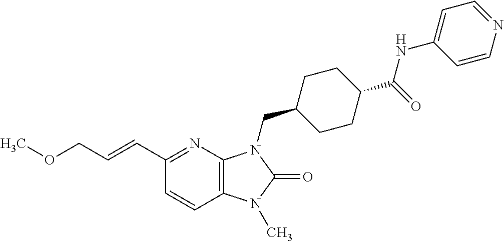 [SMILES: COC/C=C/c1ccc2c(n1)n(C[C@H]1CC[C@H](C(=O)Nc3ccncc3)CC1)c(=O)n2C] | B | 435 | 1.02 | 436 | 95 | f |
| 115 | 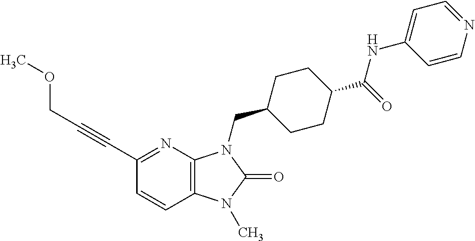 [SMILES: COCC#Cc1ccc2c(n1)n(C[C@H]1CC[C@H](C(=O)Nc3ccncc3)CC1)c(=O)n2C] | B | 433 | 1.00 | 434 | 100 | f |
| 116 | 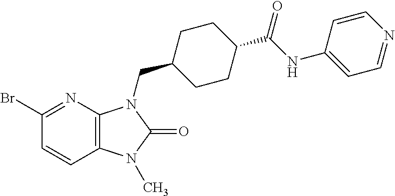 [SMILES: Cn1c(=O)n(C[C@H]2CC[C@H](C(=O)Nc3ccncc3)CC2)c2nc(Br)ccc21] | A2 | 444 | 1.05 | 445 | 95 | f |
| 117 | 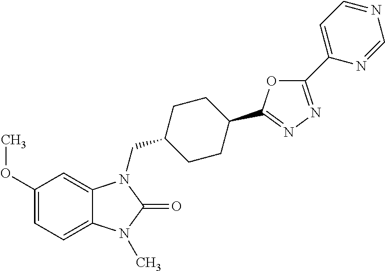 [SMILES: COc1ccc2c(c1)n(C[C@H]1CC[C@H](c3nnc(-c4ccncn4)o3)CC1)c(=O)n2C] | H2 | 420 | 1.21 | 421 | 100 | f |
| 118 | 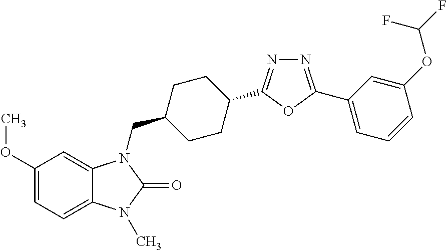 [SMILES: COc1ccc2c(c1)n(C[C@H]1CC[C@H](c3nnc(-c4cccc(OC(F)F)c4)o3)CC1)c(=O)n2C] | H2 | 484 | 1.61 | 485 | 97 | f |
| 119 | 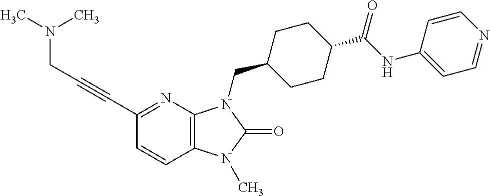 [SMILES: CN(C)CC#Cc1ccc2c(n1)n(C[C@H]1CC[C@H](C(=O)Nc3ccncc3)CC1)c(=O)n2C] | B | 446 | 0.63 | 447 | 99 | f |
| 120 | 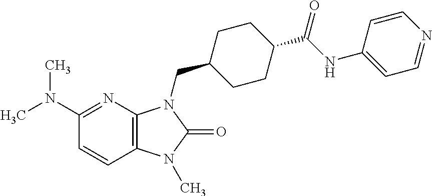 [SMILES: CN(C)c1ccc2c(n1)n(C[C@H]1CC[C@H](C(=O)Nc3ccncc3)CC1)c(=O)n2C] | B | 408 | 1.05 | 409 | 97 | f |
| 121 | 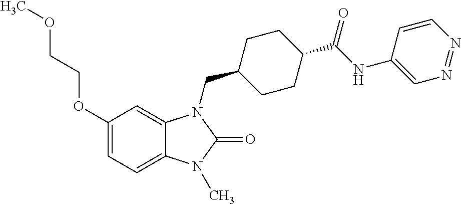 [SMILES: COCCOc1ccc2c(c1)n(C[C@H]1CC[C@H](C(=O)Nc3ccnnc3)CC1)c(=O)n2C] | D | 439 | 1.04 | 440 | 100 | f |
| 122 | 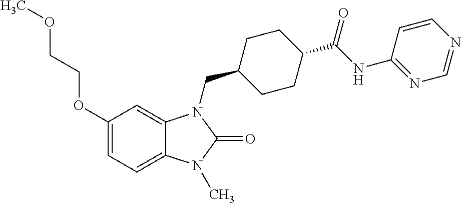 [SMILES: COCCOc1ccc2c(c1)n(C[C@H]1CC[C@H](C(=O)Nc3ccncn3)CC1)c(=O)n2C] | D | 439 | 1.15 | 440 | 98 | f |
| 123 | 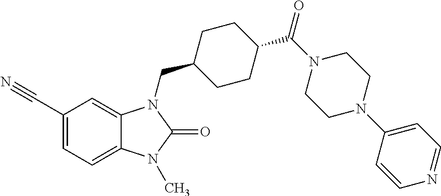 [SMILES: Cn1c(=O)n(C[C@H]2CC[C@H](C(=O)N3CCN(c4ccncc4)CC3)CC2)c2cc(C#N)ccc21] | A2 | 458 | 0.93 | 459 | 100 | f |
| 124 | 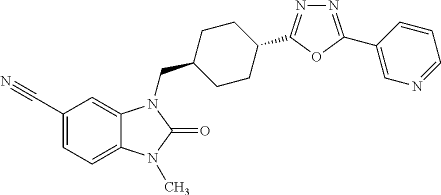 [SMILES: Cn1c(=O)n(C[C@H]2CC[C@H](c3nnc(-c4cccnc4)o3)CC2)c2cc(C#N)ccc21] | H1 | 414 | 1.21 | 415 | 100 | f |
| 125 | 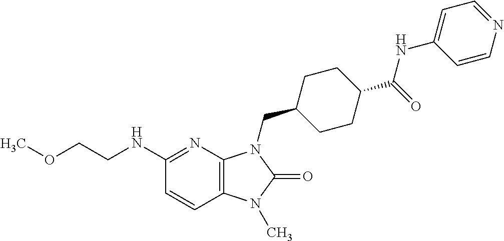 [SMILES: COCCNc1ccc2c(n1)n(C[C@H]1CC[C@H](C(=O)Nc3ccncc3)CC1)c(=O)n2C] | B | 438 | 0.91 | 439 | 97 | f |
| 126 | 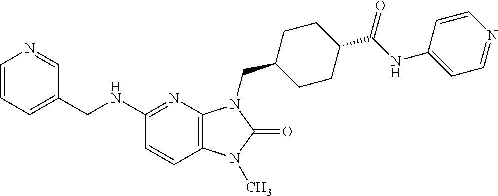 [SMILES: Cn1c(=O)n(C[C@H]2CC[C@H](C(=O)Nc3ccncc3)CC2)c2nc(NCc3cccnc3)ccc21] | B | 471 | 0.68 | 472 | 97 | f |
| 127 | 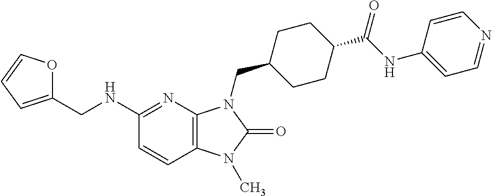 [SMILES: Cn1c(=O)n(C[C@H]2CC[C@H](C(=O)Nc3ccncc3)CC2)c2nc(NCc3ccco3)ccc21] | B | 460 | 1.07 | 461 | 98 | f |
| 128 | 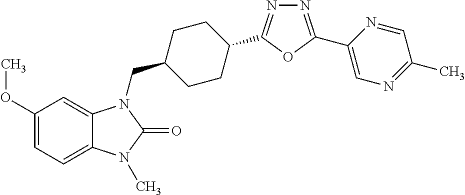 [SMILES: COc1ccc2c(c1)n(C[C@H]1CC[C@H](c3nnc(-c4cnc(C)cn4)o3)CC1)c(=O)n2C] | H2 | 434 | 1.29 | 435 | 98 | f |
| 129 | 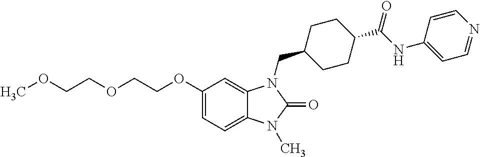 [SMILES: COCCOCCOc1ccc2c(c1)n(C[C@H]1CC[C@H](C(=O)Nc3ccncc3)CC1)c(=O)n2C] | D | 482 | 0.98 | 483 | 91 | f |
| 130 | 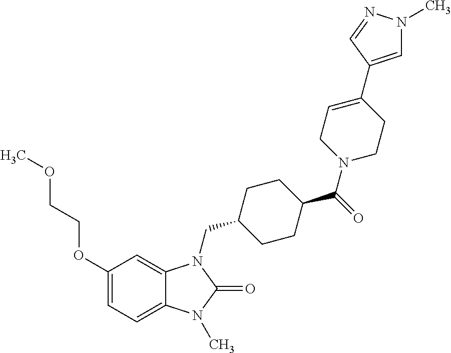 | D | 507 | 2.5 | 508 | 100 | c |
| 131 | 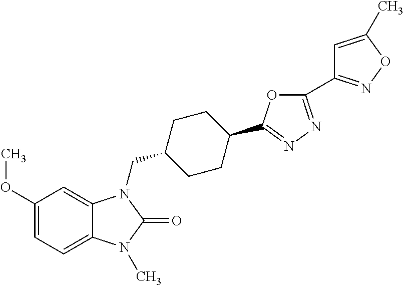 [SMILES: COc1ccc2c(c1)n(C[C@H]1CC[C@H](c3nnc(-c4cc(C)on4)o3)CC1)c(=O)n2C] | H1 | 423 | 1.4 | 424 | 96 | f |
| 132 | 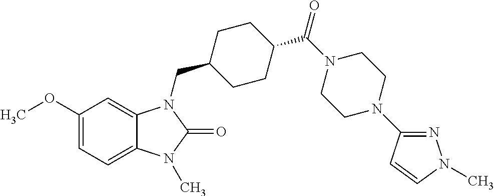 | A2 | 466 | 1.17 | 467 | 98 | f |
| 133 | 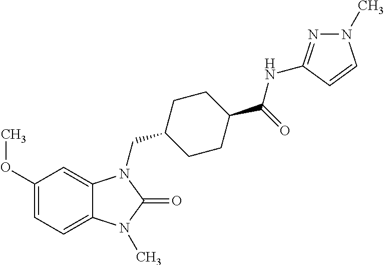 [SMILES: COc1ccc2c(c1)n(C[C@H]1CC[C@H](C(=O)Nc3ccn(C)n3)CC1)c(=O)n2C] | A2 | 397 | 1.15 | 398 | 98 | f |
| 134 | 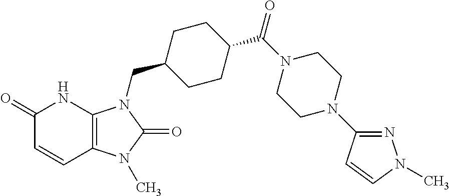 [SMILES: Cn1ccc(N2CCN(C(=O)[C@H]3CC[C@H](Cn4c(=O)n(C)c5ccc(=O)[nH]c54)CC3)CC2)n1] | G1 | 453 | 0.9 | 454 | 96 | f |
| 135 | 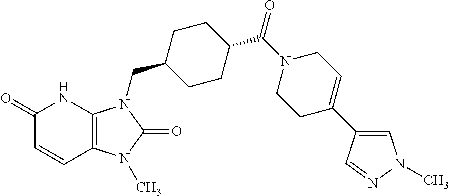 [SMILES: Cn1cc(C2=CCN(C(=O)[C@H]3CC[C@H](Cn4c(=O)n(C)c5ccc(=O)[nH]c54)CC3)CC2)cn1] | G1 | 450 | 0.97 | 451 | 100 | f |
| 136 | 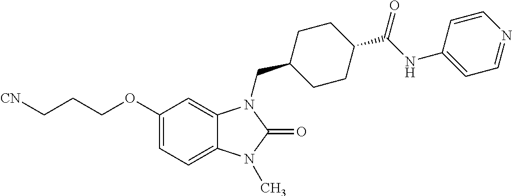 | D | 447 | 0.96 | 448 | 100 | f |
| 137 | 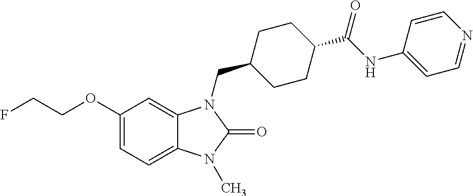 [SMILES: Cn1c(=O)n(C[C@H]2CC[C@H](C(=O)Nc3ccncc3)CC2)c2cc(OCCF)ccc21] | D | 426 | 0.96 | 427 | 95 | f |
| 138 | 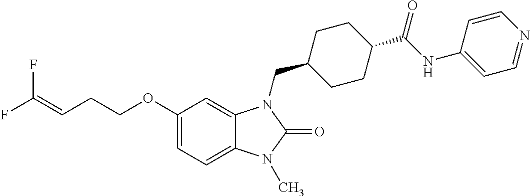 [SMILES: Cn1c(=O)n(C[C@H]2CC[C@H](C(=O)Nc3ccncc3)CC2)c2cc(OCCC=C(F)F)ccc21] | D | 470 | 1.19 | 471 | 95 | f |
| 139 | 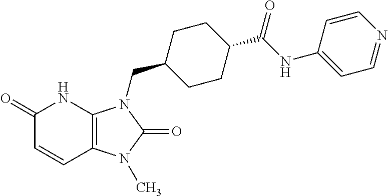 [SMILES: Cn1c(=O)n(C[C@H]2CC[C@H](C(=O)Nc3ccncc3)CC2)c2[nH]c(=O)ccc21] | G1 | 381 | 0.71 | 382 | 95 | f |
| 140 | 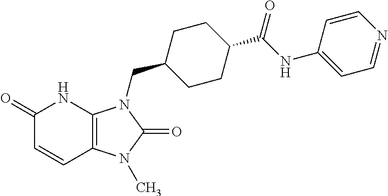 [SMILES: Cn1c(=O)n(C[C@H]2CC[C@H](C(=O)Nc3ccncc3)CC2)c2[nH]c(=O)ccc21] | G1 | 381 | 0.71 | 382 | 95 | f |
| 141 | 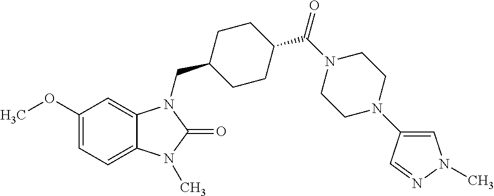 | A2 | 466 | 1.1 | 467 | 99 | f |
| 142 | 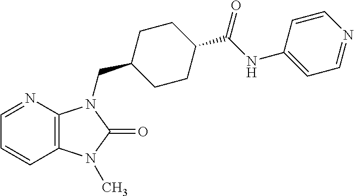 [SMILES: Cn1c(=O)n(C[C@H]2CC[C@H](C(=O)Nc3ccncc3)CC2)c2ncccc21] | B | 365 | 0.8 | 366 | 97 | f |
| 143 | 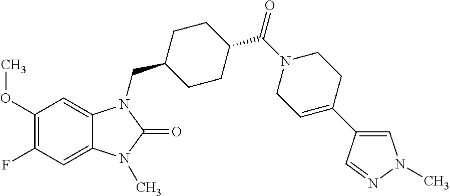 [SMILES: COc1cc2c(cc1F)n(C)c(=O)n2C[C@H]1CC[C@H](C(=O)N2CC=C(c3cnn(C)c3)CC2)CC1] | A2 | 481 | 1.24 | 482 | 100 | f |
| 144 | 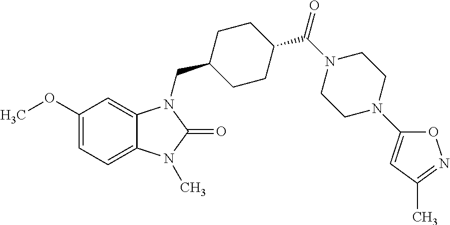 [SMILES: COc1ccc2c(c1)n(C[C@H]1CC[C@H](C(=O)N3CCN(c4cc(C)no4)CC3)CC1)c(=O)n2C] | A2 | 467 | 1.28 | 468 | 99 | f |
| 145 | 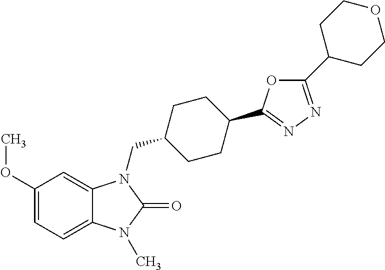 [SMILES: COc1ccc2c(c1)n(C[C@H]1CC[C@H](c3nnc(C4CCOCC4)o3)CC1)c(=O)n2C] | H1 | 426 | 2.57 | 427 | 100 | c |
| 146 | 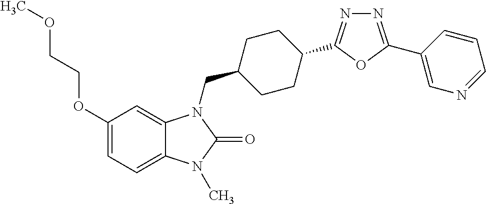 [SMILES: COCCOc1ccc2c(c1)n(C[C@H]1CC[C@H](c3nnc(-c4cccnc4)o3)CC1)c(=O)n2C] | H1 | 463 | 2.5 | 464 | 100 | c |
| 147 | 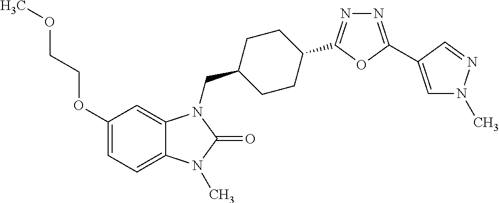 | H2 | 466 | 1.16 | 467 | 100 | f |
| 148 | 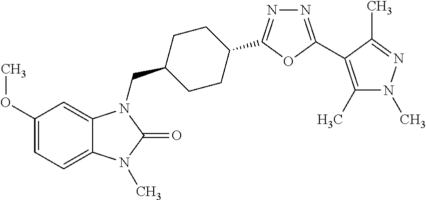 [SMILES: COc1ccc2c(c1)n(C[C@H]1CC[C@H](c3nnc(-c4c(C)nn(C)c4C)o3)CC1)c(=O)n2C] | H2 | 450 | 1.29 | 451 | 100 | f |
| 149 | 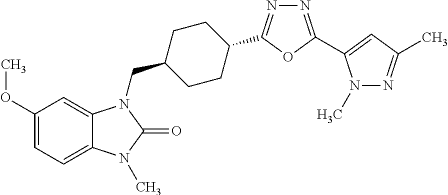 [SMILES: COc1ccc2c(c1)n(C[C@H]1CC[C@H](c3nnc(-c4cc(C)nn4C)o3)CC1)c(=O)n2C] | H2 | 436 | 1.39 | 437 | 100 | f |
| 150 | 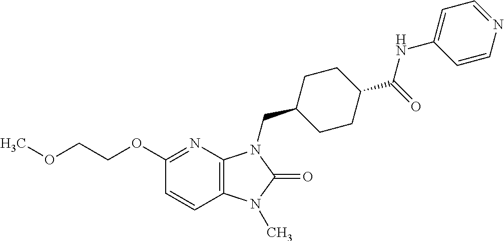 [SMILES: COCCOc1ccc2c(n1)n(C[C@H]1CC[C@H](C(=O)Nc3ccncc3)CC1)c(=O)n2C] | B | 439 | 0.93 | 440 | 96 | f |
| 151 | 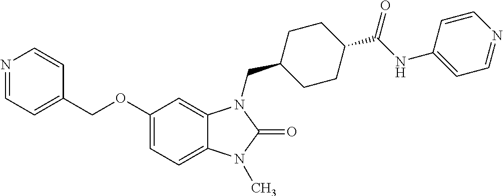 [SMILES: Cn1c(=O)n(C[C@H]2CC[C@H](C(=O)Nc3ccncc3)CC2)c2cc(OCc3ccncc3)ccc21] | D | 471 | 0.72 | 472 | 97 | f |
| 152 | 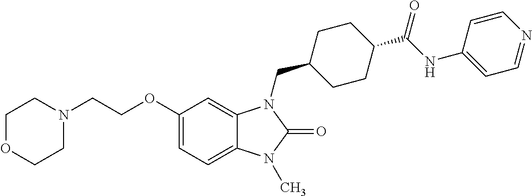 [SMILES: Cn1c(=O)n(C[C@H]2CC[C@H](C(=O)Nc3ccncc3)CC2)c2cc(OCCN3CCOCC3)ccc21] | D | 493 | 0.61 | 494 | 97 | f |
| 153 | 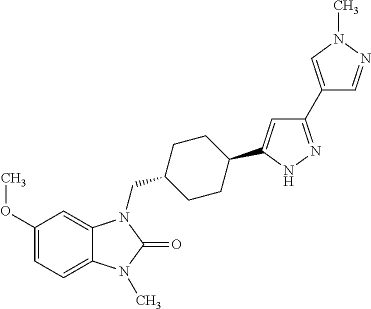 [SMILES: COc1ccc2c(c1)n(C[C@H]1CC[C@H](c3cc(-c4cnn(C)c4)n[nH]3)CC1)c(=O)n2C] | I | 420 | 1.19 | 421 | 100 | f |
| 154 | 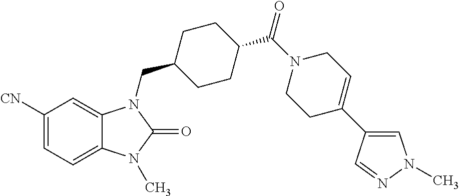 | A2 | 458 | 1.2 | 459 | 91 | f |
| 155 | 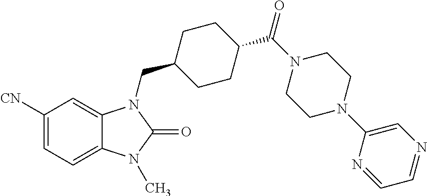 | A2 | 459 | 1.17 | 460 | 99 | f |
| 156 | 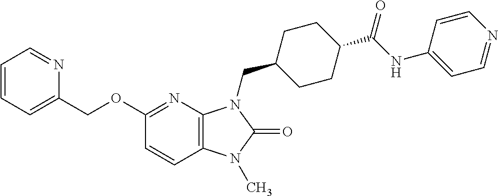 [SMILES: Cn1c(=O)n(C[C@H]2CC[C@H](C(=O)Nc3ccncc3)CC2)c2nc(OCc3ccccn3)ccc21] | G | 472 | 0.83 | 473 | 100 | f |
| 157 | 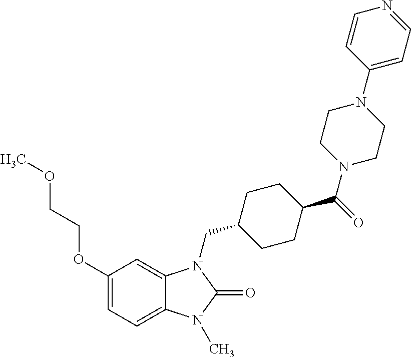 [SMILES: COCCOc1ccc2c(c1)n(C[C@H]1CC[C@H](C(=O)N3CCN(c4ccncc4)CC3)CC1)c(=O)n2C] | D | 507 | 0.9 | 508 | 96 | f |
| 158 | 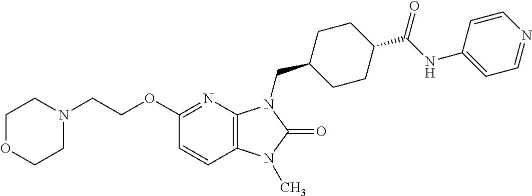 [SMILES: Cn1c(=O)n(C[C@H]2CC[C@H](C(=O)Nc3ccncc3)CC2)c2nc(OCCN3CCOCC3)ccc21] | G | 494 | 0.6 | 495 | 100 | f |
| 159 | 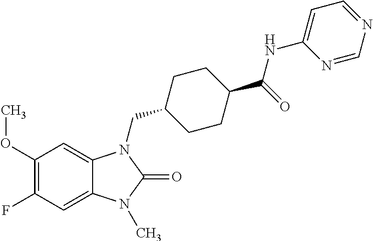 [SMILES: COc1cc2c(cc1F)n(C)c(=O)n2C[C@H]1CC[C@H](C(=O)Nc2ccncn2)CC1] | A2 | 413 | 1.18 | 414 | 100 | f |
| 160 | 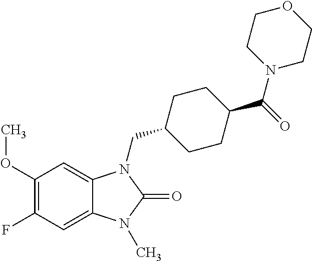 [SMILES: COc1cc2c(cc1F)n(C)c(=O)n2C[C@H]1CC[C@H](C(=O)N2CCOCC2)CC1] | A2 | 405 | 1.14 | 406 | 95 | f |
| 161 | 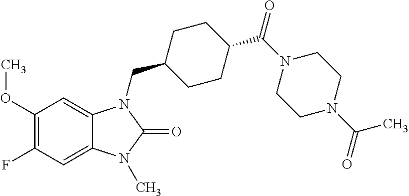 [SMILES: COc1cc2c(cc1F)n(C)c(=O)n2C[C@H]1CC[C@H](C(=O)N2CCN(C(C)=O)CC2)CC1] | A2 | 446 | 1.2 | 447 | 95 | f |
| 162 | 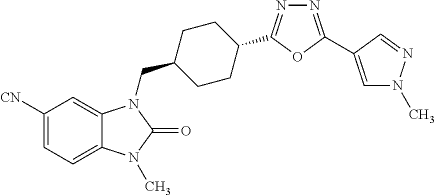 | H2 | 417 | 1.16 | 418 | 95 | f |
| 163 | 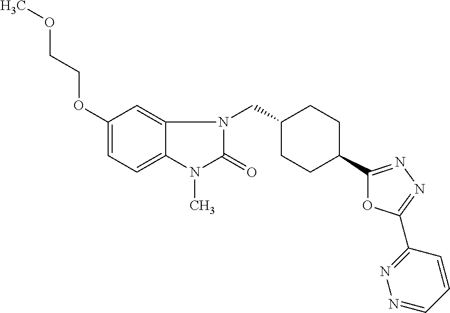 [SMILES: COCCOc1ccc2c(c1)n(C[C@H]1CC[C@H](c3nnc(-c4cccnn4)o3)CC1)c(=O)n2C] | H2 | 464 | 1.12 | 465 | 100 | f |
| 164 | 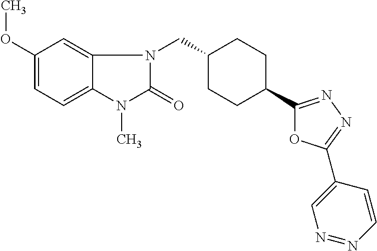 [SMILES: COc1ccc2c(c1)n(C[C@H]1CC[C@H](c3nnc(-c4ccnnc4)o3)CC1)c(=O)n2C] | H2 | 420 | 1.15 | 421 | 100 | f |
| 165 | 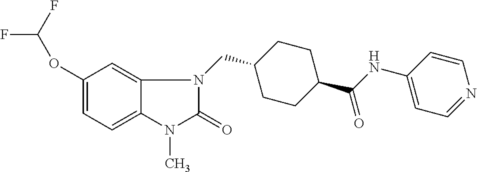 [SMILES: Cn1c(=O)n(C[C@H]2CC[C@H](C(=O)Nc3ccncc3)CC2)c2cc(OC(F)F)ccc21] | A2 | 430 | 1.05 | 431 | 98 | f |
| 166 | 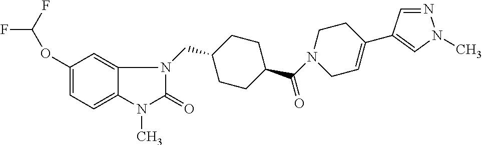 [SMILES: Cn1cc(C2=CCN(C(=O)[C@H]3CC[C@H](Cn4c(=O)n(C)c5ccc(OC(F)F)cc54)CC3)CC2)cn1] | A2 | 499 | 1.32 | 500 | 100 | f |
| 167 | 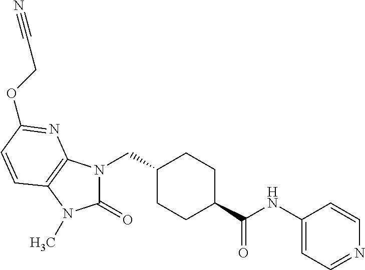 [SMILES: Cn1c(=O)n(C[C@H]2CC[C@H](C(=O)Nc3ccncc3)CC2)c2nc(OCC#N)ccc21] | G | 420 | 0.94 | 421 | 100 | f |
| 168 | 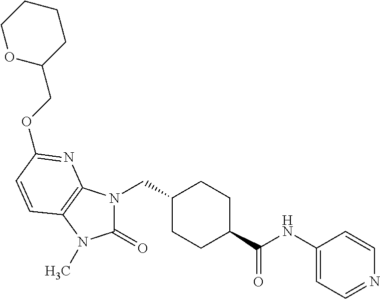 [SMILES: Cn1c(=O)n(C[C@H]2CC[C@H](C(=O)Nc3ccncc3)CC2)c2nc(OCC3CCCCO3)ccc21] | G | 479 | 3.17 | 480 | 100 | c |
| 169 | 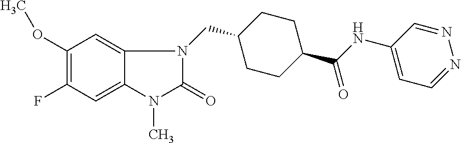 [SMILES: COc1cc2c(cc1F)n(C)c(=O)n2C[C@H]1CC[C@H](C(=O)Nc2ccnnc2)CC1] | A2 | 413 | 1.06 | 414 | 97 | f |
| 170 | 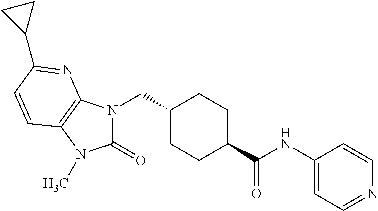 [SMILES: Cn1c(=O)n(C[C@H]2CC[C@H](C(=O)Nc3ccncc3)CC2)c2nc(C3CC3)ccc21] | B | 405 | 1.1 | 406 | 100 | f |
| 171 | 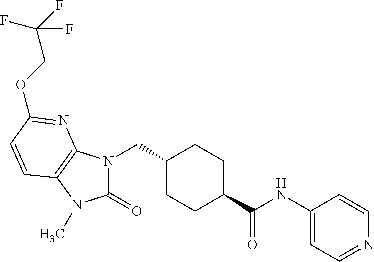 [SMILES: Cn1c(=O)n(C[C@H]2CC[C@H](C(=O)Nc3ccncc3)CC2)c2nc(OCC(F)(F)F)ccc21] | G | 463 | 1.15 | 464 | 99 | f |
| 172 | 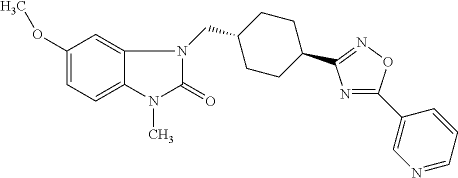 [SMILES: COc1ccc2c(c1)n(C[C@H]1CC[C@H](c3noc(-c4cccnc4)n3)CC1)c(=O)n2C] | M | 419 | 1.43 | 420 | 90 | f |
| 173 | 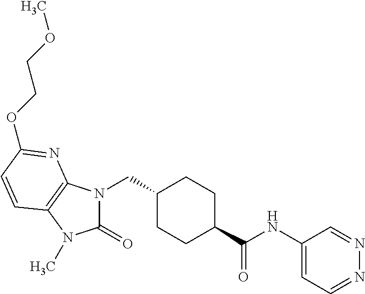 [SMILES: COCCOc1ccc2c(n1)n(C[C@H]1CC[C@H](C(=O)Nc3ccnnc3)CC1)c(=O)n2C] | A2 | 440 | 1.02 | 441 | 100 | f |
| 174 | 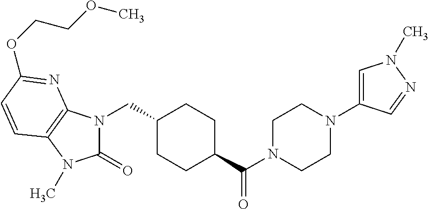 [SMILES: COCCOc1ccc2c(n1)n(C[C@H]1CC[C@H](C(=O)N3CCN(c4cnn(C)c4)CC3)CC1)c(=O)n2C] | A2 | 511 | 1.08 | 512 | 95 | f |
| 175 | 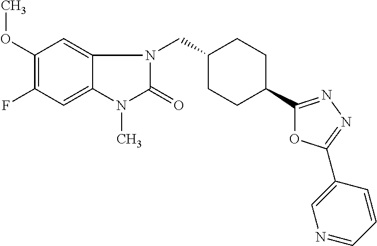 [SMILES: COc1cc2c(cc1F)n(C)c(=O)n2C[C@H]1CC[C@H](c2nnc(-c3cccnc3)o2)CC1] | H1 | 437 | 1.25 | 438 | 98 | f |
| 176 | 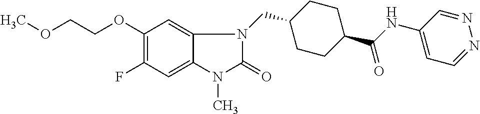 [SMILES: COCCOc1cc2c(cc1F)n(C)c(=O)n2C[C@H]1CC[C@H](C(=O)Nc2ccnnc2)CC1] | A2 | 457 | 1.06 | 458 | 100 | f |
| 177 | 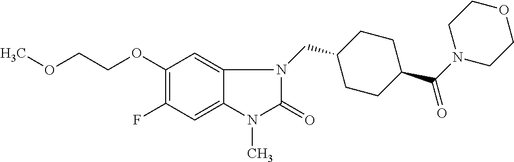 [SMILES: COCCOc1cc2c(cc1F)n(C)c(=O)n2C[C@H]1CC[C@H](C(=O)N2CCOCC2)CC1] | A2 | 449 | 1.15 | 450 | 95 | f |
| 178 | 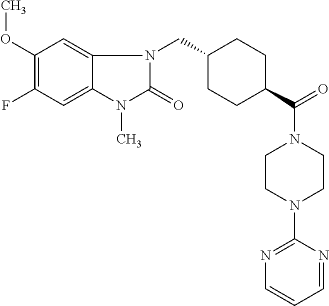 [SMILES: COc1cc2c(cc1F)n(C)c(=O)n2C[C@H]1CC[C@H](C(=O)N2CCN(c3ncccn3)CC2)CC1] | A2 | 482 | 1.28 | 483 | 100 | f |
| 179 | 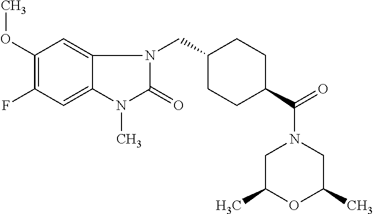 [SMILES: COc1cc2c(cc1F)n(C)c(=O)n2C[C@H]1CC[C@H](C(=O)N2C[C@@H](C)O[C@@H](C)C2)CC1] | A2 | 433 | 2.73 | 434 | 100 | c |
| 180 | 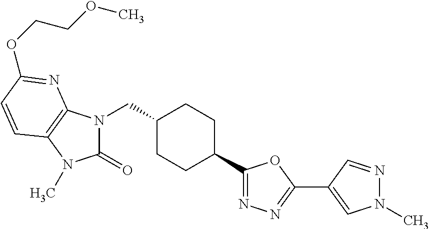 [SMILES: COCCOc1ccc2c(n1)n(C[C@H]1CC[C@H](c3nnc(-c4cnn(C)c4)o3)CC1)c(=O)n2C] | H2 | 467 | 1.19 | 468 | 100 | f |
| 181 | 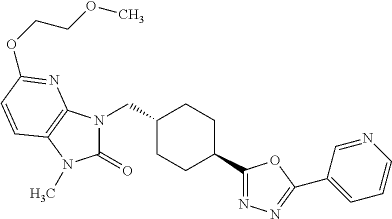 [SMILES: COCCOc1ccc2c(n1)n(C[C@H]1CC[C@H](c3nnc(-c4cccnc4)o3)CC1)c(=O)n2C] | H1 | 464 | 1.22 | 465 | 100 | f |
| 182 | 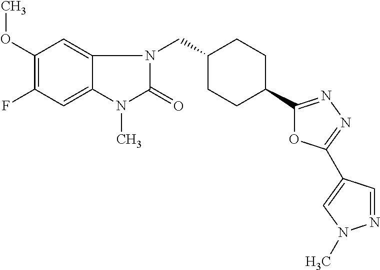 [SMILES: COc1cc2c(cc1F)n(C)c(=O)n2C[C@H]1CC[C@H](c2nnc(-c3cnn(C)c3)o2)CC1] | H2 | 440 | 1.22 | 441 | 98 | f |
| 183 | 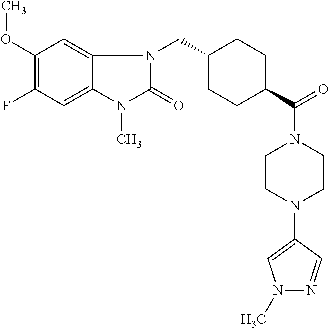 [SMILES: COc1cc2c(cc1F)n(C)c(=O)n2C[C@H]1CC[C@H](C(=O)N2CCN(c3cnn(C)c3)CC2)CC1] | A2 | 484 | 2.3 | 485 | 100 | c |
| 184 | 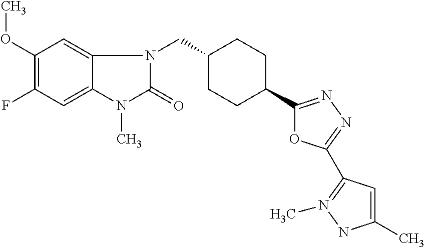 | H2 | 454 | 3.05 | 455 | 100 | c |
| 185 | 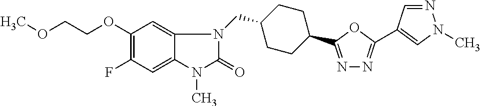 | H2 | 484 | 1.06 | 485 | 93 | f |
| 186 | 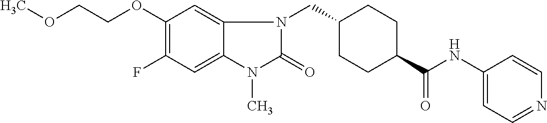 [SMILES: COCCOc1cc2c(cc1F)n(C)c(=O)n2C[C@H]1CC[C@H](C(=O)Nc2ccncc2)CC1] | A2 | 456 | 1.00 | 457 | 98 | f |
| 187 | 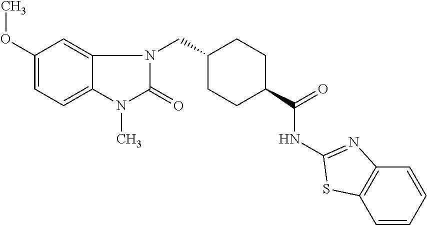 [SMILES: COc1ccc2c(c1)n(C[C@H]1CC[C@H](C(=O)Nc3nc4ccccc4s3)CC1)c(=O)n2C] | A2 | 450 | 1.57 | 451 | 95 | f |
| 188 | 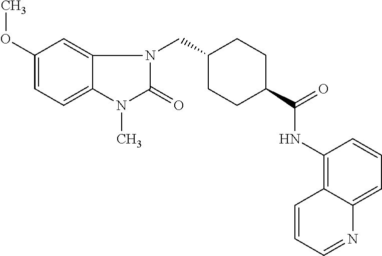 [SMILES: COc1ccc2c(c1)n(C[C@H]1CC[C@H](C(=O)Nc3cccc4ncccc34)CC1)c(=O)n2C] | A2 | 444 | 1.04 | 445 | 93 | f |

Examples 1-188, each of which constitutes a separate embodiment of this invention, display an IC₅₀ value in the above described reporter assay falling between 35 nM and 23 μM. In the renilla read out, Examples 1-188 showed a negligible effect. Moreover, selected representative compounds were assessed not to be inhibitors of the luciferase enzyme. Examples 185, 186, 153, 22, 61, 115, 72, 152, 121, 106, 147, 182, 161, 68, 92, 71, 29, 14 and 1 display an IC50 value ranging from 32 nM to 2.9 μM in the soft agar assay.

1. Compounds of formula I 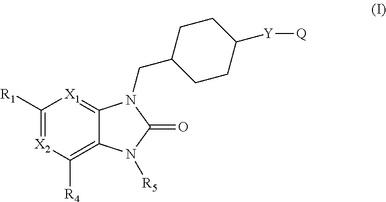
wherein, as valence and stability permit:
any carbon-bound hydrogen atom may be substituted with a fluorine atom;
X₁ is CR₂ or N;
X₂ is CR₃ or N;
-Y-Q is 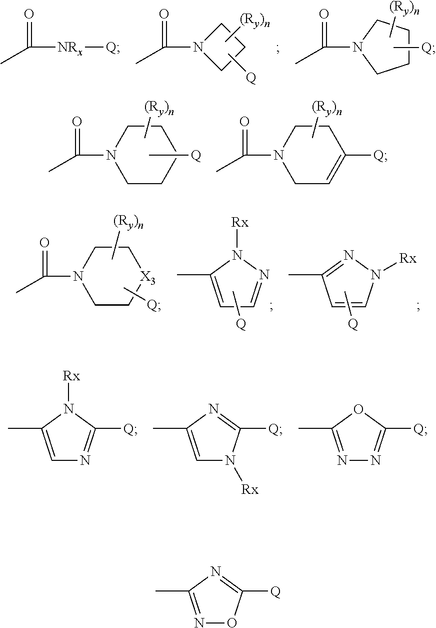
Q is C₁-C₆ linear branched or cylic alkyl, alkylcarbonyl, oxalkyl, dioxalkyl, alkylaminocarbonyl, oxalkylamminocarbonyl group wherein any methylene group may be substituted with an oxo group; a C₅-C₁₀ aryl or heteroaryl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;
R₁ is H; F; Cl; Br; OH; CN; linear branched or cyclic C₁-C₆ alkyl, alkenyl, alkynyl, oxalkyl, oxalkenyl, oxalkynil, azalkenyl, azalkynyl, alkyloxy, alkenyloxy, oxalkyloxy, dioxalkyloxy, oxazalkyloxy, azalkyloxy, dialkylamino, oxalkylamino, azalkylamino, group optionally substituted with one or more F or CN; C₅-C₆ aryl- or heteroarylmethylammino or C₅-C₆ aryl- or heterorylmethyloxy group where the aryl or heteroaryl moiety may optionally be substituted with one or more C₁-C₃ alkyl, C₁-C₃ alkoxy, halogen or CN groups;
R₂ is H or Cl;
R₃ is H, Cl or F;
R₄ is H or Cl;
R₅ is a C₁-C₃ linear, branched or cylic alkyl group;
Rx is H; a linear, branched or cyclic C₁-C₃ alkyl group;
n may be nil, 1, 2 or 3;
Ry is—independently from one another when n=2 or more—F; a linear, branched or cyclic C₁-C₃ alkyl group; or Ry, together with the carbon atom to which it is attached, forms an oxo group.
X₃ is either N, O or S;
tautomers, optical isomers and pharmaceutically acceptable salts thereof;
with the exception of

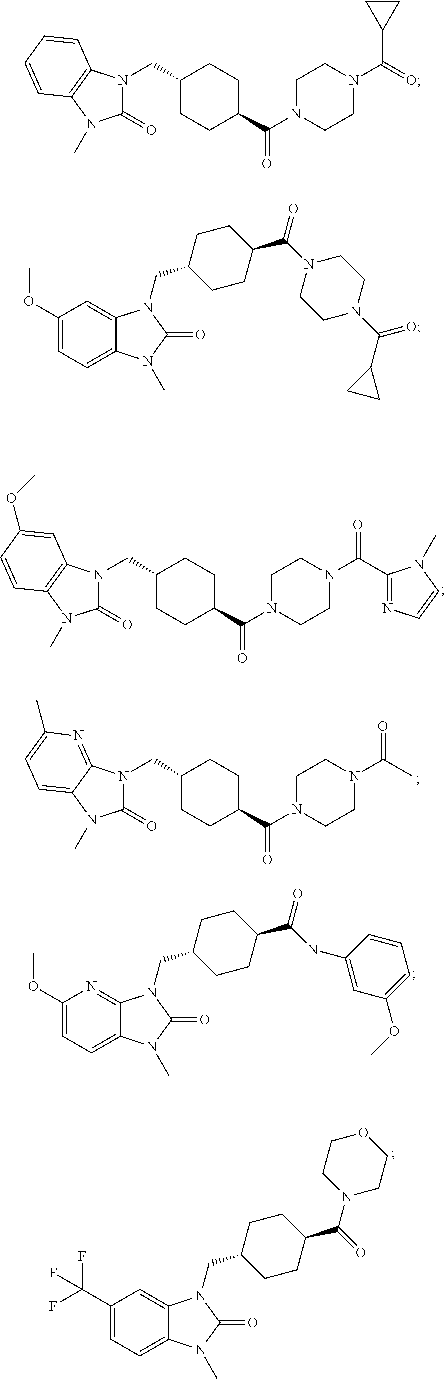

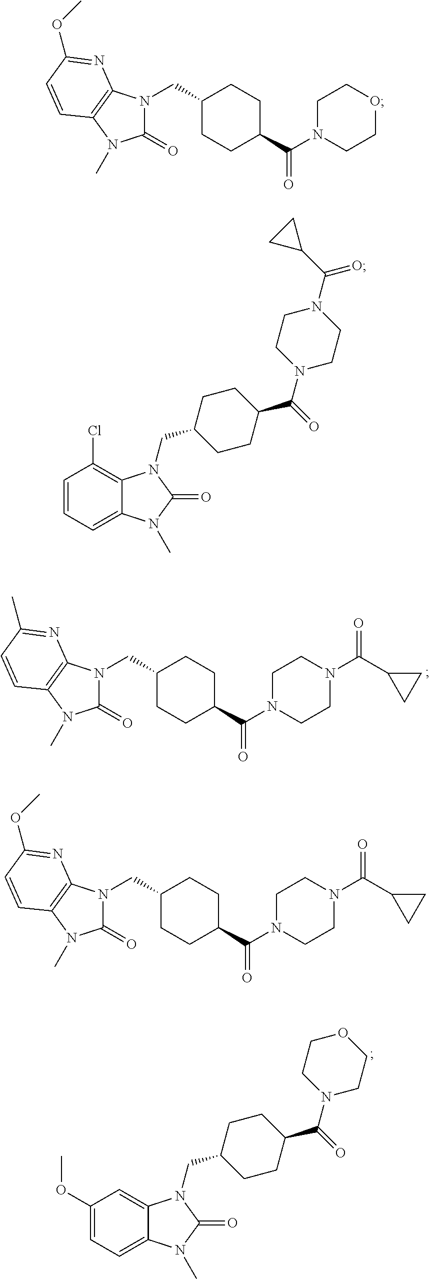

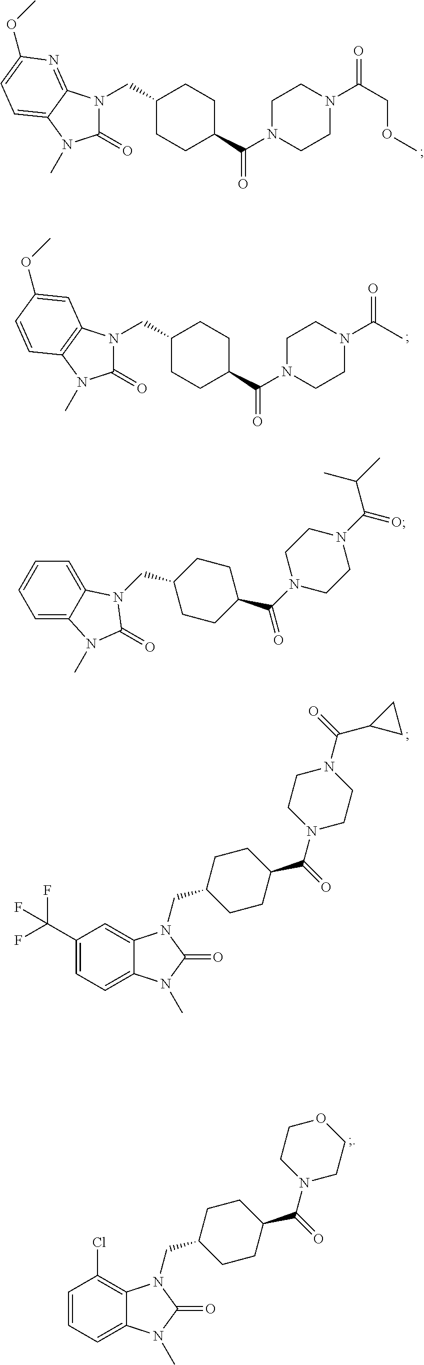 [SMILES: COCC(=O)N1CCN(C(=O)[C@H]2CC[C@H](Cn3c(=O)n(C)c4ccc(OC)nc43)CC2)CC1 ; COc1ccc2c(c1)n(C[C@H]1CC[C@H](C(=O)N3CCN(C(C)=O)CC3)CC1)c(=O)n2C ; CC(C)C(=O)N1CCN(C(=O)[C@H]2CC[C@H](Cn3c(=O)n(C)c4ccccc43)CC2)CC1 ; Cn1c(=O)n(C[C@H]2CC[C@H](C(=O)N3CCN(C(=O)C4CC4)CC3)CC2)c2cc(C(F)(F)F)ccc21 ; Cn1c(=O)n(C[C@H]2CC[C@H](C(=O)N3CCOCC3)CC2)c2c(Cl)cccc21]

2. The compounds of claim 1, of formula (I-bis): 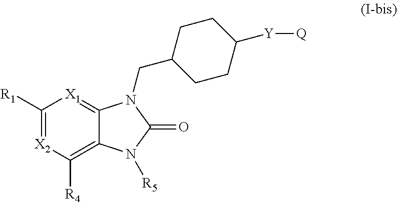
wherein, as valence and stability permit:
carbon-bound hydrogen atom may be substituted with a fluorine atom;
X₁ is CR₂;
X₂ is CR₃ or N;
-Y-Q is 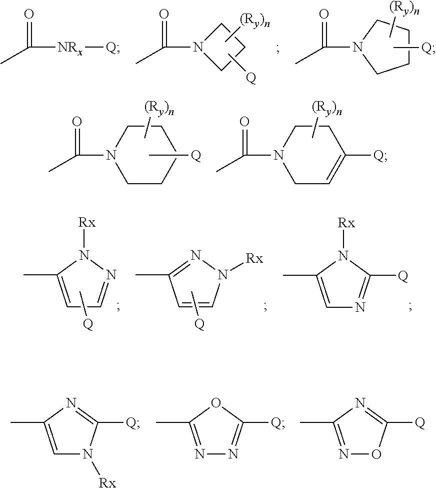
Q is C₁-C₆ linear branched or cyclic alkyl, alkylcarbonyl, oxalkyl, dioxalkyl, alkylaminocarbonyl, oxalkylamminocarbonyl group wherein any methylene group may be substituted with an oxo group; a C₅-C₁₀ aryl or heteroaryl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;
R₁ is H; F; Cl; Br; OH; CN; linear, branched or cyclic C₁-C₆ alkyl, alkenyl, alkynyl, oxalkyl, oxalkenyl, oxalkynil, azalkenyl, azalkynyl, alkyloxy, alkenyloxy, oxalkyloxy, dioxalkyloxy, oxazalkyloxy, azalkyloxy, dialkylamino, oxalkylamino, azalkylamino, group optionally substituted with one or more F or CN; C₅-C₆ aryl- or heteroarylmethylammino or C₅-C₆ aryl- or heteroarylmethyloxy group where the aryl or heteroaryl moiety may optionally be substituted with one or more C₁-C₃ alkyl, C₁-C₃ alkoxy, halogen or CN groups;
R₂ is H or Cl;
R₃ is H, Cl or F;
R₄ is H or Cl;
R₅ is a C₁-C₃ linear, branched or cyclic alkyl group;
Rx is H; a linear, branched or cyclic C₁-C₃ alkyl group;
n may be nil, 1, 2 or 3;
Ry is—independently from one another when n=2 or more—F; a linear, branched or cyclic C₁-C₃ alkyl group; or Ry, together with the carbon atom to which it is attached, forms an oxo group;
tautomers, optical isomers and pharmaceutically acceptable salts thereof.

3. The compounds of claim 1, wherein
Q is C₁-C₆ linear branched or cylic allyl, alkylcarbonyl, oxalkyl, dioxalkyl, alkylaminocarbonyl, oxalkylamminocarbonyl group wherein any methylene group may be substituted with an oxo group; a C₅-C₆ aryl or heteroaryl group optionally substituted with 1, 2 or 3 groups selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;
and wherein X₁, X₂, X₃, Y, R₁, R₂, R₃, R₄, R₅, Rx, n, Ry are as defined.

4. The compounds of claim 1 wherein
X₁ is CR₂; R₂ is H;
X₂ is CR₃;
-Y-Q is 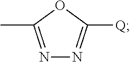
Q is a pyrazolyl group substituted with 1 to 3 C₁-C₃ alkyl wherein one or more carbon-bound hydrogen may be substituted by fluorine;
R₄ is H;
and wherein R₁, R₃ and R₅ are as defined.

5. The compounds of claim 4, selected from the list of

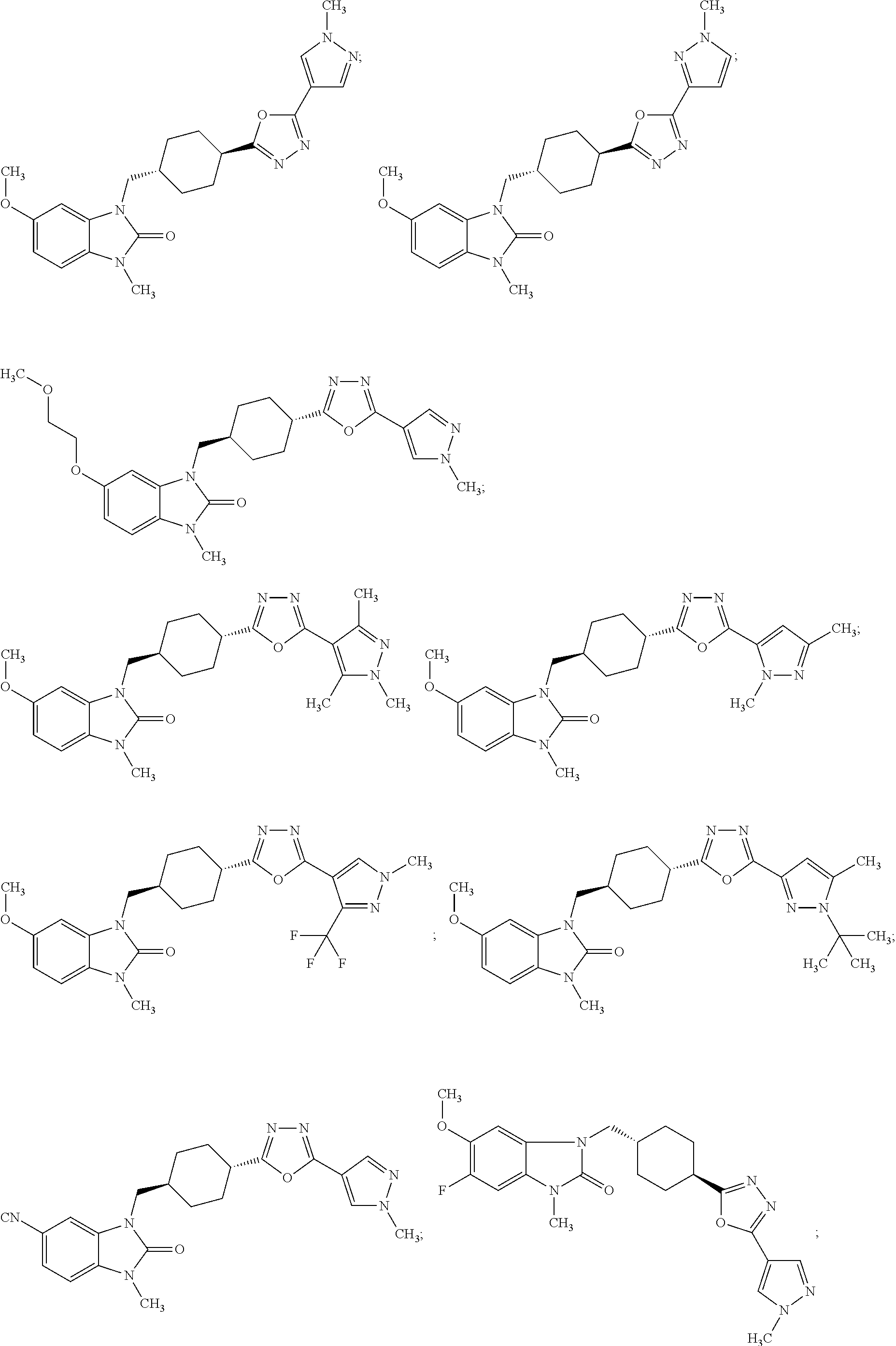

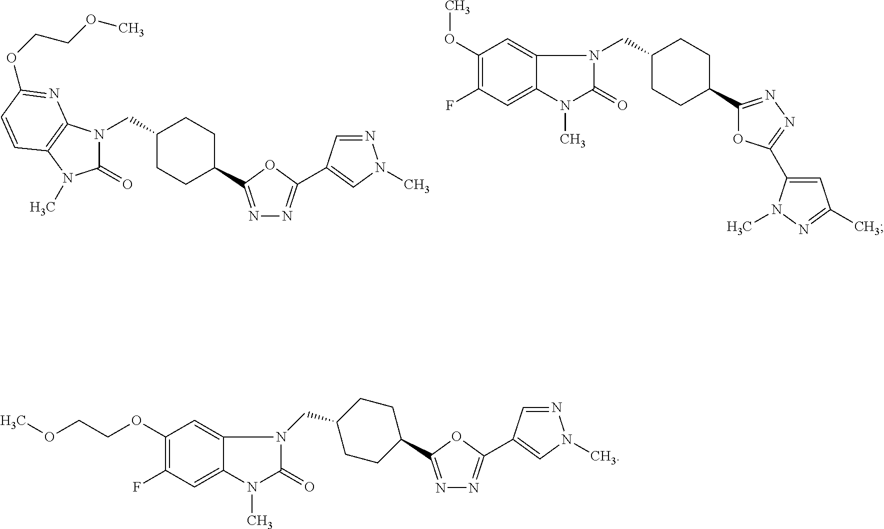 [SMILES: COCCOc1ccc2c(n1)n(C[C@H]1CC[C@H](c3nnc(-c4cnn(C)c4)o3)CC1)c(=O)n2C ; COc1cc2c(cc1F)n(C)c(=O)n2C[C@H]1CC[C@H](c2nnc(-c3cc(C)nn3C)o2)CC1 ; COCCOc1cc2c(cc1F)n(C)c(=O)n2C[C@H]1CC[C@H](c2nnc(-c3cnn(C)c3)o2)CC1]

6. The compounds of claim 1 wherein.
X₁ is CR₂; R₂ is H;
X₂ is CR₃;
-Q-Y is; 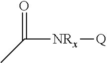
Q is pyridazinyl;
R₁ is a linear branched or cyclic C₁-C₆ oxalkyl, oxalkenyl, oxalkynyl, alkyloxy, oxalkyloxy, oxazalkyloxy, azalkyloxy group;
R₄ is H;
and wherein R₃, R₅ and Rx are as defined.

7. The compounds of claim 6, selected form the list of

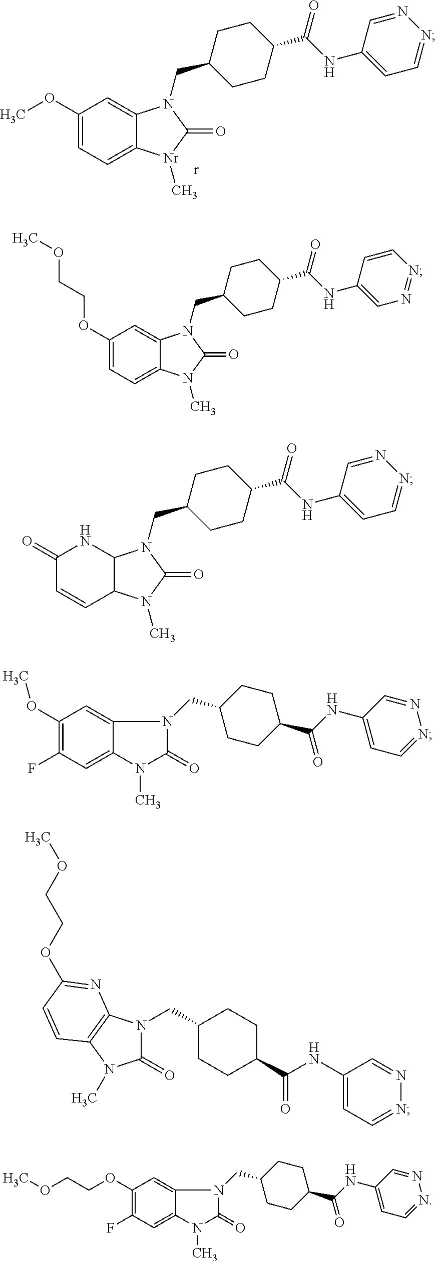

8. The compounds of claim 1 wherein
X₁ is CR₂; R₂ is H;
X₂ is CR₃;
-Q-Y is 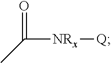
Q is 4-pyridyl;
R₁ is a linear, branched or cyclic C₁-C₆ alkyloxy, alkenyloxy, oxalkyloxy, dioxalkyloxy oxalkylammino, group optionally substituted with F or CN;
R₄ is H;
and wherein R₅ is as defined.

9. The compounds of claim 8 selected form the list of

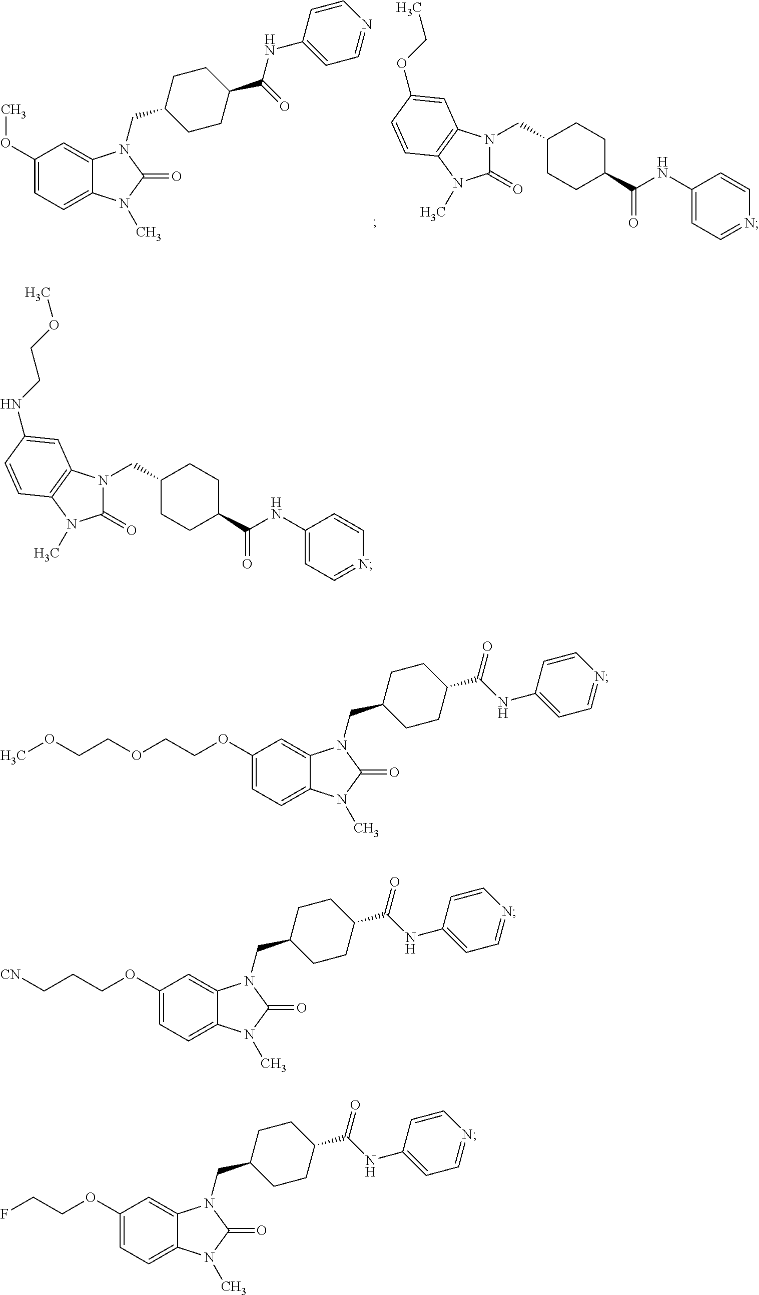

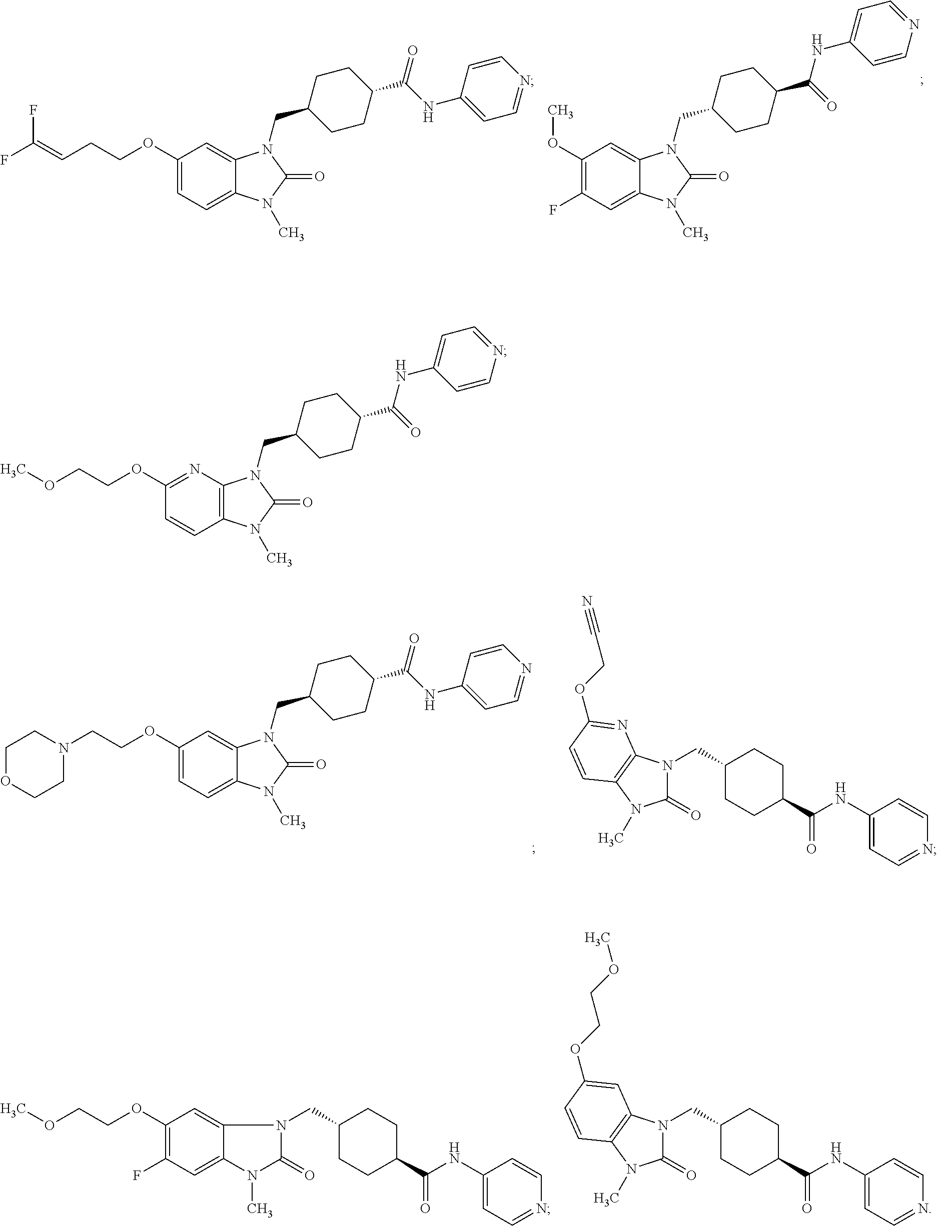

10. The compounds of claim 1 wherein
X₁ is CR₂; R₂ is H;
X₂ is CR₃;
R₁ is a linear, branched or cyclic C₁-C₆ alkoxy or oxalkyloxy;
R₃ is F;
R₄ is H;
and wherein X₃, Y-Q, R₅, Rx, n and Ry are as defined.

11. The compounds of claim 10, selected form the list of

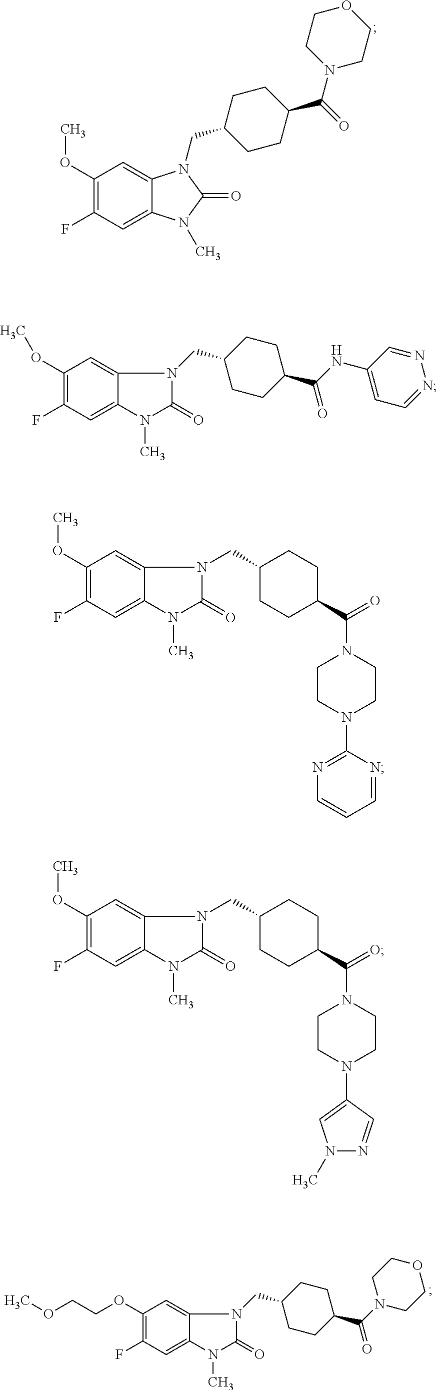 [SMILES: COc1cc2c(cc1F)n(C)c(=O)n2C[C@H]1CC[C@H](C(=O)N2CCOCC2)CC1 ; ";" ; COc1cc2c(cc1F)n(C)c(=O)n2C[C@H]1CC[C@H](C(=O)Nc2ccnnc2)CC1 ; COc1cc2c(cc1F)n(C)c(=O)n2C[C@H]1CC[C@H](C(=O)N2CCN(c3ncccn3)CC2)CC1 ; COc1cc2c(cc1F)n(C)c(=O)n2C[C@H]1CC[C@H](C(=O)N2CCN(c3cnn(C)c3)CC2)CC1 ; COCCOc1cc2c(cc1F)n(C)c(=O)n2C[C@H]1CC[C@H](C(=O)N2CCOCC2)CC1]

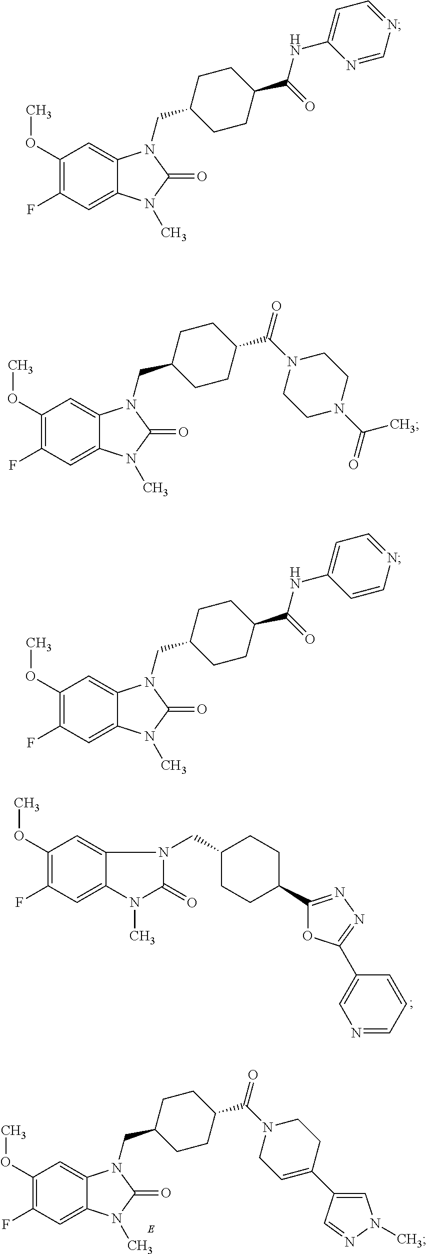 [SMILES: COc1cc2c(cc1F)n(C)c(=O)n2C[C@H]1CC[C@H](C(=O)Nc2ccncn2)CC1 ; COc1cc2c(cc1F)n(C)c(=O)n2C[C@H]1CC[C@H](C(=O)N2CCN(C(C)=O)CC2)CC1 ; COc1cc2c(cc1F)n(C)c(=O)n2C[C@H]1CC[C@H](C(=O)Nc2ccncc2)CC1 ; COc1cc2c(cc1F)n(C)c(=O)n2C[C@H]1CC[C@H](c2nnc(-c3cccnc3)o2)CC1 ; COc1cc2c(cc1F)n(C)c(=O)n2C[C@H]1CC[C@H](C(=O)N2CC=C(c3cnn(C)c3)CC2)CC1]

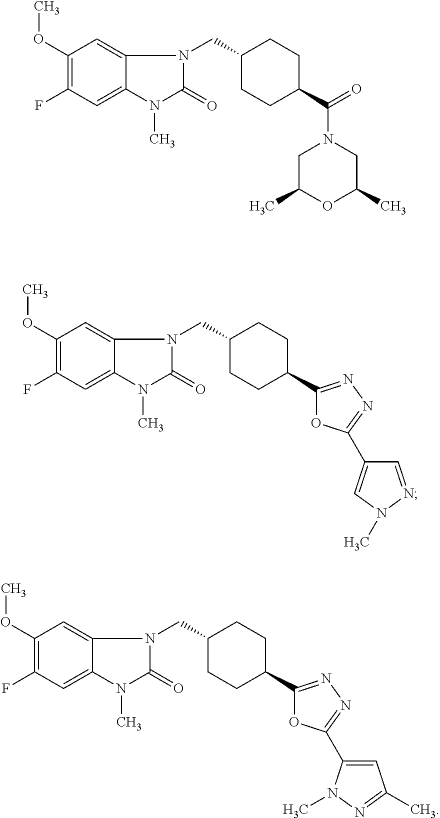 [SMILES: COc1cc2c(cc1F)n(C)c(=O)n2C[C@H]1CC[C@H](C(=O)N2C[C@@H](C)O[C@@H](C)C2)CC1 ; COc1cc2c(cc1F)n(C)c(=O)n2C[C@H]1CC[C@H](c2nnc(-c3cnn(C)c3)o2)CC1 ; COc1cc2c(cc1F)n(C)c(=O)n2C[C@H]1CC[C@H](c2nnc(-c3cc(C)nn3C)o2)CC1]

12. The compounds of claim 1, of formula (I-ter): 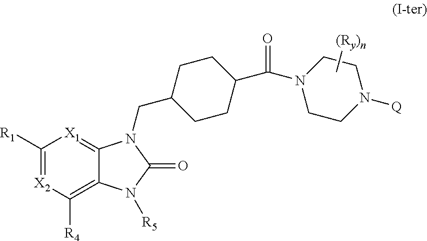
wherein, as valence and stability permit;
any carbon-bound hydrogen atom may be substituted with a fluorine atom;
X₁ is CR₂; R₂ is H;
X₂ is CR₃;
Q is a C₁-C₃ linear, branched or cyclic alkylcarbonyl;
R₁ is OH, linear branched or cyclic C₁-C₆ alkyl, alkenyl, alkynyl, oxalkyl, oxalkyloxy, oxalkylammino group;
R₄ is H;
R₃ is H, Cl, or F;
R₅ is a C₁-C₃ linear, branched or cyclic alkyl group;
n may be nil, 1, 2 or 3;
Ry is—independently from one another when n=2 or more—F; a linear, branched or cyclic C₁-C₃ alkyl group; or Ry, together with the carbon atom to which it is attached, forms an oxo group;
tautomers, optical isomers and pharmaceutically acceptable salts thereof.

13. The compounds of claim 12, wherein R₁ is a linear branched or cyclic C₁-C₆ alkyl group.

14. The compounds of claim 12, selected from the list of

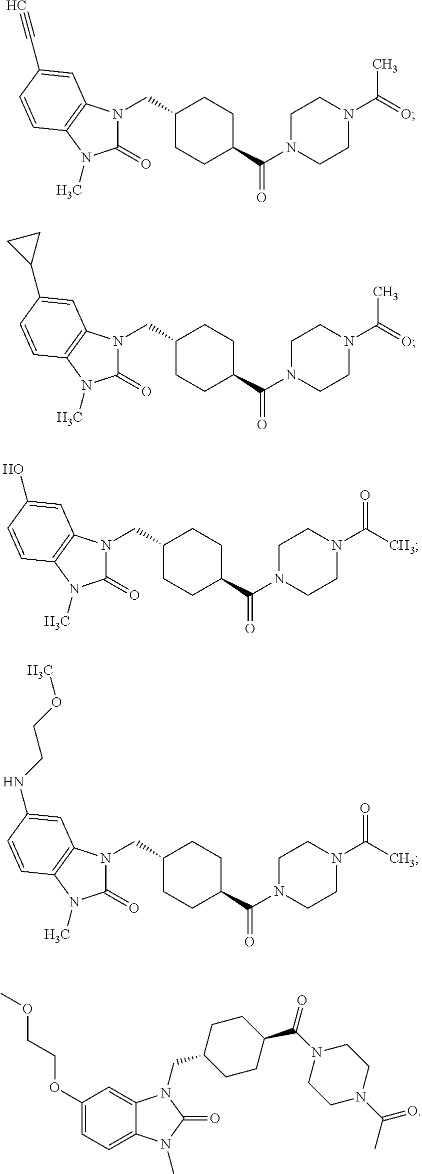 [SMILES: C#Cc1ccc2c(c1)n(C[C@H]1CC[C@H](C(=O)N3CCN(C(C)=O)CC3)CC1)c(=O)n2C ; CC(=O)N1CCN(C(=O)[C@H]2CC[C@H](Cn3c(=O)n(C)c4ccc(C5CC5)cc43)CC2)CC1 ; CC(=O)N1CCN(C(=O)[C@H]2CC[C@H](Cn3c(=O)n(C)c4ccc(O)cc43)CC2)CC1 ; COCCNc1ccc2c(c1)n(C[C@H]1CC[C@H](C(=O)N3CCN(C(C)=O)CC3)CC1)c(=O)n2C ; COCCOc1ccc2c(c1)n(C[C@H]1CC[C@H](C(=O)N3CCN(C(C)=O)CC3)CC1)c(=O)n2C]

15. The compounds of claim 1 wherein
X₁ is CR₂; R₂ is H
R₁ is a C₁-C₃ linear branched or cyclic alkoxy group
X₂ is CR₃;
R₃ is H;
R₄ is H;
-Q-Y is 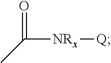
Q is a C₅-C₁₀ aryl or heteroaryl group optionally substituted with 1, 2 or 3 group selected from the list of C₁-C₆ linear branched or cyclic alkyl, oxalkyl, alkylamino, alkylaminocarbonyl, oxalkylamino, oxalkyloxy, azalkyloxy, halogen, cyano, or a C₅-C₆ aryl or heteroaryl group optionally substituted with halogen, C₁-C₃ alkyl, C₁-C₃ oxalkyl;
and wherein R₅, Rx, n are as defined.

16. The compounds of claim 15, selected form the list of

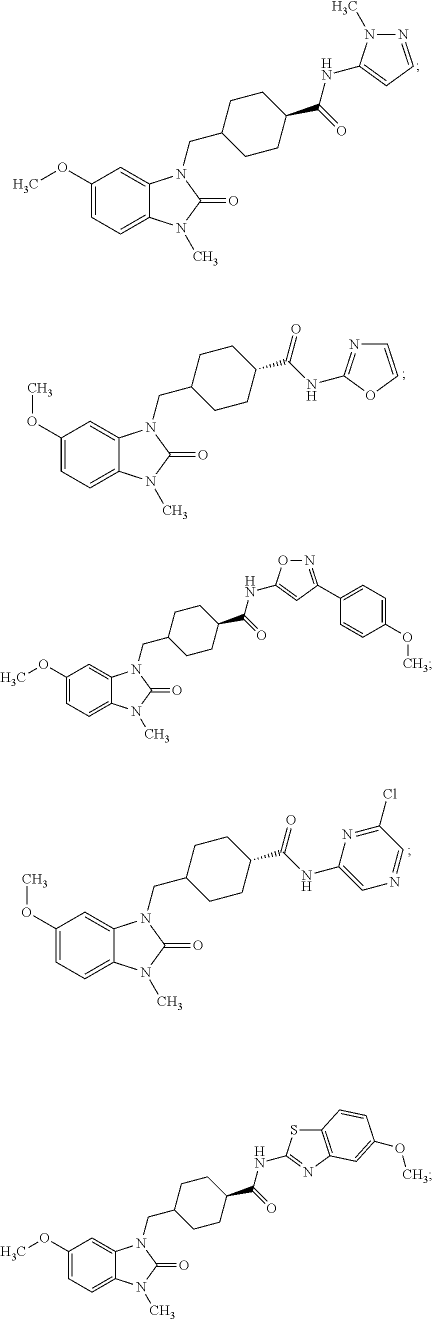

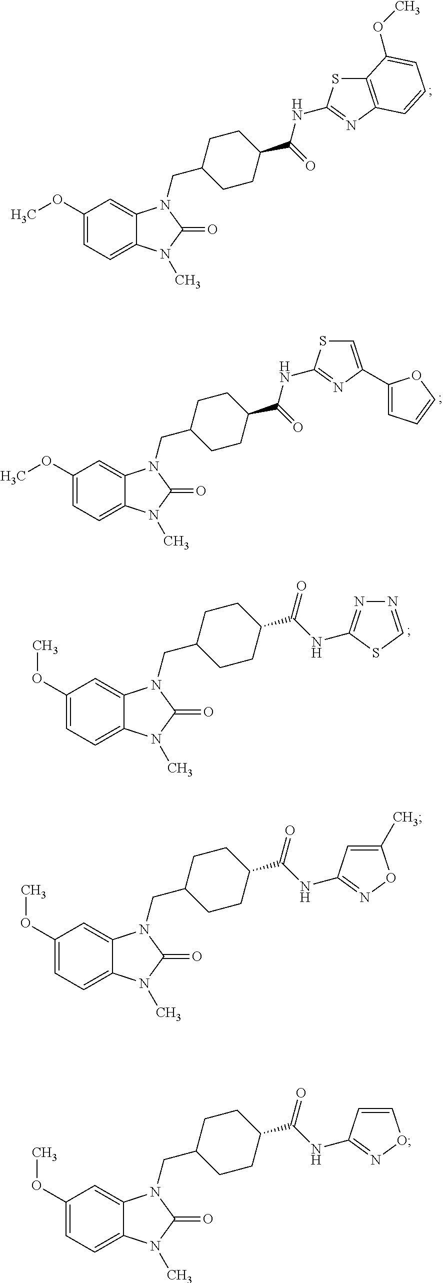

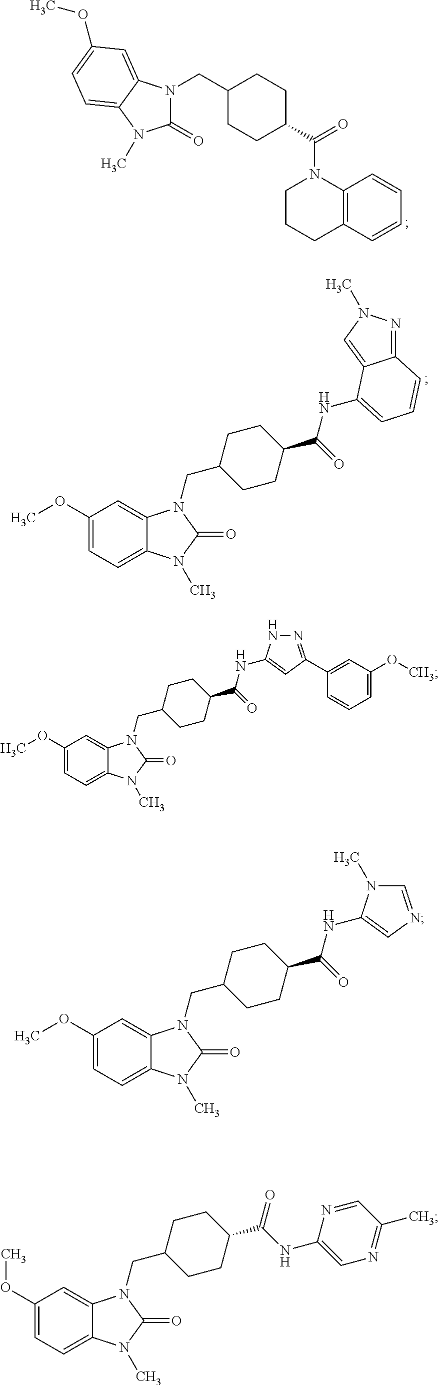

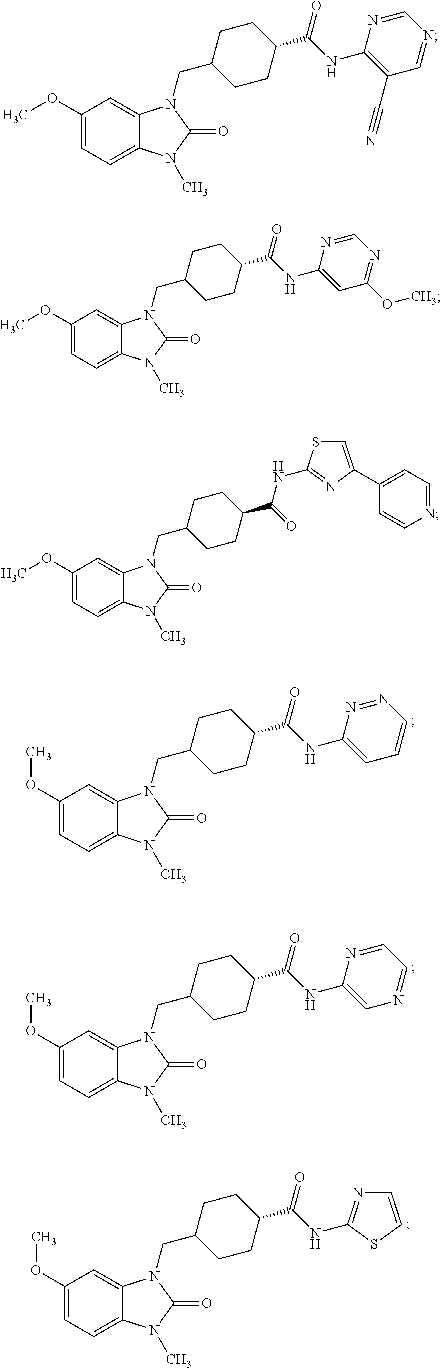

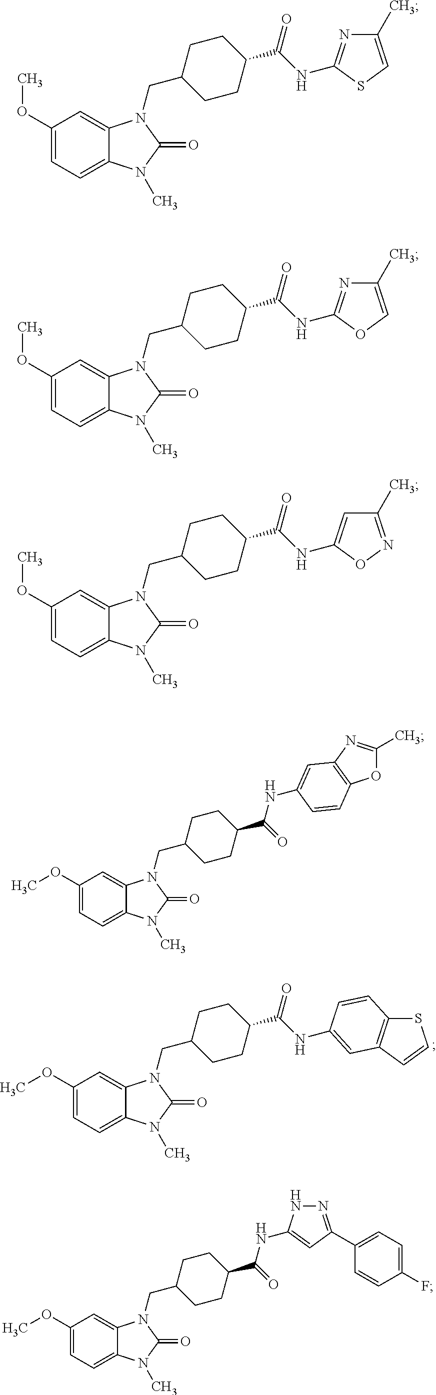

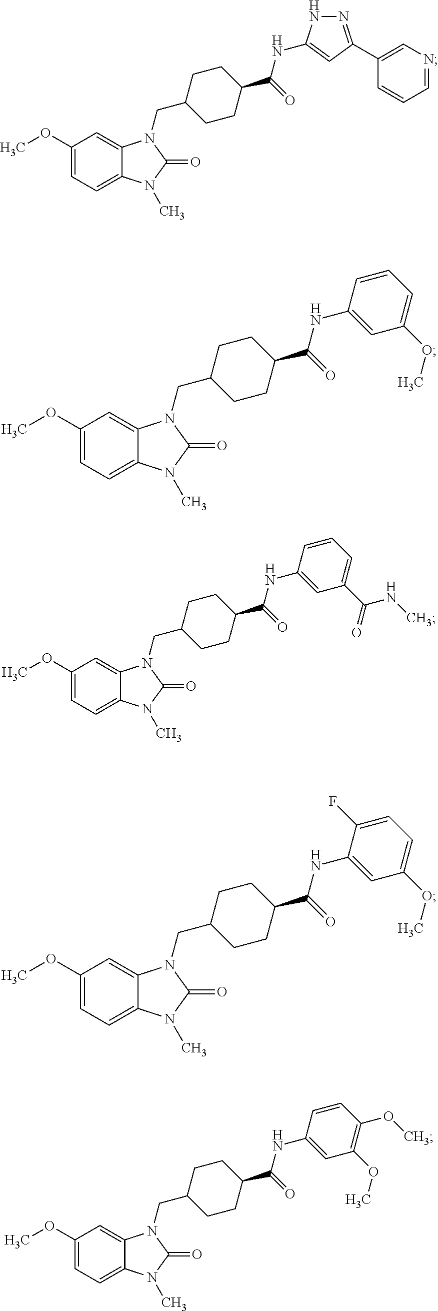

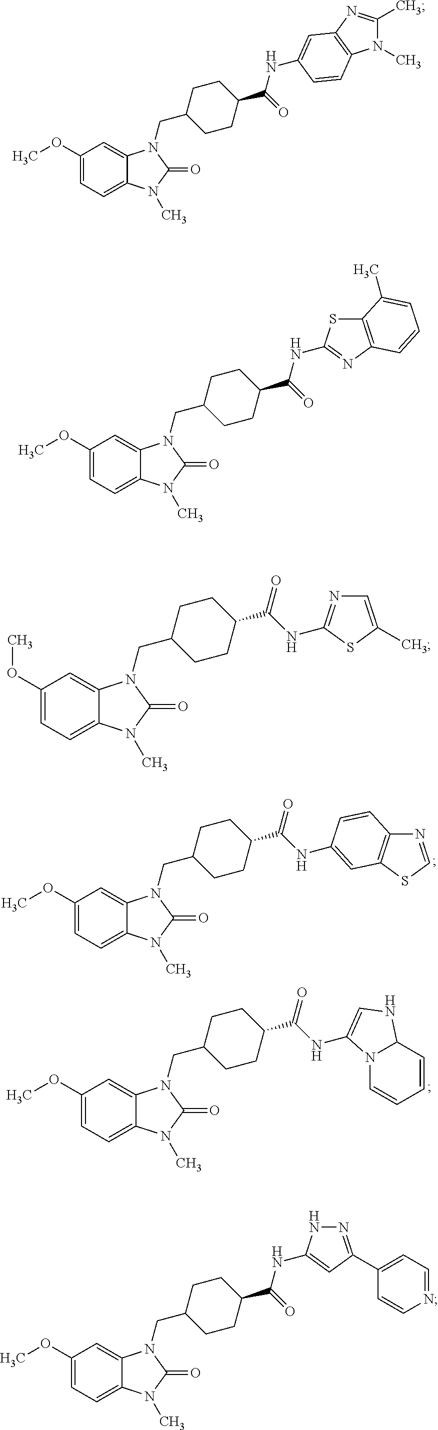

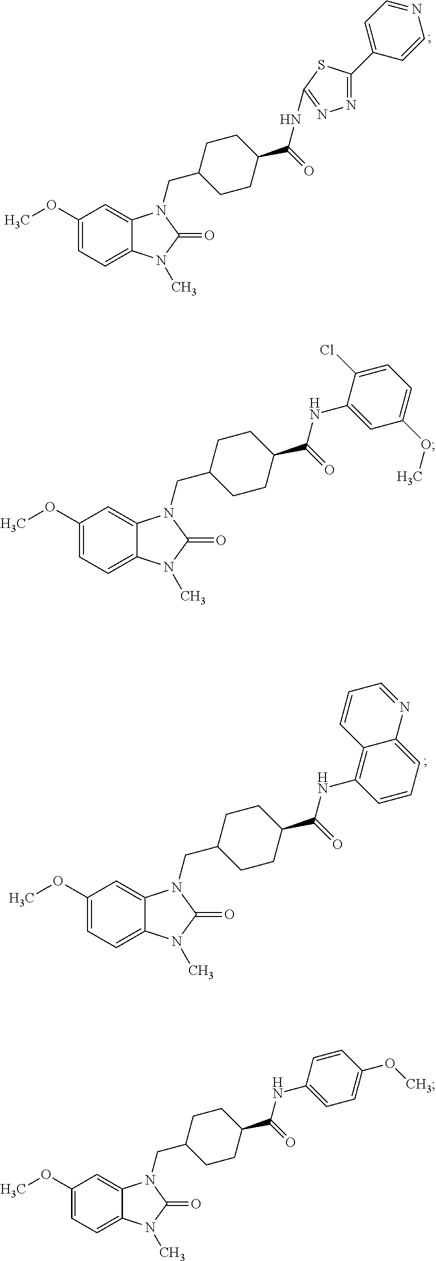

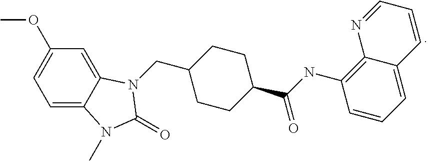

17. The compounds of claim 1 for use in the preparation of a medicament, for the treatment of cancer, pulmonary fibrosis, renal fibrosis, ischemic neural injury or multiple sclerosis.

18. The compounds of claim 1, for use in the cure of a cancer selected from the list of lung cancer; colon cancer; pancreatic cancer; breast cancer; melanoma; glioblastoma; medulloblastoma; gastric cancer; hepatocellular cancer; basal cell carcinoma; leukemia; Wilm's tumour; Familial Adenomatous Polyposis.

19. Pharmaceutical compositions containing a compound according to claim 1 in admixture with a pharmaceutically acceptable carrier or excipient.

20. A method for the treatment of diseases, conditions, or dysfunctions that benefit from the inhibition of the Wnt pathway, which comprises administering to a subject in need thereof an effective amount of a compound according to claim 1.
